(19) United States
(12) Patent Application Publication
(10) Pub. No.: US 2022/0106289 A1
(43) Pub. Date: Apr. 7, 2022

(54) NLRP3 INHIBITORS

(71) Applicant: INFLAZOME LIMITED, Dublin 2 (IE)

(72) Inventors: Matthew COOPER, Cambridge (GB); David MILLER, Cambridge (GB); Angus MACLEOD, Cambridge (GB); Jonathan SHANNON, Nottingham (GB); Stephen THOM, Nottingham (GB); Ian STRUTT, Nottingham (GB); Diana CASTAGNA, Nottingham (GB); Jokin CARRILLO ARREGUI, Nottingham (GB); Jimmy VAN WILTENBURG, Groningen (NL)

(21) Appl. No.: 17/296,151

(22) PCT Filed: Nov. 22, 2019
(86) PCT No.: PCT/EP2019/082231

§ 371 Date: May 21, 2021

(30) Foreign Application Priority Data
Nov. 23, 2018 (GB) 1819083.5

(51) Int. Cl. C07D 401/12; C07C 307/02; C07D 213/64; C07D 409/12; C07D 231/18; C07D 333/34; C07D 403/06; C07D 249/04; C07D 277/36; C07D 403/12; C07D 405/12; C07D 413/12

(52) U.S. Cl. CPC C07D 401/12; C07C 307/02; C07D 213/64; C07D 409/12; C07D 231/18; C07D 333/34; C07B 2200/05; C07D 249/04; C07D 277/36; C07D 403/12; C07D 405/12; C07D 413/12; C07D 403/06

(57) ABSTRACT

The present application relates to compounds with NLRP₃inhibitory activity and to associated salts, solvates, prodrugs and pharmaceutical compositions. The present application further relates to the use of such compounds in the treatment and prevention of medical disorders and diseases, most especially by NLRP₃inhibition.

### FIELD OF THE INVENTION

The present invention relates to compounds with NLRP3 inhibitory activity and to associated salts, solvates, prodrugs and pharmaceutical compositions. The present invention further relates to the use of such compounds in the treatment and prevention of medical disorders and diseases, most especially by NLRP3 inhibition.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signalling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-i cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-1R2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. Furthermore, NLRP3 has a role in the development of liver disease, kidney disease and aging. Many of these associations were defined using Nlrp3−/− mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

Some diarylsulfonylurea-containing compounds have been identified as cytokine release inhibitory drugs (CRIDs) (Perregaux et al.; J. Pharmacol. Exp. Ther., 299, 187-197, 2001). CRIDs are a class of diarylsulfonylurea-containing compounds that inhibit the post-translational processing of IL-1β. Post-translational processing of IL-1β is accompanied by activation of caspase-1 and cell death. CRIDs arrest activated monocytes so that caspase-1 remains inactive and plasma membrane latency is preserved.

Certain sulfonylurea-containing compounds are also disclosed as inhibitors of NLRP3 (see for example, Baldwin et al., J. Med. Chem., 59(5), 1691-1710, 2016; and WO 2016/131098 A1, WO 2017/129897 A1, WO 2017/140778 A1, WO 2017/184623 A1, WO 2017/184624 A1, WO 2018/015445 A1, WO 2018/136890 A1, WO 2018/215818 A1, WO 2019/008025 A1, WO 2019/008029 A1, WO 2019/034686 A1, WO 2019/034688 A1, WO 2019/034690 A1, WO 2019/034692 A1, WO 2019/034693 A1, WO 2019/034696 A1, WO 2019/034697 A1, WO 2019/043610 A1, WO 2019/092170 A1, WO 2019/092171 A1, and WO 2019/092172 A1). In addition, WO 2017/184604 A1 and WO 2019/079119 A1 disclose a number of sulfonylamide-containing compounds as inhibitors of NLRP3. Certain sulfoximine-containing compounds are also disclosed as inhibitors of NLRP3 (WO 2018/225018 A1, WO 2019/023145 A1, WO 2019/023147 A1, and WO 2019/068772 A1).

There is a need to provide compounds with improved pharmacological and/or physiological and/or physicochemical properties and/or those that provide a useful alternative to known compounds.

### SUMMARY OF THE INVENTION

A first aspect of the invention provides a compound of formula (I):

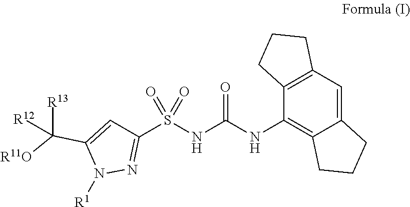

wherein:

R¹ is a C₁-C₃ alkyl group;

R¹¹ is a C₁-C₃ alkyl group;

R¹² is hydrogen or a C₁-C₃ alkyl group; and

R¹³ is hydrogen or a C₁-C₃ alkyl group; or

R¹² and R¹³ together with the carbon atom to which they are attached form a C₃-C₆ cycloalkyl group.

In one embodiment, R¹ is a methyl, ethyl, n-propyl or i-propyl group. In one embodiment, R¹ is a methyl or ethyl group. In one embodiment, R¹ is a methyl group.

In one embodiment, R¹¹ is a methyl, ethyl, n-propyl or i-propyl group. In one embodiment, R¹¹ is a methyl or ethyl group. In one embodiment, R¹¹ is a methyl group.

In one embodiment, R¹² is hydrogen or a methyl, ethyl, n-propyl or i-propyl group. In one embodiment, R¹² is hydrogen or a methyl or ethyl group. In one embodiment, R¹² is hydrogen or a methyl group. In one embodiment, R¹² is a methyl group.

In one embodiment, R¹³ is hydrogen or a methyl, ethyl, n-propyl or i-propyl group. In one embodiment, R¹³ is hydrogen or a methyl or ethyl group. In one embodiment, R¹³ is hydrogen or a methyl group.

In one embodiment, R¹² and R¹³ together with the carbon atom to which they are attached form a cyclopropyl, cyclobutyl, cyclopentyl or cyclohexyl group. In one embodiment, R¹² and R¹³ together with the carbon atom to which they are attached form a cyclobutyl or cyclopentyl group.

In one embodiment, —C(OR¹¹)R¹²R¹³ is —C(CH₃)₂(OCH₃), —CH(CH₃)(OCH₃), or

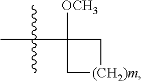

wherein m is 1 or 2.

The first aspect of the invention also provides a compound of formula (II):

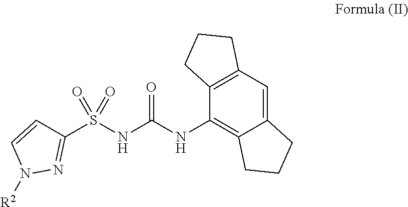

wherein R² is a C₁-C₃ haloalkyl group.

In one embodiment, R² is a C₁-C₃ alkyl group substituted with 1-7 halo atoms independently selected from fluoro and chloro atoms. In one embodiment, R² is a C₁-C₃ alkyl group substituted with 1-7 fluoro atoms. In one embodiment, R² is a C₁-C₂ alkyl group substituted with 1-5 fluoro atoms. In one embodiment, R² is an ethyl group substituted with 2 or 3 fluoro atoms. In one embodiment, R² is —CH₂CF₃ or —CH₂CHF₂.

The first aspect of the invention also provides a compound of formula (III):

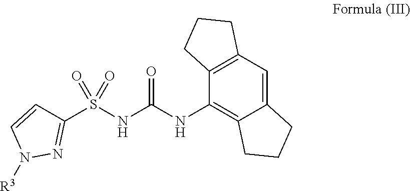

wherein:

R³ is a C₁-C₆ alkyl, C₃-C₆ cycloalkyl, (C₃-C₆ cycloalkylene)(C₁-C₂ alkyl) or (C₁-C₂ alkylene)(C₃-C₆ cycloalkyl) group, each of which is substituted with —NR^{a}R^{b};

R^{a} is a C₁-C₃ alkyl group; and

R^{b} is a C₁-C₃ alkyl group; or

R^{a} and R^{b} together with the nitrogen atom to which they are attached form a 4- to 6-membered saturated heterocycle;

provided that R³ comprises 8 or more atoms other than hydrogen or halogen.

In one embodiment, R³ is a —(C₁-C₆ alkylene)-NR^{a}R^{b} or —(C₃-C₆ cycloalkylene)(C₁-C₂ alkylene)-NR^{a}R^{b} group.

In one embodiment, R³ is

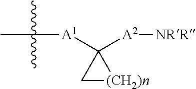

wherein A¹ and A² are each independently a bond or a C₁-C₂ alkylene group; n is 1, 2, 3 or 4; and R′ and R″ are each independently methyl or ethyl. In one embodiment, one of A¹ and A² is a bond and the other of A¹ and A² is —CH₂—; n is 2 or 3; and R′ and R″ are both methyl.

In one embodiment, R³ is

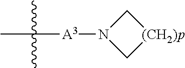

wherein A³ is a C₁-C₆ alkylene group and p is 1, 2 or 3. In one embodiment, A³ is a C₃-C₅ alkylene group and p is 1 or 2. In one embodiment, A³ is —C(CH₃)₂CH₂— and p is 1 or 2.

The first aspect of the invention also provides a compound of formula (IV):

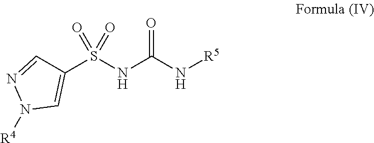

wherein:

R⁴ is a C₁-C₃ haloalkyl group;

R⁵ is selected from:

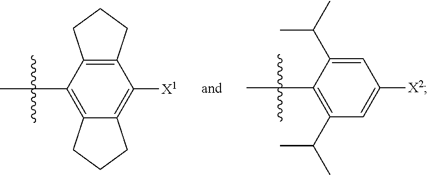

X¹ is H, F, Cl, Br or CN; and

X² is F, Cl, Br or CN.

In one embodiment, R⁴ is a C₁-C₃ alkyl group substituted with 1-7 halo atoms independently selected from fluoro and chloro atoms. In one embodiment, R⁴ is a C₁-C₃ alkyl group substituted with 1-7 fluoro atoms. In one embodiment, R⁴ is a C₁-C₂ alkyl group substituted with 1-5 fluoro atoms. In one embodiment, R⁴ is an ethyl group substituted with 2 or 3 fluoro atoms. In one embodiment, R⁴ is —CH₂CF₃ or —CH₂CHF₂.

In one embodiment, R⁵ is selected from:

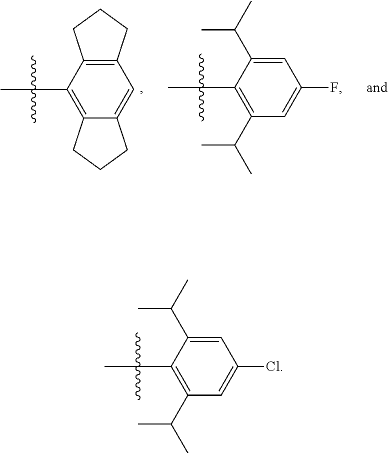 [SMILES: *c1c2c(cc3c1CCC3)CCC2 ; *c1c(C(C)C)cc(F)cc1C(C)C ; *c1c(C(C)C)cc(Cl)cc1C(C)C]

The first aspect of the invention also provides a compound of formula (V):

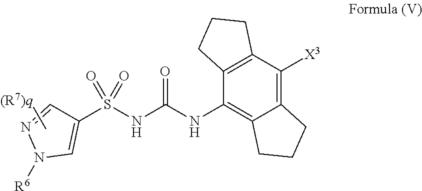

wherein:

R⁶ is a C₂-C₄ alkyl group;

R⁷ is a C₁-C₃ alkyl group;

q is 0 or 1; and

X³ is H, F, Cl, Br or CN.

In one embodiment, R⁶ is an ethyl, n-propyl or i-propyl group. In one embodiment, R⁶ is an i-propyl group.

In one embodiment, R⁷ is a methyl or ethyl group. In one embodiment, R⁷ is a methyl group. In one embodiment, q is 1. In one embodiment, q is 0.

In one embodiment, X³ is H or Br.

In one embodiment, the compound is of formula (Va):

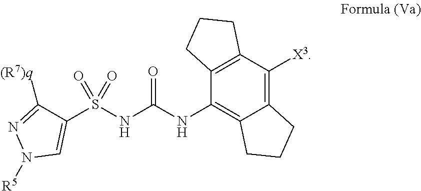

The first aspect of the invention also provides a compound of formula (VI):

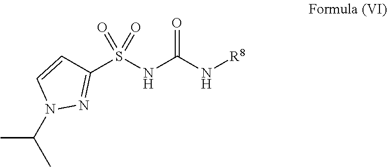

wherein:

R⁸ is selected from:

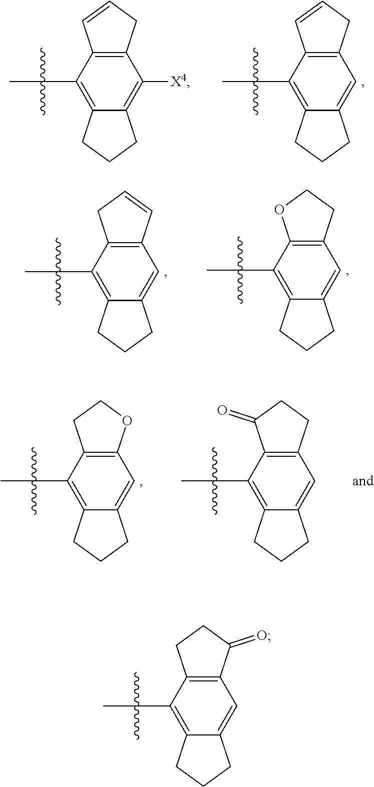

and

X⁴ is F, Cl, Br or CN.

In one embodiment, R⁸ is

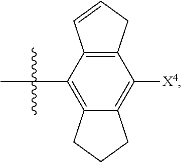

wherein X⁴ is F, Cl, Br or CN.

In one embodiment, R⁸ is

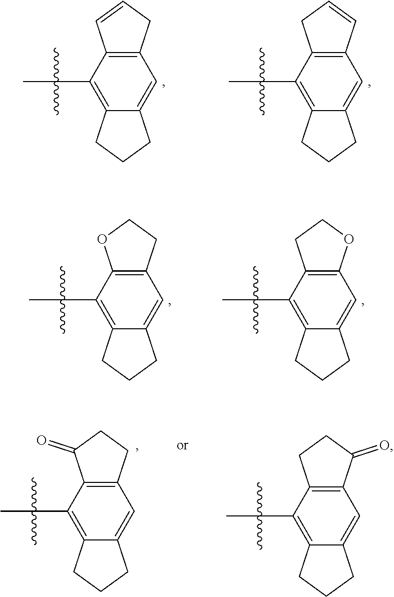 [SMILES: *c1c2c(cc3c1CCC3)CC=C2 ; *c1c2c(cc3c1CCC3)C=CC2 ; *c1c2c(cc3c1OCC3)CCC2 ; *c1c2c(cc3c1CCO3)CCC2 ; *c1c2c(cc3c1C(=O)CC3)CCC2 ; *c1c2c(cc3c1CCC3=O)CCC2]

The first aspect of the invention also provides a compound of formula (VII):

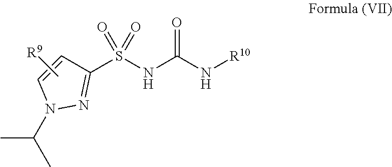

wherein:

R⁹ is halo or a group —CR⁹¹R⁹²(OH);

R⁹¹ is hydrogen or a C₁-C₃ alkyl group;

R⁹² is hydrogen or a C₁-C₃ alkyl group;

R¹⁰ is selected from:

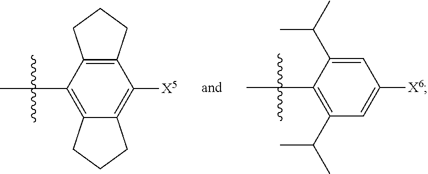

X⁵ is H, F, Cl, Br or CN; and

X⁶ is F, Cl, Br or CN.

In one embodiment, R⁹ is fluoro, chloro, or a group —CR⁹¹R⁹²(OH); wherein R⁹¹ and R⁹² are independently hydrogen or a methyl or ethyl group. In one embodiment, R⁹ is fluoro, chloro, or a group —CR⁹¹R⁹²(OH); wherein R⁹¹ and R⁹² are independently hydrogen or a methyl group. In one embodiment, R⁹ is fluoro, —C(CH₃)₂(OH), —CH(CH₃)(OH), or —CH₂(OH).

In one embodiment, R¹⁰ is selected from:

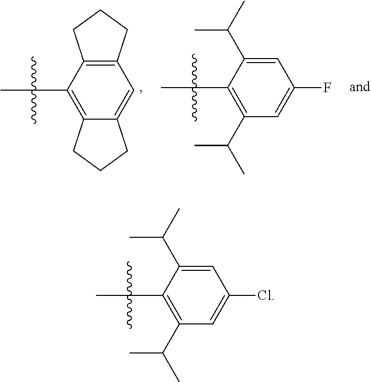 [SMILES: *c1c2c(cc3c1CCC3)CCC2 ; *c1c(C(C)C)cc(F)cc1C(C)C ; *c1c(C(C)C)cc(Cl)cc1C(C)C]

In one embodiment, the compound is of formula (VIIa):

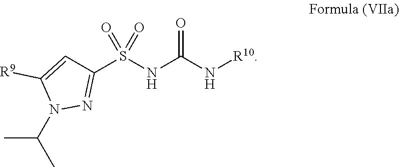

The first aspect of the invention also provides a compound of formula (VIII):

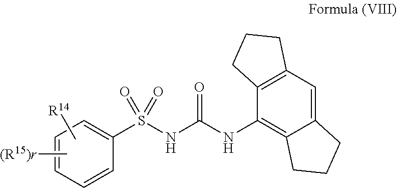

wherein:

R¹⁴ is a C₁-C₃ alkyl group substituted with —NR^{c}R^{d};

R^{c} is a C₁-C₃ alkyl group;

R^{d} is a C₁-C₃ alkyl group; or

R^{e} and R^{d} together with the nitrogen atom to which they are attached form a 4- to 6-membered saturated heterocycle;

R¹⁵ is a C₁-C₃ alkyl group; and

r is 0, 1 or 2.

In one embodiment, R¹⁴ is a C₁-C₂ alkyl group substituted with —NMe₂, —NMeEt or —NEt₂. In one embodiment, R¹⁴ is —CH₂NMe₂.

In one embodiment, R¹⁵ is methyl or ethyl, and r is 0 or 1. In one embodiment, R¹⁵ is methyl, and r is 0 or 1.

The first aspect of the invention also provides a compound of formula (IX):

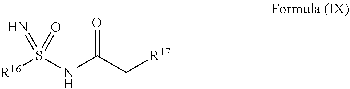

wherein:

R¹⁶ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two or three) heteroatoms N, O or S in its carbon skeleton; and

R¹⁷ is a cyclic group substituted at the a-position, wherein R¹⁷ may optionally be further substituted.

In one embodiment, R¹⁶ is a saturated or unsaturated C₁-C₂₀ (such as C₁-C₁₅ or C₁-C₁₀) hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two or three) heteroatoms N, O or S in its carbon skeleton.

In one embodiment, R¹⁶ is a 3- to 10-membered cyclic group, wherein the cyclic group may optionally be substituted. In one embodiment, R¹⁶ is a 4- to 7-membered monocyclic group, wherein the monocyclic group may optionally be substituted. In one embodiment, R¹⁶ is a 4- to 6-membered monocyclic heterocyclic group, wherein the monocyclic heterocyclic group may optionally be substituted. Typically the monocyclic heterocyclic group comprises one or more (such as one, two or three) nitrogen atoms. In one embodiment, R¹⁶ is a cycloalkyl, cycloalkenyl, non-aromatic heterocyclic, aryl or heteroaryl group, all of which may optionally be substituted. In one embodiment, R¹⁶ is a cyclopropyl, cyclobutyl, cyclopentyl, cyclohexyl, phenyl, naphthyl, azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, dithianyl, morpholinyl, thiomorpholinyl, pyridinyl, pyridazinyl, pyrimidinyl, pyrazinyl, triazinyl, pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl or thiatriazolyl group, all of which may optionally be substituted. In one embodiment, R¹⁶ is an azetidinyl, pyrrolidinyl, piperidinyl, pyrrolyl, pyrazolyl or imidazolyl group, all of which may optionally be substituted. In one embodiment, R¹⁶ is an azetidinyl or pyrazolyl group, both of which may optionally be substituted.

In one embodiment, R¹⁶ is a C₁-C₁₅ alkyl, C₂-C₁₅ alkenyl or C₂-C₁₅ alkynyl group, all of which may optionally be substituted, and all of which may optionally include one or more (such as one, two or three) heteroatoms N, O or S in their carbon skeleton.

In one embodiment, R¹⁶ is substituted with one or more (such as one, two or three) substituents independently selected from —R^{α}-halo; —R^{α}—CN; —R^{α}—NO₂; —R^{α}—N₃; —R^{α}—R^{β}; —R^{α}—OH; —R^{α}—OR^{β}; —R^{α}—SH; —R^{α}—SR^{β}; —R^{α}—SOR^{β}; —R^{α}—SO₂H; —R^{a}—SO₂R^{β}; —R^{α}—SO₂NH₂; —R^{α}—SO₂NHR^{β}; —R^{α}—SO₂N(R^{β})₂; —R^{α}—Si(R^{β})₃; —R^{α}—O—Si(R^{β})₃; —R^{α}—NH₂; —R^{α}—NHR^{β}; —R^{α}—N(R^{β})₂; —R^{α}—N⁺(R^{β})₃; —R^{α}—CHO; —R^{α}—COR^{β}; —R^{α}—COOH; —R^{α}—COOR^{β}; —R^{α}—OCOR^{β}; —R^{α}—CONH₂; —R^{α}—CONHR^{β}; —R^{α}—CON(R^{β})₂; and oxo (═O);

wherein each —R^{α}— is independently selected from a bond or an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more (such as one, two or three) heteroatoms N, O or S, and wherein the alkylene, alkenylene or alkynylene group may optionally be substituted with one or more (such as one, two or three) halo and/or —R^{β} groups; and

wherein each —R^{β} is independently selected from a C₁-C₆ alkyl, C₂-C₆ alkenyl, C₂-C₆ alkynyl or 4- to 6-membered heterocyclic group, or wherein any two —R^{β} attached to the same nitrogen atom may, together with the nitrogen atom to which they are attached, form a 4- to 6-membered heterocyclic group, and wherein any —R^{β} may optionally be substituted with one or more (such as one, two or three) C₁-C₄ alkyl, C₁-C₄ haloalkyl, C₃-C₇ cycloalkyl, C₃-C₇ halocycloalkyl, —O(C₁-C₄ alkyl), —O(C₁-C₄ haloalkyl), —O(C₃-C₇ cycloalkyl), —O(C₃-C₇ halocycloalkyl), —CO(C₁-C₄ alkyl), —CO(C₁-C₄ haloalkyl), —COO(C₁-C₄ alkyl), —COO(C₁-C₄ haloalkyl), halo, —OH, —NH₂, —CN, —C≡CH, or oxo (═O) groups.

In one embodiment, R¹⁶ is substituted with one or more (such as one, two or three) substituents independently selected from —R^{α}-halo; —R^{α}—CN; —R^{α}—NO₂; —R^{α}—N₃; —R^{α}—R^{β}; —R^{α}—OH; —R^{α}—OR^{β}; —R^{α}—SH; —R^{α}—SR^{β}; —R^{α}—SOR^{β}; —R^{a}—SO₂H; —R^{α}—SO₂R^{β}; —R^{α}—SO₂NH₂; —R^{α}—SO₂NHR^{β}; —R^{α}—SO₂N(R^{β})₂; —R^{α}—Si(R^{β})₃; —R^{α}—O—Si(R^{β})₃; —R^{α}—NH₂; —R^{a}—NHR^{β}; —R^{α}—N(R^{β})₂; —R^{α}N⁺(R^{β})₃; —R^{α}—CHO; —R^{α}—COR^{β}; —R^{a}—COOH; —R^{a}—COOR^{β}; —R^{α}OCOR^{β}; —R^{α}—CONH₂; —R^{α}—CONHR^{β}; —R^{α}—CON(R^{β})₂; and oxo (═O);

wherein each —R^{α}— is independently selected from a bond or a C₁-C₆ alkylene group; and

wherein each —R^{β} is independently selected from a C₁-C₆ alkyl group optionally substituted with one or more (such as one, two or three) halo, —OH, —NH₂, —CN, or oxo (═O) groups.

In one embodiment, R¹⁶ is substituted with one or two C₁-C₄ alkyl groups. In one embodiment, R¹⁶ is substituted with one methyl, ethyl, n-propyl or i-propyl group.

In one embodiment, R¹⁷ is an aryl or a heteroaryl group (such as a phenyl group), wherein the aryl or the heteroaryl group is substituted at the α-position, and wherein R¹⁷ may optionally be further substituted.

In one embodiment, R¹⁷ is an aryl or a heteroaryl group (such as a phenyl group), wherein the aryl or the heteroaryl group is substituted at the α and α′ positions, and wherein R¹⁷ may optionally be further substituted.

Typical substituents at the α and/or α′ positions of the parent cyclic group of R¹⁷ comprise a carbon atom.

In one embodiment, the cyclic group of R¹⁷ is substituted at the α and/or α′ positions with C₁-C₆ alkyl and/or across the α,β and/or α′,β′ positions with —(CH₂)ₛ—, wherein s is 2, 3 or 4.

In one embodiment, the cyclic group of R¹⁷ is further substituted with halo or CN.

In one embodiment, R¹⁷ is a fused aryl or a fused heteroaryl group (such as a fused phenyl group), wherein a first cycloalkyl, cycloalkenyl, non-aromatic heterocyclic, aryl or heteroaryl ring is fused to the aryl or heteroaryl group across the α,β positions and a second cycloalkyl, cycloalkenyl, non-aromatic heterocyclic, aryl or heteroaryl ring is fused to the aryl or heteroaryl group across the α′,β′ positions, and wherein R¹⁷ may optionally be further substituted, for example with halo or CN. Typically in such an embodiment, R¹⁷ is tricyclic.

In one embodiment R¹⁷ is selected from:

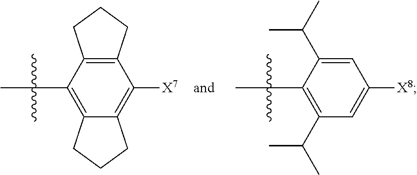

wherein

X⁷ is H, F, Cl, Br or CN; and

X⁸ is F, Cl, Br or CN.

In one embodiment, R¹⁷ is selected from:

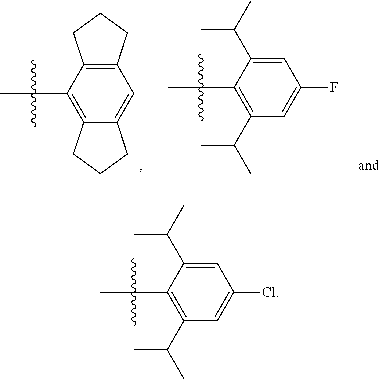 [SMILES: *c1c2c(cc3c1CCC3)CCC2 ; *c1c(C(C)C)cc(F)cc1C(C)C ; *c1c(C(C)C)cc(Cl)cc1C(C)C]

In another embodiment, R¹⁷ is a cyclic group (such as a phenyl group) substituted at the α-position with a monovalent heterocyclic group or a monovalent aromatic group, wherein a ring atom of the heterocyclic or aromatic group is directly attached to the α-ring atom of the cyclic group, wherein the heterocyclic or aromatic group may optionally be substituted, and wherein the cyclic group may optionally be further substituted.

In one embodiment, the α-substituted cyclic group of R¹⁷ is further substituted at the α′-position with C₁-C₆ alkyl or across the α′,β′ positions with —(CH₂)ₛ—, wherein s is 2, 3 or 4.

In one embodiment, the α-substituted cyclic group of R¹⁷ is substituted further still with halo or CN.

In one embodiment, the monovalent heterocyclic or aromatic group at the α-position is a phenyl, pyridinyl, pyridazinyl, pyrimidinyl, pyrazinyl, pyrrolyl, furanyl, thiophenyl, pyrazolyl, imidazolyl, oxazolyl, isoxazolyl, thiazolyl, isothiazolyl, triazolyl, oxadiazolyl, azetinyl, azetidinyl, oxetanyl, thietanyl, pyrrolidinyl, tetrahydrofuranyl, tetrahydrothiophenyl, pyrazolidinyl, imidazolidinyl, 1,3-dioxolanyl, 1,2-oxathiolanyl, 1,3-oxathiolanyl, piperidinyl, tetrahydropyranyl, piperazinyl, 1,4-dioxanyl, thianyl, morpholinyl, thiomorpholinyl or 1-methyl-2-oxo-1,2-dihydropyridinyl group, all of which may optionally be substituted. In one embodiment, the monovalent heterocyclic or aromatic group is a phenyl, pyridinyl, pyrimidinyl, pyrazolyl, imidazolyl, isoxazolyl, thiazolyl or tetrahydropyranyl group, all of which may optionally be substituted. In one embodiment, the monovalent heterocyclic or aromatic group is a phenyl, pyridinyl, pyrimidinyl or pyrazolyl group, all of which may optionally be substituted. In one embodiment, the monovalent heterocyclic or aromatic group is an optionally substituted pyridinyl group.

In one embodiment, R¹⁷ is selected from:

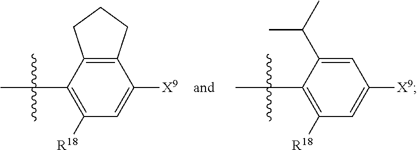

wherein

X⁹ is H, F, Cl, Br or CN; and

R¹⁸ is a phenyl, pyridinyl, pyrimidinyl, pyrazolyl, imidazolyl, isoxazolyl, thiazolyl or tetrahydropyranyl group, all of which may optionally be substituted.

In one embodiment, R¹⁸ is a phenyl, pyridinyl, pyrimidinyl or pyrazolyl group, all of which may optionally be substituted. In one embodiment, R¹⁸ is an optionally substituted pyridinyl group.

In one embodiment, the monovalent heterocyclic or aromatic group at the α-position (which may be R¹⁸) is substituted with one or two substituents independently selected from halo, —OH, —NH₂, —CN, —NO₂, —R^{γ}, —OR^{γ}, —NHR^{γ} or —N(R^{γ})₂, wherein each R^{γ} is independently selected from a C₁-C₄ alkyl, C₂-C₄ alkenyl or C₂-C₄ alkynyl group all of which may optionally be halo-substituted. In one embodiment, the monovalent heterocyclic or aromatic group (which may be R¹⁸) is substituted with one or two substituents independently selected from halo, —OH, —NH₂, —CN, C₁-C₃ alkyl or —O(C₁-C₃ alkyl).

In one embodiment, R¹⁷ is phenyl or a 5- or 6-membered heteroaryl group (such as phenyl, pyridinyl, pyridazinyl, pyrimidinyl or pyrazinyl); wherein

(i) the phenyl or 5- or 6-membered heteroaryl group is substituted at the α position with a substituent selected from —R¹⁹, —OR¹⁹ and —COR¹⁹, wherein R¹⁹ is selected from a C₁-C₆ alkyl, C₂-C₆ alkenyl, C₂-C₆ alkynyl or C₂-C₆ cyclic group and wherein R¹⁹ is optionally substituted with one or more halo groups; and

optionally the phenyl or 5- or 6-membered heteroaryl group is further substituted at the α′ position with a substituent selected from —R²⁰, —OR²⁰ and —COR²⁰, wherein R²⁰ is selected from a C₁-C₆ alkyl, C₂-C₆ alkenyl, C₂-C₆ alkynyl or C₂-C₆ cyclic group and wherein R²⁰ is optionally substituted with one or more halo groups; and

optionally the phenyl or 5- or 6-membered heteroaryl group is further substituted (typically with one, two or three substituents independently selected from halo, —NO₂, —CN, —COOR²¹, —CONH₂, —CONHR²¹ or —CON(R²¹)₂, wherein each —R²¹ is independently selected from a C₁-C₄ alkyl or C₁-C₄ haloalkyl group); or

(ii) the phenyl or 5- or 6-membered heteroaryl group is substituted with a cycloalkyl, cycloalkenyl, non-aromatic heterocyclic, aryl or heteroaryl ring which is fused to the parent phenyl or 5- or 6-membered heteroaryl group across the α,β positions and which is optionally substituted with one or more halo groups; and

optionally the phenyl or 5- or 6-membered heteroaryl group is further substituted at the α′ position with a substituent selected from —R¹⁹, —OR¹⁹ and —COR¹⁹, wherein R¹⁹ is selected from a C₁-C₆ alkyl, C₂-C₆ alkenyl, C₂-C₆ alkynyl or C₂-C₆ cyclic group and wherein R¹⁹ is optionally substituted with one or more halo groups; and

optionally the phenyl or 5- or 6-membered heteroaryl group is further substituted (typically with one or two substituents independently selected from halo, —NO₂, —CN, —COOR²¹, —CONH₂, —CONHR²¹ or —CON(R²¹)₂, wherein each —R²¹ is independently selected from a C₁-C₄ alkyl or C₁-C₄ haloalkyl group); or

(iii) the phenyl or 5- or 6-membered heteroaryl group is substituted with a first cycloalkyl, cycloalkenyl, non-aromatic heterocyclic, aryl or heteroaryl ring which is fused to the parent phenyl or 5- or 6-membered heteroaryl group across the α,β positions and which is optionally substituted with one or more halo groups; and

the phenyl or 5- or 6-membered heteroaryl group is substituted with a second cycloalkyl, cycloalkenyl, non-aromatic heterocyclic, aryl or heteroaryl ring which is fused to the parent phenyl or 5- or 6-membered heteroaryl group across the α′,β′ positions and which is optionally substituted with one or more halo groups; and

optionally the phenyl group is further substituted (typically with a substituent selected from halo, —NO₂, —CN, —COOR²¹, —CONH₂, —CONHR²¹ or —CON(R²¹)₂, wherein each —R²¹ is independently selected from a C₁-C₄ alkyl or C₁-C₄ haloalkyl group); or

(iv) the phenyl or 5- or 6-membered heteroaryl group is substituted at the α-position with a monovalent heterocyclic group or a monovalent aromatic group selected from phenyl, pyridinyl, pyrimidinyl, pyrazolyl, imidazolyl, triazolyl or tetrahydropyranyl, wherein the monovalent heterocyclic or aromatic group may optionally be substituted with one or two substituents independently selected from halo, C₁-C₃ alkyl, C₁-C₃ haloalkyl, —R²²—OR²³, —R²²—N(R²³)₂, —R²²—CN or —R²²—C≡CR²³, and wherein a ring atom of the monovalent heterocyclic or aromatic group is directly attached to the α-ring atom of the parent phenyl or 5- or 6-membered heteroaryl group; wherein R²² is independently selected from a bond or a C₁-C₃ alkylene group; and R²³ is independently selected from hydrogen or a C₁-C₃ alkyl or C₁-C₃ haloalkyl group; and

optionally the phenyl or 5- or 6-membered heteroaryl group is further substituted at the α′ position with a substituent selected from —R¹⁹, —OR¹⁹ and —COR¹⁹, wherein R¹⁹ is selected from a C₁-C₆ alkyl, C₂-C₆ alkenyl, C₂-C₆ alkynyl or C₂-C₆ cyclic group and wherein R¹⁹ is optionally substituted with one or more halo groups; and

optionally the phenyl or 5- or 6-membered heteroaryl group is further substituted (typically with one, two or three substituents independently selected from halo, —NO₂, —CN, —COOR²¹, —CONH₂, —CONHR²¹ or —CON(R²¹)₂, wherein each —R²¹ is independently selected from a C₁-C₄ alkyl or C₁-C₄ haloalkyl group); or

(v) the phenyl or 5- or 6-membered heteroaryl group is substituted at the α-position with a monovalent heterocyclic group or a monovalent aromatic group selected from phenyl, pyridinyl, pyrimidinyl, pyrazolyl, imidazolyl, triazolyl or tetrahydropyranyl, wherein the monovalent heterocyclic or aromatic group may optionally be substituted with one or two substituents independently selected from halo, C₁-C₃ alkyl, C₁-C₃ haloalkyl, —R²²—OR²³, —R²²—N(R²³)₂, —R²²—CN or —R²²—C≡CR²³, and wherein a ring atom of the monovalent heterocyclic or aromatic group is directly attached to the α-ring atom of the parent phenyl or 5- or 6-membered heteroaryl group; wherein R²² is independently selected from a bond or a C₁-C₃ alkylene group; and R²³ is independently selected from hydrogen or a C₁-C₃ alkyl or C₁-C₃ haloalkyl group; and

optionally the phenyl or 5- or 6-membered heteroaryl group is further substituted with a cycloalkyl, cycloalkenyl, non-aromatic heterocyclic, aryl or heteroaryl ring which is fused to the parent phenyl or 5- or 6-membered heteroaryl group across the α′,β′ positions and which is optionally substituted with one or more halo groups; and

optionally the phenyl or 5- or 6-membered heteroaryl group is further substituted (typically with one or two substituents independently selected from halo, —NO₂, —CN, —COOR²¹, —CONH₂, —CONHR²¹ or —CON(R²¹)₂, wherein each —R²¹ is independently selected from a C₁-C₄ alkyl or C₁-C₄ haloalkyl group).

In the context of the present specification, a “hydrocarbyl” substituent group or a hydrocarbyl moiety in a substituent group only includes carbon and hydrogen atoms but, unless stated otherwise, does not include any heteroatoms, such as N, O or S, in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O or S, in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₂₀ hydrocarbyl group. More typically a hydrocarbyl group is a C₁-C₁₅ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, i-butenyl, 2-butenyl, 1-pentenyl, i-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₂-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl groups/moieties. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O or S, in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from ₃ to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O or S, in the ring structure. Examples of heterocyclic groups include heteroaryl groups as discussed below and non-aromatic heterocyclic groups such as azetinyl, azetidinyl, oxetanyl, thietanyl, pyrrolidinyl, tetrahydrofuranyl, tetrahydrothiophenyl, pyrazolidinyl, imidazolidinyl, dioxolanyl, oxathiolanyl, piperidinyl, tetrahydropyranyl, thianyl, piperazinyl, dioxanyl, morpholinyl and thiomorpholinyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl. Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

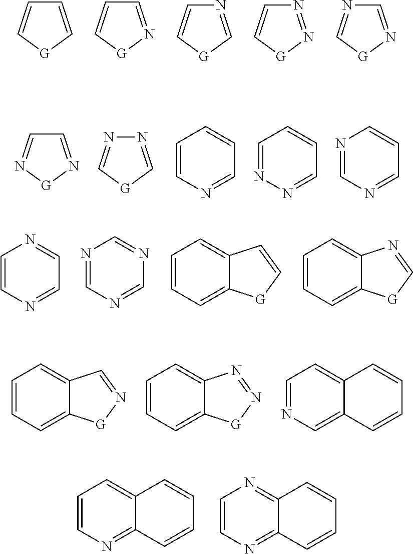

wherein G=O, S or NH.

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

Typically a substituted group comprises 1, 2, 3 or 4 substituents, more typically 1, 2 or 3 substituents, more typically 1 or 2 substituents, and more typically 1 substituent.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O or S, are to be counted as carbon atoms when calculating the number of carbon atoms in a Cₓ-C_{y} group. For example, a morpholinyl group is to be considered a C₆ heterocyclic group, not a C₄ heterocyclic group.

For the purposes of the present specification, where it is stated that a first atom or group is “directly attached” to a second atom or group it is to be understood that the first atom or group is covalently bonded to the second atom or group with no intervening atom(s) or group(s) being present. So, for example, for the group (C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

A second aspect of the invention provides a compound selected from the group consisting of:

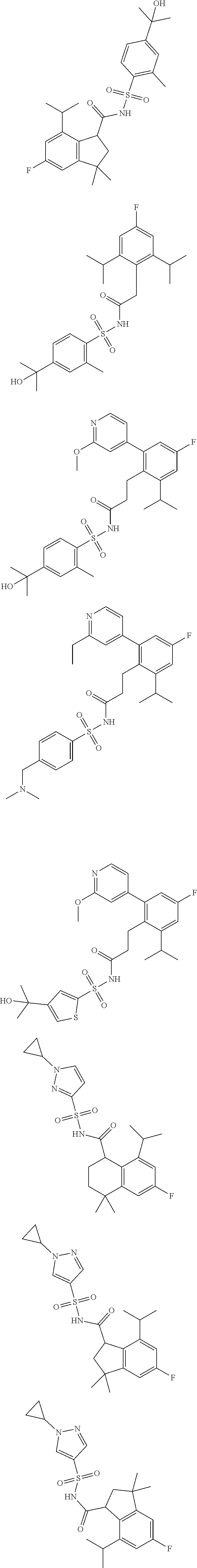

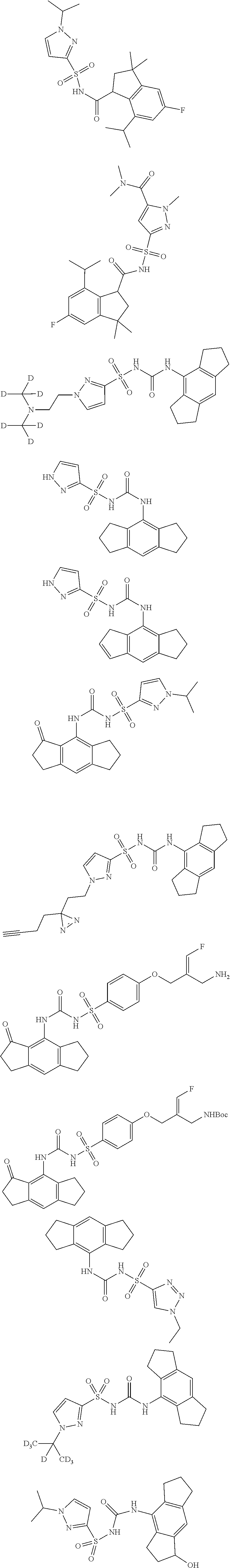

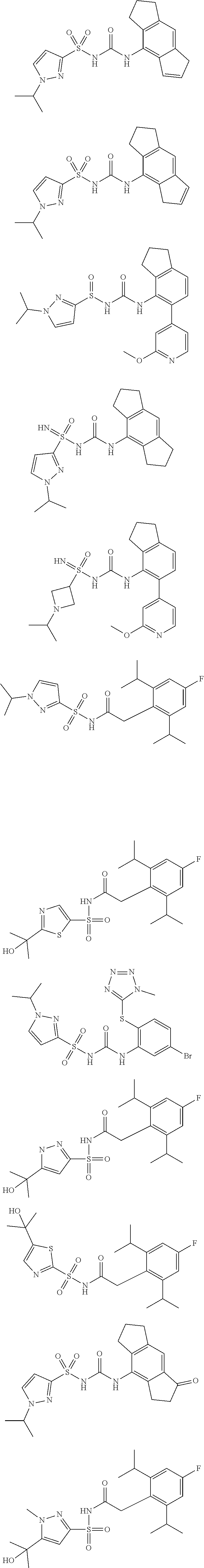

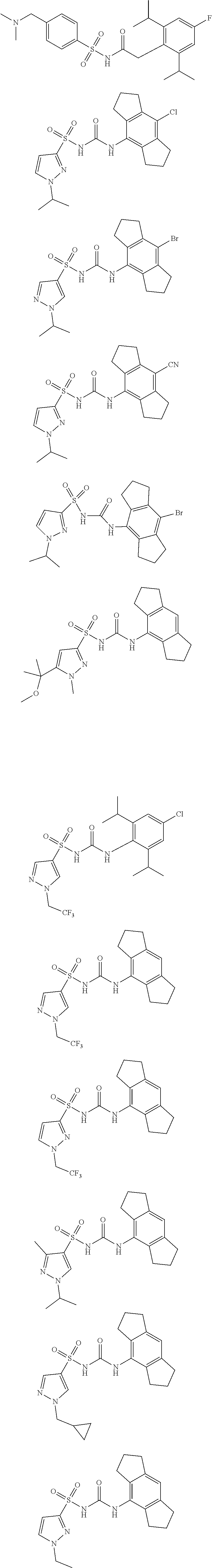

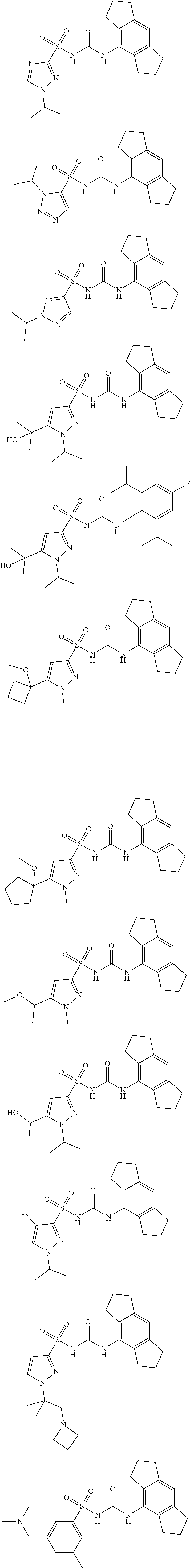

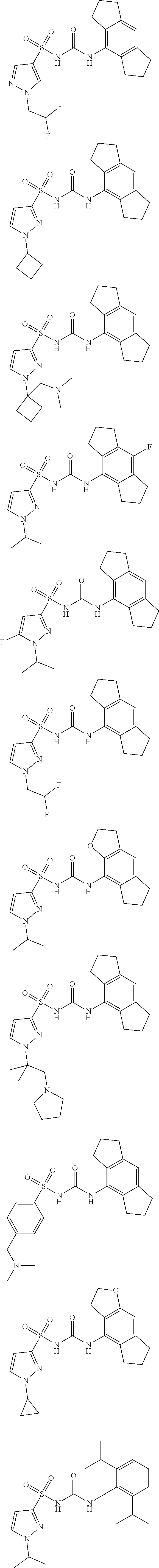

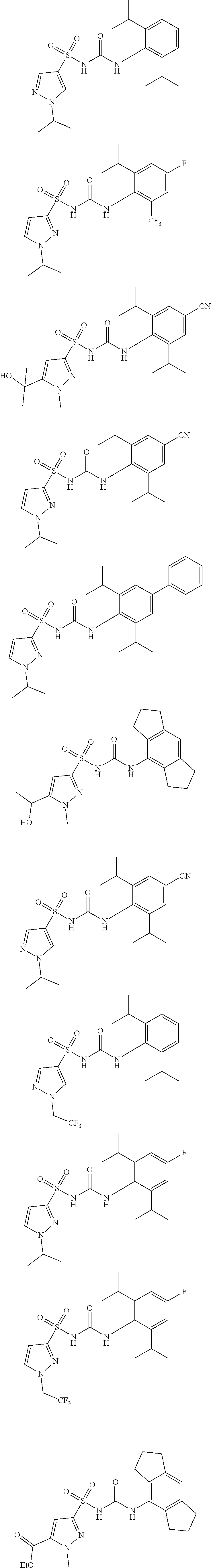

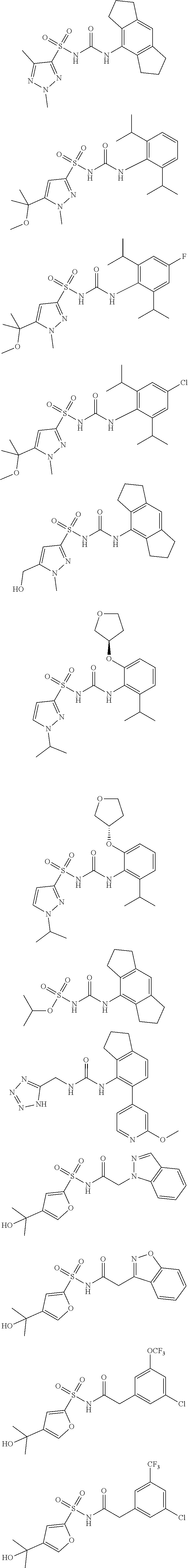

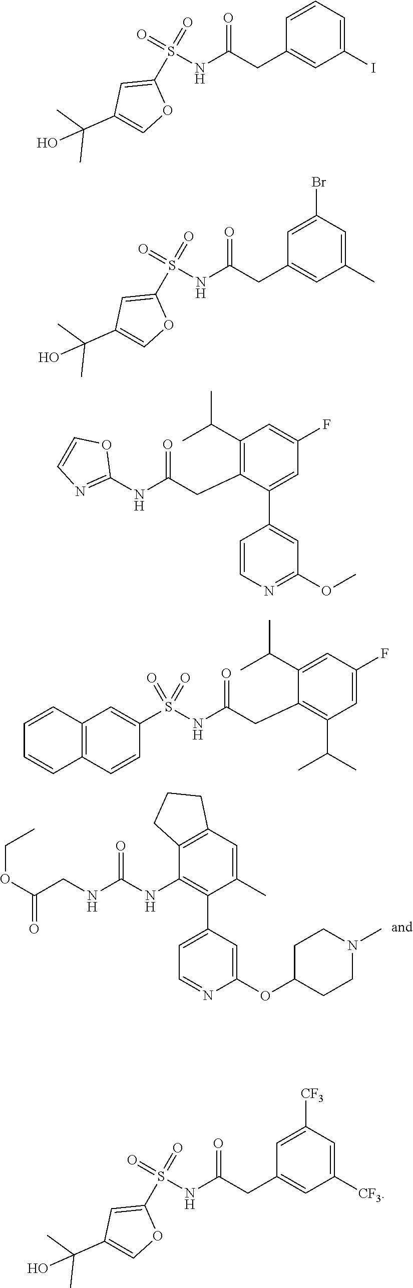

A third aspect of the invention provides a pharmaceutically acceptable salt, solvate or prodrug of any compound of the first or second aspect of the invention.

The compounds of the present invention can be used both, in their free base form and their acid addition salt form. For the purposes of this invention, a “salt” of a compound of the present invention includes an acid addition salt. Acid addition salts are preferably pharmaceutically acceptable, non-toxic addition salts with suitable acids, including but not limited to inorganic acids such as hydrohalogenic acids (for example, hydrofluoric, hydrochloric, hydrobromic or hydroiodic acid) or other inorganic acids (for example, nitric, perchloric, sulfuric or phosphoric acid); or organic acids such as organic carboxylic acids (for example, propionic, butyric, glycolic, lactic, mandelic, citric, acetic, benzoic, salicylic, succinic, malic or hydroxysuccinic, tartaric, fumaric, maleic, hydroxymaleic, mucic or galactaric, gluconic, pantothenic or pamoic acid), organic sulfonic acids (for example, methanesulfonic, trifluoromethanesulfonic, ethanesulfonic, 2-hydroxyethanesulfonic, benzenesulfonic, toluene-p-sulfonic, naphthalene-2-sulfonic or camphorsulfonic acid) or amino acids (for example, ornithinic, glutamic or aspartic acid). The acid addition salt may be a mono-, di-, tri- or multi-acid addition salt. A preferred salt is a hydrohalogenic, sulfuric, phosphoric or organic acid addition salt. A preferred salt is a hydrochloric acid addition salt.

Where a compound of the invention includes a quaternary ammonium group, typically the compound is used in its salt form. The counter ion to the quaternary ammonium group may be any pharmaceutically acceptable, non-toxic counter ion. Examples of suitable counter ions include the conjugate bases of the protic acids discussed above in relation to acid addition salts.

The compounds of the present invention can also be used both, in their free acid form and their salt form. For the purposes of this invention, a “salt” of a compound of the present invention includes one formed between a protic acid functionality (such as a carboxylic acid group) of a compound of the present invention and a suitable cation. Suitable cations include, but are not limited to lithium, sodium, potassium, magnesium, calcium and ammonium. The salt may be a mono-, di-, tri- or multi-salt. Preferably the salt is a mono- or di-lithium, sodium, potassium, magnesium, calcium or ammonium salt. More preferably the salt is a mono- or di-sodium salt or a mono- or di-potassium salt.

Preferably any salt is a pharmaceutically acceptable non-toxic salt. However, in addition to pharmaceutically acceptable salts, other salts are included in the present invention, since they have potential to serve as intermediates in the purification or preparation of other, for example, pharmaceutically acceptable salts, or are useful for identification, characterisation or purification of the free acid or base.

The compounds and/or salts of the present invention may be anhydrous or in the form of a hydrate (e.g. a hemihydrate, monohydrate, dihydrate or trihydrate) or other solvate. Such other solvates may be formed with common organic solvents, including but not limited to, alcoholic solvents e.g. methanol, ethanol or isopropanol.

In some embodiments of the present invention, therapeutically inactive prodrugs are provided. Prodrugs are compounds which, when administered to a subject such as a human, are converted in whole or in part to a compound of the invention. In most embodiments, the prodrugs are pharmacologically inert chemical derivatives that can be converted in vivo to the active drug molecules to exert a therapeutic effect. Any of the compounds described herein can be administered as a prodrug to increase the activity, bioavailability, or stability of the compound or to otherwise alter the properties of the compound. Typical examples of prodrugs include compounds that have biologically labile protecting groups on a functional moiety of the active compound. Prodrugs include, but are not limited to, compounds that can be oxidized, reduced, aminated, deaminated, hydroxylated, dehydroxylated, hydrolyzed, dehydrolyzed, alkylated, dealkylated, acylated, deacylated, phosphorylated, and/or dephosphorylated to produce the active compound. The present invention also encompasses salts and solvates of such prodrugs as described above.

The compounds, salts, solvates and prodrugs of the present invention may contain at least one chiral centre. The compounds, salts, solvates and prodrugs may therefore exist in at least two isomeric forms. The present invention encompasses racemic mixtures of the compounds, salts, solvates and prodrugs of the present invention as well as enantiomerically enriched and substantially enantiomerically pure isomers. For the purposes of this invention, a “substantially enantiomerically pure” isomer of a compound comprises less than 5% of other isomers of the same compound, more typically less than 2%, and most typically less than 0.5% by weight.

The compounds, salts, solvates and prodrugs of the present invention may contain any stable isotope including, but not limited to ¹²C, ¹³C, ¹H, ²H (D), ¹⁴N, ¹⁵N, ¹⁶O, ¹⁷O, ¹⁸O, ¹⁹F and ¹²⁷I, and any radioisotope including, but not limited to ¹¹C, ¹⁴C, ³H (T), ¹³N, ¹⁵O, ¹⁸F, ¹²³I, ¹²⁴I, ¹²⁵I and ¹³¹I.

The compounds, salts, solvates and prodrugs of the present invention may be in any polymorphic or amorphous form.

A fourth aspect of the invention provides a pharmaceutical composition comprising a compound of the first or second aspect of the invention, or a pharmaceutically acceptable salt, solvate or prodrug of the third aspect of the invention, and a pharmaceutically acceptable excipient.

Conventional procedures for the selection and preparation of suitable pharmaceutical formulations are described in, for example, “Aulton's Pharmaceutics—The Design and Manufacture of Medicines”, M. E. Aulton and K. M. G. Taylor, Churchill Livingstone Elsevier, 4^{th} Ed., 2013.

Pharmaceutically acceptable excipients including adjuvants, diluents or carriers that may be used in the pharmaceutical compositions of the invention are those conventionally employed in the field of pharmaceutical formulation, and include, but are not limited to, sugars, sugar alcohols, starches, ion exchangers, alumina, aluminium stearate, lecithin, serum proteins such as human serum albumin, buffer substances such as phosphates, glycerine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinylpyrrolidone, cellulose-based substances, polyethylene glycol, sodium carboxymethylcellulose, polyacrylates, waxes, polyethylene-polyoxypropylene-block polymers, polyethylene glycol and wool fat.

In one embodiment, the pharmaceutical composition of the fourth aspect of the invention additionally comprises one or more further active agents.

In a further embodiment, the pharmaceutical composition of the fourth aspect of the invention may be provided as a part of a kit of parts, wherein the kit of parts comprises the pharmaceutical composition of the fourth aspect of the invention and one or more further pharmaceutical compositions, wherein the one or more further pharmaceutical compositions each comprise a pharmaceutically acceptable excipient and one or more further active agents.

A fifth aspect of the invention provides a compound of the first or second aspect of the invention, or a pharmaceutically acceptable salt, solvate or prodrug of the third aspect of the invention, or a pharmaceutical composition of the fourth aspect of the invention, for use in medicine, and/or for use in the treatment or prevention of a disease, disorder or condition. Typically, the use comprises the administration of the compound, salt, solvate, prodrug or pharmaceutical composition to a subject. In one embodiment, the use comprises the co-administration of one or more further active agents.

The term “treatment” as used herein refers equally to curative therapy, and ameliorating or palliative therapy. The term includes obtaining beneficial or desired physiological results, which may or may not be established clinically. Beneficial or desired clinical results include, but are not limited to, the alleviation of symptoms, the prevention of symptoms, the diminishment of extent of disease, the stabilisation (i.e., not worsening) of a condition, the delay or slowing of progression/worsening of a condition/symptom, the amelioration or palliation of a condition/symptom, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering a compound, salt, solvate, prodrug or pharmaceutical composition of the present invention. The term “prevention” as used herein in relation to a disease, disorder or condition, relates to prophylactic or preventative therapy, as well as therapy to reduce the risk of developing the disease, disorder or condition. The term “prevention” includes both the avoidance of occurrence of the disease, disorder or condition, and the delay in onset of the disease, disorder or condition. Any statistically significant (p≤0.05) avoidance of occurrence, delay in onset or reduction in risk as measured by a controlled clinical trial may be deemed a prevention of the disease, disorder or condition. Subjects amenable to prevention include those at heightened risk of a disease, disorder or condition as identified by genetic or biochemical markers. Typically, the genetic or biochemical markers are appropriate to the disease, disorder or condition under consideration and may include for example, inflammatory biomarkers such as C-reactive protein (CRP) and monocyte chemoattractant protein 1 (MCP-1) in the case of inflammation; total cholesterol, triglycerides, insulin resistance and C-peptide in the case of NAFLD and NASH; and more generally IL-1β and IL-18 in the case of a disease, disorder or condition responsive to NLRP3 inhibition.

A sixth aspect of the invention provides the use of a compound of the first or second aspect, or a pharmaceutically effective salt, solvate or prodrug of the third aspect, in the manufacture of a medicament for the treatment or prevention of a disease, disorder or condition. Typically, the treatment or prevention comprises the administration of the compound, salt, solvate, prodrug or medicament to a subject. In one embodiment, the treatment or prevention comprises the co-administration of one or more further active agents.

A seventh aspect of the invention provides a method of treatment or prevention of a disease, disorder or condition, the method comprising the step of administering an effective amount of a compound of the first or second aspect, or a pharmaceutically acceptable salt, solvate or prodrug of the third aspect, or a pharmaceutical composition of the fourth aspect, to thereby treat or prevent the disease, disorder or condition. In one embodiment, the method further comprises the step of co-administering an effective amount of one or more further active agents. Typically, the administration is to a subject in need thereof.

An eighth aspect of the invention provides a compound of the first or second aspect of the invention, or a pharmaceutically acceptable salt, solvate or prodrug of the third aspect of the invention, or a pharmaceutical composition of the fourth aspect of the invention, for use in the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the use comprises the administration of the compound, salt, solvate, prodrug or pharmaceutical composition to the individual. In one embodiment, the use comprises the co-administration of one or more further active agents. The use may also comprise the diagnosis of an individual having a germline or somatic non-silent mutation in NLRP3, wherein the compound, salt, solvate, prodrug or pharmaceutical composition is administered to an individual on the basis of a positive diagnosis for the mutation. Typically, identification of the mutation in NLRP3 in the individual may be by any suitable genetic or biochemical means.

A ninth aspect of the invention provides the use of a compound of the first or second aspect, or a pharmaceutically effective salt, solvate or prodrug of the third aspect, in the manufacture of a medicament for the treatment or prevention of a disease, disorder or condition in an individual, wherein the individual has a germline or somatic non-silent mutation in NLRP3. The mutation may be, for example, a gain-of-function or other mutation resulting in increased NLRP3 activity. Typically, the treatment or prevention comprises the administration of the compound, salt, solvate, prodrug or medicament to the individual. In one embodiment, the treatment or prevention comprises the co-administration of one or more further active agents. The treatment or prevention may also comprise the diagnosis of an individual having a germline or somatic non-silent mutation in NLRP3, wherein the compound, salt, solvate, prodrug or medicament is administered to an individual on the basis of a positive diagnosis for the mutation. Typically, identification of the mutation in NLRP3 in the individual may be by any suitable genetic or biochemical means.

A tenth aspect of the invention provides a method of treatment or prevention of a disease, disorder or condition, the method comprising the steps of diagnosing of an individual having a germline or somatic non-silent mutation in NLRP3, and administering an effective amount of a compound of the first or second aspect, or a pharmaceutically acceptable salt, solvate or prodrug of the third aspect, or a pharmaceutical composition of the fourth aspect, to the positively diagnosed individual, to thereby treat or prevent the disease, disorder or condition. In one embodiment, the method further comprises the step of co-administering an effective amount of one or more further active agents. Typically, the administration is to a subject in need thereof.

In general embodiments, the disease, disorder or condition may be a disease, disorder or condition of the immune system, the cardiovascular system, the endocrine system, the gastrointestinal tract, the renal system, the hepatic system, the metabolic system, the respiratory system, the central nervous system, may be a cancer or other malignancy, and/or may be caused by or associated with a pathogen.

It will be appreciated that these general embodiments defined according to broad categories of diseases, disorders and conditions are not mutually exclusive. In this regard any particular disease, disorder or condition may be categorized according to more than one of the above general embodiments. A non-limiting example is type I diabetes which is an autoimmune disease and a disease of the endocrine system.

In one embodiment of the fifth, sixth, seventh, eighth, ninth or tenth aspect of the invention, the disease, disorder or condition is responsive to NLRP3 inhibition. As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

Genetic diseases in which a role for NLRP3 has been suggested include sickle cell disease (Vogel et al., Blood, 130(Suppl 1): 2234, 2017), and Valosin Containing Protein disease (Nalbandian et al., Inflammation, 40(1): 21-41, 2017).

NLRP3 has been implicated in a number of autoinflammatory diseases, including Familial Mediterranean fever (FMF), TNF receptor associated periodic syndrome (TRAPS), hyperimmunoglobulinemia D and periodic fever syndrome (HIDS), pyogenic arthritis, pyoderma gangrenosum and acne (PAPA), Sweet's syndrome, chronic nonbacterial osteomyelitis (CNO), and acne vulgaris (Cook et al., Eur. J. Immunol., 40: 595-653, 2010). In particular, NLRP3 mutations have been found to be responsible for a set of rare autoinflammatory diseases known as CAPS (Ozaki et al., J. Inflammation Research, 8: 15-27, 2015; Schroder et al., Cell, 140: 821-832, 2010; and Menu et al., Clinical and Experimental Immunology, 166: 1-15, 2011). CAPS are heritable diseases characterized by recurrent fever and inflammation and are comprised of three autoinflammatory disorders that form a clinical continuum. These diseases, in order of increasing severity, are familial cold autoinflammatory syndrome (FCAS), Muckle-Wells syndrome (MWS), and chronic infantile cutaneous neurological articular syndrome (CINCA; also called neonatal-onset multisystem inflammatory disease, NOMID), and all have been shown to result from gain-of-function mutations in the NLRP3 gene, which leads to increased secretion of IL-1β.

A number of autoimmune diseases have been shown to involve NLRP3 including, in particular, multiple sclerosis, type 1 diabetes (T1D), psoriasis, rheumatoid arthritis (RA), Behcet's disease, Schnitzler's syndrome, macrophage activation syndrome (Masters, Clin Immunol, 147(3): 223-228, 2013; Braddock et al., Nat Rev Drug Disc, 3: 1-10, 2004; Inoue et al., Immunology, 139: 11-18, 2013; Coll et al., Nat Med, 21(3): 248-55, 2015; Scott et al., Clin Exp Rheumatol, 34(1): 88-93, 2016; and Guo et al., Clin Exp Immunol, 194(2): 231-243, 2018), systemic lupus erythematosus (Lu et al., J Immunol, 198(3): 1119-29, 2017) including lupus nephritis (Zhao et al., Arthritis and Rheumatism, 65(12): 3176-3185, 2013), multiple sclerosis (Xu et al., J Cell Biochem, 120(4): 5160-5168, 2019), and systemic sclerosis (Artlett et al., Arthritis Rheum, 63(11): 3563-74, 2011).

NLRP3 has also been shown to play a role in a number of lung diseases including chronic obstructive pulmonary disorder (COPD), asthma (including steroid-resistant asthma and eosinophilic asthma), asbestosis, and silicosis (De Nardo et al., Am J Pathol, 184: 42-54, 2014; Lv et al., J Biol Chem, 293(48): 18454, 2018; and Kim et al., Am J Respir Crit Care Med, 196(3): 283-97, 2017).

NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al., Brain Behav Immun, 61: 306-316, 2017), intracranial aneurysms (Zhang et al., J Stroke & Cerebrovascular Dis, 24(5): 972-979, 2015), intracerebral haemorrhages (ICH) (Ren et al., Stroke, 49(1): 184-192, 2018), cerebral ischemia-reperfusion injuries (Fauzia et al., Front Pharmacol, 9: 1034, 2018; Hong et al., Neural Plasticity, 2018: 8, 2018; Ye et al., Experimental Neurology, 292: 46-55, 2017), general anesthesia neuroinflammation (Fan et al., Front Cell Neurosci, 12: 426, 2018), sepsis-associated encephalopathy (SAE) (Fu et al., Inflammation, 42(1): 306-318, 2019), postoperative cognitive dysfunction (POCD) (Fan et al., Front Cell Neurosci, 12: 426, 2018), early brain injury (subarachnoid haemorrhage SAH) (Luo et al., Brain Res Bull, 146: 320-326, 2019), and traumatic brain injury (Ismael et al., J Neurotrauma, 35(11): 1294-1303, 2018).

NRLP3 activity has also been shown to be involved in various metabolic diseases including type 2 diabetes (T2D), atherosclerosis, obesity, gout, pseudo-gout, metabolic syndrome (Wen et al., Nature Immunology, 13: 352-357, 2012; Duewell et al., Nature, 464: 1357-1361, 2010; Strowig et al., Nature, 481: 278-286, 2012), and non-alcoholic steatohepatitis (NASH) (Mridha et al., J Hepatol, 66(5): 1037-46, 2017).

A role for NLRP3 via IL-1β has also been suggested in atherosclerosis, myocardial infarction (van Hout et al., Eur Heart J, 38(11): 828-36, 2017), cardiovascular disease (Janoudi et al., European Heart Journal, 37(25): 1959-1967, 2016), cardiac hypertrophy and fibrosis (Gan et al., Biochim Biophys Acta, 1864(1): 1-10, 2018), heart failure (Sano et al., J Am Coll Cardiol, 71(8): 875-66, 2018), aortic aneurysm and dissection (Wu et al., Arterioscler Thromb Vasc Biol, 37(4): 694-706, 2017), cardiac injury induced by metabolic dysfunction (Pavillard et al., Oncotarget, 8(59): 99740-99756, 2017), atrial fibrillation (Yao et al., Circulation, 138(20): 2227-2242, 2018), hypertension (Gan et al., Biochim Biophys Acta, 1864(1): 1-10, 2018), and other cardiovascular events (Ridker et al., N Engl J Med, doi: 10.1056/NEJM0a1707914, 2017).

Other diseases in which NLRP3 has been shown to be involved include:

ocular diseases such as both wet and dry age-related macular degeneration (Doyle et al., Nature Medicine, 18: 791-798, 2012; and Tarallo et al., Cell, 149(4): 847-59, 2012), diabetic retinopathy (Loukovaara et al., Acta Ophthalmol, 95(8): 803-808, 2017) and optic nerve damage (Puyang et al., Sci Rep, 6: 20998, 2016 Feb. 19);

liver diseases including non-alcoholic steatohepatitis (NASH) (Henao-Meija et al., Nature, 482: 179-185, 2012), ischemia reperfusion injury of the liver (Yu et al., Transplantation, 103(2): 353-362, 2019), fulminant hepatitis (Pourcet et al., Gastroenterology, 154(5): 1449-1464, e20, 2018), liver fibrosis (Zhang et al., Parasit Vectors, 12(1): 29, 2019), and liver failure including acute liver failure (Wang et al., Hepatol Res, 48(3): E194-E202, 2018);

kidney diseases including nephrocalcinosis (Anders et al., Kidney Int, 93(3): 656-669, 2018), kidney fibrosis including chronic crystal nephropathy (Ludwig-Portugall et al., Kidney Int, 90(3): 525-39, 2016), and renal hypertension (Krishnan et al., Br J Pharmacol, 173(4): 752-65, 2016; Krishnan et al., Cardiovasc Res, 115(4): 776-787, 2019; Dinh et al., Aging, 9(6): 1595-1606, 2017);

conditions associated with diabetes including diabetic encephalopathy (Zhai et al., Molecules, 23(3): 522, 2018), diabetic retinopathy (Zhang et al., Cell Death Dis, 8(7): e2941, 2017), and diabetic hypoadiponectinemia (Zhang et al., Biochimica et Biophysica Acta (BBA)—Molecular Basis of Disease, 1863(6): 1556-1567, 2017);

inflammatory reactions in the lung and skin (Primiano et al., J Immunol, 197(6): 2421-33, 2016) including lung ischemia-reperfusion injury (Xu et al., Biochemical and Biophysical Research Communications, 503(4): 3031-3037, 2018), epithelial to mesenchymal transition (EMT) (Li et al., Experimental Cell Research, 362(2): 489-497, 2018), contact hypersensitivity (such as bullous pemphigoid (Fang et al., J Dermatol Sci, 83(2): 116-23, 2016)), atopic dermatitis (Niebuhr et al., Allergy, 69(8): 1058-67, 2014), Hidradenitis suppurativa (Alikhan et al., J Am Acad Dermatol, 60(4): 539-61, 2009), acne vulgaris (Qin et al., J Invest Dermatol, 134(2): 381-88, 2014), and sarcoidosis (Jager et al., Am J Respir Crit Care Med, 191: A5816, 2015);

inflammatory reactions in the joints (Braddock et al., Nat Rev Drug Disc, 3: 1-10, 2004) and osteoarthritis (Jin et al., PNAS, 108(36): 14867-14872, 2011);

amyotrophic lateral sclerosis (Gugliandolo et al., Inflammation, 41(1): 93-103, 2018);

cystic fibrosis (Iannitti et al., Nat Commun, 7: 10791, 2016);

stroke (Walsh et al., Nature Reviews, 15: 84-97, 2014; Ye et al., Experimental Neurology, 292: 46-55, 2017);

chronic kidney disease (Granata et al., PLoS One, 10(3): e0122272, 2015);

Sjögren's syndrome (Vakrakou et al., Journal of Autoimmunity, 91: 23-33, 2018);

sickle cell disease (Vogel et al., Blood, 130(Supp11): 2234, 2017); and

colitis and inflammatory bowel diseases including ulcerative colitis and Crohn's disease (Braddock et al., Nat Rev Drug Disc, 3: 1-10, 2004; Neudecker et al., J Exp Med, 214(6): 1737-52, 2017; Wu et al., Mediators Inflamm, 2018: 3048532, 2018; and Lazaridis et al., Dig Dis Sci, 62(9): 2348-56, 2017), and sepsis (intestinal epithelial disruption) (Zhang et al., Dig Dis Sci, 63(1): 81-91, 2018).

Genetic ablation of NLRP3 has been shown to protect from HSD (high sugar diet), HFD (high fat diet) and HSFD-induced obesity (Pavillard et al., Oncotarget, 8(59): 99740-99756, 2017).

The NLRP3 inflammasome has been found to be activated in response to oxidative stress, sunburn (Hasegawa et al., Biochemical and Biophysical Research Communications, 477(3): 329-335, 2016), and UVB irradiation (Schroder et al., Science, 327: 296-300, 2010).

NLRP3 has also been shown to be involved in inflammatory hyperalgesia (Dolunay et al., Inflammation, 40: 366-386, 2017), wound healing (Ito et al., Exp Dermatol, 27(1): 80-86, 2018), pain including multiple sclerosis-associated neuropathic pain (Khan et al., Inflammopharmacology, 26(1): 77-86, 2018), and intra-amniotic inflammation/infection associated with preterm birth (Faro et al., Biol Reprod, 100(5): 1290-1305, 2019; and Gomez-Lopez et al., Biol Reprod, 100(5): 1306-1318, 2019).

The inflammasome, and NLRP3 specifically, has also been proposed as a target for modulation by various pathogens including bacterial pathogens such as Staphylococcus aureus (Cohen et al., Cell Reports, 22(9): 2431-2441, 2018), Bacillus cereus (Mathur et al., Nat Microbiol, 4: 362-374, 2019), Salmonella typhimurium (Diamond et al., Sci Rep, 7(1): 6861, 2017), and group A streptococcus (LaRock et al., Science Immunology, 1(2): eaah3539, 2016); viruses such as DNA viruses (Amsler et al., Future Virol, 8(4): 357-370, 2013), influenza A virus (Coates et al., Front Immunol, 8: 782, 2017), chikungunya, Ross river virus, and alpha viruses (Chen et al., Nat Microbiol, 2(10): 1435-1445, 2017); fungal pathogens such as Candida albicans (Tucey et al., mSphere, 1(3), pii: e00074-16, 2016); and other pathogens such as T. gondii (Gov et al., J Immunol, 199(8): 2855-2864, 2017), helminth worms (Alhallaf et al., Cell Reports, 23(4): 1085-1098, 2018), Leishmania (Novais et al., PLoS Pathogens, 13(2): e1006196, 2017), and Plasmodium (Strangward et al., PNAS, 115(28): 7404-7409, 2018). NLRP3 has been shown to be required for the efficient control of viral, bacterial, fungal, and helminth pathogen infections (Strowig et al., Nature, 481: 278-286, 2012).

NLRP3 has also been implicated in the pathogenesis of many cancers (Menu et al., Clinical and Experimental Immunology, 166: 1-15, 2011; and Masters, Clin Immunol, 147(3): 223-228, 2013). For example, several previous studies have suggested a role for IL-1β in cancer invasiveness, growth and metastasis, and inhibition of IL-1β with canakinumab has been shown to reduce the incidence of lung cancer and total cancer mortality in a randomised, double-blind, placebo-controlled trial (Ridker et al., Lancet, S0140-6736(17)32247-X, 2017). Inhibition of the NLRP3 inflammasome or IL-1β has also been shown to inhibit the proliferation and migration of lung cancer cells in vitro (Wang et al., Oncol Rep, 35(4): 2053-64, 2016). A role for the NLRP3 inflammasome has been suggested in myelodysplastic syndromes (Basiorka et al., Blood, 128(25): 2960-2975, 2016) and also in the carcinogenesis of various other cancers including glioma (Li et al., Am J Cancer Res, 5(1): 442-449, 2015), colon cancer (Allen et al., J Exp Med, 207(5): 1045-56, 2010), melanoma (Dunn et al., Cancer Lett, 314(1): 24-33, 2012), breast cancer (Guo et al., Scientific Reports, 6: 36107, 2016), inflammation-induced tumours (Allen et al., J Exp Med, 207(5): 1045-56, 2010; and Hu et al., PNAS, 107(50): 21635-40, 2010), multiple myeloma (Li et al., Hematology, 21(3): 144-51, 2016), and squamous cell carcinoma of the head and neck (Huang et al., J Exp Clin Cancer Res, 36(1): 116, 2017). Activation of the NLRP3 inflammasome has also been shown to mediate chemoresistance of tumour cells to 5-fluorouracil (Feng et al., J Exp Clin Cancer Res, 36(1): 81, 2017), and activation of the NLRP3 inflammasome in peripheral nerves contributes to chemotherapy-induced neuropathic pain (Jia et al., Mol Pain, 13: 1-11, 2017).

Accordingly, examples of diseases, disorders or conditions which may be responsive to NLRP3 inhibition and which may be treated or prevented in accordance with the fifth, sixth, seventh, eighth, ninth or tenth aspect of the present invention include:

(i) inflammation, including inflammation occurring as a result of an inflammatory disorder, e.g. an autoinflammatory disease, inflammation occurring as a symptom of a non-inflammatory disorder, inflammation occurring as a result of infection, or inflammation secondary to trauma, injury or autoimmunity;

(ii) auto-immune diseases such as acute disseminated encephalitis, Addison's disease, ankylosing spondylitis, antiphospholipid antibody syndrome (APS), anti-synthetase syndrome, aplastic anemia, autoimmune adrenalitis, autoimmune hepatitis, autoimmune oophoritis, autoimmune polyglandular failure, autoimmune thyroiditis, Coeliac disease, Crohn's disease, type 1 diabetes (T1D), Goodpasture's syndrome, Graves' disease, Guillain-Barré syndrome (GBS), Hashimoto's disease, idiopathic thrombocytopenic purpura, Kawasaki's disease, lupus erythematosus including systemic lupus erythematosus (SLE), multiple sclerosis (MS) including primary progressive multiple sclerosis (PPMS), secondary progressive multiple sclerosis (SPMS) and relapsing remitting multiple sclerosis (RRMS), myasthenia gravis, opsoclonus myoclonus syndrome (OMS), optic neuritis, Ord's thyroiditis, pemphigus, pernicious anaemia, polyarthritis, primary biliary cirrhosis, rheumatoid arthritis (RA), psoriatic arthritis, juvenile idiopathic arthritis or Still's disease, refractory gouty arthritis, Reiter's syndrome, Sjögren's syndrome, systemic sclerosis a systemic connective tissue disorder, Takayasu's arteritis, temporal arteritis, warm autoimmune hemolytic anemia, Wegener's granulomatosis, alopecia universalis, Behcet's disease, Chagas' disease, dysautonomia, endometriosis, hidradenitis suppurativa (HS), interstitial cystitis, neuromyotonia, psoriasis, sarcoidosis, scleroderma, ulcerative colitis, Schnitzler's syndrome, macrophage activation syndrome, Blau syndrome, vitiligo or vulvodynia;

(iii) cancer including lung cancer, pancreatic cancer, gastric cancer, myelodysplastic syndrome, leukaemia including acute lymphocytic leukaemia (ALL) and acute myeloid leukaemia (AML), adrenal cancer, anal cancer, basal and squamous cell skin cancer, squamous cell carcinoma of the head and neck, bile duct cancer, bladder cancer, bone cancer, brain and spinal cord tumours, breast cancer, cervical cancer, chronic lymphocytic leukaemia (CLL), chronic myeloid leukaemia (CML), chronic myelomonocytic leukaemia (CMML), colorectal cancer, endometrial cancer, oesophagus cancer, Ewing family of tumours, eye cancer, gallbladder cancer, gastrointestinal carcinoid tumours, gastrointestinal stromal tumour (GIST), gestational trophoblastic disease, glioma, Hodgkin lymphoma, Kaposi sarcoma, kidney cancer, laryngeal and hypopharyngeal cancer, liver cancer, lung carcinoid tumour, lymphoma including cutaneous T cell lymphoma, malignant mesothelioma, melanoma skin cancer, Merkel cell skin cancer, multiple myeloma, nasal cavity and paranasal sinuses cancer, nasopharyngeal cancer, neuroblastoma, non-Hodgkin lymphoma, non-small cell lung cancer, oral cavity and oropharyngeal cancer, osteosarcoma, ovarian cancer, penile cancer, pituitary tumours, prostate cancer, retinoblastoma, rhabdomyosarcoma, salivary gland cancer, skin cancer, small cell lung cancer, small intestine cancer, soft tissue sarcoma, stomach cancer, testicular cancer, thymus cancer, thyroid cancer including anaplastic thyroid cancer, uterine sarcoma, vaginal cancer, vulvar cancer, Waldenstrom macroglobulinemia, and Wilms tumour;

(iv) infections including viral infections (e.g. from influenza virus, human immunodeficiency virus (HIV), alphavirus (such as Chikungunya and Ross River virus), flaviviruses (such as Dengue virus and Zika virus), herpes viruses (such as Epstein Barr virus, cytomegalovirus, Varicella-zoster virus, and KSHV), poxviruses (such as vaccinia virus (Modified vaccinia virus Ankara) and Myxoma virus), adenoviruses (such as Adenovirus 5), or papillomavirus), bacterial infections (e.g. from Staphylococcus aureus, Helicobacter pylori, Bacillus anthracis, Bacillus cereus, Bordatella pertussis, Burkholderia pseudomallei, Corynebacterium diptheriae, Clostridium tetani, Clostridium botulinum, Streptococcus pneumoniae, Streptococcus pyogenes, Listeria monocytogenes, Hemophilus influenzae, Pasteurella multicida, Shigella dysenteriae, Mycobacterium tuberculosis, Mycobacterium leprae, Mycoplasma pneumoniae, Mycoplasma hominis, Neisseria meningitidis, Neisseria gonorrhoeae, Rickettsia rickettsii, Legionella pneumophila, Klebsiella pneumoniae, Pseudomonas aeruginosa, Propionibacterium acnes, Treponema pallidum, Chlamydia trachomatis, Vibrio cholerae, Salmonella typhimurium, Salmonella typhi, Borrelia burgdorferi, Uropathogenic Escherichia coli (UPEC) or Yersinia pestis), fungal infections (e.g. from Candida or Aspergillus species), protozoan infections (e.g. from Plasmodium, Babesia, Giardia, Entamoeba, Leishmania or Trypanosomes), helminth infections (e.g. from schistosoma, roundworms, tapeworms or flukes) and prion infections;

(v) central nervous system diseases such as Parkinson's disease, Alzheimer's disease, dementia, motor neuron disease, Huntington's disease, cerebral malaria, brain injury from pneumococcal meningitis, intracranial aneurysms, intracerebral haemorrhages, sepsis-associated encephalopathy, postoperative cognitive dysfunction, early brain injury, traumatic brain injury, cerebral ischemia-reperfusion injury, stroke, general anesthesia neuroinflammation and amyotrophic lateral sclerosis;

(vi) metabolic diseases such as type 2 diabetes (T2D), atherosclerosis, obesity, gout, and pseudo-gout;

(vii) cardiovascular diseases such as hypertension, ischaemia, reperfusion injury including post-MI ischemic reperfusion injury, stroke including ischemic stroke, transient ischemic attack, myocardial infarction including recurrent myocardial infarction, heart failure including congestive heart failure and heart failure with preserved ejection fraction, cardiac hypertrophy and fibrosis, embolism, aneurysms including abdominal aortic aneurysm, and pericarditis including Dressler's syndrome;

(viii) respiratory diseases including chronic obstructive pulmonary disorder (COPD), asthma such as allergic asthma, eosinophilic asthma, and steroid-resistant asthma, asbestosis, silicosis, nanoparticle induced inflammation, cystic fibrosis and idiopathic pulmonary fibrosis;

(ix) liver diseases including non-alcoholic fatty liver disease (NAFLD) and non-alcoholic steatohepatitis (NASH) including advanced fibrosis stages F3 and F4, alcoholic fatty liver disease (AFLD), alcoholic steatohepatitis (ASH), ischemia reperfusion injury of the liver, fulminant hepatitis, liver fibrosis, and liver failure including acute liver failure;

(x) renal diseases including chronic kidney disease, oxalate nephropathy, nephrocalcinosis, glomerulonephritis, diabetic nephropathy, kidney fibrosis including chronic crystal nephropathy, and renal hypertension;

(xi) ocular diseases including those of the ocular epithelium, age-related macular degeneration (AMD) (dry and wet), Sjögren's syndrome, uveitis, corneal infection, diabetic retinopathy, optic nerve damage, dry eye, and glaucoma;

(xii) skin diseases including dermatitis such as contact dermatitis and atopic dermatitis, contact hypersensitivity, psoriasis, sunburn, skin lesions, hidradenitis suppurativa (HS), other cyst-causing skin diseases, pyoderma gangrenosum, and acne vulgaris including acne conglobata;

(xiii) lymphatic conditions such as lymphangitis and Castleman's disease;

(xiv) psychological disorders such as depression and psychological stress;

(xv) graft versus host disease;

(xvi) allodynia including mechanical allodynia;

(xvii) conditions associated with diabetes including diabetic encephalopathy, diabetic retinopathy, and diabetic hypoadiponectinemia; and

(xviii) any disease where an individual has been determined to carry a germline or somatic non-silent mutation in NLRP3.

In one embodiment, the disease, disorder or condition is selected from:

(i) cancer;

(ii) an infection;

(iii) a central nervous system disease;

(iv) a cardiovascular disease;

(v) a liver disease;

(vi) an ocular disease; or

(vii) a skin disease.

More typically, the disease, disorder or condition is selected from:

(i) cancer;

(ii) an infection;

(iii) a central nervous system disease; or

(iv) a cardiovascular disease.

In one embodiment, the disease, disorder or condition is selected from:

(i) acne conglobata;

(iii) Alzheimer's disease;

(iv) amyotrophic lateral sclerosis;

(v) age-related macular degeneration (AMD);

(vi) anaplastic thyroid cancer;

(vii) cryopyrin-associated periodic syndromes (CAPS);

(viii) contact dermatitis;

(ix) cystic fibrosis;

(x) congestive heart failure;

(xi) chronic kidney disease;

(xii) Crohn's disease;

(xiii) familial cold autoinflammatory syndrome (FCAS);

(xiv) Huntington's disease;

(xv) heart failure;

(xvi) heart failure with preserved ejection fraction;

(xvii) ischemic reperfusion injury;

(xviii) juvenile idiopathic arthritis;

(xix) myocardial infarction;

(xx) macrophage activation syndrome;

(xxi) myelodysplastic syndrome;

(xxii) multiple myeloma;

(xxiii) motor neuron disease;

(xxiv) multiple sclerosis;

(xxv) Muckle-Wells syndrome;

(xxvi) non-alcoholic steatohepatitis (NASH);

(xxvii) neonatal-onset multisystem inflammatory disease (NOMID);

(xxviii) Parkinson's disease;

(xxix) sickle cell disease;

(xxx) systemic juvenile idiopathic arthritis;

(xxxi) systemic lupus erythematosus;

(xxxii) traumatic brain injury;

(xxxiii) transient ischemic attack;

(xxxiv) ulcerative colitis; or

(xxxv) Valosin Containing Protein disease.

In a further typical embodiment of the invention, the disease, disorder or condition is inflammation. Examples of inflammation that may be treated or prevented in accordance with the fifth, sixth, seventh, eighth, ninth or tenth aspect of the present invention include inflammatory responses occurring in connection with, or as a result of:

(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;

(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);

(iii) a muscular condition such as polymyositis or myasthenia gravis;

(iv) a gastrointestinal tract condition such as inflammatory bowel disease (including Crohn's disease and ulcerative colitis), colitis, gastric ulcer, coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);

(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, adult respiratory distress syndrome, hypersensitivity pneumonitis, or idiopathic interstitial pneumonia;

(vi) a vascular condition such as atherosclerosis, Behcet's disease, vasculitides, or Wegener's granulomatosis;

(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;

(viii) an ocular condition such as uveitis, allergic conjunctivitis, or vernal conjunctivitis;

(ix) a nervous condition such as multiple sclerosis or encephalomyelitis;

(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, mycobacterium tuberculosis, mycobacterium avium intracellulare, pneumocystis carinii pneumonia, orchitis/epidydimitis, legionella, Lyme disease, influenza A, Epstein-Barr virus infection, viral encephalitis/aseptic meningitis, or pelvic inflammatory disease;

(xi) a renal condition such as mesangial proliferative glomerulonephritis, nephrotic syndrome, nephritis, glomerular nephritis, acute renal failure, uremia, nephritic syndrome, kidney fibrosis including chronic crystal nephropathy, or renal hypertension;

(xii) a lymphatic condition such as Castleman's disease;

(xiii) a condition of, or involving, the immune system, such as hyper IgE syndrome, lepromatous leprosy, familial hemophagocytic lymphohistiocytosis, or graft versus host disease;

(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;

(xv) a cancer, including those cancers listed above;

(xvi) a burn, wound, trauma, haemorrhage or stroke;

(xvii) radiation exposure;

(xviii) obesity; and/or

(xix) pain such as inflammatory hyperalgesia.

In one embodiment of the fifth, sixth, seventh, eighth, ninth or tenth aspect of the present invention, the disease, disorder or condition is an autoinflammatory disease such as cryopyrin-associated periodic syndromes (CAPS), Muckle-Wells syndrome (MWS), familial cold autoinflammatory syndrome (FCAS), familial Mediterranean fever (FMF), neonatal onset multisystem inflammatory disease (NOMID), Tumour Necrosis Factor (TNF) Receptor-Associated Periodic Syndrome (TRAPS), hyperimmunoglobulinemia D and periodic fever syndrome (HIDS), deficiency of interleukin 1 receptor antagonist (DIRA), Majeed syndrome, pyogenic arthritis, pyoderma gangrenosum and acne syndrome (PAPA), adult-onset Still's disease (AOSD), haploinsufficiency of A20 (HA20), pediatric granulomatous arthritis (PGA), PLCG2-associated antibody deficiency and immune dysregulation (PLAID), PLCG2-associated autoinflammatory, antibody deficiency and immune dysregulation (APLAID), or sideroblastic anaemia with B-cell immunodeficiency, periodic fevers and developmental delay (SIFD).

Examples of diseases, disorders or conditions which may be responsive to NLRP3 inhibition and which may be treated or prevented in accordance with the fifth, sixth, seventh, eighth, ninth or tenth aspect of the present invention are listed above. Some of these diseases, disorders or conditions are substantially or entirely mediated by NLRP3 inflammasome activity, and NLRP3-induced IL-1β and/or IL-18. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), neonatal onset multisystem inflammatory disease (NOMID), familial Mediterranean fever (FMF), pyogenic arthritis, pyoderma gangrenosum and acne syndrome (PAPA), hyperimmunoglobulinemia D and periodic fever syndrome (HIDS), Tumour Necrosis Factor (TNF) Receptor-Associated Periodic Syndrome (TRAPS), systemic juvenile idiopathic arthritis, adult-onset Still's disease (AOSD), relapsing polychondritis, Schnitzler's syndrome, Sweet's syndrome, Behcet's disease, anti-synthetase syndrome, deficiency of interleukin 1 receptor antagonist (DIRA), and haploinsufficiency of A20 (HA20).

Moreover, some of the diseases, disorders or conditions mentioned above arise due to mutations in NLRP3, in particular, resulting in increased NLRP3 activity. As a result, such diseases, disorders or conditions may be particularly responsive to NLRP3 inhibition and may be particularly suitable for treatment or prevention in accordance with the fifth, sixth, seventh, eighth, ninth or tenth aspect of the present invention. Examples of such diseases, disorders or conditions include cryopyrin-associated periodic syndromes (CAPS), Muckle-Wells syndrome (MWS), familial cold autoinflammatory syndrome (FCAS), and neonatal onset multisystem inflammatory disease (NOMID).

An eleventh aspect of the invention provides a method of inhibiting NLRP3, the method comprising the use of a compound of the first or second aspect of the invention, or a pharmaceutically acceptable salt, solvate or prodrug of the third aspect of the invention, or a pharmaceutical composition of the fourth aspect of the invention, to inhibit NLRP3.

In one embodiment of the eleventh aspect of the present invention, the method comprises the use of a compound of the first or second aspect of the invention, or a pharmaceutically acceptable salt, solvate or prodrug of the third aspect of the invention, or a pharmaceutical composition of the fourth aspect of the invention, in combination with one or more further active agents.

In one embodiment of the eleventh aspect of the present invention, the method is performed ex vivo or in vitro, for example in order to analyse the effect on cells of NLRP3 inhibition.

In another embodiment of the eleventh aspect of the present invention, the method is performed in vivo. For example, the method may comprise the step of administering an effective amount of a compound of the first or second aspect, or a pharmaceutically acceptable salt, solvate or prodrug of the third aspect, or a pharmaceutical composition of the fourth aspect, to thereby inhibit NLRP3. In one embodiment, the method further comprises the step of co-administering an effective amount of one or more further active agents. Typically, the administration is to a subject in need thereof.

Alternately, the method of the eleventh aspect of the invention may be a method of inhibiting NLRP3 in a non-human animal subject, the method comprising the steps of administering the compound, salt, solvate, prodrug or pharmaceutical composition to the non-human animal subject and optionally subsequently mutilating or sacrificing the non-human animal subject. Typically, such a method further comprises the step of analysing one or more tissue or fluid samples from the optionally mutilated or sacrificed non-human animal subject. In one embodiment, the method further comprises the step of co-administering an effective amount of one or more further active agents.

A twelfth aspect of the invention provides a compound of the first or second aspect of the invention, or a pharmaceutically acceptable salt, solvate or prodrug of the third aspect of the invention, or a pharmaceutical composition of the fourth aspect of the invention, for use in the inhibition of NLRP3. Typically, the use comprises the administration of the compound, salt, solvate, prodrug or pharmaceutical composition to a subject. In one embodiment, the compound, salt, solvate, prodrug or pharmaceutical composition is co-administered with one or more further active agents.

A thirteenth aspect of the invention provides the use of a compound of the first or second aspect of the invention, or a pharmaceutically effective salt, solvate or prodrug of the third aspect of the invention, in the manufacture of a medicament for the inhibition of NLRP3. Typically, the inhibition comprises the administration of the compound, salt, solvate, prodrug or medicament to a subject. In one embodiment, the compound, salt, solvate, prodrug or medicament is co-administered with one or more further active agents.

In any embodiment of any of the fifth to thirteenth aspects of the present invention that comprises the use or co-administration of one or more further active agents, the one or more further active agents may comprise for example one, two or three different further active agents.

The one or more further active agents may be used or administered prior to, simultaneously with, sequentially with or subsequent to each other and/or to the compound of the first or second aspect of the invention, the pharmaceutically acceptable salt, solvate or prodrug of the third aspect of the invention, or the pharmaceutical composition of the fourth aspect of the invention. Where the one or more further active agents are administered simultaneously with the compound of the first or second aspect of the invention, or the pharmaceutically acceptable salt, solvate or prodrug of the third aspect of the invention, a pharmaceutical composition of the fourth aspect of the invention may be administered wherein the pharmaceutical composition additionally comprises the one or more further active agents.

In one embodiment of any of the fifth to thirteenth aspects of the present invention that comprises the use or co-administration of one or more further active agents, the one or more further active agents are selected from:

(i) chemotherapeutic agents;

(ii) antibodies;

(iii) alkylating agents;

(iv) anti-metabolites;

(v) anti-angiogenic agents;

(vi) plant alkaloids and/or terpenoids;

(vii) topoisomerase inhibitors;

(viii) mTOR inhibitors;

(ix) stilbenoids;

(x) STING agonists;

(xi) cancer vaccines;

(xii) immunomodulatory agents;

(xiii) antibiotics;

(xiv) anti-fungal agents;

(xv) anti-helminthic agents; and/or

(xvi) other active agents.

It will be appreciated that these general embodiments defined according to broad categories of active agents are not mutually exclusive. In this regard any particular active agent may be categorized according to more than one of the above general embodiments. A non-limiting example is urelumab which is an antibody that is an immunomodulatory agent for the treatment of cancer.

In some embodiments, the one or more chemotherapeutic agents are selected from abiraterone acetate, altretamine, amsacrine, anhydrovinblastine, auristatin, azathioprine, adriamycin, bexarotene, bicalutamide, BMS 184476, bleomycin, N,N-dimethyl-L-valyl-L-valyl-N-methyl-L-valyl-L-prolyl-L-proline-t-butylamide, cisplatin, carboplatin, carboplatin cyclophosphamide, chlorambucil, cachectin, cemadotin, cyclophosphamide, carmustine, cryptophycin, cytarabine, docetaxel, doxetaxel, doxorubicin, dacarbazine (DTIC), dactinomycin, daunorubicin, decitabine, dolastatin, etoposide, etoposide phosphate, enzalutamide (MDV3100), 5-fluorouracil, fludarabine, flutamide, gemcitabine, hydroxyurea and hydroxyureataxanes, idarubicin, ifosfamide, irinotecan, leucovorin,lonidamine,lomustine (CCNU), larotaxel (RPR109881), mechlorethamine, mercaptopurine, methotrexate, mitomycin C, mitoxantrone, melphalan, mivobulin, 3′,4′-didehydro-4′-deoxy-8′-norvin-caleukoblastine, nilutamide, oxaliplatin, onapristone, prednimustine, procarbazine, paclitaxel, platinum-containing anti-cancer agents, 2,3,4,5,6-pentafluoro-N-(3-fluoro-4-methoxyphenyl)benzene sulfonamide, prednimustine, procarbazine, rhizoxin, sertenef, streptozocin, stramustine phosphate, tretinoin, tasonermin, taxol, topotecan, tamoxifen, teniposide, taxane, tegafur/uracil, vincristine, vinblastine, yinorelbine, vindesine, vindesine sulfate, and/or vinflunine.

Alternatively or in addition, the one or more chemotherapeutic agents may be selected from CD59 complement fragment, fibronectin fragment, gro-beta (CXCL2), heparinases, heparin hexasaccharide fragment, human chorionic gonadotropin (hCG), interferon alpha, interferon beta, interferon gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β) vasculostatin, vasostatin (calreticulin fragment), and/or cytokines (including interleukins, such as interleukin-2 (IL-2), or IL-10).

In some embodiments, the one or more antibodies may comprise one or more monoclonal antibodies. In some embodiments, the one or more antibodies are selected from abciximab, adalimumab, alemtuzumab, atlizumab, basiliximab, belimumab, bevacizumab, bretuximab vedotin, canakinumab, cetuximab, ceertolizumab pegol, daclizumab, denosumab, eculizumab, efalizumab, gemtuzumab, golimumab, ibritumomab tiuxetan, infliximab, ipilimumab, muromonab-CD₃, natalizumab, ofatumumab, omalizumab, paliyizumab, panitumuab, ranibizumab, rituximab, tocilizumab, tositumomab, and/or trastuzumab.

In some embodiments, the one or more alkylating agents may comprise an agent capable of alkylating nucleophilic functional groups under conditions present in cells, including, for example, cancer cells. In some embodiments, the one or more alkylating agents are selected from cisplatin, carboplatin, mechlorethamine, cyclophosphamide, chlorambucil, ifosfamide and/or oxaliplatin. In some embodiments, the alkylating agent may function by impairing cell function by forming covalent bonds with amino, carboxyl, sulfhydryl, and/or phosphate groups in biologically important molecules. In some embodiments, the alkylating agent may function by modifying a cell's DNA.

In some embodiments, the one or more anti-metabolites may comprise an agent capable of affecting or preventing RNA or DNA synthesis. In some embodiments, the one or more anti-metabolites are selected from azathioprine and/or mercaptopurine.

In some embodiments, the one or more anti-angiogenic agents are selected from endostatin, angiogenin inhibitors, angiostatin, angioarrestin, angiostatin (plasminogen fragment), basement-membrane collagen-derived anti-angiogenic factors (tumstatin, canstatin, or arrestin), anti-angiogenic antithrombin III, and/or cartilage-derived inhibitor (CDI).

In some embodiments, the one or more plant alkaloids and/or terpenoids may prevent microtubule function. In some embodiments, the one or more plant alkaloids and/or terpenoids are selected from a vinca alkaloid, a podophyllotoxin and/or a taxane. In some embodiments, the one or more vinca alkaloids may be derived from the Madagascar periwinkle, Catharanthus roseus (formerly known as Vinca rosea), and may be selected from vincristine, vinblastine, vinorelbine and/or vindesine. In some embodiments, the one or more taxanes are selected from taxol, paclitaxel, docetaxel and/or ortataxel. In some embodiments, the one or more podophyllotoxins are selected from an etoposide and/or teniposide.

In some embodiments, the one or more topoisomerase inhibitors are selected from a type I topoisomerase inhibitor and/or a type II topoisomerase inhibitor, and may interfere with transcription and/or replication of DNA by interfering with DNA supercoiling. In some embodiments, the one or more type I topoisomerase inhibitors may comprise a camptothecin, which may be selected from exatecan, irinotecan, lurtotecan, topotecan, BNP 1350, CKD 602, DB 67 (AR67) and/or ST 1481. In some embodiments, the one or more type II topoisomerase inhibitors may comprise an epipodophyllotoxin, which may be selected from an amsacrine, etoposid, etoposide phosphate and/or teniposide.

In some embodiments, the one or more mTOR (mammalian target of rapamycin, also known as the mechanistic target of rapamycin) inhibitors are selected from rapamycin, everolimus, temsirolimus and/or deforolimus.

In some embodiments, the one or more stilbenoids are selected from resveratrol, piceatannol, pinosylvin, pterostilbene, alpha-viniferin, ampelopsin A, ampelopsin E, diptoindonesin C, diptoindonesin F, epsilon-vinferin, flexuosol A, gnetin H, hemsleyanol D, hopeaphenol, trans-diptoindonesin B, astringin, piceid and/or diptoindonesin A.

In some embodiments, the one or more STING (Stimulator of interferon genes, also known as transmembrane protein (TMEM) 173) agonists may comprise cyclic di-nucleotides, such as cAMP, cGMP, and cGAMP, and/or modified cyclic di-nucleotides that may include one or more of the following modification features: 2′-O/3′-O linkage, phosphorothioate linkage, adenine and/or guanine analogue, and/or 2′-OH modification (e.g. protection of the 2′-OH with a methyl group or replacement of the 2′-OH by —F or —N₃).

In some embodiments, the one or more cancer vaccines are selected from an HPV vaccine, a hepatitis B vaccine, Oncophage, and/or Provenge.

In some embodiments, the one or more immunomodulatory agents may comprise an immune checkpoint inhibitor. The immune checkpoint inhibitor may target an immune checkpoint receptor, or combination of receptors comprising, for example, CTLA-4, PD-1, PD-L1, PD-L2, T cell immunoglobulin and mucin 3 (TIM3 or HAVCR2), galectin 9, phosphatidylserine, lymphocyte activation gene 3 protein (LAG3), MHC class I, MHC class II, 4-1BB, 4-1BBL, OX40, OX₄₀L, GITR, GITRL, CD27, CD70, TNFRSF25, TL1A, CD40, CD40L, HVEM, LIGHT, BTLA, CD160, CD80, CD244, CD48, ICOS, ICOSL, B7-H3, B7-H4, VISTA, TMIGD2, HHLA2, TMIGD2, a butyrophilin (including BTNL2), a Siglec family member, TIGIT, PVR, a killer-cell immunoglobulin-like receptor, an ILT, a leukocyte immunoglobulin-like receptor, NKG2D, NKG2A, MICA, MICB, CD28, CD86, SIRPA, CD47, VEGF, neuropilin, CD30, CD39, CD73, CXCR₄, and/or CXCL12.

In some embodiments, the immune checkpoint inhibitor is selected from urelumab, PF-05082566, MEDI6469, TRX518, varlilumab, CP-870893, pembrolizumab (PD1), nivolumab (PD1), atezolizumab (formerly MPDL3280A) (PD-L1), MEDI4736 (PD-L1), avelumab (PD-L1), PDR001 (PD1), BMS-986016, MGA271, lirilumab, IPH2201, emactuzumab, INCB024360, galunisertib, ulocuplumab, BKT140, bavituximab, CC-90002, bevacizumab, and/or MNRP1685A.

In some embodiments, the one or more antibiotics are selected from amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem, cilastatin, meropenem, cefadroxil, cefazolin, cefalotin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, calvulanate, ampicillin, subbactam, tazobactam, ticarcillin, clavulanate, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethoxazole, sulfanamide, sulfasalazine, sulfisoxazole, trimethoprim-sulfamethoxazole, sulfonamideochrysoidine, demeclocycline, minocycline, oytetracycline, tetracycline, clofazimine, dapsone, dapreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin, dalopristin, thiamphenicol, tigecycyline, tinidazole, trimethoprim, and/or teixobactin.

In some embodiments, the one or more antibiotics may comprise one or more cytotoxic antibiotics. In some embodiments, the one or more cytotoxic antibiotics are selected from an actinomycin, an anthracenedione, an anthracycline, thalidomide, dichloroacetic acid, nicotinic acid, 2-deoxyglucose, and/or chlofazimine. In some embodiments, the one or more actinomycins are selected from actinomycin D, bacitracin, colistin (polymyxin E) and/or polymyxin B. In some embodiments, the one or more antracenediones are selected from mitoxantrone and/or pixantrone. In some embodiments, the one or more anthracyclines are selected from bleomycin, doxorubicin (Adriamycin), daunorubicin (daunomycin), epirubicin, idarubicin, mitomycin, plicamycin and/or valrubicin.

In some embodiments, the one or more anti-fungal agents are selected from bifonazole, butoconazole, clotrimazole, econazole, ketoconazole, luliconazole, miconazole, omoconazole, oxiconazole, sertaconazole, sulconazole, tioconazole, albaconazole, efinaconazole, epoziconazole, fluconazole, isavuconazole, itraconazole, posaconazole, propiconazole, ravusconazole, terconazole, voriconazole, abafungin, amorolfin, butenafine, naftifine, terbinafine, anidulafungin, caspofungin, micafungin, benzoic acid, ciclopirox, flucytosine, 5-fluorocytosine, griseofulvin, haloprogin, tolnaflate, undecylenic acid, and/or balsam of Peru.

In some embodiments, the one or more anti-helminthic agents are selected from benzimidazoles (including albendazole, mebendazole, thiabendazole, fenbendazole, triclabendazole, and flubendazole), abamectin, diethylcarbamazine, ivermectin, suramin, pyrantel pamoate, levamisole, salicylanilides (including niclosamide and oxyclozanide), and/or nitazoxanide.

In some embodiments, other active agents are selected from growth inhibitory agents, anti-inflammatory agents (including nonsteroidal anti-inflammatory agents), anti-psoriatic agents (including anthralin and its derivatives), vitamins and vitamin-derivatives (including retinoinds, and VDR receptor ligands), corticosteroids, ion channel blockers (including potassium channel blockers), immune system regulators (including cyclosporin, FK 506, and glucocorticoids), lutenizing hormone releasing hormone agonists (such as leuprolidine, goserelin, triptorelin, histrelin, bicalutamide, flutamide and/or nilutamide), and/or hormones (including estrogen).

Unless stated otherwise, in any of the fifth to thirteenth aspects of the invention, the subject may be any human or other animal. Typically, the subject is a mammal, more typically a human or a domesticated mammal such as a cow, pig, lamb, sheep, goat, horse, cat, dog, rabbit, mouse etc. Most typically, the subject is a human.

Any of the medicaments employed in the present invention can be administered by oral, parenteral (including intravenous, subcutaneous, intramuscular, intradermal, intratracheal, intraperitoneal, intraarticular, intracranial and epidural), airway (aerosol), rectal, vaginal, ocular or topical (including transdermal, buccal, mucosal, sublingual and topical ocular) administration.

Typically, the mode of administration selected is that most appropriate to the disorder, disease or condition to be treated or prevented. Where one or more further active agents are administered, the mode of administration may be the same as or different to the mode of administration of the compound, salt, solvate, prodrug or pharmaceutical composition of the invention.

For oral administration, the compounds, salts, solvates or prodrugs of the present invention will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose. Corn starch and alginic acid are suitable disintegrating agents. Binding agents may include starch and gelatine. The lubricating agent, if present, may be magnesium stearate, stearic acid or talc. If desired, the tablets may be coated with a material, such as glyceryl monostearate or glyceryl distearate, to delay absorption in the gastrointestinal tract. Tablets may also be effervescent and/or dissolving tablets.

Capsules for oral use include hard gelatine capsules in which the active ingredient is mixed with a solid diluent, and soft gelatine capsules wherein the active ingredient is mixed with water or an oil such as peanut oil, liquid paraffin or olive oil.

Powders or granules for oral use may be provided in sachets or tubs. Aqueous solutions, suspensions or dispersions may be prepared by the addition of water to powders, granules or tablets.

Any form suitable for oral administration may optionally include sweetening agents such as sugar, flavouring agents, colouring agents and/or preservatives.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the compounds, salts, solvates or prodrugs of the present invention will generally be provided in a sterile aqueous solution or suspension, buffered to an appropriate pH and isotonicity. Suitable aqueous vehicles include Ringer's solution and isotonic sodium chloride or glucose. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and n-propyl p-hydroxybenzoate. The compounds of the invention may also be presented as liposome formulations.

For ocular administration, the compounds, salts, solvates or prodrugs of the invention will generally be provided in a form suitable for topical administration, e.g. as eye drops. Suitable forms may include ophthalmic solutions, gel-forming solutions, sterile powders for reconstitution, ophthalmic suspensions, ophthalmic ointments, ophthalmic emulsions, ophthalmic gels and ocular inserts. Alternatively, the compounds, salts, solvates or prodrugs of the invention may be provided in a form suitable for other types of ocular administration, for example as intraocular preparations (including as irrigating solutions, as intraocular, intravitreal or juxtascleral injection formulations, or as intravitreal implants), as packs or corneal shields, as intracameral, subconjunctival or retrobulbar injection formulations, or as iontophoresis formulations.

For transdermal and other topical administration, the compounds, salts, solvates or prodrugs of the invention will generally be provided in the form of ointments, cataplasms (poultices), pastes, powders, dressings, creams, plasters or patches.

Suitable suspensions and solutions can be used in inhalers for airway (aerosol) administration.

The dose of the compounds, salts, solvates or prodrugs of the present invention will, of course, vary with the disease, disorder or condition to be treated or prevented. In general, a suitable dose will be in the range of 0.01 to 500 mg per kilogram body weight of the recipient per day. The desired dose may be presented at an appropriate interval such as once every other day, once a day, twice a day, three times a day or four times a day. The desired dose may be administered in unit dosage form, for example, containing 1 mg to 50 g of active ingredient per unit dosage form.

For the avoidance of doubt, insofar as is practicable any embodiment of a given aspect of the present invention may occur in combination with any other embodiment of the same aspect of the present invention. In addition, insofar as is practicable it is to be understood that any preferred, typical or optional embodiment of any aspect of the present invention should also be considered as a preferred, typical or optional embodiment of any other aspect of the present invention.

### EXAMPLES

### Compound Synthesis

All solvents, reagents and compounds were purchased and used without further purification unless stated otherwise.

### Abbreviations

2-MeTHF 2-methyltetrahydrofuran
Ac₂O acetic anhydride
AcOH acetic acid
AIBN azobisisobutyronitrile
aq aqueous
Boc tert-butyloxycarbonyl
br broad
Cbz carboxybenzyl
CDI 1,1-carbonyl-diimidazole
conc concentrated
d doublet
DABCO 1,4-diazabicyclo[2.2.2]octane
DCE 1,2-dichloroethane, also called ethylene dichloride
DCM dichloromethane
DIPEA N,N-diisopropylethylamine, also called Hünig's base
DMA dimethylacetamide
DMAP 4-dimethylaminopyridine, also called N,N-dimethylpyridin-4-amine
DME dimethoxyethane
DMF N,N-dimethylformamide
DMSO dimethyl sulfoxide
EDC or EDCI N-(3-dimethylaminopropyl)-N′-ethylcarbodiimide, also called 1-ethyl-3-(3-dimethylaminopropyl)carbodiimide
eq or equiv equivalent
(ES⁺) electrospray ionization, positive mode
Et ethyl
EtOAc ethyl acetate
EtOH ethanol
h hour(s)
HATU 1-[bis(dimethylamino)methylene]-1H-1,2,3-triazolo[4,5-b]Pyridinium 3-oxid hexafluorophosphate
HPLC high performance liquid chromatography
KO^{t}Bu potassium tert-butoxide
LC liquid chromatography
m multiplet
m-CPBA 3-chloroperoxybenzoic acid
Me methyl
MeCN acetonitrile
MeOH methanol
(M+H)⁺ protonated molecular ion
MHz megahertz
min minute(s)
MS mass spectrometry
Ms mesyl, also called methanesulfonyl
MsCl mesyl chloride, also called methanesulfonyl chloride
MTBE methyl tert-butyl ether, also called tert-butyl methyl ether
m/z mass-to-charge ratio
NaO^{t}Bu sodium tert-butoxide
NBS 1-bromopyrrolidine-2,5-dione, also called N-bromosuccinimide
NCS 1-chloropyrrolidine-2,5-dione, also called N-chlorosuccinimide
NMP N-methylpyrrolidine
NMR nuclear magnetic resonance (spectroscopy)
Pd(crotyl)(XPhos)Cl chloro(crotyl)(2-dicyclohexylphosphino-2′,4′,6′-triisopropybiphenyl)palladium(II)
Pd₂(dba)₃ tris(dibenzylideneacetone) dipalladium(0)
Pd(dba)₂ bis(dibenzylideneacetone) palladium(0)
Pd(dba)₃ tris(dibenzylideneacetone) dipalladium(0)
Pd(dppf)Cl₂ [1,1′-bis(diphenylphosphino)ferrocene] dichloropalladium(II)
PE petroleum ether
Ph phenyl
PMB p-methoxybenzyl, also called 4-methoxybenzyl
prep-HPLC preparative high performance liquid chromatography
prep-TLC preparative thin layer chromatography
PTSA p-toluenesulfonic acid
q quartet
RP reversed phase
RT room temperature
s singlet
sat saturated
SCX solid supported cation exchange (resin)
Selectfluor® 1-chloromethyl-4-fluoro-1,4-diazoniabicyclo[2.2.2]octane bis(tetrafluoroborate), also called N-chloromethyl-N′-fluorotriethylenediammonium bis(tetrafluoroborate)
sept septuplet
t triplet
T3P propylphosphonic anhydride
TBME tert-butyl methyl ether, also called methyl tert-butyl ether
TEA triethylamine
TFA 2,2,2-trifluoroacetic acid
THF tetrahydrofuran
TLC thin layer chromatography
TMSCl trimethylsilyl chloride
wt % weight percent or percent by weight
Xantphos® 4,5-bis(diphenylphosphino)-9,9-dimethylxanthene
Xphos® 2-dicyclohexylphosphino-2′,4′,6′-triisopropylbiphenyl

Experimental Methods

Nuclear Magnetic Resonance

NMR spectra were recorded at 300, 400 or 500 MHz. Spectra were measured at 298 K, unless indicated otherwise, and were referenced relative to the solvent resonance. The chemical shifts are reported in parts per million. Spectra were recorded using one of the following machines:
a Bruker Avance III spectrometer at 400 MHz fitted with a BBO 5 mm liquid probe,
a Bruker 400 MHz spectrometer using ICON-NMR, under TopSpin program control,
a Bruker Avance III HD spectrometer at 500 MHz, equipped with a Bruker 5 mm SmartProbe™,
an Agilent VNMRS 300 instrument fitted with a 7.05 Tesla magnet from Oxford instruments, indirect detection probe and direct drive console including PFG module, or
an Agilent MercuryPlus 300 instrument fitted with a 7.05 Tesla magnet from Oxford instruments, 4 nuclei auto-switchable probe and Mercury plus console.

LC-MS

LC-MS Methods: Using SHIMADZU LCMS-2020, Agilent 1200 LC/G1956A MSD and Agilent 1200\G6110A, Agilent 1200 LC & Agilent 6110 MSD. Mobile Phase: A: 0.025% NH₃.H₂O in water (v/v); B: acetonitrile. Column: Kinetex EVO C18 2.1×30 mm, 5 μm.

Reversed Phase HPLC Conditions for the LCMS Analytical Methods

Methods 1a and 1b: Waters Xselect CSH C18 XP column (4.6×30 mm, 2.5 μm) at 40° C.; flow rate 2.5-4.5 mL min⁻¹ eluted with a H₂O-MeCN gradient containing either 0.1% v/v formic acid (Method 1a) or 10 mM NH₄HCO₃ in water (Method 1b) over 4 min employing UV detection at 254 nm. Gradient information: 0-3.00 min, ramped from 95% water-5% acetonitrile to 5% water-95% acetonitrile; 3.00-3.01 min, held at 5% water-95% acetonitrile, flow rate increased to 4.5 mL min⁻¹; 3.01-3.50 min, held at 5% water-95% acetonitrile; 3.50-3.60 min, returned to 95% water-5% acetonitrile, flow rate reduced to 3.50 mL min⁻¹; 3.60-3.90 min, held at 95% water-5% acetonitrile; 3.90-4.00 min, held at 95% water-5% acetonitrile, flow rate reduced to 2.5 mL min⁻¹.

Methods 1a′ and 1b′: UPLC/MS analysis was carried out using either a Waters Acquity CSH C18 or BEH C18 column (2.1×30 mm) maintained at a temperature of 40° C. and eluted with a linear acetonitrile gradient appropriate for the lipophilicity of the compound over 3 or 10 minutes at a constant flow rate of 0.77 ml/min. The aqueous portion of the mobile phase was either 0.1% v/v formic acid (CSH C18 column) (Method 1a′) or 10 mM ammonium bicarbonate (BEH C18 column) (Method 1b′). LC-LTV chromatograms were recorded using a Waters Acquity PDA detector between 210 and 400 nm. Mass spectra were recorded using a Waters Acquity QDa detector with electrospray ionisation switching between positive and negative ion mode. Sample concentration was adjusted to give adequate UV response.

Method 1c: Agilent 1290 series with UV detector and HP 6130 MSD mass detector using Waters XBridge BEH C18 XP column (2.1×50 mm, 2.5 μm) at 35° C.; flow rate 0.6 mL/min; mobile phase A: ammonium acetate (10 mM); water/MeOH/acetonitrile (900:60:40); mobile phase B: ammonium acetate (10 mM); water/MeOH/acetonitrile (100:540:360); over 4 min employing UV detection at 215 and 238 nm. Gradient information: 0-0.5 min, held at 80% A-20% B; 0.5-2.0 min, ramped from 80% A-20% B to 100% B.

Preparative Reversed Phase HPLC General Methods

Method 1 (basic preparation): Waters X-Bridge Prep column C18, 5 μm (19×50 mm), flow rate 28 mL min⁻¹ eluting with a 10 mM NH₄HCO₃/MeCN gradient over 6.5 min using UV detection at 254 nm. Gradient information: 0.0-0.2 min, 10% MeCN; 0.2-5.5 min, ramped from 10% MeCN to 40% MeCN; 5.5-5.6 min, ramped from 40% MeCN to 95% MeCN; 5.6-6.5 min, held at 95% MeCN.

Method 2: Revelis C18 reversed-phase 12 g cartridge [carbon loading 18%; surface area 568 m²/g; pore diameter 65 Angstrom; pH (5% slurry) 5.1; average particle size 40 μm], flow rate=30 mL/min eluting with a water/methanol gradient over 35 min using UV detection at 215, 235, 254 and 280 nm. Gradient information: 0-5 min, held at 0% methanol; 5-30 min, ramped from 0% to 70% methanol; 30-30.1 min, ramped from 70% to 100% methanol; 30.1-35 min, held at 100% methanol.

Method 3: XSelect CSH Prep C18 OBD, 5 μm (100×30 mm), eluting with a 10 mM ammonium acetate/MeCN gradient over 12 min. Gradient information: 0-2.5 min, 15% MeCN; 2.5-10 min, ramped from 15% to 35% MeCN; 10-10.1 min, ramped from 35% to 95% MeCN; 10.1-12 min, held at 95% MeCN.

Method 4 (acidic preparation): Waters X-Select CSH column C18, 5 μm (19×50 mm), flow rate 28 mL min⁻¹ eluting with H₂O-MeCN gradient containing 0.1% v/v formic acid over 6.5 min using UV detection at 254 nm. Gradient information: 0.0-0.2 min, 25% MeCN; 0.2-5.5 min, ramped from 25% MeCN to 55% MeCN; 5.5-5.6 min, ramped from 55% MeCN to 95% MeCN; 5.6-6.5 min, held at 95% MeCN.

Synthesis of Intermediates

### Intermediate L1: 4-(2-Hydroxypropan-2-yl)-2-methylbenzenesulfonamide

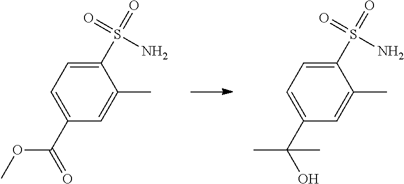

Methyl 3-methyl-4-sulfamoylbenzoate (486 mg, 2.12 mmol) was stirred in THF (20 mL) and a solution of methyl magnesium bromide in Et₂O (3M, 4 mL, 12 mmol) was added dropwise. The mixture was stirred at room temperature for 2 days and poured into sat aq NaHCO₃ (aqueous, 20 mL), and concentrated partially (THF removal) and filtered. EtOAc (20 mL) was used to wash the residual solid and extract the filtrate. The organic phase was separated, dried (Na₂SO₄), filtered and concentrated to afford the title compound (0.48 g, 99%) as a yellow solid.

¹H NMR (300 MHz, Methanol-d₄) δ 7.89 (d, 1H), 7.47 (s, 1H), 7.45-7.39 (d, 1H), 2.66 (s, 3H), 1.52 (s, 6H).

### Intermediate L2: 1-(Propan-2-yl-d7)-1H-pyrazole-3-sulfonamide

### Step A: N,N-Bis(4-methoxybenzyl)-1-(propan-2-yl-d7)-1H-pyrazole-3-sulfonamide

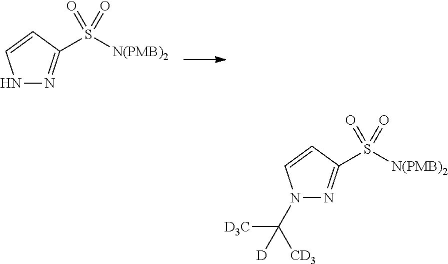

To a solution of N,N-bis(4-methoxybenzyl)-1H-pyrazole-3-sulfonamide (Intermediate L19, Step C) (200 mg, 0.52 mmol) in dimethylformamide (2 mL), was added K₂CO₃(100 mg, 0.72 mmol) and 2-iodopropane-d7 (110 mg, (0.62 mmol). The mixture was stirred for 7 days at room temperature in the dark. The reaction mixture was poured into water (10 mL) and the water layer was extracted with ethyl acetate. The organic layer was washed with water (4 times) and brine. The organic layer was dried (sodium sulfate), filtered and evaporated. The residue was purified over silica, using ethyl acetate/heptane (1:1) as the eluent, to afford the title compound as a colourless oil (140 mg, 62%).

¹H NMR (300 MHz, CDCl₃) δ 7.46 (s, 1H), 7.08 (d, 4H), 6.78 (d, 4H), 6.68 (d, 1H), 4.33 (s, 4H), 3.80 (s, 6H).

### Step B: 1-(Propan-2-yl-d7)-1H-pyrazole-3-sulfonamide

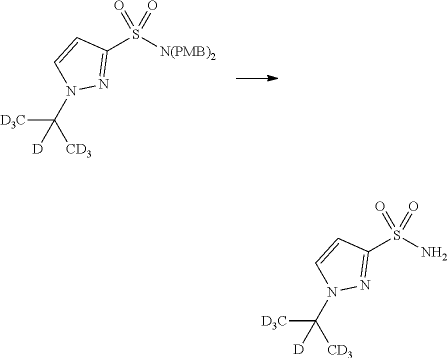

N,N-Bis(4-methoxybenzyl)-1-(propan-2-yl-d7)-1H-pyrazole-3-sulfonamide (140 mg, 0.32 mmol) was dissolved in dichloromethane (5 mL). Trifluoroacetic acid (1 mL) was added and the reaction was stirred for 3 days at room temperature. The solvents were evaporated and the residue was triturated with water. The water layer was filtered and lyophilized to afford the title compound as a white solid (67 mg, 100%).

¹H NMR (300 MHz, D₂O) δ 7.73 (d, 1H), 7.67 (d, 1H).

### Intermediate L3: tert-Butyl 3-sulfamoyl-1H-pyrazole-1-carboxylate

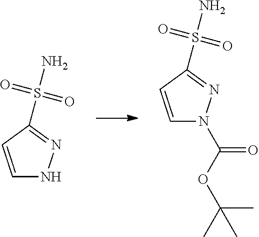 [SMILES: NS(=O)(=O)c1cc[nH]n1 ; CC(C)(C)OC(=O)n1ccc(S(N)(=O)=O)n1]

To a suspension of 1H-pyrazole-3-sulfonamide (103 mg, 0.69 mmol) and Et₃N (0.14 mL, 1.05 mmol) in anhydrous DCM (2 mL) was added di-tert-butyl dicarbonate (188 mg, 0.83 mmol) and the reaction mixture was stirred at room temperature overnight. A sat aq solution of NaHCO₃ was added to the mixture and the aqueous phase extracted with DCM. The organic layer was washed with water and dried over Na₂SO₄. Concentration under reduced pressure gave the title compound as a yellow oil (170 mg, 98%) that was used without further purification.

¹H NMR (CD₃OD) δ 7.74 (d, 1H), 6.70 (d, 1H), 1.51 (s, 9H).

### Intermediate L4: 1-(2-(3-(But-3-yn-1-yl)-3H-diazirin-3-yl)ethyl)-1H-pyrazole-3-sulfonamide, 2,2,2-trifluoroacetate salt

### Step A: 1-(2-(3-(But-3-yn-1-yl)-3H-diazirin-3-yl)ethyl)-N,N-bis(4-methoxybenzyl)-1H-pyrazole-3-sulfonamide

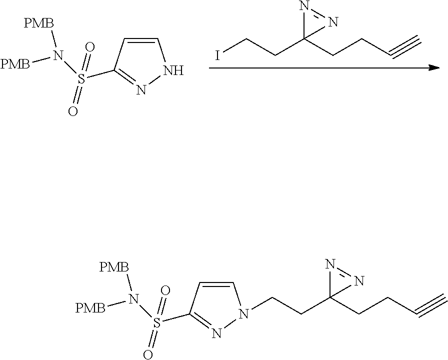

To a solution of N,N-bis(4-methoxybenzyl)-1H-pyrazole-3-sulfonamide (Intermediate L19, Step C) (58 mg, 0.15 mmol, 1.25 eq) in acetonitrile (3 mL) was added 3-(but-3-yn-1-yl)-3-(2-iodoethyl)-3H-diazirine (30 mg, 0.12 mol, 1 eq) and potassium carbonate (62 mg, 0.45 mmol, 3.75 eq). The reaction mixture was covered in aluminium foil and then stirred at room temperature. After stirring overnight, extra N,N-bis(4-methoxybenzyl)-1H-pyrazole-3-sulfonamide (Intermediate L19, Step C) (23 mg, 0.06 mmol, 0.5 eq) in acetonitrile (0.5 mL) was added to the reaction mixture. After stirring for 3 hours, the reaction mixture was diluted with water, and then extracted twice with dichloromethane. The organic layers were combined, dried over sodium sulfate, filtered and then concentrated in vacuo. The crude product was submitted to normal phase purification on silica using heptane and ethyl acetate as eluent to afford the title compound (24 mg, 47 μmol, 39%).

¹H NMR (CDCl₃) δ 7.49 (d, 1H), 7.07 (d, 4H), 6.77 (d, 4H), 6.65 (d, 1H), 4.30 (s, 4H), 4.02 (t, 2H), 3.78 (s, 6H), 2.04 (t, 2H), 2.01-1.96 (m, 1H), 1.93 (dd, 2H), 1.57-1.47 (m, 2H).

### Step B: 1-(2-(3-(But-3-yn-1-yl)-3H-diazirin-3-yl)ethyl)-1H-pyrazole-3-sulfonamide, 2,2,2-trifluoroacetate salt

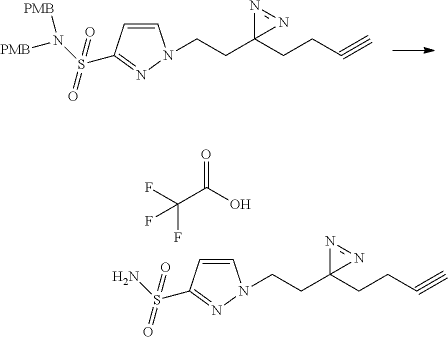

A solution of 1-(2-(3-(but-3-yn-1-yl)-3H-diazirin-3-yl)ethyl)-N,N-bis(4-methoxybenzyl)-1H-pyrazole-3-sulfonamide (24 mg, 47 μmol, 1 eq) in trifluoroacetic acid (1.0 mL, 13.05 mmol, 277 eq) was stirred at room temperature. After four hours, toluene was added to the reaction mixture and then the mixture was concentrated in vacuo to afford the title compound (17 mg, 47 μmol, quantitative yield), which was used as such for the following reaction.

¹H NMR (CD₃OD) δ 7.78 (d, 1H), 6.66 (d, 1H), 4.13 (t, 2H), 2.32-2.22 (m, 1H), 2.05-1.90 (m, 4H), 1.52 (t, 2H).

### Intermediate L5: 1-(2-(Bis(methyl-d3)amino)ethyl)-1H-pyrazole-3-sulfonamide, 2,2,2-trifluoroacetate salt

### Step A: 1-(2-Hydroxyethyl)-N,N-bis(4-methoxybenzyl)-1H-pyrazole-3-sulfonamide

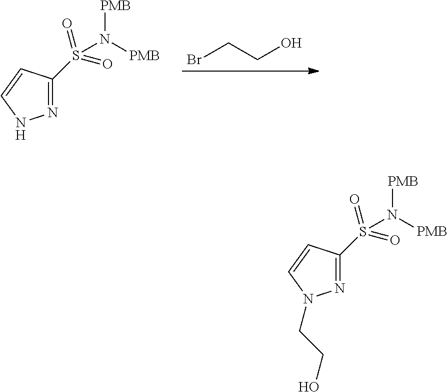

To a solution of N,N-bis(4-methoxybenzyl)-1H-pyrazole-3-sulfonamide (Intermediate L19, Step C) (1.5 g, 3.9 mmol, 1 eq) in acetonitrile (20 mL) was added 2-bromoethan-1-ol (0.57 mL, 7.7 mmol, 2 eq), potassium iodide (64 mg, 0.39 mmol, 0.1 eq) and potassium carbonate (1.6 g, 12.0 mmol, 3 eq). The reaction mixture was heated to 60° C. After stirring overnight, extra 2-bromoethan-1-ol (0.14 mL, 1.95 mmol, 0.5 eq) was added. After stirring for a further 4 hours, the reaction mixture was cooled to room temperature and then diluted with water. The mixture was extracted with dichloromethane. The organic layer was washed with brine, dried over sodium sulfate, filtered and then concentrated in vacuo. The crude material was submitted to normal phase flash chromatography using heptane and ethyl acetate as eluent to afford the title compound (1.08 g, 2.50 mmol, 65%).

¹H NMR (CDCl₃) δ 7.56-7.44 (m, 1H), 7.07 (dd, 4H), 6.85-6.73 (m, 4H), 6.70-6.54 (m, 1H), 4.36-4.20 (m, 6H), 4.02-3.88 (m, 2H), 3.78 (s, 6H), 2.21 (s, 1H).

### Step B: 2-(3-(N,N-Bis(4-methoxybenzyl)sulfamoyl)-1H-pyrazol-1-yl)ethyl methanesulfonate

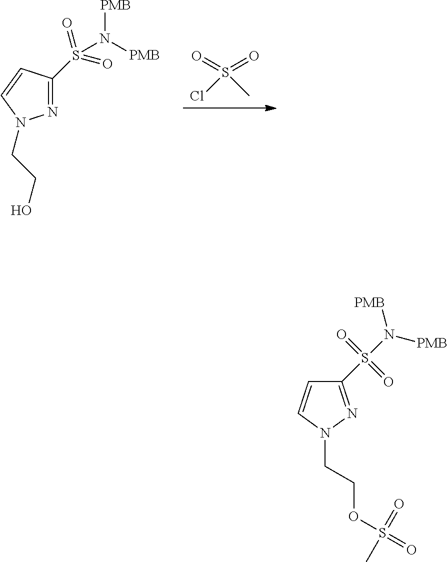

To a solution of 1-(2-hydroxyethyl)-N,N-bis(4-methoxybenzyl)-1H-pyrazole-3-sulfonamide (780 mg, 1.81 mmol, 1 eq) and N,N-diisopropylethylamine (0.44 mL, 2.53 mmol, 1.4 eq) in dichloromethane (25 mL) was added dropwise methanesulfonyl chloride (0.16 mL, 2.17 mmol, 1.2 eq) at room temperature. After stirring for 40 minutes, the reaction mixture was diluted with dichloromethane (50 mL), then washed twice with sat aq sodium bicarbonate, once with brine, dried over sodium sulfate, filtered and concentrated in vacuo to afford the title compound (921 mg, 1.81 mmol, quantitative yield), which was used as such in the following reaction.

¹H NMR (CDCl₃) δ 7.54 (q, 1H), 7.12-7.00 (m, 4H), 6.84-6.69 (m, 4H), 6.65 (q, 1H), 4.65-4.46 (m, 4H), 4.31 (s, 4H), 3.78 (s, 6H), 2.92 (s, 3H).

### Step C: 1-(2-(Bis(methyl-d3)amino)ethyl)-N,N-bis(4-methoxybenzyl)-1H-pyrazole-3-sulfonamide

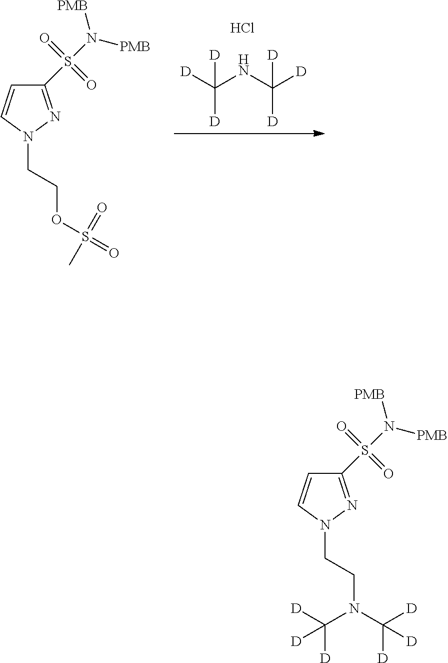

A microwave vial was charged with 2-(3-(N,N-bis(4-methoxybenzyl)sulfamoyl)-1H-pyrazol-1-yl)ethyl methanesulfonate (922 mg, 1.81 mmol, 1 eq), bis(methyl-d3)amine hydrochloride (634 mg, 7.24 mmol, 4 eq), potassium iodide (601 mg, 3.62 mmol, 2 eq), N,N-diisopropylethylamine (3.2 mL, 18.1 mmol, 10 eq) and acetonitrile (15 mL). The microwave vial was capped and then heated in a sand bath set to 100° C. After 1 hour, the reaction mixture was cooled to room temperature and then water was added. The mixture was extracted with dichloromethane. The organic layer was washed with brine, dried over sodium sulfate, filtered and then concentrated in vacuo. The crude product was submitted to normal phase flash chromatography using dichloromethane and methanol as eluent to afford the title compound (541 mg, 1.16 mmol, 64%).

¹H NMR (CDCl₃) δ 7.53 (d, 1H), 7.11-7.00 (m, 4H), 6.82-6.70 (m, 4H), 6.62 (d, 1H), 4.30 (s, 4H), 4.26 (t, 2H), 3.78 (s, 6H), 2.74 (t, 2H).

### Step D: 1-(2-(Bis(methyl-d3)amino)ethyl)-1H-pyrazole-3-sulfonamide, 2,2,2-trifluoroacetate salt

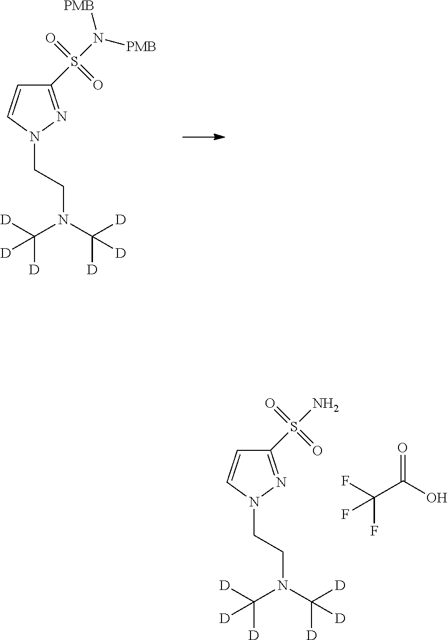

To a solution of 1-(2-(bis(methyl-d3)amino)ethyl)-N,N-bis(4-methoxybenzyl)-1H-pyrazole-3-sulfonamide (541 mg, 1.16 mmol, 1 eq) in dichloromethane (5 mL) was added trifluoroacetic acid (5.0 mL, 64.9 mmol, 56 eq). The reaction mixture was stirred at room temperature. After stirring overnight, the mixture was concentrated in vacuo. The crude product was suspended in methanol, filtered and the residue was washed with methanol. The filtrates were combined and concentrated in vacuo to afford the title compound as a trifluoroacetate salt (394 mg, 1.16 mmol, quantitative yield), which was used as such in the following reaction.

¹H NMR (CD₃OD) δ 7.85 (d, 1H), 6.75 (d, 1H), 4.72-4.61 (m, 2H), 3.76-3.61 (m, 2H).

### Intermediate L6: 1-Isopropyl-1H-pyrazole-3-sulfinamide

### Step A: 1-Isopropyl-3-nitro-1H-pyrazole

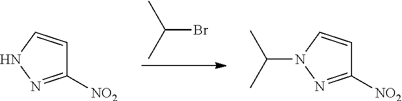

To a solution of 3-nitro-1H-pyrazole (75 g, 663.3 mmol, 1 eq) in anhydrous DMF (500 mL) was added NaH (29 g, 729.6 mmol, 60% purity in mineral oil, 1.1 eq) under nitrogen at 0° C. The reaction mixture was stirred for 30 minutes. 2-Bromopropane (98 g, 795.9 mmol, 1.2 eq) was added. Stirring was continued at 0° C. for 30 minutes under nitrogen. The ice bath was removed and the reaction mixture was stirred at 20° C. for 15 hours. The resulting mixture was quenched with water (500 mL) and extracted with ethyl acetate (2 L). The organic layer was washed with water (2×500 mL) and brine (2×500 mL), dried over anhydrous Na₂SO₄, filtered and concentrated in vacuo. The residue was purified by column chromatography (SiO₂, petroleum ether:ethyl acetate, 50:1 to 5:1) to give the title compound (60 g, 58%) as a white solid.

¹H NMR (400 MHz, CDCl₃) δ 7.49 (d, 1H), 6.82 (d, 1H), 4.59-4.46 (m, 1H) and 1.51 (d, 6H).

### Step B: 1-Isopropyl-1H-pyrazol-3-amine

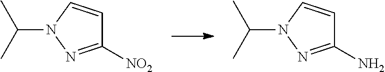

To a solution of 1-isopropyl-3-nitro-1H-pyrazole (50 g, 322.26 mmol, 1 eq) and NH₄Cl (86 g, 1.61 mol, 5 eq) in EtOH (500 mL) and H₂O (300 mL) was added Fe (36 g, 644.52 mmol, 2 eq) as powder in portions at 60° C. The reaction mixture was stirred at 60° C. for 16 hours, and then concentrated under reduced pressure to remove EtOH. The residue was diluted with H₂O (1 L) and extracted with EtOAc (3×1 L). The combined organic layers were washed with brine (2×500 mL), dried over Na₂SO₄, filtered and concentrated under reduced pressure to give the title compound (39 g, crude) as a blue oil, which was used directly into the next step.

¹H NMR (CDCl₃) δ 7.15 (d, 1H), 5.55 (d, 1H), 4.31-4.20 (m, 1H), 3.60 (br s, 2H) and 1.43 (d, 6H).

LCMS: m/z 126.2 (M+H)⁺ (ES⁺).

### Step C: 1-Isopropyl-1H-pyrazole-3-sulfonyl chloride

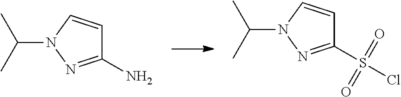

A solution of 1-isopropyl-1H-pyrazol-3-amine (23 g, 183.75 mmol, 1 eq) in MeCN (500 mL) at 0° C. was treated with a solution of concentrated HCl (50 mL, 36 wt % in H₂O) in H₂O (50 mL), followed slowly by an aqueous solution of NaNO₂ (15.2 g, 220.50 mmol, 1.2 eq) in H₂O (50 mL). The resulting mixture was stirred at 0° C. for 40 minutes. AcOH (50 mL), CuCl₂ (12.4 g, 91.87 mmol, 0.5 eq), and CuCl (909 mg, 9.19 mmol, 0.05 eq) were added. Then SO₂ gas (15 psi) was bubbled into the mixture at 0° C. for 20 minutes. The reaction mixture was stirred at 0° C. for 1 hour, and then concentrated under reduced pressure to remove most of MeCN and AcOH. The residue was diluted with H₂O (500 mL) and extracted with EtOAc (3×500 mL). The combined organic layers were washed with brine (2×300 mL), dried over Na₂SO₄, filtered and concentrated under reduced pressure. The residue was purified by column chromatography (SiO₂, petroleum ether:ethyl acetate, 30:1 to 10:1) to give the title compound (16.5 g, 43%) as a yellow oil.

¹H NMR (400 MHz, CDCl₃) δ 7.59-7.57 (m, 1H), 6.87-6.85 (m, 1H), 4.70-4.59 (m, 1H) and 1.57 (dd, 6H).

### Step D: Sodium 1-isopropyl-1H-pyrazole-3-sulfinate

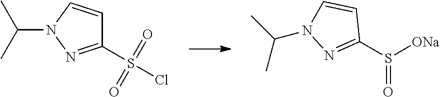

A solution of Na₂SO₃ (4.35 g, 34.50 mmol, 2 eq) in H₂O (12 mL) was stirred at 20° C. for 10 minutes. Then Na₂CO₃ (3.66 g, 34.50 mmol, 2 eq) was added. The resulting mixture was stirred at 50° C. for 10 minutes. 1-Isopropyl-1H-pyrazole-3-sulfonyl chloride (3.6 g, 17.25 mmol, 1 eq) was added dropwise. The resulting mixture was stirred at 50° C. for 2 hours, and then evaporated in vacuo. The residue was treated with EtOH (24 mL). The suspension was allowed to stir at 20° C. for 10 minutes. The suspension was filtered and the filtrate was evaporated to afford a white solid. The white solid was treated with ethyl acetate (20 mL) for 10 minutes and then the mixture was filtered. The filter cake was collected and dried to afford the title compound (2.4 g, 67% yield, 95% purity on LCMS) as a white solid.

¹H NMR (400 MHz, DMSO-d₆) δ7.58 (s, 1H), 6.17 (s, 1H), 4.46-4.43 (m, 1H) and 1.37 (d, 6H).

LCMS: m/z 197 (M+H)⁺(ES⁺).

### Step E: 1-Isopropyl-1H-pyrazole-3-sulfinamide

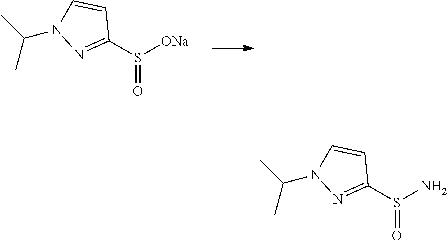

To a solution of sodium 1-isopropyl-1H-pyrazole-3-sulfinate (2.4 g, 12.23 mmol, 1 eq) in THF (15 mL) was added dropwise oxalyl dichloride (3.11 g, 24.46 mmol, 2 eq) at 0° C. After being stirred at 20° C. for 1 hour, the reaction mixture was added to NH₃.H₂O (15 mL, 25 wt % in H₂O) at 0° C. and then stirred at 20° C. for 1 hour. The reaction mixture was concentrated under reduced pressure. The residue was treated with DCM (20 mL). The mixture was stirred at 20° C. for 20 minutes, and then filtered. The filtrate was concentrated under reduced pressure to give the title compound (1.2 g, 51% yield, 90% purity on LCMS) as a yellow oil.

¹H NMR (400 MHz, DMSO-d₆) δ 7.87 (d, 1H), 6.55 (d, 1H), 6.26 (s, 2H), 4.58-4.51 (m, 1H) and 1.42 (d, 6H).

LCMS: m/z 196 (M+Na)⁺(ES⁺).

### Intermediate L7: tert-Butyl 3-sulfinamoylazetidine-1-carboxylate

### Step A: tert-Butyl 3-((methylsulfonyl)oxy)azetidine-1-carboxylate

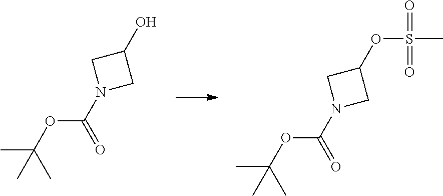 [SMILES: CC(C)(C)OC(=O)N1CC(O)C1 ; CC(C)(C)OC(=O)N1CC(OS(C)(=O)=O)C1]

To a solution of tert-butyl 3-hydroxyazetidine-1-carboxylate (50 g, 288.67 mmol, 1 eq) and TEA (87.63 g, 866.01 mmol, 3 eq) in THF (500 mL) was added methanesulfonyl chloride (40 g, 346.40 mmol, 1.2 eq) at 0° C. The reaction mixture was stirred at 25° C. for 12 hours, and then diluted with ethyl acetate (2 L). The organic layer was washed with water (3×1.5 L), brine (3×1.5 L), dried over anhydrous Na₂SO₄, filtered and concentrated in vacuo to give the title compound (70 g, 97%) as a yellow oil.

¹H NMR (400 MHz, CDCl₃) δ5.23-5.19 (m, 1H), 4.30-4.26 (m, 2H), 4.13-4.11 (m, ₂H), 3.08 (s, 3H) and 1.45 (s, 9H).

### Step B: tert-Butyl 3-(acetylthio)azetidine-1-carboxylate

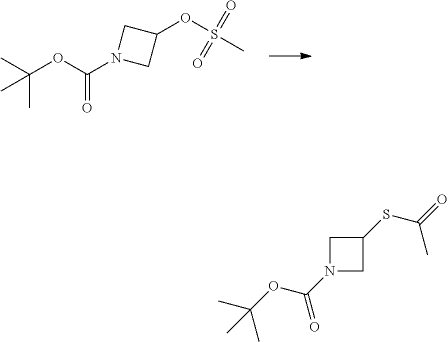 [SMILES: CC(C)(C)OC(=O)N1CC(OS(C)(=O)=O)C1 ; CC(=O)SC1CN(C(=O)OC(C)(C)C)C1]

To a solution of tert-butyl 3-((methylsulfonyl)oxy)azetidine-1-carboxylate (35 g, 139.28 mmol, 1 eq) in DMF (360 mL) was added potassium ethanethioate (19 g, 167.13 mmol, 1.2 eq). The reaction mixture was stirred at 80° C. for 12 hours, and then diluted with ethyl acetate (1.5 L). The organic layer was washed with saturated aqueous NH₄Cl solution (3×1 L), brine (3×1 L), dried over anhydrous Na₂SO₄, filtered and concentrated to give crude product. The crude product was purified by column chromatography (SiO₂, petroleum ether:ethyl acetate, 100:1 to 20:1) to give the title compound (26 g, 81%) as a yellow oil.

¹H NMR (400 MHz, CDCl₃) δ 4.30 (t, 2H), 4.11-4.07 (m, 1H), 3.76-3.72 (m, 2H), 2.27 (s, 3H) and 1.36 (s, 9H).

### Step C: tert-Butyl 3-mercaptoazetidine-1-carboxylate

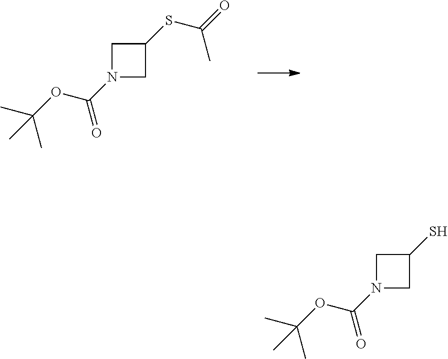

To a solution of tert-butyl 3-(acetylthio)azetidine-1-carboxylate (20 g, 86.46 mmol, 1 eq) in MeOH (80 mL), THF (80 mL) and H₂O (40 mL) was added LiOH.H₂O (3.63 g, 86.46 mmol, 1 eq). The reaction mixture was stirred at 70° C. for 2 hours, poured into water (200 mL), and extracted with ethyl acetate (3×200 mL). The combined organic layers were dried over anhydrous Na₂SO₄, filtered and concentrated to give the title compound (14 g, 86%) as a yellow oil.

¹H NMR (400 MHz, CDCl₃) δ 4.37-4.32 (m, 2H), 3.82-3.77 (m, 2H), 3.70-3.59 (m, 1H) and 1.44 (s, 9H).

### Step D: tert-Butyl 3-(methoxysulfinyl)azetidine-1-carboxylate

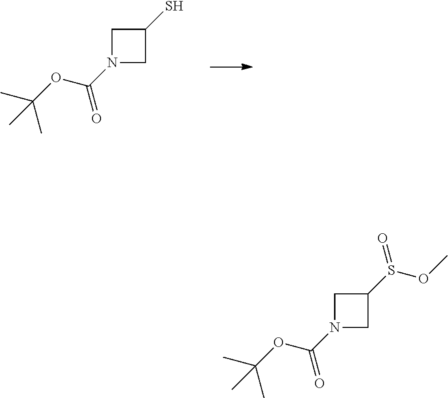

To a solution of tert-butyl 3-mercaptoazetidine-1-carboxylate (6.5 g, 34.34 mmol, 1 eq) in MeOH (130 mL) was added NBS (12.2 g, 68.68 mmol, 2 eq). The reaction mixture was stirred at 25° C. for 10 minutes, quenched with saturated aqueous Na₂SO₃ solution (200 mL), and extracted with ethyl acetate (3×200 mL). The combined organic layers were dried over anhydrous Na₂SO₄, filtered and concentrated to give crude product. The crude product was purified by column chromatography (SiO₂, petroleum ether:ethyl acetate, 10:1 to 5:1) to give the title compound (5 g, 62%) as a yellow oil.

¹H NMR (400 MHz, CDCl₃) δ 4.33-4.29 (m, 1H), 4.14-4.08 (m, 3H), 3.83 (s, 3H), 3.66-3.62 (m, 1H) and 1.45 (s, 9H).

LCMS: m/z 258.1 (M+Na)⁺ (ES⁺).

### Step E: Sodium 1-(tert-butoxycarbonyl)azetidine-3-sulfinate

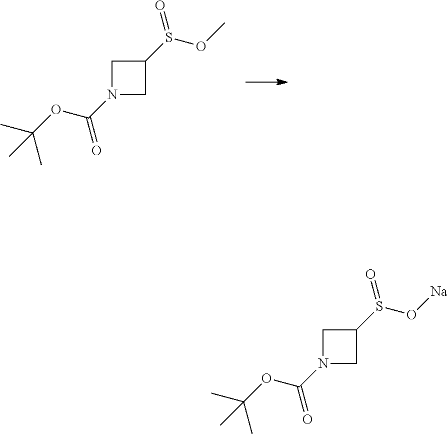

To a solution of tert-butyl 3-(methoxysulfinyl)azetidine-1-carboxylate (5 g, 21.25 mmol, 1 eq) in MeOH (50 mL) was added NaOH (1 M, 31.87 mL, 1.5 eq). The reaction mixture was stirred at 25° C. for 1 hour, adjusted to pH=7 with 1 N aqueous HCl solution, and concentrated in vacuo to remove MeOH. The residue was purified by column chromatography (SiO₂, petroleum ether:ethyl acetate, 1:1 to 0:1) to give the title compound (5 g, 97%) as a white solid.

¹H NMR (400 MHz, DMSO-d₆) δ 3.82-3.80 (m, 2H), 3.66-3.64 (m, 2H), 2.72-2.65 (m, 1H) and 1.36 (s, 9H).

### Step F: tert-Butyl 3-sulfinamoylazetidine-1-carboxylate

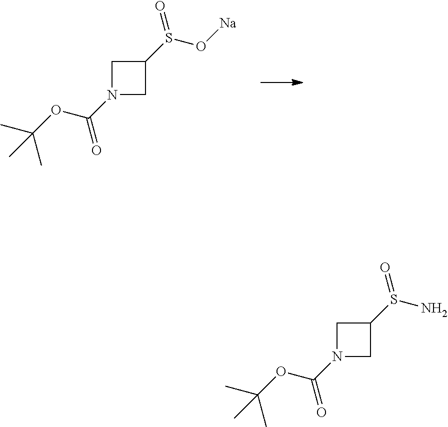

To a solution of sodium 1-(tert-butoxycarbonyl)azetidine-3-sulfinate (5 g, 20.55 mmol, 1 eq) in THF (350 mL) was added (COCl)₂ (5.2 g, 41.11 mmol, 2 eq). The reaction mixture was stirred at 25° C. for 2 hours. In a different vessel, NH₃ (15 psi) was bubbled into THF (20 mL) at −78° C. for 10 minutes. Then the above reaction mixture was added into NH₃/THF (200 mL) at −78° C. The resulting reaction mixture was stirred at 25° C. for another 50 minutes. The reaction mixture was filtered, and the filtrate was concentrated in vacuo. The residue was purified by column chromatography (SiO₂, petroleum ether:ethyl acetate, 1:1 to 0:1) to give the title compound (2.5 g, 55%) as a colourless oil.

¹H NMR (400 MHz, DMSO-d₆) δ 5.84 (s, 2H), 4.08-3.99 (m, 3H), 3.77-3.75 (m, 1H), 3.63-3.58 (m, 1H) and 1.38 (s, 9H).

### Intermediate L8: 1-Isopropyl-1H-pyrazole-3-sulfonamide

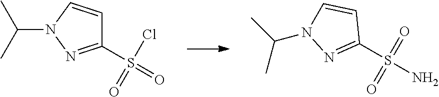

NH₃ gas (15 psi) was bubbled into a solution of 1-isopropyl-1H-pyrazole-3-sulfonyl chloride (Intermediate L6, Step C) (20 g, 47.9 mmol, 1 eq) in THF (300 mL) at 0° C. for 15 minutes. The reaction mixture was stirred at 0° C. for 30 minutes, and then allowed to warm to 20° C. and stirred for another 2 hours. The resulting mixture was filtered, and the filtrate was concentrated under reduced pressure. The residue was purified by column chromatography (SiO₂, petroleum ether:ethyl acetate, 5:1 to 1:1) to give the title compound (6.7 g, 74%) as a yellow solid.

¹H NMR (400 MHz, DMSO-d₆) δ 7.91 (d, 1H), 7.35 (s, 2H), 6.57 (d, 1H), 4.63-4.52 (m, 1H) and 1.43 (d, 6H).

LCMS: m/z 190 (M+H)⁺(ES⁺).

### Intermediate L9: 2-(2-Hydroxypropan-2-yl)thiazole-5-sulfonamide

### Step A: 2-(1,1-Dimethoxyethyl)thiazole

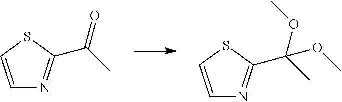

To a solution of 1-(thiazol-2-yl)ethanone (25 g, 196.60 mmol, 1 eq) in MeOH (350 mL) was added trimethoxymethane (121 g, 1.14 mol, 5.80 eq) and 4-methylbenzenesulfonic acid (35.55 g, 206.43 mmol, 1.05 eq) at 25° C. Then the reaction mixture was stirred at 50° C. for 12 hours, poured into H₂O (400 mL), and concentrated in vacuo to remove MeOH. The residue was quenched with saturated aqueous Na₂CO₃ solution (200 mL) and the mixture was extracted with EtOAc (3×100 mL). The organic combined layers were washed with brine (2×200 mL), dried over anhydrous Na₂SO₄, filtered and concentrated in vacuo to give the title compound (27 g, crude) as a red oil.

¹H NMR (400 MHz, CDCl₃) δ 7.84 (d, 1H), 7.33 (d, 1H), 3.27 (s, 6H) and 1.75 (s, 3H).

### Step B: 5-Bromo-2-(1,1-dimethoxyethyl)thiazole

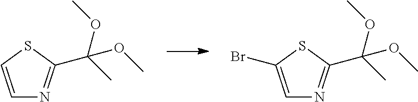

To a solution of 2-(1,1-dimethoxyethyl)thiazole (54 g, 311.72 mmol, 1 eq) in THF (1000 mL) was added dropwise n-BuLi (2.5 M, 137.16 mL, 1.1 eq) at −78° C. under N₂. The mixture was stirred at −78° C. for 0.5 hour. Then a solution of CBr₄ (113.71 g, 342.89 mmol, 1.1 eq) in THF (250 mL) was added dropwise over 10 minutes. The reaction mixture was filtered and the filtrate was poured into saturated aqueous NH₄Cl solution (200 mL) and water (200 mL). The aqueous phase was extracted with ethyl acetate (2×200 mL). The combined organic phases were washed with brine (2×500 mL), dried over anhydrous Na₂SO₄, filtered and concentrated in vacuo. The residue was purified by column chromatography (SiO₂, petroleum ether:ethyl acetate, 1:0 to 10:1) to afford the title compound (70 g, 83% yield, 90% purity on ¹H NMR) as a yellow oil.

¹H NMR (400 MHz, CDCl₃) δ 7.70 (s, 1H), 3.25 (s, 6H) and 1.70 (s, 3H).

### Step C: 1-(5-Bromothiazol-2-yl)ethanone

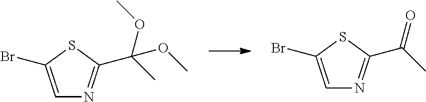

To a solution of 5-bromo-2-(1,1-dimethoxyethyl)thiazole (70 g, 277.64 mmol, 1 eq) in DCM (500 mL) was added TFA (462 g, 4.05 mol, 14.59 eq) and H₂O (10 g, 555.08 mmol, 2.0 eq) at 25° C. Then the reaction mixture was stirred at 25° C. for 12 hours, and concentrated in vacuo. The residue was purified by column chromatography (SiO₂, petroleum ether:ethyl acetate, 1:0 to 15:1) to give the title compound (51 g, 89%) as a red solid.

¹H NMR (400 MHz, DMSO-d₆) δ 8.23 (s, 1H) and 2.60 (s, 3H).

### Step D: 1-(5-Benzylsulfanylthiazol-2-yl)ethanone

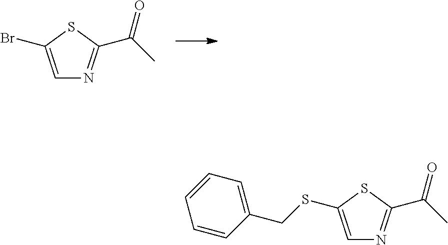

To a solution of 1-(5-bromothiazol-2-yl)ethanone (20 g, 97.06 mmol, 1 eq), phenylmethanethiol (13.26 g, 106.76 mmol, 1.1 eq), DIPEA (25.09 g, 194.12 mmol, 2 eq) and XantPhos® (2.81 g, 4.85 mmol, 0.05 eq) in dioxane (200 mL) was added Pd(dba)₂ (2.79 g, 4.85 mmol, 0.05 eq) at 25° C. The reaction mixture was stirred at 100° C. for 12 hours under N₂, and then at 25° C. for 30 minutes. The reaction mixture was filtered and the filtrate was concentrated in vacuo. The residue was purified by column chromatography (SiO₂, petroleum ether:ethyl acetate, 1:0 to 30:1) to afford the title compound (20 g, 67% yield, 81.5% purity on LCMS) as a yellow solid.

¹H NMR (400 MHz, DMSO-d₆) δ 7.95 (s, 1H), 7.46-7.27 (m, 5H), 4.30 (s, 2H) and 2.56 (s, 3H).

LCMS: m/z 250.0 (M+H)⁺(ES⁺).

### Step E: 2-Acetylthiazole-5-sulfonyl chloride

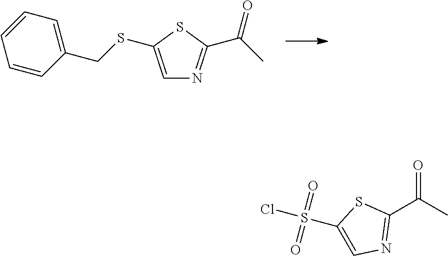 [SMILES: CC(=O)c1ncc(SCc2ccccc2)s1 ; CC(=O)c1ncc(S(=O)(=O)Cl)s1]

Cl₂ gas (15 psi) was bubbled into a solution of 1-(5-benzylsulfanylthiazol-2-yl)ethanone (20 g, 80.21 mmol, 1 eq) in AcOH (360 mL) and H₂O (40 mL) at 0° C. for 45 minutes. The reaction mixture was stirred at 0° C. for 1 hour, and then poured into water (500 mL). The aqueous phase was extracted with ethyl acetate (2×200 mL). The combined organic phases were washed with brine (2×200 mL), dried over anhydrous Na₂SO₄, filtered and concentrated in vacuo to give the title compound (18 g, crude) as a yellow oil, which was used directly in the next step.

### Step F: 2-Acetylthiazole-5-sulfonamide

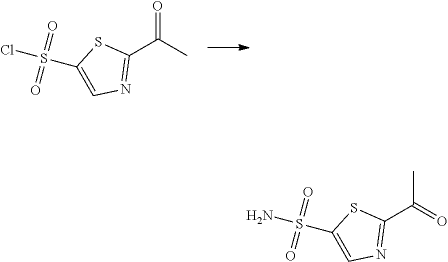

NH₃ gas (15 psi) was bubbled into THF (300 mL) at −78° C. for 15 minutes. Then a solution of 2-acetylthiazole-5-sulfonyl chloride (18 g, 79.76 mmol, 1 eq) in THF (50 mL) was added dropwise into the NH₃/THF solution at −78° C. The reaction mixture was stirred at 25° C. for 30 minutes, and then concentrated in vacuo. The residue was purified by column chromatography (SiO₂, petroleum ether:ethyl acetate, 10:1 to 1:1) to afford the title compound (6.8 g, 41%) as a yellow solid.

¹H NMR (400 MHz, DMSO-d₆) δ 8.41 (s, 1H), 8.17 (br s, 2H) and 2.65 (s, 3H).

LCMS: m/z 206.9 (M+H)⁺ (ES⁺).

### Step G: 2-(2-Hydroxypropan-2-yl)thiazole-5-sulfonamide

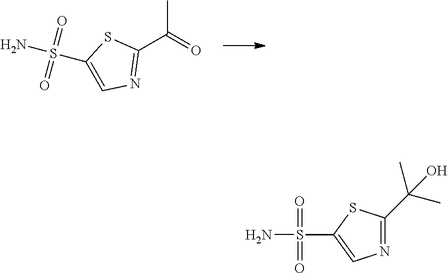

To the solution of 2-acetylthiazole-5-sulfonamide (8.6 g, 41.70 mmol, 1 eq) in THF (200 mL) was added MeMgBr (3 M, 55.60 mL, 4 eq) at −10° C. under N₂. The reaction mixture was stirred at 0° C. for 30 minutes, and then stirred at 20° C. for 2 hours. The reaction mixture was poured into aqueous NH₄Cl solution (500 mL). The aqueous phase was extracted with ethyl acetate (2×100 mL). The combined organic phases were washed with brine (2×200 mL), dried over anhydrous Na₂SO₄, filtered and concentrated in vacuo. The residue was purified by column chromatography (SiO₂, petroleum ether:ethyl acetate, 10:1 to 1:1) to give the title compound (3.5 g, 38%) as a yellow solid.

¹H NMR (400 MHz, DMSO-d₆) δ 8.01 (s, 1H), 7.81 (br s, 2H), 6.29 (br s, 1H) and 1.51 (s, 6H).

LCMS: m/z 223.0 (M+H)⁺ (ES⁺).

### Intermediate L10: 5-(2-Hydroxypropan-2-yl)thiazole-2-sulfonamide

### Step A: Methyl 2-mercaptothiazole-5-carboxylate

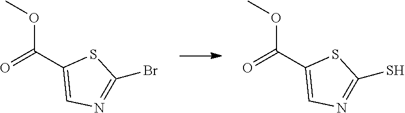

A solution of methyl 2-bromothiazole-5-carboxylate (10 g, 45.03 mmol, 1 eq) and NaHS (7.21 g, 90.07 mmol, 70 wt % purity (contained 30% H₂O), 2 eq) in EtOH (100 mL) was stirred at 80° C. for 2 hours. The mixture was poured into ice-water (300 mL) and extracted with ethyl acetate (2×300 mL). The combined organic layers were washed with brine (2×500 mL), dried over anhydrous Na₂SO₄, filtered and concentrated in vacuo to give the title compound (7.82 g, 90% yield, 91% purity on LCMS) as a light yellow solid, which was used in the next step without further purification.

¹H NMR (400 MHz, DMSO-d₆) δ 13.83 (br s, 1H), 8.12 (s, 1H) and 3.77 (s, 3H).

LCMS: m/z 176.6 (M+H)⁺ (ES⁺).

### Step B: Methyl 2-(chlorosulfonyl)thiazole-5-carboxylate

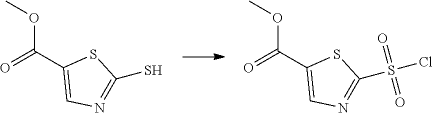

To a solution of methyl 2-mercaptothiazole-5-carboxylate (5.5 g, 28.56 mmol, 1 eq) in DCM (60 mL) was added NCS (11.44 g, 85.69 mmol, 3 eq) at 0° C. The reaction mixture was stirred at 25° C. for 1 hour, and then poured into ice-water (100 mL) and extracted with DCM (2×70 mL). The combined organic layers were washed with brine (2×100 mL), dried over anhydrous Na₂SO₄, filtered and concentrated in vacuo to give the title compound (6.90 g, crude) as a light yellow oil, which was used in the next step without further purification.

### Step C: Methyl 2-sulfamoylthiazole-5-carboxylate

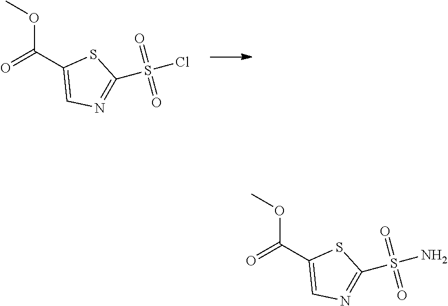

To a stirred solution of methyl 2-(chlorosulfonyl)thiazole-5-carboxylate (6.90 g, 28.55 mmol, 1 eq) in THF (80 mL) was bubbled NH₃ gas (15 psi) at 0° C. for 0.25 hour. The reaction mixture was stirred at 25° C. for 0.5 hour, and then filtered. The filtrate was concentrated in vacuo. The residue was purified by column chromatography (SiO₂, petroleum ether:ethyl acetate, 5:1 to 3:1) to give the title compound (1.1 g, 16% yield, 92% purity on LCMS) as a light yellow solid.

¹H NMR (400 MHz, DMSO-d₆) δ 8.62 (s, 1H), 8.38 (s, 2H) and 3.89 (s, 3H).

LCMS: m/z 223.5 (M+H)⁺ (ES⁺).

### Step D: 5(2-Hydroxypropan-2-yl)thiazole-2-sulfonamide

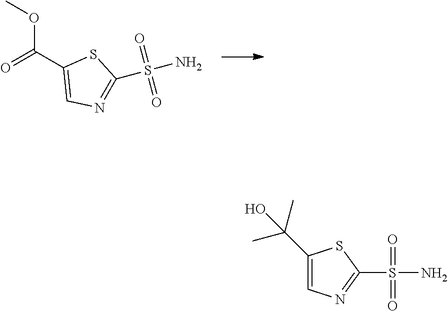

To a solution of methyl 2-sulfamoylthiazole-5-carboxylate (1 g, 4.50 mmol, 1 eq) in THF (20 mL) was added dropwise MeMgBr (3 M, 6.75 mL, 4.5 eq) at −10° C. under N₂. The reaction mixture was stirred at 0° C. for 0.5 hour, and then warmed to 25° C. and stirred for 15 hours. The reaction mixture was quenched slowly with a saturated aqueous NH₄Cl solution (50 mL) and extracted with EtOAc (2×50 mL). The combined organic layers were washed with brine (2×70 mL), dried over anhydrous Na₂SO₄, filtered and concentrated in vacuo. The residue was purified by column chromatography (SiO₂, petroleum ether:ethyl acetate, 10:1 to 3:1) to give the title compound (0.37 g, 35% yield, 95% purity on LCMS) as a light yellow solid.

¹H NMR (400 MHz, DMSO-d₆) δ 7.99 (s, 2H), 7.81 (s, 1H), 5.92 (s, 1H) and 1.55 (s, 6H).

LCMS: m/z 223.5 (M+H)⁺ (ES⁺ ).

### Intermediate L11: 1-Cyclopropyl-1H-pyrazole-3-sulfonyl chloride

### Step A: 1-Cyclopropyl-3-nitro-1H-pyrazole

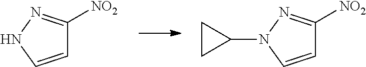 [SMILES: O=[N+]([O-])c1cc[nH]n1 ; O=[N+]([O-])c1ccn(C2CC2)n1]

To a solution of cyclopropylboronic acid (36.77 g, 428.04 mmol, 1.1 eq) in DCE (500 mL) was added 3-nitro-1H-pyrazole (44 g, 389.12 mmol, 1 eq), 2,2-bipyridine (60.77 g, 389.12 mmol, 1 eq) and Na₂CO₃ (64.59 g, 609.44 mmol, 1.57 eq) at 25° C. The reaction mixture was stirred at 25° C. for 30 minutes. Then Cu(OAc)₂ (70.68 g, 389.12 mmol, 1 eq) was added. The resulting reaction mixture was heated to 70° C., stirred for 15.5 hours, and concentrated under reduced pressure. The residue was purified by column chromatography (SiO₂, petroleum ether:ethyl acetate, 30:1 to 3:1) to give impure product (26.7 g). The impure product was dissolved in pyrrolidine (10 mL). The resulting mixture was stirred at 70° C. for 2 hours, and then concentrated under reduced pressure to remove pyrrolidine. The residue was diluted with H₂O (33 mL) and adjusted to pH=5-6 with 1M aqueous HCl solution. The mixture was extracted with EtOAc (3×50 mL). The combined organic layers were washed with brine (2×33 mL), dried over Na₂SO₄, filtered and concentrated under reduced pressure to give the title compound (17.7 g, 30%) as a yellow oil.

¹H NMR (400 MHz, CDCl₃) δ 7.54 (d, 1H), 6.84 (d, 1H), 3.73-3.67 (m, 1H), 1.24-1.22 (m, 2H) and 1.13-1.07 (m, 2H).

### Step B: 1-Cyclopropyl-1H-pyrazol-3-amine

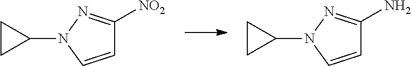 [SMILES: O=[N+]([O-])c1ccn(C2CC2)n1 ; Nc1ccn(C2CC2)n1]

To a solution of 1-cyclopropyl-3-nitro-1H-pyrazole (36 g, 235.08 mmol, 1 eq) in EtOH (400 mL) was added a solution of NH₄Cl (62.87 g, 1.18 mol, 5 eq) in H₂O (150 mL). Then the reaction mixture was heated to 60° C. and iron powder (39.38 g, 705.24 mmol, 3 eq) was added in portions. The reaction mixture was stirred at 60° C. for 16 hours, and then concentrated under reduced pressure. The residue was diluted with H₂O (500 mL) and the mixture was extracted with EtOAc (3×500 mL). The combined organic layers were washed with brine (2×250 mL), dried over Na₂SO₄, filtered and concentrated under reduced pressure. The residue was purified by column chromatography (SiO₂, petroleum ether:ethyl acetate, 30:1 to 1:1) to give the title compound (20 g, 69%) as a yellow oil.

¹H NMR (400 MHz, CDCl₃) δ 7.14 (d, 1H), 5.11 (d, 1H), 3.57 (br s, 2H), 3.38-3.32 (m, 1H), 0.99-0.95 (m, 2H) and 0.90-0.87 (m, 2H).

LCMS: m/z 124.2 (M+H)⁺ (ES⁺).

### Step C: 1-Cyclopropyl-1H-pyrazole-3-sulfonyl chloride

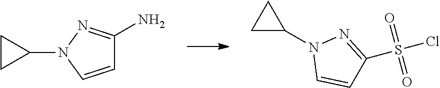 [SMILES: Nc1ccn(C2CC2)n1 ; O=S(=O)(Cl)c1ccn(C2CC2)n1]

To a solution of 1-cyclopropyl-1H-pyrazol-3-amine (19 g, 154.28 mmol, 1 eq) in MeCN (500 mL) and H₂O (50 mL) at 0° C. was added a solution of concentrated HCl (50 mL, 36 wt % in H₂O). Then an aqueous solution of NaNO₂ (12.77 g, 185.13 mmol, 1.2 eq) in H₂O (50 mL) was added slowly. The resulting mixture was stirred at 0° C. for 40 minutes. AcOH (50 mL), CuCl₂ (10.37 g, 77.14 mmol, 0.5 eq) and CuCl (763 mg, 7.71 mmol, 0.05 eq) were added. Then SO₂ gas (15 psi) was bubbled into the resulting mixture at 0° C. for 20 minutes. The reaction mixture was stirred at 0° C. for 1 hour, and then concentrated under reduced pressure. The residue was diluted with H₂O (250 mL) and extracted with EtOAc (3×250 mL). The combined organic layers were washed with brine (2×150 mL), dried over Na₂SO₄, filtered and concentrated under reduced pressure. The residue was purified by column chromatography (SiO₂, petroleum ether:ethyl acetate, 100:0 to 1:1) to give the title compound (14 g, 44%) as a yellow oil.

¹H NMR (400 MHz, CDCl₃) δ 7.62 (d, 1H), 6.83 (d, 1H), 3.78-3.72 (m, 1H), 1.28-1.24 (m, 2H) and 1.16-1.12 (m, 2H).

Intermediate L12: 5-(2-Hydroxypropan-2-yl)-1-methyl-1H-pyrazole-3-sulfonamide

Step A: 1-Methyl-1H-pyrazole-3-sulfonyl chloride

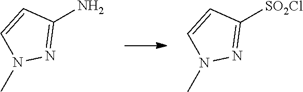

A solution of 1-methyl-1H-pyrazol-3-amine (20 g, 205.93 mmol, 1 eq) in MeCN (500 mL) at 0° C. was treated with aqueous HCl solution (1 N, 50 mL), followed slowly by an aqueous solution of NaNO₂ (17.05 g, 247.12 mmol, 1.2 eq) in H₂O (50 mL). The resulting solution was stirred at 0° C. for 40 minutes. Then AcOH (50 mL), CuCl₂ (13.84 g, 102.97 mmol, 0.5 eq) and CuCl (1.02 g, 10.30 mmol, 0.05 eq) were added. SO₂ gas (15 psi) was bubbled into the mixture at 0° C. for 20 minutes. The reaction mixture was stirred at 0° C. for 1 hour, and then concentrated under reduced pressure. The residue was diluted with H₂O (400 mL) and extracted with ethyl acetate (3×400 mL). The combined organic layers were washed with brine (2×200 mL), dried over Na₂SO₄, filtered and concentrated under reduced pressure. The residue was purified by column chromatography (SiO₂, petroleum ether:ethyl acetate, 30:1 to 1:1) to give the title compound (12 g, 32%) as a yellow oil.

¹H NMR (400 MHz, CDCl₃) δ 7.53 (d, 1H), 6.86 (d, 1H) and 4.05 (s, 3H).

### Step B: N,N-Bis(4-methoxybenzyl)-1-methyl-1H-pyrazole-3-sulfonamide

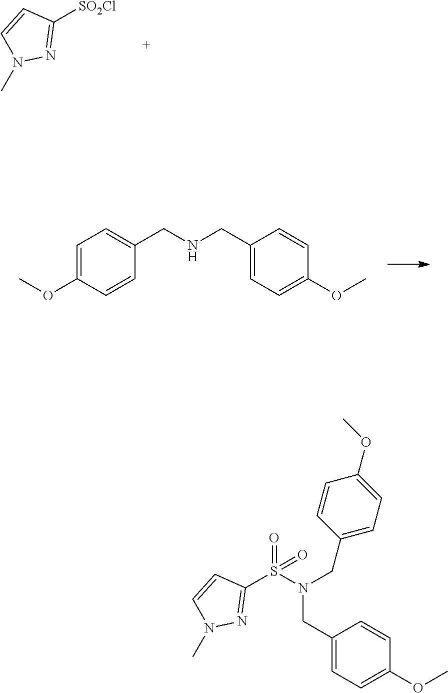

To a solution of bis(4-methoxybenzyl)amine (60 g, 233.17 mmol, 0.7 eq) in THF (600 mL) were added TEA (67.23 g, 664.41 mmol, 2 eq) and 1-methyl-1H-pyrazole-3-sulfonyl chloride (60 g, 332.20 mmol, 1 eq). The reaction mixture was stirred at 25° C. for 1 hour, and then diluted with H₂O (1 L). The pH was adjusted to pH=5-6 with aqueous HCl solution (1 N). The mixture was extracted with EtOAc (3×1 L). The combined organic layers were washed with brine (2×300 mL), dried over Na₂SO₄, filtered and concentrated under reduced pressure. The residue was triturated with EtOAc (200 mL) to give the title compound (50 g, 34% yield, 90% purity on LCMS) as a white solid.

¹H NMR (400 MHz, CDCl₃) δ7.41 (d, 1H), 7.07-7.04 (m, 4H), 6.78-6.75 (m, 4H), 6.62 (d, 1H), 4.31 (s, 4H), 3.96 (s, 3H) and 3.78 (s, 6H).

LCMS: m/z 402.2 (M+H)⁺ (ES⁺).

### Step C: 3-(N,N-Bis(4-methoxybenzyl)sulfamoyl)-1-methyl-1H-pyrazole-5-carboxylic acid

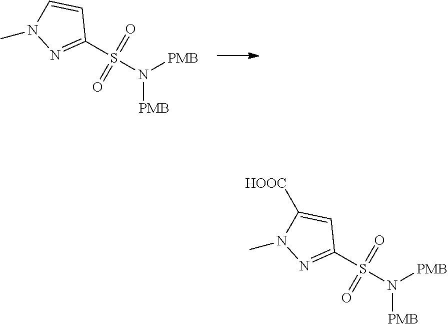

To a solution of N,N-bis(4-methoxybenzyl)-1-methyl-1H-pyrazole-3-sulfonamide (100 g, 249.08 mmol, 1 eq) in THF (1.35 L) was added dropwise n-BuLi (2.5 M, 104.61 mL, 1.05 eq) at −70° C. The reaction mixture was stirred at −70° C. for 1 hour. Then CO₂ gas (15 psi) was bubbled into the reaction mixture for 15 minutes. The resulting mixture was stirred at −70° C. for another 1 hour, quenched with H₂O (1.2 L), adjusted to pH=3 with aqueous HCl solution (1 N), and extracted with EtOAc (2×1 L). The combined organic phases were washed with brine (2×1 L), dried over anhydrous Na₂SO₄, filtered and concentrated in vacuo. The residue was triturated with a mixture of petroleum ether and ethyl acetate (300 mL, petroleum ether:ethyl acetate, 1:1) to give the title compound (94 g, 84% yield, 99% purity on LCMS) as a white solid.

¹H NMR (400 MHz, DMSO-d₆) δ 6.98-7.16 (m, 5H), 6.82 (d, 4H), 4.25 (s, 4H), 4.15 (s, 3H) and 3.72 (s, 6H).

LCMS: m/z 468.2 (M+Na)⁺ (ES⁺).

### Step D: Ethyl 3-(N,N-bis(4-methoxybenzyl)sulfamoyl)-1-methyl-1H-pyrazole-5-carboxylate

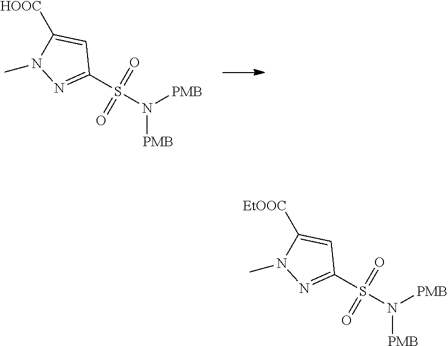

To a solution of 3-(N,N-bis(4-methoxybenzyl)sulfamoyl)-1-methyl-1H-pyrazole-5-carboxylic acid (42.75 g, 95.96 mmol, 1 eq) and DMF (701 mg, 9.60 mmol, 0.1 eq) in DCM (500 mL) was added (COCl)₂ (37 g, 287.89 mmol, 3 eq) at 0° C. under N₂. The reaction mixture was stirred at 25° C. for 0.5 hour, and then added dropwise into EtOH (100 mL) at 0° C. The resulting mixture was stirred at 25° C. for 1.5 hours, and then concentrated in vacuo. The residue was purified by column chromatography (SiO₂, petroleum ether:ethyl acetate, 15:1 to 10:1) to give the title compound (37.5 g, 80% yield, 97.4% purity on LCMS) as a white solid.

¹H NMR (400 MHz, DMSO-d₆) δ 7.08 (s, 1H), 7.06 (d, 4H), 6.80 (d, 4H), 4.35-4.29 (m, 2H), 4.26 (s, 4H), 4.15 (s, 3H), 3.71 (s, 6H) and 1.32 (t, 3H).

LCMS: m/z 496.1 (M+Na)⁺ (ES⁺).

### Step E: Ethyl 1-methyl-3-sulfamoyl-1H-pyrazole-5-carboxylate

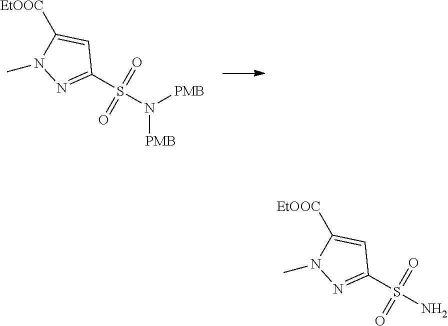

A solution of ethyl 3-(N,N-bis(4-methoxybenzyl)sulfamoyl)-1-methyl-1H-pyrazole-5-carboxylate (35.9 g, 75.81 mmol, 1 eq) in DCM (200 mL) and TFA (100 mL) was stirred at 25° C. for 15 hours. The reaction mixture was concentrated in vacuo at 25° C. The residue was treated with MeOH (200 mL), with some solid not dissolving. The mixture was filtered and the filtrate was concentrated in vacuo. The residue was purified by column chromatography (SiO₂, petroleum ether:ethyl acetate:DCM, 10:1:1 to 1:1:1) to give the title compound (14.5 g, 81% yield, 98.7% purity on LCMS) as a white solid.

¹H NMR (400 MHz, DMSO-d₆) δ 7.58 (s, 2H), 7.09 (s, 1H), 4.32 (q, 2H), 4.14 (s, 3H) and 1.32 (t, 3H).

LCMS: m/z 233.9 (M+H)⁺ (ES⁺).

### Step F: 5-(2-Hydroxypropan-2-yl)-1-methyl-1H-pyrazole-3-sulfonamide

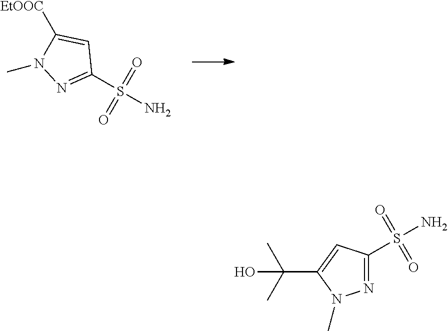

To a mixture of ethyl 1-methyl-3-sulfamoyl-1H-pyrazole-5-carboxylate (13.5 g, 57.88 mmol, 1 eq) in THF (400 mL) was added dropwise methylmagnesium bromide (3 M, 96.47 mL, 5 eq) at −10° C. over a period of 0.5 hour under N₂. The reaction mixture was stirred at 0° C. for 0.5 hour, then warmed to 25° C. and stirred for 15 hours. The reaction mixture was quenched slowly with a saturated aqueous NH₄Cl solution (300 mL) and extracted with EtOAc (2×300 mL). The combined organic layers were washed with brine (2×500 mL), dried over anhydrous Na₂SO₄, filtered and concentrated in vacuo. The residue was purified by column chromatography (SiO₂, petroleum ether:ethyl acetate, 2:1 to 0:1) to give the title compound (5.52 g, 43% yield, 98.7% purity on LCMS) as a colourless oil.

¹H NMR (400 MHz, DMSO-d₆) δ 7.32 (s, 2H), 6.40 (s, 1H), 5.47 (s, 1H), 4.01 (s, 3H) and 1.50 (s, 6H).

LCMS: m/z 220.1 (M+H)⁺ (ES⁺).

### Intermediate L13: 4-((Dimethylamino)methyl)benzenesulfonamide

### Step A: 4-(Bromomethyl)benzenesulfonamide

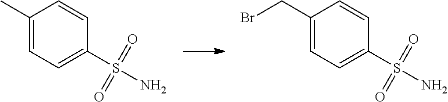

To a solution of 4-methylbenzenesulfonamide (2 g, 11.68 mmol, 1 eq) and NBS (2.2 g, 12.27 mmol, 1.05 eq) in carbon tetrachloride (20 mL) was added AIBN (192 mg, 1.17 mmol, 0.1 eq) at 20° C. The reaction mixture was stirred at 80° C. for 2 hours, cooled to room temperature and poured into ice-water (20 mL). The aqueous phase was extracted with ethyl acetate (3×20 mL). The combined organic layers were washed with brine (20 mL), dried over anhydrous sodium sulfate, filtered and concentrated in vacuo to give the title compound (3 g, crude) as a yellow solid.

¹H NMR (400 MHz, CD₃OD) δ 7.87 (d, 2H), 7.59 (d, 2H) and 4.62 (s, 2H).

### Step B: 4-((Dimethylamino)methyl)benzenesulfonamide

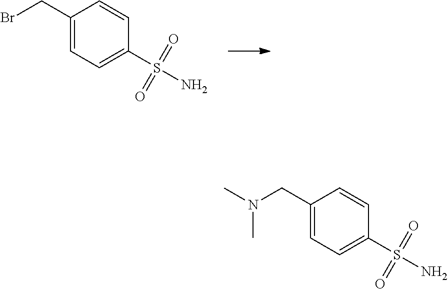

A mixture of 4-(bromomethyl)benzenesulfonamide (1.5 g, 6.00 mmol, 1 eq) and dimethylamine (10 mL, 33 wt % in H₂O, 10 eq) in THF (5 mL) was stirred at 20° C. for 12 hours, and then concentrated in vacuo. The residue was purified by reversed phase flash chromatography (0.1% NH₃.H₂O-MeCN) to give the title compound (0.6 g, 47%) as a yellow solid.

¹H NMR (400 MHz, CD₃OD) δ 7.87 (d, 2H), 7.49 (d, 2H), 3.55 (s, 2H) and 2.25 (s, 6H).

LCMS: m/z 215.1 (M+H)⁺(ES⁺).

### Intermediate L14: 5-(2-Methoxypropan-2-yl)-1-methyl-1H-pyrazole-3-sulfonamide

### Step A: Ethyl 3-(N,N-bis(4-methoxybenzyl)sulfamoyl)-1-methyl-1H-pyrazole-5-carboxylate

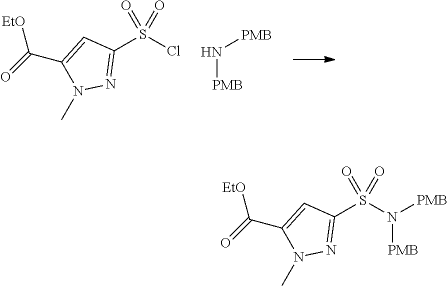

Ethyl 3-(chlorosulfonyl)-1-methyl-1H-pyrazole-5-carboxylate (9.2 g, 36.4 mmol) was added dropwise to a solution of bis(4-methoxybenzyl)amine (9.4 g, 36.5 mmol) and triethylamine (10 mL, 71.7 mmol) in DCM (200 mL), cooled in an ice bath. The resulting mixture was stirred for 30 minutes, warmed to room temperature and stirred for 90 minutes before being washed with water (200 mL), aqueous hydrochloric acid (1 M, 200 mL), water (200 mL), dried (MgSO₄), filtered and evaporated to give a yellow oil. This was purified by chromatography on silica gel (220 g column, 0-60% ethyl acetate/isohexane) to afford the title compound (15.9 g, 91%) as a white solid.

¹H NMR (DMSO-d₆) δ 7.19-7.00 (m, 5H), 6.85-6.77 (m, 4H), 4.33 (q, 2H), 4.25 (s, 4H), 4.15 (s, 3H), 3.71 (s, 6H) and 1.33 (t, 3H).

LCMS m/z 496.4 (M+Na)+(ES⁺).

### Step B: 5-(2-Hydroxypropan-2-yl)-N,N-bis(4-methoxybenzyl)-1-methyl-1H-pyrazole-3-sulfonamide

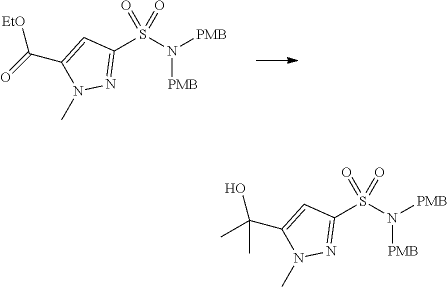

Ethyl 3-(N,N-bis(4-methoxybenzyl)sulfamoyl)-1-methyl-1H-pyrazole-5-carboxylate (1.4 g, 2.96 mmol) was dissolved in dry THF (50 mL) and cooled to −78° C. in a dry ice/acetone bath. Methyl magnesium chloride (3 M in THF, 5 mL, 15.0 mmol) was added slowly via syringe over the course of 15 minutes. The reaction mixture was allowed to reach room temperature and stirred overnight before being cooled in an ice bath and quenched slowly with portions of aqueous ammonium chloride (20 mL). The mixture was extracted into ethyl acetate (3×50 mL) and the combined organic extracts were washed with brine (10 mL), dried (Na₂SO₄), filtered and concentrated in vacuo to afford a colourless oil. The crude product was purified by chromatography on silica gel (40 g column, 0-50% ethyl acetate/isohexane) to afford the title compound (1.11 g, 67%) as a thick colourless oil.

¹H NMR (DMSO-d₆) δ 7.09-7.03 (m, 4H), 6.85-6.80 (m, 4H), 6.41 (s, 1H), 4.21 (s, 4H), 4.04 (s, 3H), 3.72 (s, 6H) and 1.50 (s, 6H). One exchangeable proton not observed.

LCMS m/z 460 (M+H)⁺(ES⁺); 458 (M−H)⁻ (ES⁻).

### Step C: N,N-bis-(4-Methoxybenzyl)-5-(2-methoxypropan-2-yl)-1-methyl-1H-pyrazole-3-sulfonamide

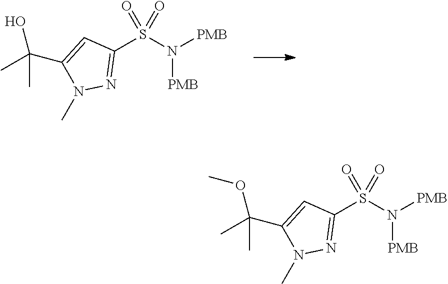

5-(2-Hydroxypropan-2-yl)-N,N-bis(4-methoxybenzyl)-1-methyl-1H-pyrazole-3-sulfonamide (2.5 g, 5.33 mmol) was dissolved in dry DMF (50 mL) under a nitrogen atmosphere. After cooling in an ice bath, sodium hydride (60% in mineral oil, 0.25 g, 6.25 mmol) was added in a single portion and the cloudy yellow mixture was stirred for 30 minutes. Iodomethane (1.5 mL, 24.1 mmol) was added in a single portion and the mixture was stirred for a further 2 hours, while warming to room temperature. The reaction mixture was quenched by slow addition of sat aq ammonium chloride (10 mL) and then partitioned between ethyl acetate (100 mL) and water (50 mL). The aqueous phase was extracted with ethyl acetate (4×50 mL). The combined organic portions were washed with brine (20 mL), dried (Na₂SO₄), filtered and concentrated in vacuo to give a yellow oil. The crude product was purified by chromatography on silica (40 g column, 0-100% ethyl acetate/isohexane) to afford, after drying in vacuo, the title compound (2.41 g, 94%) as a colourless solid.

¹H NMR (DMSO-d₆) δ 7.10-7.04 (m, 4H), 6.85-6.80 (m, 4H), 6.48 (s, 1H), 4.23 (s, 4H), 3.97 (s, 3H), 3.72 (s, 6H), 2.97 (s, 3H) and 1.50 (s, 6H).

LCMS m/z 474 (M+H)⁺ (ES⁺); 472 (M−H)⁻ (ES⁻).

### Step D: 5-(2-Methoxypropan-2-yl)-1-methyl-1H-pyrazole-3-sulfonamide

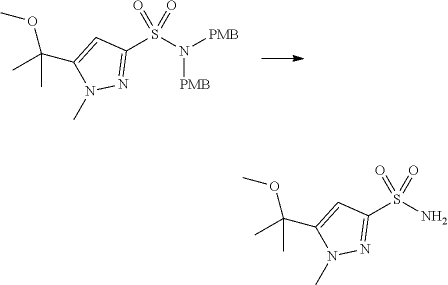

N,N-bis-(4-Methoxybenzyl)-5-(2-methoxypropan-2-yl)-1-methyl-1H-pyrazole-3-sulfonamide (2.4 g, 5.02 mmol) was dissolved in acetonitrile (40 mL). A solution of ceric ammonium nitrate (15 g, 27.4 mmol) in water (10 mL) was added in a single portion and the dark red reaction mixture was stirred at room temperature for 4 hours. Water (10 mL) and DCM (250 mL) were added and the organic phase was separated, dried by passing through a hydrophobic frit and concentrated in vacuo to give an orange oil (about 2.5 g). The crude product was purified by chromatography on silica gel (40 g column, 0-20% methanol/dichloromethane) to afford an orange oil. Trituration of this material in TBME (10 mL) and isohexane (5 mL) gave a tan precipitate which was further purified by chromatography on silica gel (24 g, 20-100% ethyl acetate in hexanes) to afford the title compound (383 mg, 31%) as a yellow solid.

¹H NMR (CDCl₃) δ 6.57 (s, 1H), 5.08 (s, 2H), 4.06 (s, 3H), 3.08 (s, 3H) and 1.57 (s, 6H).

### Intermediate L15: 1-(2,2,2-Trifluoroethyl)-1H-pyrazole-4-sulfonamide

### Step A: 1-(2,2,2-Trifluoroethyl)-1H-pyrazol-4-amine

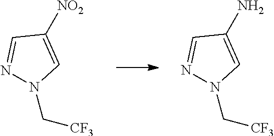 [SMILES: O=[N+]([O-])c1cnn(CC(F)(F)F)c1 ; Nc1cnn(CC(F)(F)F)c1]

4-Nitro-1-(2,2,2-trifluoroethyl)-1H-pyrazole (1 g, 5.13 mmol) and 10% Pd/C (wet) Type 87 L (27 mg, 0.256 mmol) were suspended with MeOH (12.8 mL) and EtOAc (12.8 mL). The vessel was purged with N₂ three times, then filled with H₂ three times. The reaction mixture was stirred at room temperature under 5 bar H₂ for 18 hours. The reaction mixture was filtered through a pad of Celite. The filter cake was washed with EtOAc (2×5 mL) and the combined filtrates were evaporated in vacuo to give the title compound as a red oil (0.85 g, 100%).

¹H NMR (Chloroform-d) δ 7.25 (d, J=0.9 Hz, 1H), 7.09 (d, J=0.8 Hz, 1H), 4.57 (q, J=8.4 Hz, 2H), 2.43 (br s, 2H).

¹⁹F NMR (Chloroform-d) δ −71.88 (t, J=8.4 Hz).

LCMS m/z 166.0 (M+H)⁺(ES⁺).

### Step B: 1-(2,2,2-Trifluoroethyl)-1H-pyrazole-4-sulfonyl chloride

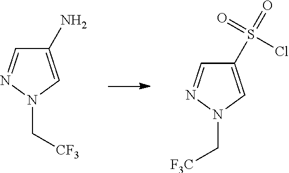 [SMILES: Nc1cnn(CC(F)(F)F)c1 ; O=S(=O)(Cl)c1cnn(CC(F)(F)F)c1]

A mixture of conc HCl (1.9 mL) in water (1.3 mL) and acetonitrile (6.5 mL) was cooled to −10° C. (acetone/dry ice bath) and treated with a solution of sodium nitrite (426 mg, 6.18 mmol) in water (0.7 mL) dropwise, maintaining internal temperature below 0° C. A yellow solution formed which was stirred for 10 minutes and then treated at 0° C. over 15 minutes with a solution of 1-(2,2,2-trifluoroethyl)-1H-pyrazol-4-amine (850 mg, 5.15 mmol) in MeCN (6.5 mL), which was pre-cooled to 0° C. The resulting reaction mixture was stirred at 0° C. for 45 minutes. Cold acetic acid (2.6 mL), copper(II) chloride (346 mg, 2.57 mmol) and copper(I) chloride (25.5 mg, 0.257 mmol) were sequentially added to the reaction mixture. Then the reaction mixture was purged with sulfur dioxide gas for 70 minutes at 0° C. The reaction mixture was diluted with water (15 mL) and extracted with EtOAc (3×30 mL), then dried (MgSO₄), filtered and concentrated to dryness to give a brown paste. The crude product was purified by chromatography on silica gel (40 g column, 0-50% DCM/isohexane) to afford the title compound (420 mg, 16%) as a clear yellow oil.

¹H NMR (Chloroform-d) δ 8.20 (s, 1H), 8.06 (s, 1H), 4.81 (q, J=8.1 Hz, 2H).

### Step C: 1-(2,2,2-Trifluoroethyl)-1H-pyrazole-4-sulfonamide

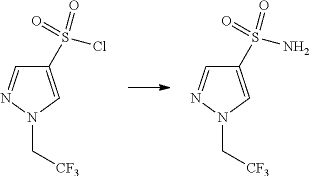 [SMILES: O=S(=O)(Cl)c1cnn(CC(F)(F)F)c1 ; NS(=O)(=O)c1cnn(CC(F)(F)F)c1]

A solution of 1-(2,2,2-trifluoroethyl)-1H-pyrazole-4-sulfonyl chloride (420 mg, 0.845 mmol) (50 wt % purity) in THF (1.4 mL) was treated with NH₃ (0.5 M in dioxane, 5 mL). The reaction mixture was then stirred at room temperature for 17 hours. The solvent was removed in vacuo and the residue partitioned between water (5 mL) and DCM (15 mL). The organic layer was separated and the aqueous layer was extracted with DCM (2×15 mL). The combined organic layers were dried (MgSO₄) and concentrated in vacuo to give an orange oil, which was co-evaporated twice with 3:1 isohexane/DCM until the dry mixture solidified. The solid product was then dissolved in DCM (3 mL) and isohexane (9 mL) was added. The supernatant liquid was decanted off and the solid washed with 3:1 isohexane/DCM (1×3 mL) and isohexane (2×3 mL). The solid was collected and dried in vacuo to afford the title compound (159 mg, 78%) as an orange powder.

¹H NMR (DMSO-d₆) δ 8.32 (d, J=0.7 Hz, 1H), 7.87 (d, J=0.7 Hz, 1H), 7.42 (s, 2H), 5.24 (q, J=9.0 Hz, 2H).

¹⁹F NMR (DMSO-d₆) δ −70.16 (t, J=9.0 Hz).

LCMS m/z 227.9/228.9 (M−H)⁻ (ES⁻).

### Intermediate L16: 1-(2,2,2-Trifluoroethyl)-1H-pyrazole-3-sulfonamide

### Step A: 1-(2,2,2-Trifluoroethyl)-1H-pyrazol-3-amine

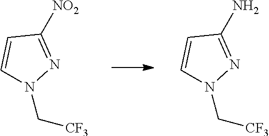 [SMILES: O=[N+]([O-])c1ccn(CC(F)(F)F)n1 ; Nc1ccn(CC(F)(F)F)n1]

Prepared according to the general procedure of 1-(2,2,2-trifluoroethyl)-1H-pyrazol-4-amine (Intermediate L15, Step A) from 3-nitro-1-(2,2,2-trifluoroethyl)-1H-pyrazole to afford the title compound (420 mg, 98%) as a yellow solid.

¹H NMR (Chloroform-d) δ 7.23 (d, J=2.4 Hz, 1H), 5.72 (d, J=2.4 Hz, 1H), 4.47 (q, J=8.4 Hz, 2H), 2.96-2.27 (br s, 2H).

LCMS m/z 166.0 (M+H)⁺ (ES⁺).

### Step B: 1-(2,2,2-Trifluoroethyl)-1H-pyrazole-3-sulfonyl chloride

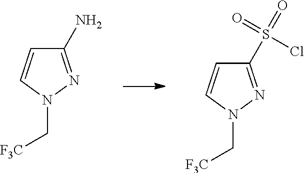 [SMILES: Nc1ccn(CC(F)(F)F)n1 ; O=S(=O)(Cl)c1ccn(CC(F)(F)F)n1]

Prepared according to the general procedure of 1-(2,2,2-trifluoroethyl)-1H-pyrazole-4-sulfonyl chloride (Intermediate L15, Step B) from 1-(2,2,2-trifluoroethyl)-1H-pyrazol-3-amine to afford the title compound (756 mg, 50%) as a clear orange oil.

¹H NMR (Chloroform-d) δ 7.69 (d, J=2.5 Hz, 1H), 7.00 (d, J=2.5 Hz, 1H), 4.86 (q, J=8.1 Hz, 2H).

¹⁹F NMR (Chloroform-d) δ −71.16 (t, J=8.1 Hz).

LCMS m/z 246.8 (M−H)⁻ (ES⁻).

### Step C: 1-(2,2,2-Trifluoroethyl)-1H-pyrazole-3-sulfonamide

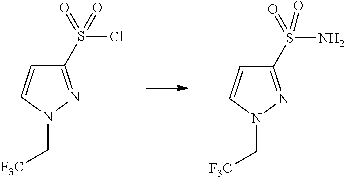 [SMILES: O=S(=O)(Cl)c1ccn(CC(F)(F)F)n1 ; NS(=O)(=O)c1ccn(CC(F)(F)F)n1]

Prepared according to the general procedure of 1-(2,2,2-trifluoroethyl)-1H-pyrazole-4-sulfonamide (Intermediate L15, Step C) from 1-(2,2,2-trifluoroethyl)-1H-pyrazole-3-sulfonyl chloride to afford the title compound (394 mg, 67%) as a white powder.

¹H NMR (DMSO-d₆) δ 8.00 (d, J=2.4 Hz, 1H), 7.55 (s, 2H), 6.70 (d, J=2.4 Hz, 1H), 5.26 (q, J=9.1 Hz, 2H).

¹⁹F NMR (DMSO-d₆) δ −70.08 (t, J=9.2 Hz).

LCMS m/z 229.1 (M+H)⁻ (ES⁻); 227.9 (M−H)⁻ (ES⁻).

### Intermediate L17: 1-Isopropyl-3-methyl-1H-pyrazole-4-sulfonamide

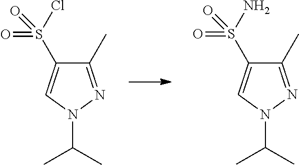

Prepared according to the general procedure of 1-(2,2,2-trifluoroethyl)-1H-pyrazole-4-sulfonamide (Intermediate L15, Step C) from 1-isopropyl-3-methyl-1H-pyrazole-4-sulfonyl chloride to afford the title compound (541 mg, 59%) as a white crystalline solid.

¹H NMR (DMSO-d₆) δ 8.06 (s, 1H), 7.15 (s, 2H), 4.46 (sept, J=6.6 Hz, 1H), 2.29 (s, 3H), 1.38 (d, J=6.7 Hz, 6H).

LCMS m/z 204.0 (M+H)⁺ (ES⁺).

### Intermediate L18: 1-(Cyclopropylmethyl)-1H-pyrazole-4-sulfonamide

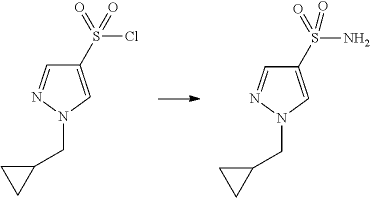 [SMILES: O=S(=O)(Cl)c1cnn(CC2CC2)c1 ; NS(=O)(=O)c1cnn(CC2CC2)c1]

Prepared according to the general procedure of 1-(2,2,2-trifluoroethyl)-1H-pyrazole-4-sulfonamide (Intermediate L15, Step C) from 1-(cyclopropylmethyl)-1H-pyrazole-4-sulfonyl chloride to afford the title compound (153 mg, 66%) as a peach-coloured solid.

¹H NMR (DMSO-d₆) δ 8.21 (d, J=0.7 Hz, 1H), 7.71 (d, J=0.8 Hz, 1H), 7.25 (s, 2H), 4.00 (d, J=7.2 Hz, 2H), δ 1.33-1.17 (m, 1H), 0.63-0.49 (m, 2H), 0.41-0.36 (m, 2H).

LCMS m/z 202.0 (M+H)⁺ (ES⁺).

### Intermediate L19: 1-Ethyl-1H-pyrazole-3-sulfonamide

### Step A: Lithium 1-(tetrahydro-2H-pyran-2-yl)-1H-pyrazole-5-sulfinate

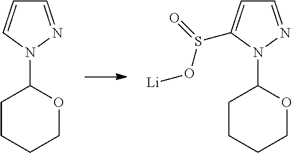

A solution of BuLi (100 mL, 250 mmol, 2.5 M in hexanes) was added slowly to a solution of 1-(tetrahydro-2H-pyran-2-yl)-1H-pyrazole (36.2 g, 238 mmol) in THF (500 mL) keeping the temperature below −65° C. The mixture was stirred for 1.5 hours, then sulfur dioxide was bubbled through for 10 minutes. The mixture was warmed to room temperature, the solvent evaporated and the residue triturated with TBME (300 mL) and filtered. The solid was washed with TBME and isohexanes and dried to afford the crude title compound (54.9 g, 99%).

¹H NMR (DMSO-d₆) δ 7.26 (d, J=1.6 Hz, 1H), 6.10 (d, J=1.7 Hz, 1H), 5.99 (dd, J=10.0, 2.5 Hz, 1H), 3.92-3.87 (m, 1H), 3.56-3.49 (m, 1H), 2.25-2.15 (m, 1H), 2.00-1.91 (m, 1H), 1.75-1.69 (m, 1H), 1.66-1.46 (m, 3H).

LCMS m/z 215 (M−H)⁻ (ES⁻).

### Step B: N,N-bis(4-Methoxybenzyl)-1-(tetrahydro-2H-pyran-2-yl)-1H-pyrazole-5-sulfonamide

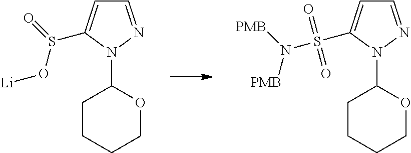

NCS (12.0 g, 90 mmol) was added to a suspension of lithium 1-(tetrahydro-2H-pyran-2-yl)-1H-pyrazole-5-sulfinate (20 g, 90 mmol) in DCM (250 mL), cooled in an ice bath. The mixture was stirred for 4 hours, quenched with water (100 mL), and then partitioned between DCM (300 mL) and water (200 mL). The organic phase was washed with water (200 mL), dried (MgSO₄) and evaporated to about 50 mL. The solution was added to a mixture of bis(4-methoxybenzyl)amine (24 g, 93 mmol) and triethylamine (40 mL, 287 mmol) in DCM (300 mL), cooled in an ice bath. After stirring for 1 hour, the mixture was warmed to room temperature, and partitioned between DCM (300 mL) and water (250 mL). The organic layer was washed with water (250 mL), aq 1 M HCl (2×250 mL), water (250 mL), dried (MgSO₄) and evaporated to afford the title compound (41.0 g, 97%) as a brown oil.

LCMS m/z 494.2 (M+Na)+ (ES⁺).

### Step C: N,N-bis(4-Methoxybenzyl)-1H-pyrazole-3-sulfonamide

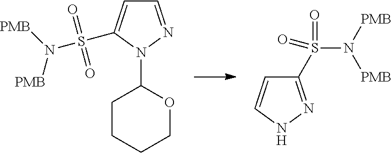

A mixture of N,N-bis(4-methoxybenzyl)-1-(tetrahydro-2H-pyran-2-yl)-1H-pyrazole-5-sulfonamide (41 g, 87 mmol) and aq 1 M HCl (30 mL) in THF (300 mL) and MeOH (50 mL) was stirred at room temperature for 18 hours. The solvent was evaporated and the residue partitioned between EtOAc (400 mL) and aq 1 M HCl (200 mL). The organic layer was washed with 10% brine (200 mL), dried (MgSO₄) and evaporated. The residue was triturated with TBME, filtered and dried to afford the title compound (24.9 g, 69%) as an off white solid.

¹H NMR (CDCl₃) δ 7.88 (d, J=2.4 Hz, 1H), 7.06-7.02 (m, 4H), 6.79-6.75 (m, 4H), 6.63 (d, J=2.4 Hz, 1H), 4.31 (s, 4H), 3.78 (s, 6H). One exchangeable proton not observed.

LCMS m/z 388 (M+H)⁺ (ES⁺); 386 (M−H)⁻ (ES⁻).

### Step D: 1-Ethyl-N,N-bis(4-methoxybenzyl)-1H-pyrazole-3-sulfonamide

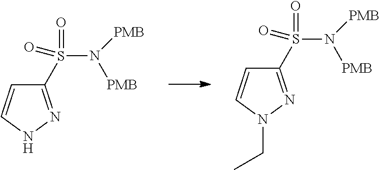

Under nitrogen, N,N-bis(4-methoxybenzyl)-1H-pyrazole-3-sulfonamide (0.347 g, 0.896 mmol) was dissolved in dry MeCN (10 mL) and K₂CO₃ (0.619 g, 4.48 mmol) was added. Bromoethane (0.267 mL, 3.58 mmol) was added in a single portion and the cloudy mixture was heated to 60° C. for 30 hours. The mixture was diluted with water (5 mL) and extracted with EtOAc (3×25 mL). The organic phase was dried (MgSO₄) and concentrated in vacuo. The crude product was purified by chromatography on silica gel (40 g column, 0-100% EtOAc/isohexane) to afford the title compound (280 mg, 69%) as a colourless oil.

LCMS m/z 416 (M+H)⁺ (ES⁺).

### Step E: 1-Ethyl-1H-pyrazole-3-sulfonamide

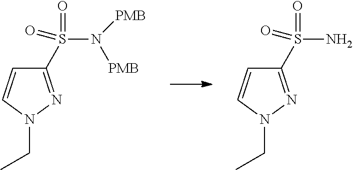

1-Ethyl-N,N-bis(4-methoxybenzyl)-1H-pyrazole-3-sulfonamide (280 mg, 0.674 mmol) was dissolved in DCM (1 mL), and water (0.5 mL) and TFA (2 mL) were added. The reaction mixture was stirred at room temperature for 15 hours. The solution was concentrated in vacuo and the residue purified by reversed phase chromatography on RP Flash C18 (12 g column, 1-30% MeCN/10 mM ammonium bicarbonate) to afford the title compound (102 mg, 83%) as white solid.

¹H NMR (DMSO-d₆) δ 7.88 (d, J=2.3 Hz, 1H), 7.36 (br s, 2H), 6.57 (d, J=2.3 Hz, 1H), 4.20 (q, J=7.3 Hz, 2H), 1.40 (t, J=7.3 Hz, 3H).

### Intermediate L20: 1-Isopropyl-1H-1,2,4-triazole-3-sulfonamide

### Step A: 3-(Benzylthio)-1-isopropyl-1H-1,2,4-triazole

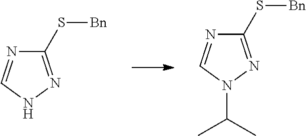 [SMILES: c1ccc(CSc2nc[nH]n2)cc1 ; CC(C)n1cnc(SCc2ccccc2)n1]

2-Iodopropane (3.00 mL, 30.0 mmol) was added to a mixture of 3-(benzylthio)-1H-1,2,4-triazole (8.2 g, 30.0 mmol) and K₂CO₃ (8.30 g, 60.0 mmol) in DMF (100 mL), cooled in an ice bath. The mixture was stirred for 2 hours, then warmed to room temperature and stirred for 20 hours. The mixture was partitioned between EtOAc (200 mL) and water (100 mL), the organic layer washed with water (2×100 mL), dried (MgSO₄) and evaporated. The residue was purified by chromatography on silica gel (80 g column, 0-50% EtOAc/isohexane) to afford the title compound (2.8 g, 40%) as an oil.

¹H NMR (DMSO-d₆) δ 8.53 (s, 1H), 7.41-7.35 (m, 2H), 7.33-7.19 (m, 3H), 4.54 (sept, J=6.8 Hz, 1H), 4.31 (s, 2H), 1.42 (d, J=6.6 Hz, 6H).

LCMS m/z 234.4 (M+H)⁺ (ES⁺).

### Step B: 1-Isopropyl-1H-1,2,4-triazole-3-sulfonyl chloride

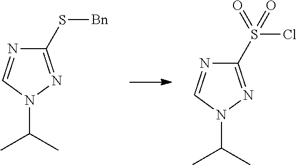 [SMILES: CC(C)n1cnc(SCc2ccccc2)n1 ; CC(C)n1cnc(S(=O)(=O)Cl)n1]

NCS (5.59 g, 41.8 mmol) was added to a solution of 3-(benzylthio)-1-isopropyl-1H-1,2,4-triazole (2 g, 10.46 mmol) in AcOH (40 mL) and water (20 mL). The mixture was stirred for 2 hours, then partitioned between EtOAc (200 mL) and water (200 mL). The organic layer was washed with sat aq NaHCO₃(100 mL), brine (50 mL), dried (MgSO₄), filtered and evaporated. TBME (20 mL) was added to the residue, the solid filtered off and the filtrate evaporated to afford the title compound (1.80 g) as a yellow oil that was used without purification in the next step.

### Step C: 1-Isopropyl-1H-1,2,4-triazole-3-sulfonamide

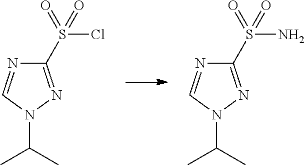

Prepared according to the general procedure of 1-(2,2,2-trifluoroethyl)-1H-pyrazole-4-sulfonamide (Intermediate L15, Step C) from 1-isopropyl-1H-1,2,4-triazole-3-sulfonyl chloride to afford the title compound (770 mg, 37% yield over 2 steps) as a colourless solid.

¹H NMR (DMSO-d₆) δ 8.76 (s, 1H), 7.71 (s, 2H), 4.69 (sept, J=6.7 Hz, 1H), 1.47 (d, J=6.7 Hz, 6H).

### Intermediate L21: (4-(Dimethylamino)pyridin-1-ium-1-carbonyl)((1-isopropyl-1H-pyrazol-3-yl)sulfonyl)amide

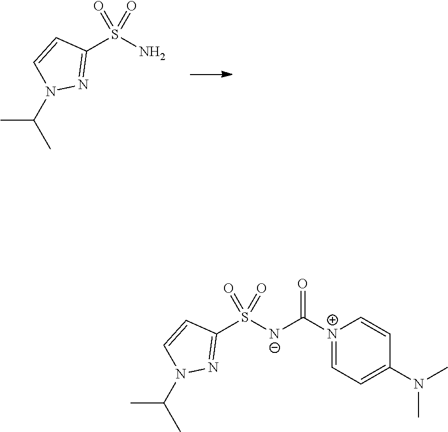 [SMILES: CC(C)n1ccc(S(N)(=O)=O)n1 ; CC(C)n1ccc(S(=O)(=O)[N-]C(=O)[n+]2ccc(N(C)C)cc2)n1]

A solution of 1-isopropyl-1H-pyrazole-3-sulfonamide (Intermediate L8) (712 mg, 3.76 mmol) in MeCN (4.4 mL) was treated with N,N-dimethylpyridin-4-amine (919 mg, 7.53 mmol) and the reaction mixture was stirred at room temperature until the sulfonamide had dissolved. Diphenyl carbonate (887 mg, 4.14 mmol) was added and the reaction mixture was left for 16 hours at room temperature. The resulting precipitate was separated by filtration, washed with MTBE and dried to afford the title compound (776 mg, 61%) as a white solid, which was used without further purification.

¹H NMR (CDCl₃) δ 8.95 (d, J=7.5 Hz, 2H), 7.35 (d, J=2.3 Hz, 1H), 6.83 (d, J=2.3 Hz, 1H), 6.62 (d, J=7.5 Hz, 2H), 4.58-4.43 (m, 1H), 3.24 (s, 6H), 1.42 (d, J=6.7 Hz, 6H).

### Intermediate L22: 1-Isopropyl-1H-1,2,3-triazole-5-sulfonamide

### Step A: 4-(Benzylthio)-1H-1,2,3-triazole

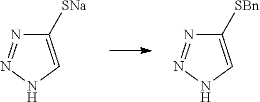

Benzyl bromide (24 mL, 202 mmol) was added dropwise to a suspension of sodium 1H-1,2,3-triazole-4-thiolate (25 g, 203 mmol) in EtOH (300 mL), cooled in an ice bath. The mixture was stirred for 48 hours, then the solvent was evaporated. The residue was partitioned between EtOAc (500 mL) and water (300 mL), the organic layer washed with brine (200 mL), dried (MgSO₄) and evaporated. The residue was triturated with TBME/isohexane to afford the title compound (35.1 g, 88%) as a white solid.

¹H NMR (CDCl₃) δ 7.40-7.24 (m, 7H), 4.16 (s, 2H).

LCMS m/z 192 (M+H)⁺ (ES⁺); 190 (M−H)⁻ (ES⁻).

### Step B: 5-(Benzylthio)-1-isopropyl-1H-1,2,3-triazole

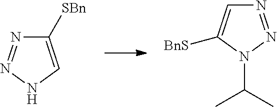

2-Iodopropane (7 mL, 70.1 mmol) was added to a mixture of 4-(benzylthio)-1H-1,2,3-triazole (12 g, 62.7 mmol) and K₂CO₃ (18 g, 130 mmol) in DMF (150 mL), cooled in an ice bath. The mixture was stirred for 2 hours, then warmed to room temperature and stirred for 20 hours. The mixture was partitioned between EtOAc (400 mL) and water (400 mL). The organic layer was washed with water (2×300 mL), dried (MgSO₄) and evaporated in vacuo. The residue was purified by chromatography on silica gel (220 g column, 0-50% EtOAc/isohexane) to afford the title compound (1.95 g, 13%) as an oil.

¹H NMR (CDCl₃) δ 7.62 (s, 1H), 7.32-7.26 (m, 3H), 7.14-7.10 (m, 2H), 4.64 (sept, J=6.7 Hz, 1H), 3.94 (s, 2H), 1.41 (d, J=6.7 Hz, 6H).

4-(Benzylthio)-1-isopropyl-1H-1,2,3-triazole (2.39 g, 16%) and 4-(benzylthio)-2-isopropyl-2H-1,2,3-triazole (9.16 g, 61%) were also isolated from this reaction.

### Step C: 1-Isopropyl-1H-1,2,3-triazole-5-sulfonyl chloride

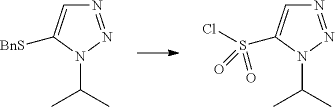

Prepared according to the general procedure of 1-isopropyl-1H-1,2,4-triazole-3-sulfonyl chloride (Intermediate L20, Step B) from 5-(benzylthio)-1-isopropyl-1H-1,2,3-triazole to afford an oil which was used without purification in the next step.

Step D: 1-Isopropyl-1H-1,2,3-triazole-5-sulfonamide

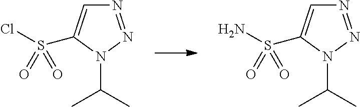

Prepared according to the general procedure of 1-(2,2,2-trifluoroethyl)-1H-pyrazole-4-sulfonamide (Intermediate L15, Step C) from 1-isopropyl-1H-1,2,3-triazole-5-sulfonyl chloride to afford the title compound (757 mg, 65%) as an off white solid.

¹H NMR (DMSO-d₆) δ 8.32 (br s, 2H), 8.04 (s, 1H), 5.15 (sept, J=6.6 Hz, 1H), 1.56 (d, J=6.6 Hz, 6H).

### Intermediate L23: 2-Isopropyl-2H-1,2,3-triazole-4-sulfonamide

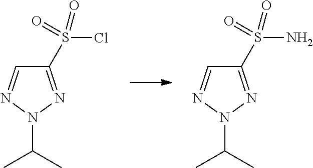

Prepared according to the general procedure of 1-(2,2,2-trifluoroethyl)-1H-pyrazole-4-sulfonamide (Intermediate L15, Step C) from 2-isopropyl-2H-1,2,3-triazole-4-sulfonyl chloride to afford the title compound (1.17 g, 71%) as a white solid.

¹H NMR (CDCl₃) δ 7.97 (s, 1H), 5.18 (br s, 2H), 4.92 (sept, J=6.7 Hz, 1H), 1.64 (d, J=6.7 Hz, 6H).

### Intermediate L24: 5-(2-Hydroxypropan-2-yl)-1-isopropyl-1H-pyrazole-3-sulfonamide

### Step A: 1-Isopropyl-N,N-bis(4-methoxybenzyl)-1H-pyrazole-3-sulfonamide

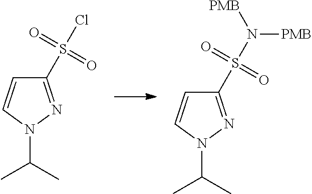

Prepared according to the general procedure of ethyl 3-(N,N-bis(4-methoxybenzyl)sulfamoyl)-1-methyl-1H-pyrazole-5-carboxylate (Intermediate L14, Step A) from 1-isopropyl-1H-pyrazole-3-sulfonyl chloride (Intermediate L6, Step C) to afford the title compound (16.6 g, 80%) as a white solid.

¹H NMR (DMSO-d₆) δ 8.00 (d, J=2.4 Hz, 1H), 7.07-6.96 (m, 4H), 6.85-6.76 (m, 4H), 6.70 (d, J=2.4 Hz, 1H), 4.61 (sept, J=6.7 Hz, 1H), 4.20 (s, 4H), 3.71 (s, 6H), 1.44 (d, J=6.7 Hz, 6H).

LCMS m/z 452.2 (M+Na)+ (ES⁺).

### Step B: 5-(2-Hydroxypropan-2-yl)-1-isopropyl-N,N-bis(4-methoxybenzyl)-1H-pyrazole-3-sulfonamide

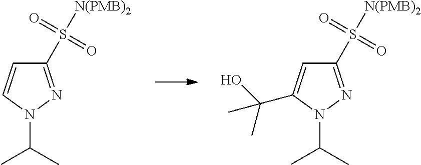

A solution of 1-isopropyl-N,N-bis(4-methoxybenzyl)-1H-pyrazole-3-sulfonamide (2.5 g, 5.82 mmol) in THF (30 mL) was cooled to −78° C. and BuLi (2.5 M in THF, 2.4 mL, 6.00 mmol) was added slowly via syringe. Upon complete addition, the mixture was stirred at −78° C. for 1 hour, before propan-2-one (0.52 mL, 7.08 mmol) was added slowly via syringe. The mixture was warmed to room temperature and stirred for 1 hour. The reaction was quenched with sat aq NH₄Cl (25 mL) and extracted with EtOAc (3×75 mL). The combined organic extracts were washed with brine (50 mL), dried (phase separator) and evaporated to give a yellow oil. The crude product was purified by chromatography on silica gel (80 g column, 0-100% EtOAc/isohexane) to afford the title compound (2.80 g, 49%) as a clear yellow oil that solidified slowly.

LCMS m/z 510.5 (M+Na)+ (ES⁺).

### Step C: 5-(2-Hydroxypropan-2-yl)-1-isopropyl-1H-pyrazole-3-sulfonamide

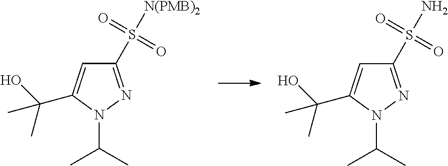

Prepared according to the general procedure of 5-(2-methoxypropan-2-yl)-1-methyl-1H-pyrazole-3-sulfonamide (Intermediate L14, Step D) from 5-(2-hydroxypropan-2-yl)-1-isopropyl-N,N-bis(4-methoxybenzyl)-1H-pyrazole-3-sulfonamide to afford the title compound (72.5 mg, 28%) as a pale yellow solid.

¹H NMR (DMSO-d₆) δ 7.32 (s, 2H), 6.34 (s, 1H), 5.51 (s, 1H), 5.27 (sept, J=6.6 Hz, 1H), 1.51 (s, 6H), 1.39 (d, J=6.6 Hz, 6H).

LCMS m/z 248.4 (M+H)⁺ (ES⁺).

### Intermediate L25: 5-(1-Methoxycyclobutyl)-1-methyl-1H-pyrazole-3-sulfonamide

### Step A: 5-(1-Hydroxycyclobutyl)-N,N-bis(4-methoxybenzyl)-1-methyl-1H-pyrazole-3-sulfonamide

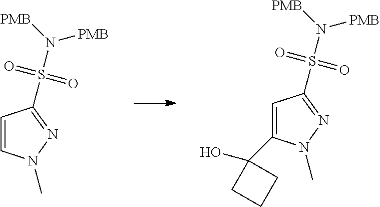

Prepared according to the general procedure of 5-(2-hydroxypropan-2-yl)-1-isopropyl-N,N-bis(4-methoxybenzyl)-1H-pyrazole-3-sulfonamide (Intermediate L24, Step B) from N,N-bis(4-methoxybenzyl)-1-methyl-1H-pyrazole-3-sulfonamide (Intermediate L12, Step B) and cyclobutanone to afford a 3:2 mixture of the title compound (1.09 g, 61%) and the starting material.

LCMS m/z 472.5 (M+H)⁺ (ES⁺).

### Step B: N,N-bis(4-Methoxybenzyl)-5-(1-methoxycyclobutyl)-1-methyl-1H-pyrazole-3-sulfonamide

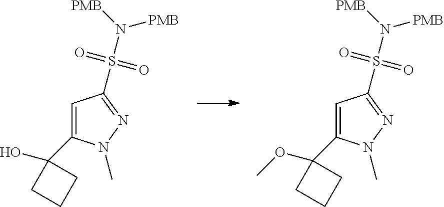

Prepared according to the general procedure of N,N-bis-(4-methoxybenzyl)-5-(2-methoxypropan-2-yl)-1-methyl-1H-pyrazole-3-sulfonamide (Intermediate L14, Step C) from 5-(1-hydroxycyclobutyl)-N,N-bis(4-methoxybenzyl)-1-methyl-1H-pyrazole-3-sulfonamide to afford the title compound (0.41 g, 91%) as a colourless oil.

¹H NMR (DMSO-d₆) δ=7.09-7.04 (m, 4H), 6.85-6.79 (m, 4H), 6.75 (s, 1H), 4.24 (s, 4H), 3.81 (s, 3H), 3.71 (s, 6H), 2.85 (s, 3H), 2.44-2.25 (m, 4H), 1.89-1.76 (m, 1H), 1.62-1.49 (m, 1H).

LCMS m/z 508.5 (M+Na)⁺, 486.5 (M+H)⁺ (ES⁺).

### Step C: 5-(1-Methoxycyclobutyl)-1-methyl-1H-pyrazole-3-sulfonamide

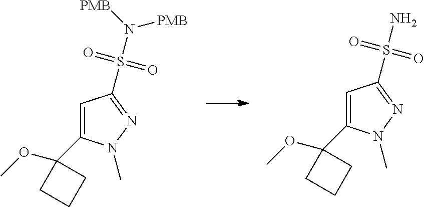

Prepared according to the general procedure of 1-ethyl-1H-pyrazole-3-sulfonamide (Intermediate L19, Step E) from N,N-bis(4-methoxybenzyl)-5-(1-methoxycyclobutyl)-1-methyl-1H-pyrazole-3-sulfonamide to afford the title compound (0.198 g, 89%) as a pale brown solid.

LCMS m/z 245.8 (M+H)⁺ (ES⁺).

### Intermediate L26: 5-(1-Methoxycyclopentyl)-1-methyl-1H-pyrazole-3-sulfonamide

### Step A: 5-(1-Hydroxycyclopentyl)-N,N-bis(4-methoxybenzyl)-1-methyl-1H-pyrazole-3-sulfonamide

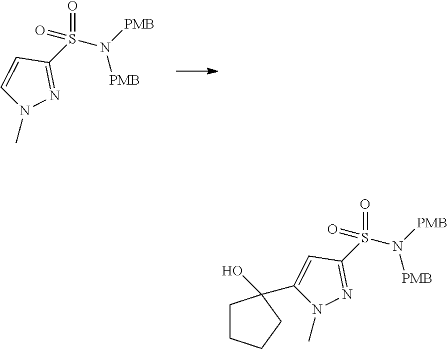

Prepared according to the general procedure of 5-(2-hydroxypropan-2-yl)-1-isopropyl-N,N-bis(4-methoxybenzyl)-1H-pyrazole-3-sulfonamide (Intermediate L24, Step B) from N,N-bis(4-methoxybenzyl)-1-methyl-1H-pyrazole-3-sulfonamide (Intermediate L12, Step B) and cyclopentanone to afford a 63:37 mixture (by HPLC) of starting material and title compound (1.96 g, 55%) as a cream coloured solid.

LCMS m/z 486.2 (M+H)⁺ (ES⁺).

### Step B: N,N-bis(4-Methoxybenzyl)-5-(1-methoxycyclopentyl)-1-methyl-1H-pyrazole-3-sulfonamide

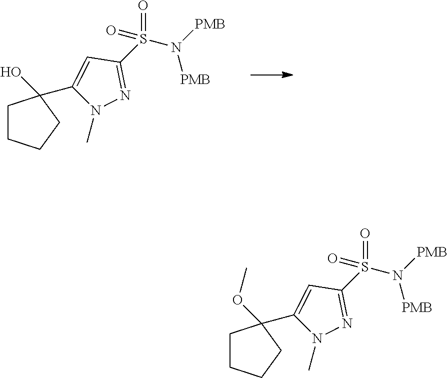

Prepared according to the general method of N,N-bis-(4-methoxybenzyl)-5-(2-methoxypropan-2-yl)-1-methyl-1H-pyrazole-3-sulfonamide (Intermediate L14, Step C) from 5-(1-hydroxycyclopentyl)-N,N-bis(4-methoxybenzyl)-1-methyl-1H-pyrazole-3-sulfonamide to afford the title compound (0.61 g, 93%) as a colourless oil.

LCMS m/z 500.2 (M+H)⁺ (ES⁺).

### Step C: 5-(1-Methoxycyclopentyl)-1-methyl-1H-pyrazole-3-sulfonamide

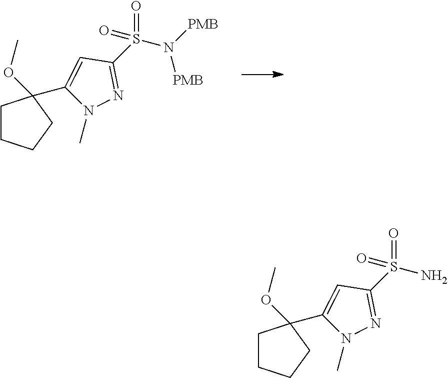

Prepared according to the general method of 5-(2-methoxypropan-2-yl)-1-methyl-1H-pyrazole-3-sulfonamide (Intermediate L14, Step D) from N,N-bis(4-methoxybenzyl)-5-(1-methoxycyclopentyl)-1-methyl-1H-pyrazole-3-sulfonamide to afford the title compound (0.22 g, 35%) as an orange solid.

LCMS m/z 260.3 (M+H)⁺ (ES⁺).

### Intermediate L27: 5-(1-Methoxyethyl)-1-methyl-1H-pyrazole-3-sulfonamide

### Step A: 5-(1-Hydroxyethyl)-N,N-bis(4-methoxybenzyl)-1-methyl-1H-pyrazole-3-sulfonamide

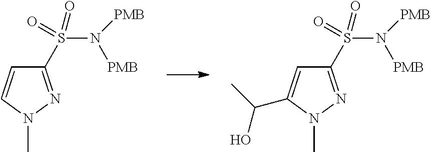

Prepared according to the general procedure of 5-(2-hydroxypropan-2-yl)-1-isopropyl-N,N-bis(4-methoxybenzyl)-1H-pyrazole-3-sulfonamide (Intermediate L24, Step B) from N,N-bis(4-methoxybenzyl)-1-methyl-1H-pyrazole-3-sulfonamide (Intermediate L12, Step B) and acetaldehyde to afford the title compound (2.12 g, 60%) as a viscous colourless oil.

¹H NMR (DMSO-d₆) δ 7.09-7.01 (m, 4H), 6.86-6.77 (m, 4H), 6.54 (s, 1H), 5.51 (d, J=5.7 Hz, 1H), 4.86 (p, J=6.4 Hz, 1H), 4.20 (s, 4H), 3.91 (s, 3H), 3.72 (s, 6H), 1.43 (d, J=6.5 Hz, 3H).

LCMS m/z 446 (M+H)⁺ (ES⁺).

### Step B: N,N-bis(4-Methoxybenzyl)-5-(1-methoxyethyl)-1-methyl-1H-pyrazole-3-sulfonamide

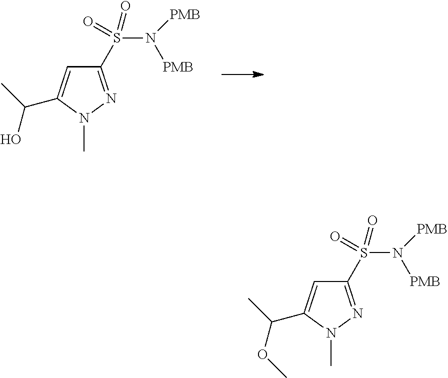

Prepared according to the general procedure of N,N-bis-(4-methoxybenzyl)-5-(2-methoxypropan-2-yl)-1-methyl-1H-pyrazole-3-sulfonamide (Intermediate L14, Step C) from 5-(1-hydroxyethyl)-N,N-bis(4-methoxybenzyl)-1-methyl-1H-pyrazole-3-sulfonamide to afford the title compound (858 mg, 99%) as an oil.

¹H NMR (CDCl₃) δ 7.12-7.08 (m, 4H), 6.81-6.76 (m, 4H), 6.51 (s, 1H), 4.53 (q, J=6.6 Hz, 1H), 4.34 (s, 4H), 3.95 (s, 3H), 3.80 (s, 6H), 3.29 (s, 3H), 1.53 (d, J=6.6 Hz, 3H).

LCMS m/z 460.1 (M+H)⁺ (ES⁺).

### Step C: 5-(1-Methoxyethyl)-1-methyl-1H-pyrazole-3-sulfonamide

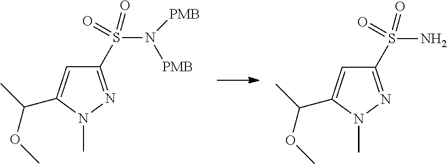

Prepared according to the general procedure of 1-ethyl-1H-pyrazole-3-sulfonamide (Intermediate L19, Step E) from N,N-bis(4-methoxybenzyl)-5-(1-methoxyethyl)-1-methyl-1H-pyrazole-3-sulfonamide to afford the title compound (395 mg, 93%) as a brown solid.

¹H NMR (DMSO-d₆) δ 7.37 (s, 2H), 6.53 (s, 1H), 4.64 (q, J=6.5 Hz, 1H), 3.87 (s, 3H), 3.22 (s, 3H), 1.43 (d, J=6.5 Hz, 3H).

LCMS m/z 220.2 (M+H)⁺ (ES⁺).

### Intermediate L28: 5-(1-Hydroxyethyl)-1-isopropyl-1H-pyrazole-3-sulfonamide

### Step A: 5-(1-Hydroxyethyl)-1-isopropyl-N,N-bis(4-methoxybenzyl)-1H-pyrazole-3-sulfonamide

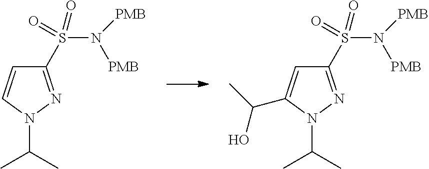

Prepared according to the general procedure of 5-(2-hydroxypropan-2-yl)-1-isopropyl-N,N-bis(4-methoxybenzyl)-1H-pyrazole-3-sulfonamide (Intermediate L24, Step B) from 1-isopropyl-N,N-bis(4-methoxybenzyl)-1H-pyrazole-3-sulfonamide (Intermediate L24, Step A) and acetaldehyde to afford the title compound (2.14 g, 65%) as a white solid.

¹H NMR (DMSO-d₆) δ 7.07-6.99 (m, 4H), 6.84-6.78 (m, 4H), 6.51 (s, 1H), 5.49 (d, J=6.0 Hz, 1H), 4.96-4.76 (m, 2H), 4.19 (s, 4H), 3.72 (s, 6H), 1.44 (d, J=6.5 Hz, 3H), 1.39 (app t, J=6.4 Hz, 6H).

LCMS m/z 496.4 (M+Na)⁺ (ES⁺).

### Step B: 5-(1-Hydroxyethyl)-1-isopropyl-1H-pyrazole-3-sulfonamide

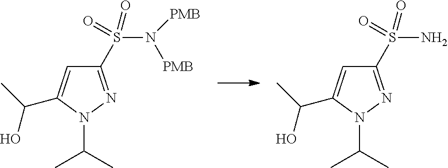

Prepared according to the general procedure of 1-ethyl-1H-pyrazole-3-sulfonamide (Intermediate L19, Step E) from 5-(1-hydroxyethyl)-1-isopropyl-N,N-bis(4-methoxybenzyl)-1H-pyrazole-3-sulfonamide to afford the title compound (0.09 g, 36%) as a white solid.

LCMS 234.3 (M+H)⁻ (ES⁻).

### Intermediate L29: 4-Fluoro-1-isopropyl-1H-pyrazole-3-sulfonamide

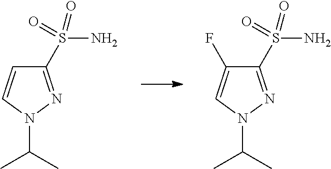

Selectfluor® (2.81 g, 7.93 mmol) was added to a solution of 1-isopropyl-1H-pyrazole-3-sulfonamide (Intermediate L8) (0.5 g, 2.64 mmol) in dry acetonitrile (8 mL) portionwise at room temperature, then the solution was stirred at 66° C. for 24 hours. A further portion of Selectfluor® (2.81 g, 7.93 mmol) was added and the reaction was stirred at 66° C. for a further 24 hours. The reaction was diluted with MeOH (50 mL), filtered and concentrated under reduced pressure. The residue was taken up in dry MeCN (10 mL), Selectfluor® (4.68 g, 13.2 mmol) was added portionwise and the reaction was stirred at 66° C. for 24 hours, cooled to room temperature, taken up in MeOH (50 mL), filtered and concentrated under reduced pressure. The residue was taken up in dry MeCN (20 mL), further Selectfluoro (3.7 g, 10.6 mmol) was added portionwise and the reaction was stirred at 66° C. for a further 18 hours. The volatiles were removed under reduced pressure and the residue was taken up in DCM/water (1:1, 150 mL). The organic phase separated, the aqueous phase further extracted with EtOAc (70 mL) and the combined organic phases dried (MgSO₄), filtered and concentrated under reduced pressure. The residue was purified by reversed phase prep-HPLC method 1 to afford the title compound (36 mg, 6%) as a white solid.

LCMS m/z 208.0 (M+H)⁺ (ES⁺).

### Intermediate L30: 1-(1-(Azetidin-1-yl)-2-methylpropan-2-yl)-1H-pyrazole-3-sulfonamide

### Step A: Methyl 2-(3-(N,N-bis(4-methoxybenzyl)sulfamoyl)-1H-pyrazol-1-yl)-2-methylpropanoate

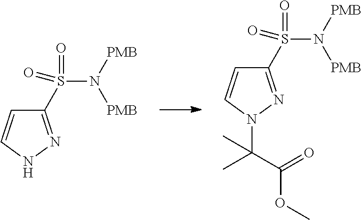

Prepared according to the general procedure of 1-ethyl-N,N-bis(4-methoxybenzyl)-1H-pyrazole-3-sulfonamide (Intermediate L19, Step D) from N,N-bis(4-methoxybenzyl)-1H-pyrazole-3-sulfonamide (Intermediate L19, Step C) and methyl 2-bromo-2-methylpropanoate to afford the title compound (2.45 g, 94%) as a clear colourless oil.

¹H NMR (DMSO-d₆) δ 8.18 (d, J=2.5 Hz, 1H), 7.05-6.95 (m, 4H), 6.85-6.78 (m, 4H), 6.78 (d, J=2.5 Hz, 1H), 4.18 (s, 4H), 3.72 (s, 6H), 3.65 (s, 3H), 1.81 (s, 6H).

LCMS m/z 511 (M+Na)⁺ (ES⁺).

### Step B: 2-(3-(N,N-bis(4-Methoxybenzyl)sulfamoyl)-1H-pyrazol-1-yl)-2-methylpropanoic acid

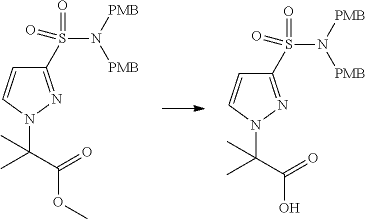

A mixture of methyl 2-(3-(N,N-bis(4-methoxybenzyl)sulfamoyl)-1H-pyrazol-1-yl)-2-methylpropanoate (2.4 g, 4.92 mmol) and aq 2 M NaOH (5 mL, 10 mmol) in THF (5 mL) and MeOH (3 mL) was stirred at room temperature for 20 hours. The mixture was partitioned between EtOAc (100 mL) and aq 1 M HCl (100 mL), the organic layer washed with brine (50 mL), dried (MgSO₄) and evaporated to afford the title compound (2.38 g, 95%) as a gum that solidified on standing.

¹H NMR (CDCl₃) δ 7.64 (d, J=2.5 Hz, 1H), 7.09-7.05 (m, 4H), 6.80-6.77 (m, 4H), 6.73 (d, J=2.5 Hz, 1H), 4.32 (s, 4H), 3.80 (s, 6H), 1.91 (s, 6H). One exchangeable proton not observed.

LCMS m/z 472 (M−H)⁻ (ES⁻).

### Step C: 1-(1-(Azetidin-1-yl)-2-methyl-1-oxopropan-2-yl)-N,N-bis(4-methoxybenzyl)-1H-pyrazole-3-sulfonamide

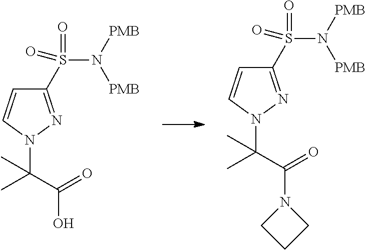

A mixture of 2-(3-(N,N-bis(4-methoxybenzyl)sulfamoyl)-1H-pyrazol-1-yl)-2-methylpropanoic acid (1.15 g, 2.23 mmol), Hunig's Base (1.56 mL, 8.91 mmol) and HATU (0.921 g, 2.42 mmol) in DMF (6.5 mL) was stirred at 0-5° C. for 10 minutes. Then azetidine HCl (0.272 g, 2.90 mmol) was added. The mixture was warmed to room temperature and stirred for 20 hours. Further HATU (0.263 g, 1.12 mmol) was added, followed by further Hunig's Base (0.390 mL, 2.23 mmol). The mixture was cooled to 0-5° C. for 10 minutes, then further azetidine HCl (0.064 g, 1.12 mmol) was added. The mixture was warmed to room temperature, stirred for a further 1 hour, then partitioned between TBME (75 mL) and water (40 mL). The organic layer was washed with aq 1 M HCl (40 mL), water (25 mL), dried (MgSO₄), evaporated and purified by chromatography on silica gel (120 g column, 0-100% TBME/isohexane) to afford the title compound (615 mg, 51%) as a clear gum.

¹H NMR (CDCl₃) δ 7.56 (d, J=2.4 Hz, 1H), 7.13-7.09 (m, 4H), 6.80-6.76 (m, 5H), 4.32 (s, 4H), 3.99 (t, J=7.8 Hz, 2H), 3.79 (s, 6H), 3.23 (t, J=7.7 Hz, 2H), 2.08-2.01 (m, 2H), 1.78 (s, 6H).

LCMS m/z 513.1 (M+H)⁺ (ES⁺).

### Step D: 1-(1-(Azetidin-1-yl)-2-methylpropan-2-yl)-N,N-bis(4-methoxybenzyl)-1H-pyrazole-3-sulfonamide

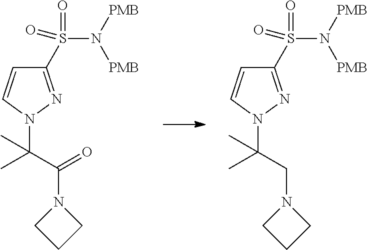

BH₃.THF (1 M in THF, 21.5 mL, 21.5 mmol) was added to a solution of 1-(1-(azetidin-1-yl)-2-methyl-1-oxopropan-2-yl)-N,N-bis(4-methoxybenzyl)-1H-pyrazole-3-sulfonamide (3.15 g, 6.15 mmol) in THF (26.3 mL). The mixture was stirred for 3 minutes, then heated to reflux over the weekend. The reaction was cooled to room temperature, before being placed in an ice-bath. MeOH (50 mL) was added dropwise and the mixture was heated at 60° C. for 3 hours, then cooled to room temperature overnight. The mixture was concentrated in vacuo and loaded onto a column of SCX (30 g) in MeOH (50 mL). The column was washed with MeOH (100 mL), 0.7 M ammonia in MeOH (100 mL) and the product was eluted with 7 M ammonia in MeOH (100 mL). The resultant mixture was concentrated in vacuo to afford the title compound (2.89 g, 85%) as a colourless viscous oil.

¹H NMR (DMSO-d₆) δ 7.98 (d, J=2.5 Hz, 1H), 7.07-7.02 (m, 4H), 6.84-6.79 (m, 4H), 6.69 (d, J=2.4 Hz, 1H), 4.19 (s, 4H), 3.72 (s, 6H), 2.92 (t, J=7.0 Hz, 4H), 2.68 (s, 2H), 1.84 (p, J=7.0 Hz, 2H), 1.48 (s, 6H).

LCMS m/z 499.2 (M+H)⁻ (ES⁻).

### Step E: 1-(1-(Azetidin-1-yl)-2-Methylpropan-2-yl)-1H-pyrazole-3-sulfonamide

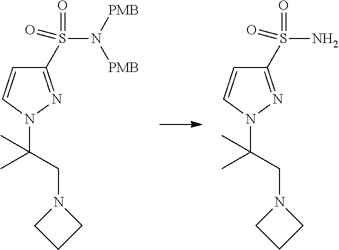

Prepared according to the general procedure of 1-ethyl-1H-pyrazole-3-sulfonamide (Intermediate L19, Step E) from 1-(1-(azetidin-1-yl)-2-Methylpropan-2-yl)-N,N-bis(4-methoxybenzyl)-1H-pyrazole-3-sulfonamide to afford the title compound (1.06 g, 69%) as a white solid.

¹H NMR (DMSO-d₆) δ 7.89 (d, J=2.5 Hz, 1H), 7.34 (s, 2H), 6.54 (d, J=2.4 Hz, 1H), 2.94 (t, J=7.0 Hz, 4H), 2.68 (s, 2H), 1.84 (p, J=7.0 Hz, 2H), 1.47 (s, 6H).

LCMS m/z 259.1 (M+H)⁺ (ES⁺).

### Intermediate L31: 3-((Dimethylamino)methyl)-5-methylbenzenesulfonamide

### Step A: 3-Bromo-N,N-bis(4-methoxybenzyl)-5-methylbenzenesulfonamide

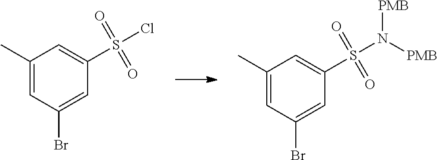

Prepared according to the general procedure of ethyl 3-(N,N-bis(4-methoxybenzyl)sulfamoyl)-1-methyl-1H-pyrazole-5-carboxylate (Intermediate L14, Step A) from 3-bromo-5-methylbenzene-1-sulfonyl chloride to afford the title compound (1.56 g, 77%) as a pale orange oil which solidified upon standing.

¹H NMR (Chloroform-d) δ 7.70-7.65 (m, 1H), 7.53-7.49 (m, 1H), 7.49-7.45 (m, 1H), 7.10-7.02 (m, 4H), 6.86-6.78 (m, 4H), 4.29 (s, 4H), 3.81 (s, 6H), 2.38 (s, 3H).

### Step B: 3-((Dimethylamino)methyl)-N,N-bis(4-methoxybenzyl)-5-methylbenzenesulfonamide

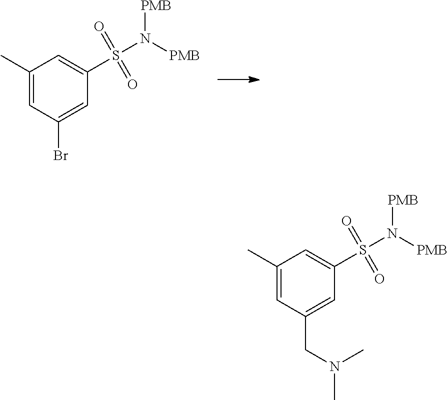

A solution of 3-bromo-N,N-bis(4-methoxybenzyl)-5-methylbenzenesulfonamide (290 mg, 0.591 mmol), potassium (N,N-dimethylaminomethyl)trifluoroboronate (117 mg, 0.710 mmol) and cesium carbonate (578 mg, 1.77 mmol) in THF (10 mL) and water (1 mL) was degassed with nitrogen for 5 minutes. Pd(crotyl)(XPhos)Cl (20 mg, 0.030 mmol) was added and the reaction mixture was stirred at reflux for 21 hours. The reaction mixture was cooled to room temperature and more potassium (N,N-dimethylaminomethyl)trifluoroboronate (117 mg, 0.710 mmol) and Pd(crotyl)(XPhos)Cl (20 mg, 0.030 mmol) were added. The reaction mixture was again stirred at reflux for 3 hours. After this time, the reaction mixture was partitioned between EtOAc (20 mL) and sat aq NH₄Cl (20 mL). The aqueous layer was separated and extracted with EtOAc (2×20 mL). The combined organics were washed with brine (30 mL), dried (MgSO₄) and concentrated in vacuo to afford an orange oil (322 mg). The crude product was loaded onto a column of SCX (2 g) in MeOH. The column was washed with MeOH and the product was eluted with 0.7 M ammonia in MeOH. The resultant mixture was concentrated in vacuo to afford the title compound (118 mg, 38%) as a sticky orange oil.

¹H NMR (Chloroform-d) δ 7.57-7.48 (m, 3H), 7.03-6.97 (m, 4H), 6.81-6.75 (m, 4H), 4.28 (s, 4H), 3.80 (s, 6H), 3.54 (s, 2H), 2.42 (s, 3H), 2.33 (s, 6H).

LCMS m/z 469.5 (M+H)⁺ (ES⁺).

### Step C: 3-((Dimethylamino)methyl)-5-methylbenzenesulfonamide

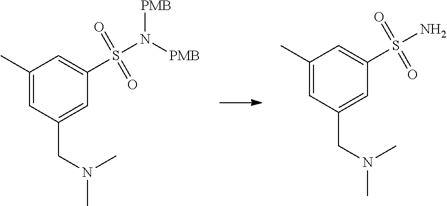

Prepared according to the general procedure of 1-ethyl-1H-pyrazole-3-sulfonamide (Intermediate L19, Step E) from 3-((dimethylamino)methyl)-N,N-bis(4-methoxybenzyl)-5-methylbenzenesulfonamide to afford the title compound (45 mg, 64%) as an orange gum.

¹H NMR (DMSO-d₆) δ 7.60-7.51 (m, 2H), 7.32 (s, 1H), 7.28 (s, 2H), 3.43 (s, 2H), 2.38 (s, 3H), 2.17 (s, 6H).

LCMS m/z 229.1 (M+H)⁺ (ES⁺).

### Intermediate L32: 1-(2,2-Difluoroethyl)-1H-pyrazole-4-sulfonamide

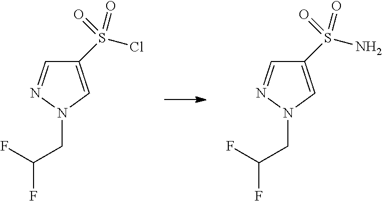 [SMILES: O=S(=O)(Cl)c1cnn(CC(F)F)c1 ; NS(=O)(=O)c1cnn(CC(F)F)c1]

Prepared according to the general procedure of 1-(2,2,2-trifluoroethyl)-1H-pyrazole-4-sulfonamide (Intermediate L15, Step C) from 1-(2,2-difluoroethyl)-1H-pyrazole-4-sulfonyl chloride to afford the title compound (422 mg, 92%) as a pale yellow solid.

¹H NMR (DMSO-d₆) δ 8.24 (d, J=0.7 Hz, 1H), 7.80 (d, J=0.7 Hz, 1H), 7.33 (s, 2H), 6.40 (tt, J=54.6, 3.6 Hz, 1H), 4.71 (td, J=15.2, 3.6 Hz, 2H).

¹⁹F NMR (DMSO-d₆) δ −123.15 (dt, J=54.5, 15.1 Hz).

LCMS m/z 212.0 (M+H)⁺ (ES⁺).

### Intermediate L33: 1-Cyclobutyl-1H-pyrazole-3-sulfonamide

### Step A: 1-Cyclobutyl-N,N-bis(4-methoxybenzyl)-1H-pyrazole-3-sulfonamide

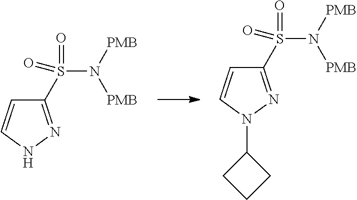

A solution of N,N-bis(4-methoxybenzyl)-1H-pyrazole-3-sulfonamide (Intermediate L19, Step C) (5 g, 12.9 mmol) in DMF (60 mL) was cooled to 0° C., before NaH (60 wt % in mineral oil, 0.671 g, 16.8 mmol) was added. The mixture was warmed to room temperature and stirred for 30 minutes, before bromocyclobutane (1.3 mL, 13.8 mmol) was added slowly via syringe. The resulting mixture was stirred at 50 ° C. over the weekend. The mixture was diluted with EtOAc (100 mL). Water (100 mL) was added and the layers were separated. The aqueous layer was extracted with EtOAc (2×100 mL). The combined organic extracts were washed with brine (3×80 mL), dried (phase separator) and concentrated in vacuo. The residue was loaded onto silica and purified by chromatography on silica gel (80 g column, 0-100% EtOAc/isohexane) to afford the title compound (4.72 g, 75%) as a pale yellow oil.

¹H NMR (DMSO-d₆) δ 8.03 (d, J=2.4 Hz, 1H), 7.04 (d, J=8.6 Hz, 4H), 6.81 (d, J=8.6 Hz, 4H), 6.71 (d, J=2.3 Hz, 1H), 4.94 (p, J=8.4 Hz, 1H), 4.22 (s, 4H), 3.72 (s, 6H), 2.49-2.38 (m, 4H), 1.87-1.77 (m, 2H).

LCMS m/z 464.2 (M+Na)⁺ (ES⁺).

### Step B: 1-Cyclobutyl-1H-pyrazole-3-sulfonamide

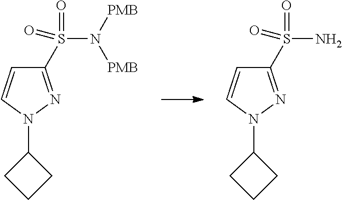

Prepared according to the general procedure of 1-ethyl-1H-pyrazole-3-sulfonamide (Intermediate L19, Step E) from 1-cyclobutyl-N,N-bis(4-methoxybenzyl)-1H-pyrazole-3-sulfonamide to afford the title compound (1.5 g, 66%) as a pale white solid.

¹H NMR (DMSO-d₆) δ 7.96 (d, J=2.4 Hz, 1H), 7.39 (s, 2H), 6.59 (d, J=2.4 Hz, 1H), 4.96-4.86 (m, 1H), 2.50-2.44 (m, 2H), 2.44-2.36 (m, 2H), 1.85-1.77 (m, 2H). LCMS m/z 202.0 (M+H)⁺ (ES⁺).

### Intermediate L34: 1-(1-((Dimethylamino)methyl)cyclobutyl)-1H-pyrazole-3-sulfonamide

Step A: Ethyl 1-(3-(N,N-bis(4-methoxybenzyl)sulfamoyl)-1H-pyrazol-1-yl)cyclobutane-1-carboxylate and ethyl 1-(5-(N,N-bis(4-methoxybenzyl)sulfamoyl)-1H-pyrazol-1-yl)cyclobutane-1-carboxylate

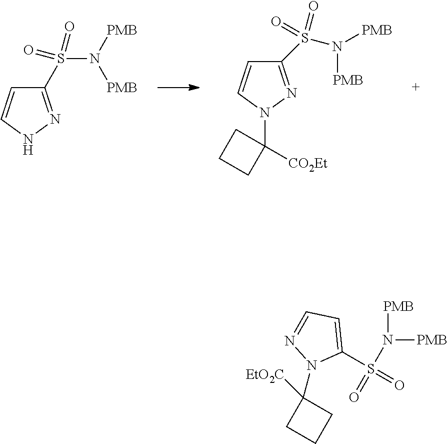

Prepared according to the general procedure of 1-cyclobutyl-N,N-bis(4-methoxybenzyl)-1H-pyrazole-3-sulfonamide (Intermediate L33, Step A) from N,N-bis(4-methoxybenzyl)-1H-pyrazole-3-sulfonamide (Intermediate L19, Step C) and ethyl 1-bromocyclobutanecarboxylate to afford the title compounds (1.26 g, 23%) as a mixture of regioisomers (3:1 ratio ethyl 1-(3-(N,N-bis(4-methoxybenzyl)sulfamoyl)-1H-pyrazol-1-yl)cyclobutane-1-carboxylate:ethyl 1-(5-(N,N-bis(4-methoxybenzyl)sulfamoyl)-1H-pyrazol-1-yl)cyclobutane-1-carboxylate) as a clear yellow oil.

LCMS m/z 536.2 (M+Na)⁺ (ES⁺).

### Step B: 1-(1-(Hydroxymethyl)cyclobutyl)-N,N-bis(4-methoxybenzyl)-1H-pyrazole-3-sulfonamide

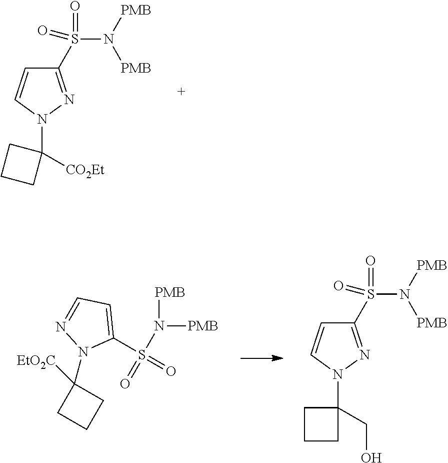

To a solution of a 3:1 mixture of ethyl 1-(3-(N,N-bis(4-methoxybenzyl)sulfamoyl)-1H-pyrazol-1-yl)cyclobutane-1-carboxylate and ethyl 1-(5-(N,N-bis(4-methoxybenzyl)sulfamoyl)-1H-pyrazol-1-yl)cyclobutane-1-carboxylate (710 mg, 1.04 mmol) in THF (20 mL) at 0° C. was added slowly LiAlH₄ (2 M in THF, 2.1 mL, 4.20 mmol). Then the mixture was warmed to room temperature and stirred overnight. The reaction was sequentially quenched with H₂O (0.2 mL), 2 M aq NaOH (0.5 mL) and H₂O (1 mL). Na₂SO₄ was added, the mixture was stirred for 30 minutes and then filtered through a plug of Celite, rinsing with EtOAc. The filtrate was evaporated and the residue loaded onto silica and purified by chromatography (40 g column, 15-100% EtOAc/isohexane) to afford the title compound (410 mg, 83%) as a clear colourless oil.

¹H NMR (DMSO-d₆) δ 7.89 (d, J=2.4 Hz, 1H), 7.01 (d, J=8.7 Hz, 4H), 6.82 (d, J=8.7 Hz, 4H), 6.70 (d, J=2.4 Hz, 1H), 5.20 (t, J=5.6 Hz, 1H), 4.20 (s, 4H), 3.75 (d, J=5.6 Hz, 2H), 3.72 (s, 6H), 2.48-2.39 (m, 2H), 2.39-2.27 (m, 2H), 1.95-1.80 (m, 2H).

LCMS m/z 494.0 (M+Na)+ (ES⁺).

### Step C: 1-(1-((Dimethylamino)methyl)cyclobutyl)-N,N-bis(4-methoxybenzyl)-1H-pyrazole-3-sulfonamide

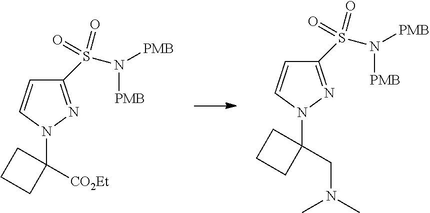

To an ice cooled solution of 1-(1-(hydroxymethyl)cyclobutyl)-N,N-bis(4-methoxybenzyl)-1H-pyrazole-3-sulfonamide (472 mg, 1.00 mmol) and Et₃N (0.3 mL, 2.15 mmol) in DCM (1 mL) was added MsCl (0.1 mL, 1.28 mmol). The reaction was warmed to room temperature and stirred for 1 hour, before being diluted with DCM (10 mL), washed with NaHCO₃ (10 mL) and brine (10 mL), dried (phase separator) and concentrated in vacuo. The residue was dissolved in THF (1 mL) and dimethylamine (5.00 mL, 10.0 mmol) was added. The reaction was heated to 80° C. in a sealed vial over the weekend. Dimethylamine (5.00 mL, 10.0 mmol) and KI (166 mg, 1.00 mmol) were added to the reaction mixture which was stirred at 85° C. overnight. A further portion of diethylamine (5.00 mL, 10.0 mmol) was added to the reaction mixture and this was heated at 80° C. overnight. The reaction mixture was cooled to room temperature and water (30 mL) and EtOAc (30 mL) were added. The organic phase was separated and the aqueous phase was extracted with EtOAc (3×20 mL). The organic phases were combined, dried (phase separator) and concentrated in vacuo. The crude product was loaded onto a column of SCX (about 10 g) in MeOH. The column was washed with MeOH (50 mL) and the product was eluted with 0.7 M ammonia in MeOH (50 mL). The resultant mixture was concentrated in vacuo to afford the title compound (187 mg, 36%) as a clear yellow oil.

LCMS m/z 499.3 (M+H)⁺ (ES⁺).

### Step D: 1-(1-((Dimethylamino)methyl)cyclobutyl)-1H-pyrazole-3-sulfonamide

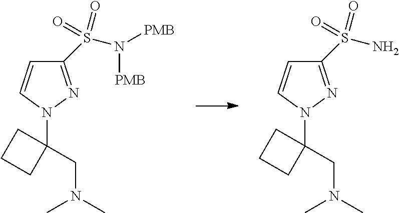

Prepared according to the general procedure of 1-ethyl-1H-pyrazole-3-sulfonamide (Intermediate L19, Step E) from 1-(1-((dimethylamino)methyl)cyclobutyl)-N,N-bis(4-methoxybenzyl)-1H-pyrazole-3-sulfonamide to afford the title compound (65 mg, 64%) as a sticky yellow oil.

LCMS m/z 259.1 (M+H)⁺ (ES⁺).

### Intermediate L35: 5-Fluoro-1-isopropyl-1H-pyrazole-3-sulfonamide

### Step A: 5-Fluoro-1-isopropyl-N,N-bis(4-methoxybenzyl)-1H-pyrazole-3-sulfonamide

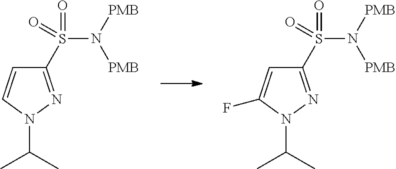

To a stirred solution of 1-isopropyl-N,N-bis(4-methoxybenzyl)-1H-pyrazole-3-sulfonamide (Intermediate L24, Step A) (0.500 g, 1.16 mmol) in THF (8 mL) at −78° C. under N₂ was added n-BuLi (2.5 M in hexanes, 0.500 mL, 1.25 mmol) dropwise over 15 minutes. The resulting mixture was stirred at −78° C. for 1 hour, then N-fluoro-N-(phenylsulfonyl)benzenesulfonamide (NFSI) (0.394 g, 1.25 mmol) in THF (2 mL) was added dropwise over 15 minutes. The resulting reaction mixture was stirred at −78° C. for 2 hours and then at room temperature overnight. The reaction mixture was quenched by the addition of water (20 mL) and extracted with DCM (3×20 mL). The combined organics were washed with sat aq NaHCO₃ (30 mL), brine (30 mL), dried (phase separator) and concentrated in vacuo to afford a yellow oil. The crude product was purified by chromatography on silica gel (40 g cartridge, 0-30% EtOAc/isohexane) to afford a 1.6:1 mixture of the title compound and 4,5-difluoro-1-isopropyl-N,N-bis(4-methoxybenzyl)-1H-pyrazole-3-sulfonamide (124 mg) as a yellow oil which solidified is upon standing.

¹H NMR (DMSO-d₆) δ 7.08-7.02 (m, 4H), 6.87-6.80 (m, 4H), 6.53 (d, J=5.6 Hz, 1H), 4.69-4.57 (m, 1H), 4.23 (s, 4H), 3.73 (s, 6H), 1.41 (d, J=6.7 Hz, 6H).

LCMS m/z 470.3 (M+Na)⁺ (ES⁺), 62% and 488.2 (M+Na)⁺ (ES⁺).

### Step B: 5-Fluoro-1-isopropyl-1H-pyrazole-3-sulfonamide

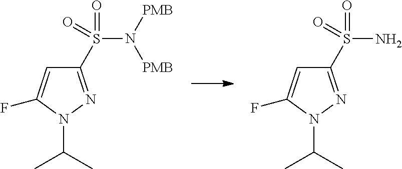

Prepared according to the general procedure of 1-ethyl-1H-pyrazole-3-sulfonamide (Intermediate L19, Step E) from a 1.6:1 mixture of 5-fluoro-1-isopropyl-N,N-bis(4-methoxybenzyl)-1H-pyrazole-3-sulfonamide and 4,5-difluoro-1-isopropyl-N,N-bis(4-methoxybenzyl)-1H-pyrazole-3-sulfonamide to afford the title compound (39 mg, 16% over 2 steps) as a white solid.

¹H NMR (DMSO-d₆) δ 7.52 (s, ²H), 6.36 (d, J=5.7 Hz, 1H), 4.67-4.57 (m, 1H), 1.41 (d, J=6.7 Hz, 6H).

LCMS m/z 208.2 (M+H)⁺ (ES⁺).

4,5-Difluoro-1-isopropyl-1H-pyrazole-3-sulfonamide (18 mg, 6% over 2 steps) was also isolated as a white solid.

### Intermediate L36: ((1-(2,2-Difluoroethyl)-1H-pyrazol-3-yl)sulfonyl)(4-(dimethylamino)pyridin-1-ium-1-carbonyl)amide

### Step A: 1-(2,2-Difluoroethyl)-N,N-bis(4-methoxybenzyl)-1H-pyrazole-3-sulfonamide

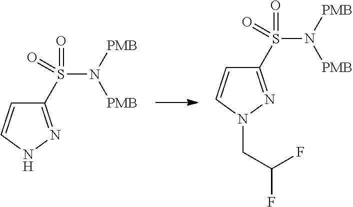

Prepared according to the general procedure of 1-ethyl-N,N-bis(4-methoxybenzyl)-1H-pyrazole-3-sulfonamide (Intermediate L19, Step D) from N,N-bis(4-methoxybenzyl)-1H-pyrazole-3-sulfonamide (Intermediate L19, Step C) and 1,1-difluoro-2-iodoethane to afford the title compound (775 mg, 57%) as a clear colourless oil which solidified upon standing.

¹H NMR (DMSO-d₆) δ 8.02 (d, J=2.4 Hz, 1H), 7.04-6.98 (m, 4H), 6.85-6.75 (m, 5H), 6.55-6.29 (m, 1H), 4.85-4.74 (m, 2H), 4.21 (s, 4H), 3.72 (s, 6H).

LCMS m/z 474.3 (M+Na)⁺ (ES⁺).

### Step B: 1-(2,2-Difluoroethyl)-1H-pyrazole-3-sulfonamide

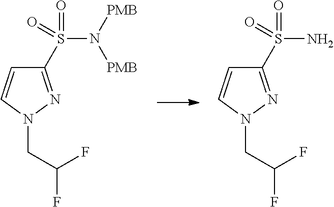

Prepared according to the general procedure of 1-ethyl-1H-pyrazole-3-sulfonamide (Intermediate L19, Step E) from 1-(2,2-difluoroethyl)-N,N-bis(4-methoxybenzyl)-1H-pyrazole-3-sulfonamide to afford the title compound (360 mg, 99%) as a white solid.

¹H NMR (DMSO-d₆) δ 7.93 (d, J=2.4 Hz, 1H), 7.49 (s, 2H), 6.66 (d, J=2.4 Hz, 1H), 6.40 (tt, J=54.6, 3.6 Hz, 1H), 4.73 (td, J=J=15.3, 3.6 Hz, 2H).

### Step C: ((1-(2,2-Difluoroethyl)-1H-pyrazol-3-yl)sulfonyl)(4-(dimethylamino)pyridin-1-ium-1-carbonyl)amide

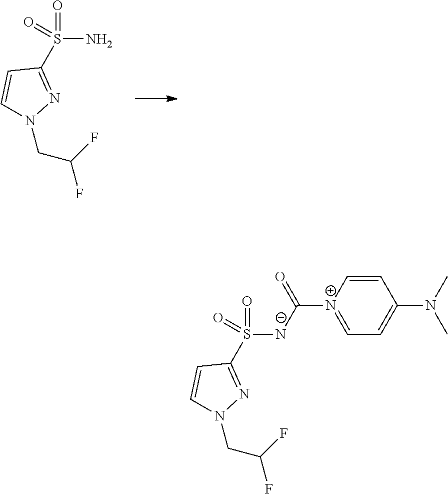 [SMILES: NS(=O)(=O)c1ccn(CC(F)F)n1 ; CN(C)c1cc[n+](C(=O)[N-]S(=O)(=O)c2ccn(CC(F)F)n2)cc1]

Prepared according to the general procedure of (4-(dimethylamino)pyridin-1-ium-1-carbonyl)((1-isopropyl-1H-pyrazol-3-yl)sulfonyl)amide (Intermediate L21) from 1-(2,2-difluoroethyl)-1H-pyrazole-3-sulfonamide to afford the title compound (83 mg, 11%) as a white solid. The crude product was used without further purification or analysis.

### Intermediate L37: 1-(2-Methyl-1-(pyrrolidin-1-yl)propan-2-yl)-1H-pyrazole-3-sulfonamide

### Step A: N,N-bis(4-Methoxybenzyl)-1-(2-methyl-1-oxo-1-(pyrrolidin-1-yl)propan-2-yl)-1H-pyrazole-3-sulfonamide

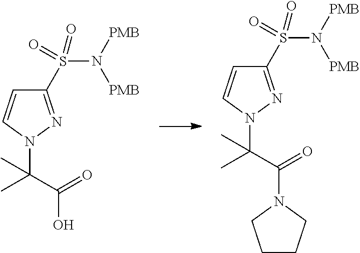

Prepared according to the general procedure of 1-(1-(azetidin-1-yl)-2-methyl-1-oxopropan-2-yl)-N,N-bis(4-methoxybenzyl)-1H-pyrazole-3-sulfonamide (Intermediate L30, Step C) from 2-(3-(N,N-bis(4-methoxybenzyl)sulfamoyl)-1H-pyrazol-1-yl)-2-methylpropanoic acid (Intermediate L30, Step B) and pyrrolidine to afford the title compound (862 mg, 73%) as a pale white solid.

¹H NMR (DMSO-d₆) δ 8.16 (d, J=2.5 Hz, 1H), 7.06 (d, J=8.6 Hz, 4H), 6.85 (d, J=2.5 Hz, 1H), 6.82 (d, J=8.7 Hz, 4H), 4.17 (s, 4H), 3.72 (s, 6H), 3.37-3.33 (m, 2H), 2.38-2.32 (m, 2H), 1.73 (s, 6H), 1.59 (br s, 4H).

### Step B: N,N-bis(4-Methoxybenzyl)-1-(2-methyl-1-(pyrrolidin-1-yl)propan-2-yl)-1H-pyrazole-3-sulfonamide

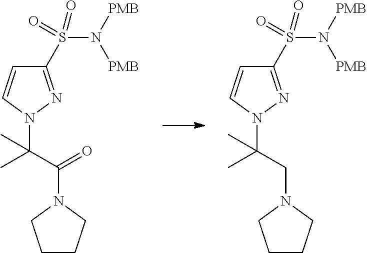

Prepared according to the general procedure of 1-(1-(azetidin-1-yl)-2-methylpropan-2-yl)-N,N-bis(4-methoxybenzyl)-1H-pyrazole-3-sulfonamide (Intermediate L30, Step D) from N,N-bis(4-methoxybenzyl)-1-(2-methyl-1-oxo-1-(pyrrolidin-1-yl)propan-2-yl)-1H-pyrazole-3-sulfonamide to afford the title compound (388 mg, 42%).

¹H NMR (DMSO-d₆) δ 8.01 (d, J=2.5 Hz, 1H), 7.05 (d, J=8.6 Hz, 4H), 6.82 (d, J=8.6 Hz, 4H), 6.71 (d, J=2.4 Hz, 1H), 4.18 (s, 4H), 3.72 (s, 6H), 2.81 (s, 2H), 2.24-2.15 (m, 4H), 1.55 (s, 6H), 1.54-1.51 (m, 4H).

LCMS m/z 513.0 (M+H)⁺ (ES⁺).

### Step C: 1-(2-Methyl-1-(pyrrolidin-1-yl)propan-2-yl)-1H-pyrazole-3-sulfonamide

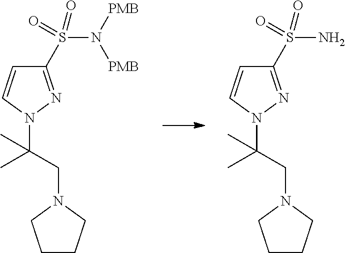

Prepared according to the general procedure of 1-ethyl-1H-pyrazole-3-sulfonamide (Intermediate L19, Step E) from N,N-bis(4-methoxybenzyl)-1-(2-methyl-1-(pyrrolidin-1-yl)propan-2-yl)-1H-pyrazole-3-sulfonamide to afford the title compound (136 mg, 59%) as a sticky colourless oil.

LCMS m/z 273.1 (M+H)⁺ (ES⁺).

### Intermediate L38: ((1-Cyclopropyl-1H-pyrazol-3-yl)sulfonyl)(4-(dimethylamino)pyridin-1-ium-1-carbonyl)amide

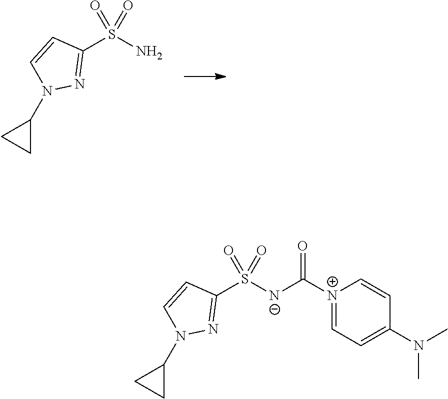 [SMILES: NS(=O)(=O)c1ccn(C2CC2)n1 ; CN(C)c1cc[n+](C(=O)[N-]S(=O)(=O)c2ccn(C3CC3)n2)cc1]

Prepared according to the general procedure of (4-(dimethylamino)pyridin-1-ium-1-carbonyl)((1-isopropyl-1H-pyrazol-3-yl)sulfonyl)amide (Intermediate L21) from 1-cyclopropyl-1H-pyrazole-3-sulfonamide to afford the title compound (1.57 g, 55%) as a solid.

¹H NMR (DMSO-d₆) δ 8.82-8.63 (m, 2H), 7.81 (d, J=2.3 Hz, 1H), 7.04-6.86 (m, 2H), 6.57 (d, J=2.4 Hz, 1H), 3.76 (m, 1H), 3.25 (s, 6H), 1.07-1.01 (m, 2H), 1.00-0.95 (m, 2H).

### Intermediate R1: 4-Isocyanato-1,2,3,5,6,7-hexahydro-s-indacene

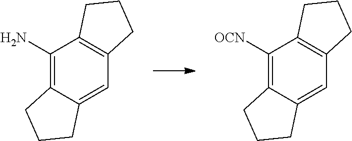

To a solution of phosgene (4.45 mL, 20% weight in toluene, 8.4 mmol) in ethyl acetate (90 mL) was added dropwise a solution of 1,2,3,5,6,7-hexahydro-s-indacen-4-amine (589 mg, 3.4 mmol) in ethyl acetate (45 mL) at ambient temperature. The resulting reaction mixture was then heated to reflux for 3 hours and upon cooling was filtered and concentrated in vacuo to afford the title compound as a brown oil (756 mg, 100%). The crude product was used directly in the next step without further purification.

¹H NMR (300 MHz, CDCl₃) δ 6.8 (s, 1H), 2.89 (m, 8H) and 2.09 (m, 4H).

### Intermediate R2: 8-Isocyanato-1,2,3,5-tetrahydro-s-indacene

### Step A: 1,2,3,7-Tetrahydro-s-indacen-4-amine

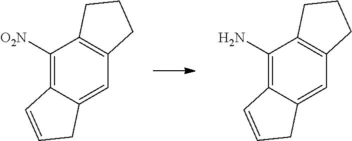 [SMILES: O=[N+]([O-])c1c2c(cc3c1CCC3)CC=C2 ; Nc1c2c(cc3c1CCC3)CC=C2]

To a solution of 8-nitro-1,2,3,5-tetrahydro-s-indacene (Salla et al, ACS Med Chem Lett, 2016, vol. 7(12), pages 1034-1038) (700 mg, 3.48 mmol) in a mixture of dioxane/ethanol/water (10 ml/6 mL/4 mL) was added iron powder (1.17 g, 20.9 mmol) and ammonium chloride (0.93 g, 17.4 mmol). The mixture was refluxed for 15 minutes. Ethyl acetate (50 mL) was added and the mixture was filtered over Celite. The solids were washed with ethyl acetate. The combined ethyl acetate layers were evaporated. The crude product was filtered over silica, using ethyl acetate as the eluent, to afford the title compound (97%) as a brown oil that solidified upon standing.

¹H NMR (300 MHz, CDCl₃) δ 6.88 (s, 1H), 6.85 (m, 1H), 6.39 (m, 1H), 3.68 (s, br, 2H) 3.36 (s, 2H), 2.93 (t, 2H), 2.75 (t, 2H), 2.14 (m, 2H).

### Step B: 8-Isocyanato-1,2,3,5-tetrahydro-s-indacene

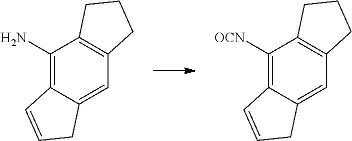

To a solution of phosgene (0.23 mL, 20% weight in toluene, 0.44 mmol) in ethyl acetate (5 mL) was added dropwise a solution of 1,2,3,7-tetrahydro-s-indacen-4-amine (30 mg, 0.18 mmol) in ethyl acetate (5 mL) at ambient temperature. The resulting reaction mixture was then heated to reflux for 2 hours. The mixture was filtered and concentrated in vacuo to afford the title compound (35 mg, 100%) as a brown oil which solidified upon standing. The crude product was used directly in the next step without further purification.

¹H NMR (CDCl₃) δ 7.12 (s, 1H), 6.80 (m, 1H), 6.51 (m, 1H), 3.35 (q, 2H), 2.96 (m, 4H), 2.14 (p, 2H).

### Intermediate R3: 4-Isocyanato-3,5,6,7-tetrahydro-s-indacen-1(2H)-one

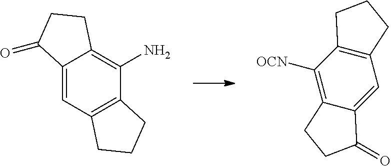

A solution of 4-amino-3,5,6,7-tetrahydro-s-indacen-1(2H)-one (Salla et al, ACS Med Chem Lett, 2016, vol. 7(12), pages 1034-1038) (40 mg, 0.21 mmol) in ethyl acetate (5 mL) was added dropwise over 10 minutes to a solution of phosgene (20% in toluene, 0.5 mL, 0.54 mmol) in ethyl acetate (5 mL). The mixture was stirred at reflux for 1 hour. Then the mixture was concentrated to afford the title compound (45 mg, 100%) as an oil, which was used as such for the next step.

### Intermediate R4: 8-Isocyanato-3,5,6,7-tetrahydro-s-indacen-1(2H)-one

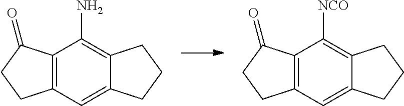

To a solution of 8-amino-3,5,6,7-tetrahydro-s-indacen-1(2H)-one (57 mg, 0.30 mmol) in toluene was added a solution of phosgene (20% in toluene, 0.39 mL, 0.75 mmol) and the mixture was refluxed for 30 minutes. Concentration under reduced pressure gave the title compound (64.6 mg, 99%) as a green oil that was used without further purification.

¹H NMR (CDCl₃) δ 7.10 (s, 1H), 3.03 (m, 2H), 2.96 (t, 2H), 2.89 (t, 2H), 2.71 (m, 2H), 2.12 (p, 2H).

### Intermediate R5: 3-(4-Fluoro-2-isopropyl-6-(2-methoxypyridin-4-yl)phenyl)propanoyl chloride

Step A: tert-Butyl 3-(4-fluoro-2-isopropyl-6-(2-methoxypyridin-4-yl)phenyl)acrylate

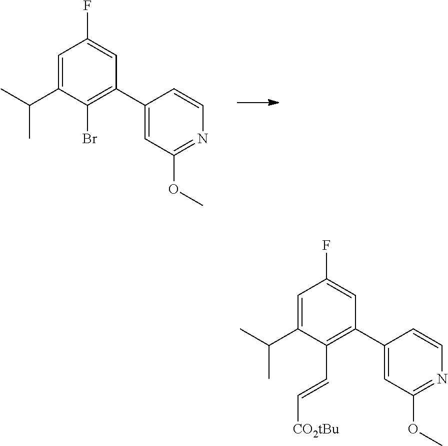 [SMILES: COc1cc(-c2cc(F)cc(C(C)C)c2Br)ccn1 ; COc1cc(-c2cc(F)cc(C(C)C)c2/C=C/C(=O)OC(C)(C)C)ccn1]

A solution of 4-(2-bromo-5-fluoro-3-isopropylphenyl)-2-methoxypyridine (0.9 g, 3 mmol) and tert-butyl acrylate (1 g, 3 eq, 8 mmol) in DMF (10 ml) was purged with nitrogen. Potassium carbonate (0.8 g, 6 mmol), triphenylphosphine (0.1 g, 0.6 mmol) and palladium acetate (67 mg, 0.30 mmol) were added. The mixture was stirred for 18 hours at 120° C. The mixture was cooled and ethyl acetate was added. The organic layer was washed with water (4 times) and brine, dried (sodium sulfate), filtered and evaporated. The residue was purified over silica using ethyl acetate/heptane as the eluent to afford the title compound (0.6 g, 58%) as a pale red oil.

¹H NMR (300 MHz, CDCl₃) δ 8.14 (d, 1H), 7.58 (d, 1H), 7.07 (dd, 1H), 6.85 (dd, 1H), 6.75 (dd, 1H), 6.66 (s, 1H), 5.58 (d, 1H), 3.98 (s, 3H), 3.24 (m, 1H), 1.44 (s, 9H), 1.25 (d, 6H).

### Step B: tert-Butyl 3-(4-fluoro-2-isopropyl-6-(2-methoxypyridin-4-yl)phenyl)propanoate

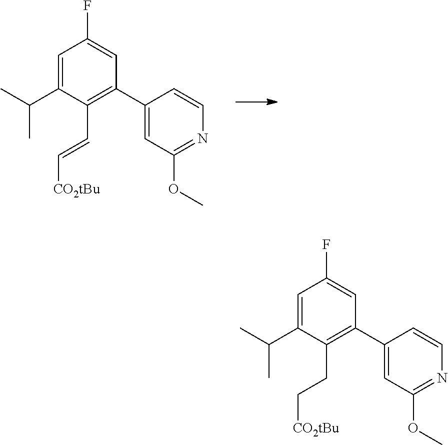 [SMILES: COc1cc(-c2cc(F)cc(C(C)C)c2/C=C/C(=O)OC(C)(C)C)ccn1 ; COc1cc(-c2cc(F)cc(C(C)C)c2CCC(=O)OC(C)(C)C)ccn1]

A mixture of tert-butyl 3-(4-fluoro-2-isopropyl-6-(2-methoxypyridin-4-yl)phenyl)acrylate (0.6 g, 1.6 mmol) and Pd/C (10%, 50 mg) in ethyl acetate was stirred for 18 hours under hydrogen atmosphere (balloon). The mixture was filtered (Celite) and evaporated to afford the title compound (0.6 g, quantitative yield) as an oil.

¹H NMR (300 MHz, CDCl₃) δ 8.20 (d, 7.02 (dd, 6.80 (d, 6.70 (dd, 6.64 (s, 1H), 3.98 (s, 3H), 3.21 (m, 2.81 (t, 2H), 2.20 (t, 2H), 1.37 (s, 9H), 1.27 (d, 6H).

### Step C: 3-(4-Fluoro-2-isopropyl-6-(2-methoxypyridin-4-yl)phenyl)propanoic acid

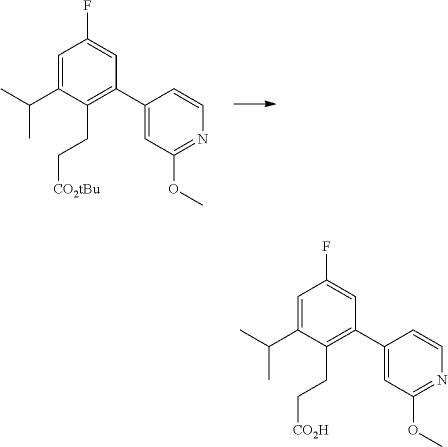 [SMILES: COc1cc(-c2cc(F)cc(C(C)C)c2CCC(=O)OC(C)(C)C)ccn1 ; COc1cc(-c2cc(F)cc(C(C)C)c2CCC(=O)O)ccn1]

To a solution of tert-butyl 3-(4-fluoro-2-isopropyl-6-(2-methoxypyridin-4-yl)phenyl)propanoate (0.60 g, 1.6 mmol) in dichloromethane (5 mL) was added TFA (5 mL). The mixture was stirred for 18 hours at room temperature. The solvents were evaporated to afford the title compound as TFA salt (0.63 g, 100%) as a colourless oil.

¹H NMR (300 MHz, CDCl₃) δ 8.44 (d, 1H), 7.29 (d, 1H), 7.15 (dd, 1H), 7.11 (s, 1H), 6.73 (dd, 1H), 4.21 (s, 3H), 3.16 (m, 1H), 2.90 (t, 2H), 2.41 (t, 2H), 1.30 (d, 6H).

### Step D: 3-(4-Fluoro-2-isopropyl-6-(2-methoxypyridin-4-yl)phenyl)propanoyl chloride

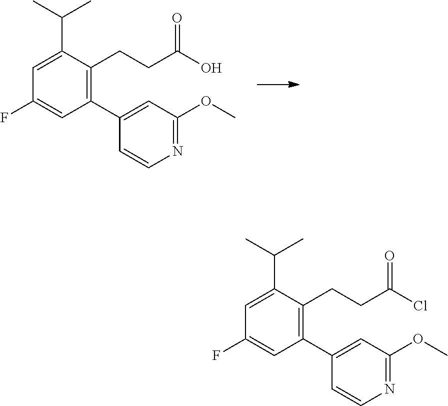 [SMILES: COc1cc(-c2cc(F)cc(C(C)C)c2CCC(=O)O)ccn1 ; COc1cc(-c2cc(F)cc(C(C)C)c2CCC(=O)Cl)ccn1]

3-(4-Fluoro-2-isopropyl-6-(2-methoxypyridin-4-yl)phenyl)propanoic acid (285 mg, 62 wt %, 0.56 mmol) was stirred in DCM (10 mL) and a drop of DMF was added, followed by the dropwise addition of oxalylchloride (0.24 mL, 2.8 mmol). The solution was stirred at room temperature for 4 hours and concentrated thoroughly to afford the title compound (190 mg, 99%) as a yellow oil.

¹H NMR (300 MHz, Chloroform-d) δ 8.45 (d, 1H), 7.34 (d, 1H), 7.17-7.07 (m, 2H), 6.72 (dd, 1H), 4.38 (s, 3H), 3.19 (dd, 1H), 2.96-2.82 (m, 2H), 2.41 (d, 2H), 1.28 (d, 6H).

### Intermediate R6: 6-Fluoro-8-isopropyl-4,4-dimethyl-1,2,3,4-tetrahydronaphthalene-1-carbonyl chloride

### Step A: 2-(4-Fluoro-2-(prop-1-en-2-yl)phenyl)acetic acid mixed with 2,3-dimethylbutane-2,3-diol (1:1)

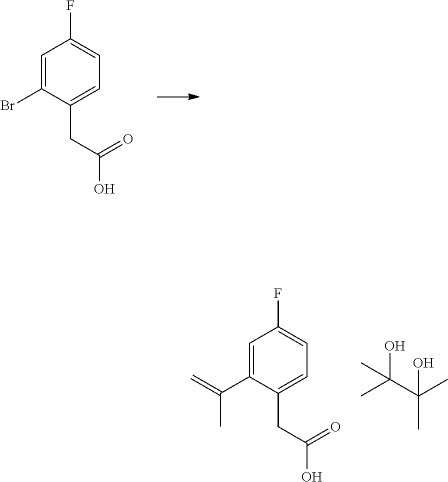

Potassium carbonate (120 g, 0.89 mol) was dissolved in water (100 mL) and added to a solution of 2-(2-bromo-4-fluorophenyl)acetic acid (69 g, 1 eq, 0.30 mol) and 4,4,5,5-tetramethyl-2-(prop-1-en-2-yl)-1,3,2-dioxaborolane (50 g, 1 eq, 0.30 mol) in 1,4-dioxane (100 mL). The mixture was brought under N₂ atmosphere and PdCl₂(dPPf)-CH₂Cl₂ adduct (4.9 g, 6.0 mmol) was added, after which the mixture was refluxed for 48 hours. The mixture was cooled to room temperature. Water was added until all salts were dissolved. The layers were separated and the aqueous layer was extracted with ethyl acetate (2×100 mL). The combined organic layers were dried over sodium sulfate and solvent was removed by rotary evaporation, yielding the product (87 g, 94%) as a brown oil.

¹H NMR (CDCl₃) δ 7.22 (dd, 1H), 6.95 (dd, 1H), 6.88 (dd, 1H), 5.23 (s, 1H), 4.84 (s, 1H), 3.68 (s, 2H), 2.00 (s, 3H), 1.24 (s, 6H), 1.21 (s, 6H).

LCMS: m/z 193 (M−H)⁻ (ES⁻).

### Step B: 2-(4-Fluoro-2-isopropylphenyl)acetic acid

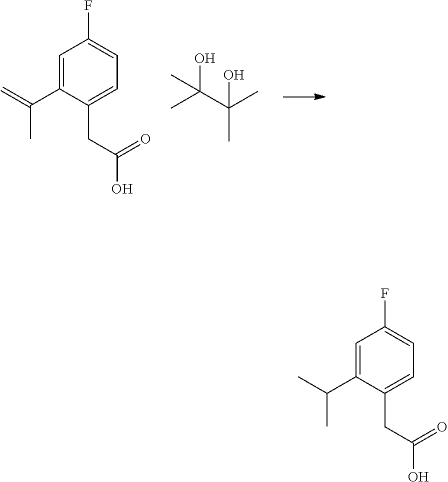

A mixture of 2-(4-fluoro-2-(prop-1-en-2-yl)phenyl)acetic acid with 2,3-dimethylbutane-2,3-diol (1:1) (80 g, 0.26 mol) and platinum(IV) oxide (1 g, 4 mmol) were dissolved in ethanol (200 mL) and stirred overnight under 3 bar H₂ pressure. Further platinum(IV) oxide (0.1 g, 0.4 mmol) was added, and the mixture was stirred for another 5 hours under 3 bar H₂. The mixture was filtered over Celite and ethanol was removed by rotary evaporation. The resulting oil was subjected to falling film distillation to remove the pinacol. The residue was taken up in DCM (200 mL) and washed with 0.5 M aqueous HCl (100 mL). The organic layer was dried over sodium sulfate and plugged over SiO₂ (d=10 cm, h=5 cm). The SiO₂ was flushed with DCM (2 L). Rotary evaporation of the collected eluate yielded the product (45 g, 90%) as a light orange solid.

¹H NMR (CDCl₃) δ 7.18 (dd, 1H), 7.00 (dd, 1H), 6.82 (dt, 1H), 3.67 (s, 2H), 3.07 (m, 1H), 1.20 (d, 6H).

LCMS: m/z 195 (M−H)⁻ (ES⁻).

### Step C: Ethyl 2-(4-fluoro-2-isopropylphenyl)acetate

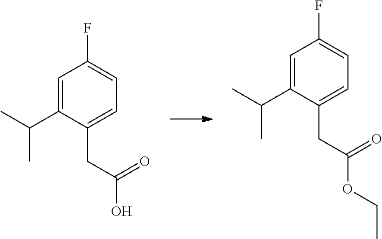 [SMILES: CC(C)c1cc(F)ccc1CC(=O)O ; CCOC(=O)Cc1ccc(F)cc1C(C)C]

2-(4-Fluoro-2-isopropylphenyl)acetic acid (1.8 g, 9.2 mmol) was dissolved in ethanol (25 mL) under N₂ atmosphere and cooled to 0° C. Thionyl chloride (1.6 g, 14 mmol) was added dropwise and the mixture was stirred overnight at room temperature. The mixture was evaporated to dryness to yield the title compound (2.1 g, 100%) as a clear oil.

¹H NMR (CDCl₃) δ 7.18 (dd, 1H), 6.99 (dd, 1H), 6.82 (dt, 1H), 4.15 (q, 2H), 3.62 (s, 2H), 3.09 (m, 1H), 1.24 (t, 3H), 1.21 (d, 6H).

### Step D: Ethyl 2-(4-fluoro-2-isopropylphenyl)-5-methylhex-4-enoate

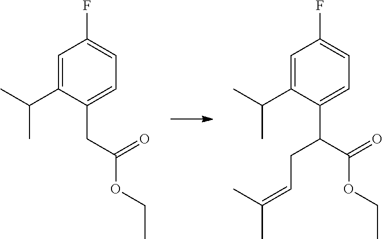 [SMILES: CCOC(=O)Cc1ccc(F)cc1C(C)C ; CCOC(=O)C(CC=C(C)C)c1ccc(F)cc1C(C)C]

Ethyl 2-(4-fluoro-2-isopropylphenyl)acetate (1.6 g, 7.1 mmol) was dissolved in THF (10 mL) under N₂ atmosphere and cooled to −78° C. LiHMDS (1.3 g, 8 mL, 7.8 mmol) was added and the mixture was stirred for 15 minutes at −78° C. 1-Bromo-3-methylbut-2-ene (1.2 g, 7.8 mmol) was added and the mixture was allowed to reach room temperature over the weekend. The mixture was evaporated to near dryness and 1 M HCl (20 mL) and ethyl acetate (20 mL) were added. The layers were separated and the aqueous layer was extracted with ethyl acetate (2×20 mL). The combined organic layers were dried over sodium sulfate and evaporated to dryness to yield the title compound (2.0 g, 96%) as a clear oil.

¹H NMR (CDCl₃) δ 7.32 (dd, 1H), 6.95 (dd, 1H), 6.82 (dt, 1H), 5.02 (t, 1H), 4.10 (q, 2H), 3.82 (t, 1H), 3.22 (m, 1H), 2.75 (m, 1H), 2.38 (m, 1H), 1.62 (s, 3H), 1.56 (s, 3H), 1.23 (t, 3H), 1.20 (d, 6H).

LCMS: m/z 293 (M+H)⁺ (ES⁺).

### Step E: Ethyl 6-fluoro-8-isopropyl-4,4-dimethyl-1,2,3,4-tetrahydronaphthalene-1-carboxylate

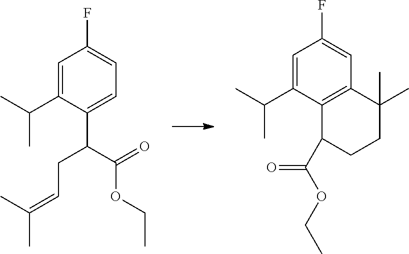 [SMILES: CCOC(=O)C(CC=C(C)C)c1ccc(F)cc1C(C)C ; CCOC(=O)C1CCC(C)(C)c2cc(F)cc(C(C)C)c21]

Ethyl 2-(4-fluoro-2-isopropylphenyl)-5-methylhex-4-enoate (2.0 g, 7 mmol) was dissolved in dichloroethane (40 mL) under N₂ atmosphere. Bismuth(III) trifluoromethanesulfonate (0.2 g, 0.3 mmol) was added and the mixture was refluxed for 2 hours. The mixture was filtered over Celite, evaporated to dryness and subjected to column chromatography on silica (heptanes with an ethyl acetate gradient from 0 to 15%) yielding the title product (1.0 g, 48%) as a colourless oil.

¹H NMR (CDCl₃) δ 6.91 (dd, 1H), 6.80 (dd, 1H), 4.12 (q, 2H), 3.92 (m, 1H), 2.93 (m, 1H), 2.14 (m, 1H), 2.04 (m, 1H), 1.75 (m, 1H), 1.58 (m, 1H), 1.35 (s, 3H), 1.28 (s, 3H), 1.22 (t, 3H), 1.18 (d, 6H).

### Step F: 6-Fluoro-8-isopropyl-4,4-dimethyl-1,2,3,4-tetrahydronaphthalene-1-carboxylic acid

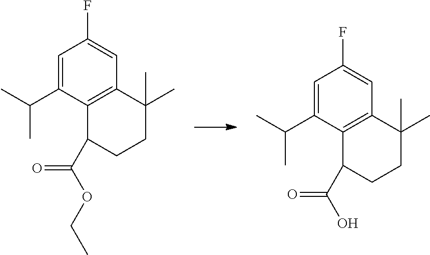 [SMILES: CCOC(=O)C1CCC(C)(C)c2cc(F)cc(C(C)C)c21 ; CC(C)c1cc(F)cc2c1C(C(=O)O)CCC2(C)C]

Ethyl 6-fluoro-8-isopropyl-4,4-dimethyl-1,2,3,4-tetrahydronaphthalene-1-carboxylate (0.8 g, 3 mmol) was dissolved in methanol (20 mL). Lithium hydroxide (1.0 g, 42 mmol) in water (10 mL) was added and the mixture was refluxed overnight. The reaction mixture was concentrated by rotary evaporation and partitioned between TBME (20 mL) and water (20 mL). The aqueous phase was acidified to pH 1 with 5 N HCl and extracted with DCM (2×20 mL). The combined organic layers were dried over Na₂SO₄, filtered and concentrated, yielding the title product (254 mg, 35%) as a white solid.

¹H NMR (CDCl₃) δ 6.91 (dd, 1H), 6.80 (dd, 1H), 3.97 (m, 1H), 2.97 (m, 1H), 2.20 (m, 1H), 2.04 (m, 1H), 1.72 (dt, 1H), 1.58 (m, 1H), 1.32 (s, 3H), 1.23 (s, 3H), 1.18 (dd, 6H).

LCMS: m/z 263 (M−H)⁻ (ES⁻).

### Step G: 6-Fluoro-8-isopropyl-4,4-dimethyl-1,2,3,4-tetrahydronaphthalene-1-carbonyl chloride

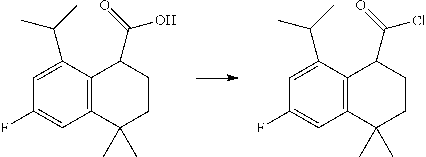 [SMILES: CC(C)c1cc(F)cc2c1C(C(=O)O)CCC2(C)C ; CC(C)c1cc(F)cc2c1C(C(=O)Cl)CCC2(C)C]

6-Fluoro-8-isopropyl-4,4-dimethyl-1,2,3,4-tetrahydronaphthalene-1-carboxylic acid (100 mg, 0.38 mmol) was stirred in DCM (10 mL) and a drop of DMF was added, followed by the dropwise addition of oxalylchloride (0.17 mL, 1.89 mmol). The solution was stirred at room temperature for 4 hours and concentrated thoroughly to afford the title compound (107 mg, 99%) as a yellow oil.

¹H NMR (300 MHz, Chloroform-d) δ 6.91 (dd, 1H), 6.84 (dd, 1H), 4.31 (dd, 1H), 2.96-2.82 (m, 1H), 2.56-2.39 (m, 1H), 2.27-2.05 (m, 1H), 1.72-1.62 (m, 2H), 1.32 (d, 6H), 1.26-1.19 (m, 6H).

### Intermediate R7: 5-Fluoro-7-isopropyl-3,3-dimethyl-2,3-dihydro-1H-indene-1-carbonyl chloride

### Step A: 3-(4-Fluoro-2-isopropylphenyl)-5,5-dimethyldihydrofuran-2(3H)-one

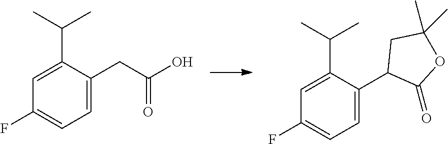 [SMILES: CC(C)c1cc(F)ccc1CC(=O)O ; CC(C)c1cc(F)ccc1C1CC(C)(C)OC1=O]

A solution of n-BuLi (2.5 M in hexanes, 0.69 mL, 4.3 mL, 11 mmol) was added via syringe to a cooled (−20° C.) solution of diisopropylamine (1.1 g, 1.5 mL, 11 mmol) in THF (15 mL). The reaction mixture was stirred at −20° C. for 20 minutes, then cooled to −78° C. and a solution of 3-(4-fluoro-2-isopropylphenyl)-4,4-dimethyldihydrofuran-2(3H)-one (1 g, 5.1 mmol) in THF (5 mL) was added via cannula. The reaction mixture was stirred at −78° C. for 20 minutes, then warmed to room temperature and stirred for 2 hours. The reaction mixture was then cooled to 0° C., and neat 2,2-dimethyloxirane (0.37 mmol, 0.45 mL, 5.1 mmol) was added via syringe, resulting in a clear yellow solution, which was stirred for 12 hours at room temperature. Water (7 mL) was added to the reaction mixture. The reaction mixture was heated for 1 hour towards reflux, and then allowed to cool. Most of the THF was removed by concentration. Then the reaction mixture was washed with TBME (2×). The aqueous phase was treated with EtOH (12 mL), acidified with 37% aqueous HCl (3.1 mL), stirred towards reflux for 3 hours, and then left standing overnight. The reaction mixture was extracted with CHCl₃ (3×25 mL). The combined organic phases were washed with sat aq NaHCO₃ (2×15 mL), then dried (Na₂SO₄), filtered and concentrated to obtain an oil (1.2 g) which crystallized partially upon standing. The material was stirred in TBME:heptanes (6:1) for 30 minutes, filtered and dried (430 mg). The filtrate was concentrated to an oil to give slightly less pure material (530 mg). Combined this afforded the title compound (0.96 g, 75%) as a yellow oil.

¹H NMR (300 MHz, Chloroform-d) δ 7.11 (dd, 1H), 6.99 (dd, 1H), 6.89 (td, 1H), 4.23 (dd, 1H), 3.13-2.93 (m, 1H), 2.55 (dd, 1H), 2.08 (t, 1H), 1.61-1.45 (m, 6H), 1.34-1.17 (m, 6H).

### Step B: 5-Fluoro-7-isopropyl-3,3-dimethyl-2,3-dihydro-1H-indene-1-carboxylic acid

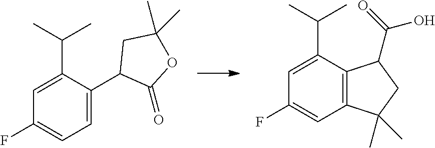 [SMILES: CC(C)c1cc(F)ccc1C1CC(C)(C)OC1=O ; CC(C)c1cc(F)cc2c1C(C(=O)O)CC2(C)C]

3-(4-Fluoro-2-isopropylphenyl)-5,5-dimethyldihydrofuran-2(3H)-one (430 mg, 1.72 mmol) was dissolved in 1,2-dichloroethane (2 mL) and added dropwise over 1 hour to an ice cooled solution of AlCl₃ (458 mg, 3.44 mmol) in 1,2-dichloroethane (2 mL). The mixture was stirred for 2 hours and poured in ice/water (10 mL). CHCl₃ (10-15 mL) was used to rinse the reaction vessel. The reaction mixture was filtered over Celite and the layers were separated. The organic phase was dried over anhydrous sodium sulfate, filtered and the solution was concentrated to afford the title compound (0.43 g, 99%) as a yellow oil.

¹H NMR (300 MHz, Chloroform-d) δ 6.82 (dd, 1H), 6.68 (dd, 1H), 4.07 (dd, 1H), 2.98-2.77 (m, 1H), 2.48-2.20 (m, 2H), 1.29 (m, 6H), 1.25-1.11 (m, 6H).

### Step C: 5-Fluoro-7-isopropyl-3,3-dimethyl-2,3-dihydro-1H-indene-1-carbonyl chloride

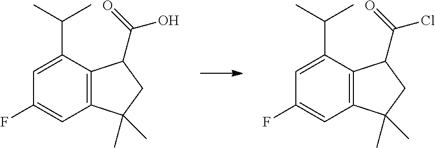 [SMILES: CC(C)c1cc(F)cc2c1C(C(=O)O)CC2(C)C ; CC(C)c1cc(F)cc2c1C(C(=O)Cl)CC2(C)C]

5-Fluoro-7-isopropyl-3,3-dimethyl-2,3-dihydro-1H-indene-1-carboxylic acid (65 mg, 0.26 mmol) was stirred in DCM (10 mL) and a drop of DMF was added, followed by the dropwise addition of oxalylchloride (0.11 mL, 1.3 mmol). The solution was stirred at room temperature for 3 hours and concentrated thoroughly to afford the title compound (70 mg, 99%) as a yellow oil.

¹H NMR (300 MHz, Chloroform-d) δ 6.84 (dd, 1H), 6.69 (dd, 1H), 4.45 (ddd, 1H), 2.78 (pd, 1H), 2.57-2.34 (m, 2H), 1.36-1.23 (m, 9H), 1.19 (d, 3H).

### Intermediate R8: 4-(4-Isocyanato-2,3-dihydro-1H-inden-5-yl)-2-methoxypyridine

### Step A: 4-Nitro-2,3-dihydro-1H-indene

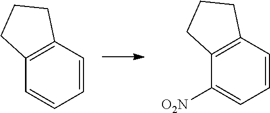 [SMILES: c1ccc2c(c1)CCC2 ; O=[N+]([O-])c1cccc2c1CCC2]

To a mixture of 2,3-dihydro-1H-indene (60 g, 507.72 mmol, 1 eq) in concentrated H₂SO₄ (30 mL) was added dropwise a solution of HNO₃ (so mL, 69 wt % in aqueous solution) in concentrated H₂SO₄ (50 mL) at 0° C. over a period of 3.5 hours. The reaction mixture was stirred at 0° C. for 0.5 hour, and then poured into ice-water (600 mL) and extracted with ethyl acetate (2×400 mL). The combined organic layers were washed with water (500 mL), saturated aqueous NaHCO₃ solution (500 mL) and brine (2×500 mL), dried over anhydrous Na₂SO₄, filtered and concentrated. The residue was purified by column chromatography (SiO₂, petroleum ether:ethyl acetate, 1:0 to 100:1) to give the title compound (55 g, contained another regio-isomer) as a colourless oil.

¹H NMR (400 MHz, CDCl₃) δ 7.98 (d, 1H), 7.51 (d, 1H), 7.30 (t, 1H), 3.41 (t, 2H), 302 (t, 2H) and 2.22-2.20 (m, 2H).

### Step B: 2,3-Dihydro-1H-inden-4-amine

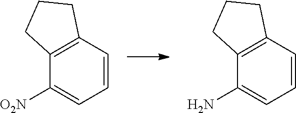 [SMILES: O=[N+]([O-])c1cccc2c1CCC2 ; Nc1cccc2c1CCC2]

To a solution of 4-nitro-2,3-dihydro-1H-indene (55 g, contained another regio-isomer) in MeOH (500 mL) was added Pd/C (5 g, 10 wt % loading on activated carbon) under N₂. The suspension was degassed in vacuo and purged with H₂ several times. The reaction mixture was stirred at 20° C. for 12 hours under H₂ (50 psi). The reaction mixture was filtered and the filtrate was concentrated in vacuo. The residue was purified by column chromatography (SiO₂, petroleum ether:ethyl acetate, 1:0 to 100:4) to give the title compound (19.82 g, 43% yield, 96.4% purity on LCMS) as a brown oil.

¹H NMR (400 MHz, CDCl₃) δ 7.01 (t, 1H), 6.71 (d, 1H), 6.51 (d, 1H), 3.57 (br s, 2H), 2.93 (t, 2H), 2.75 (t, 2H) and 2.16-2.08 (m, 2H).

LCMS: m/z 134.2 (M+H)⁺ (ES⁺).

### Step C: N-(2,3-Dihydro-1H-inden-4-yl)acetamide

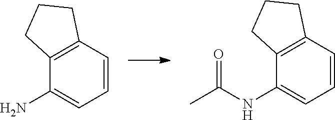

To a solution of 2,3-dihydro-1H-inden-4-amine (19.8 g, 148.66 mmol, 1 eq) and TEA (19.56 g, 193.26 mmol, 1.3 eq) in DCM (300 mL) was added dropwise Ac₂O (17.45 g, 170.96 mmol, 1.15 eq) at 0° C. over 0.1 hour. Then the reaction mixture was warmed to 16° C., stirred for 1.4 hours, poured into water (500 mL) and extracted with DCM (2×300 mL). The combined organic phases were washed with brine (2×500 mL), dried over anhydrous Na₂SO₄, filtered and concentrated in vacuo to give the title compound (25.74 g, 96% yield, 96.7% purity on LCMS) as a white solid.

¹H NMR (400 MHz, CDCl₃) δ 7.70 (d, 1H), 7.15 (t, 1H), 7.02 (d, 1H), 2.95 (t, 2H), 2.81 (t, 2H), 2.18 (s, 3H) and 2.15-2.08 (m, 2H).

LCMS: m/z 176.2 (M+H)⁺ (ES⁺).

### Step D: N-(5-Bromo-2,3-dihydro-1H-inden-4-yl)acetamide

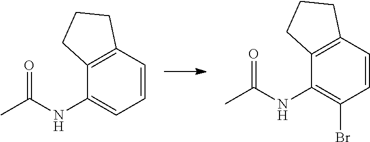

A mixture of N-(2,3-dihydro-1H-inden-4-yl)acetamide (34.6 g, 197.46 mmol, 1 eq), 4-methylbenzenesulfonic acid (18.70 g, 108.60 mmol, 0.55 eq) and Pd(OAc)₂ (2.22 g, 9.87 mmol, 0.05 eq) were suspended in toluene (400 mL) and stirred at 20° C. for 0.5 hour under air atmosphere. NBS (38.66 g, 217.20 mmol, 1.1 eq) was added. The resulting reaction mixture was stirred at 20° C. for 2 hours, poured into water (500 mL) and extracted with ethyl acetate (2×500 mL). The combined organic phases were washed with brine (2×500 mL), dried over anhydrous Na₂SO₄, filtered and concentrated in vacuo. The residue was purified by column chromatography (SiO₂, petroleum ether:ethyl acetate, 10:1 to 2:1) to give the title compound (13.9 g, 27% yield, 98.1% purity on LCMS) as a white solid.

¹H NMR (400 MHz, CDCl₃) δ 7.33 (d, 1H), 7.16 (s, 1H), 6.98 (d, 1H), 2.92-2.83 (m, 4H), 2.21 (s, 3H) and 2.10-2.02 (m, 2H).

LCMS: m/z 254.1 (M+H)⁺ (ES⁺).

### Step E: 5-Bromo-2,3-dihydro-1H-inden-4-amine, hydrochloride salt

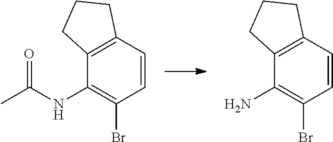

A mixture of N-(5-bromo-2,3-dihydro-1H-inden-4-yl)acetamide (45.68 g, 179.76 mmol, 1 eq) in EtOH (200 mL) and concentrated HCl (300 mL, 36 wt % in water) was stirred at 80° C. for 36 hours. The reaction mixture was cooled to 0° C. in an ice bath and some solid precipitated out. The suspension was filtered. The filter cake was washed with ice-water (50 mL) and dried in vacuo to give the title compound (34.1 g, 72% yield, 94.1% purity on LCMS) as a grey solid.

¹H NMR (400 MHz, DMSO-d₆) δ 7.67 (br s, 2H), 7.24 (d, 1H), 6.69 (d, 1H), 2.85 (t, 2H), 2.79 (t, 2H) and 2.04-1.96 (m, 2H).

LCMS: m/z 212.0 (M+H)⁺ (ES⁺).

### Step F: 5-(2-Methoxypyridin-4-yl)-2,3-dihydro-1H-inden-4-amine

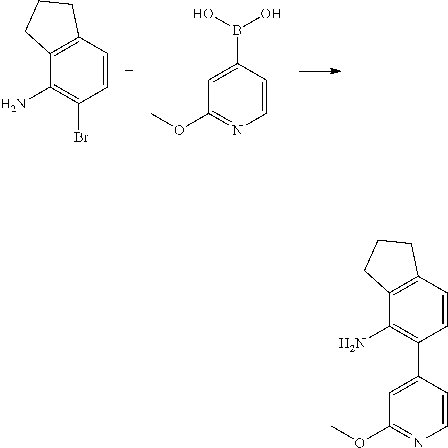 [SMILES: Nc1c(Br)ccc2c1CCC2 ; COc1cc(B(O)O)ccn1 ; COc1cc(-c2ccc3c(c2N)CCC3)ccn1]

A mixture of (2-methoxypyridin-4-yl)boronic acid (25.11 g, 164.15 mmol, 1.2 eq), 5-bromo-2,3-dihydro-1H-inden-4-amine, hydrochloride salt (34 g, 136.80 mmol, 1 eq) and K₂CO₃ (60.50 g, 437.74 mmol, 3.2 eq) in dioxane (500 mL) and H₂O (100 mL) was degassed with nitrogen for 15 minutes. Pd(dppeCl₂.CH₂Cl₂ (6 g, 7.35 mmol, 0.053 eq) was added. The reaction mixture was heated to 80° C. for 12 hours under nitrogen, poured into water (500 mL) and extracted with ethyl acetate (2×500 mL). The combined organic phases were washed with brine (2×700 mL), dried over anhydrous Na₂SO₄, filtered and concentrated in vacuo. The residue was purified by column chromatography (SiO₂, petroleum ether:ethyl acetate, 0:1 to 1:10) to give the title compound (27.4 g, 79% yield, 95% purity on LCMS) as a white solid.

¹H NMR (400 MHz, CDCl₃) δ 8.22 (d, 1H), 7.03-7.00 (m, 1H), 6.99 (d, 1H), 6.87 (s, 1H), 6.77 (d, 1H), 3.99 (s, 3H), 3.77 (br s, 2H), 2.97 (t, 2H), 2.77 (t, 2H) and 2.21-2.13 (m, 2H).

LCMS: m/z 241.2 (M+H)⁺ (ES⁺).

### Step G: 4-(4-Isocyanato-2,3-dihydro-1H-inden-5-yl)-2-methoxypyridine

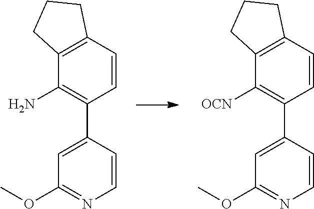

To a solution of 5-(2-methoxypyridin-4-yl)-2,3-dihydro-1H-inden-4-amine (11 g, 45.78 mmol, 1 eq) and TEA (5.10 g, 50.35 mmol, 1.1 eq) in THF (275 mL) was added in portions bis(trichloromethyl) carbonate (4.93 g, 16.61 mmol, 0.36 eq) at 0° C. The reaction mixture was stirred at 16° C. for 0.5 hour. The reaction mixture was filtered through a pad of silica gel and the filter cake was washed with THF (2 L). The filtrate was concentrated in vacuo to give the title compound (9.04 g, 74%) as a light yellow solid.

¹H NMR (400 MHz, CDCl₃) δ 8.28 (d, 1H), 7.20-7.16 (m, 3H), 7.02 (s, 1H), 4.16 (s, 3H), 3.04-2.99 (m, 4H) and 2.23-2.15 (m, 2H).

### Intermediate R9: 2-(1,2,3,5,6,7-Hexahydro-s-indacen-4-yl)acetyl chloride

### Step A: 3-Chloro-1-(2,3-dihydro-1H-inden-5-yl)propan-1-one

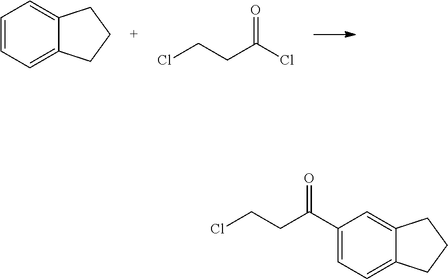 [SMILES: c1ccc2c(c1)CCC2 ; O=C(Cl)CCCl ; O=C(CCCl)c1ccc2c(c1)CCC2]

A suspension of AlCl₃ (225.67 g, 1.69 mol, 1 eq) in DCM (1 L) was cooled to −10° C. under N₂ atmosphere. Then a mixture of 2,3-dihydro-1H-indene (200 g, 1.69 mol, 1 eq) and 3-chloropropanoyl chloride (214.88 g, 1.69 mol, 1 eq) in DCM (400 mL) was added dropwise. The reaction mixture was warmed to 27° C. and stirred for 2 hours. Then the reaction mixture was added slowly to an aqueous HCl solution (2 N, 2.8 L) below 10° C. The layers were separated and the aqueous layer was extracted with DCM (1 L). The combined organic layers were washed with water (1 L), saturated aqueous NaHCO₃ solution (1 L) and brine (500 mL), dried over anhydrous Na₂SO₄, filtered and concentrated in vacuo. The residue was triturated with petroleum ether (500 mL) to give the title compound (260.44 g, 74%) as a white solid.

¹H NMR (400 MHz, CDCl₃) δ 7.84 (s, 1H), 7.79-7.76 (m, 1H), 7.33 (d, 1H), 3.94 (t, 2H), 3.45 (t, 2H), 2.98 (t, 4H), 2.18-2.11 (m, 2H).

LCMS: m/z 209.1 (M+H)⁺ (ES⁺).

### Step B: 2,3,6,7-Tetrahydro-s-indacen-1(5H)-one

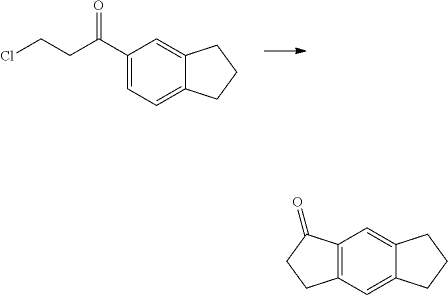 [SMILES: O=C(CCCl)c1ccc2c(c1)CCC2 ; O=C1CCc2cc3c(cc21)CCC3]

To a concentrated H₂SO₄ solution (1.84 kg, 18.39 mol, 98 wt % in aqueous solution, 37.25 eq) was added to 3-chloro-1-(2,3-dihydro-1H-inden-5-yl)propan-1-one (103 g, 493.57 mmol, 1 eq). The reaction mixture was stirred at 70° C. for 12 hours, poured into ice-water (4.5 L) and filtered. The filter cake was dissolved in EtOAc (500 mL) and saturated aqueous Na₂CO₃solution (500 mL) was added. The organic layer was separated and the aqueous layer was extracted with EtOAc (3×200 mL). The combined organic layers were concentrated in vacuo to give the title compound (60 g, 69% yield, 98% purity on LCMS) as a yellow solid.

¹H NMR (400 MHz, CDCl₃) δ 7.58 (s, 1H), 7.30 (s, 1H), 3.08-2.96 (m, 2H), 2.95-2.91 (m, 4H), 2.70 (t, 2H) and 2.15-2.05 (m, 2H).

LCMS: m/z 173.2 (M+H)⁺ (ES⁺).

### Step C: 1,2,3,5,6,7-Hexahydro-s-indacene

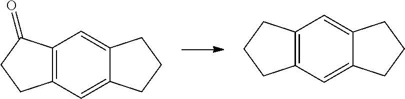 [SMILES: O=C1CCc2cc3c(cc21)CCC3 ; c1c2c(cc3c1CCC3)CCC2]

To a solution of 2,3,6,7-tetrahydro-s-indacen-1(5H)-one (15 g, 87.10 mmol, 1 eq) in MeOH (200 mL) was added with MeSO₃H (16.94 g, 176.22 mmol, 2.02 eq) and Pd(OH)₂/C (3.2 g, 20 wt % loading on activated carbon). The reaction mixture was degassed and purged with H₂ three times. The resulting mixture was stirred at 25° C. for 12 hours under H₂ (15 psi). The reaction mixture was filtered, and the filtrate was concentrated in vacuo. The residue was purified by column chromatography (SiO₂, only eluting with petroleum ether) to give the title compound (12 g, 85%) as a white solid.

¹H NMR (400 MHz, CDCl₃) δ 7.14 (s, 2H), 3.00-2.85 (m, 8H) and 2.16-2.09 (m, 4H).

### Step D: 4-Bromo-1,2,3,5,6,7-hexahydro-s-indacene

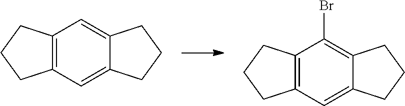 [SMILES: c1c2c(cc3c1CCC3)CCC2 ; Brc1c2c(cc3c1CCC3)CCC2]

To a solution of 1,2,3,5,6,7-hexahydro-s-indacene (11.5 g, 72.67 mmol, 1 eq) in CCl₄ (200 mL) was added with 12 (922 mg, 3.63 mmol, 0.05 eq). Then a solution of Br₂ (12.19 g, 76.31 mmol, 1.05 eq) in CCl₄ (50 mL) was added dropwise at 0° C. The resulting mixture was stirred at 0° C. for 2 hours, quenched with saturated aqueous NH₄Cl solution (100 mL), and extracted with DCM (3×200 mL). The combined organic layers were dried over anhydrous Na₂SO₄, filtered and concentrated in vacuo. The residue was purified by column chromatography (SiO₂, only eluting with petroleum ether) to give the title compound (15 g, 87%) as a red oil.

¹H NMR (400 MHz, CDCl₃) δ 7.00 (s, 1H), 3.10-2.83 (m, 8H) and 2.11 (m, 4H).

### Step E: (2-(tert-Butoxy)-2-oxoethyl) zinc (II) bromide

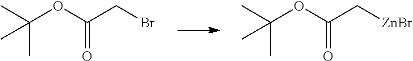

A mixture of Zn (80 g) in HCl (1 M, 308 mL) was stirred at 25° C. for 30 minutes. Then the mixture was filtered and the filter cake was dried in vacuo. To a mixture of this Zn (55 g, 841.11 mmol, 2.98 eq) in THF (550 mL) was added TMSCl (3.06 g, 28.20 mmol, 0.1 eq) and 1,2-dibromoethane (5.30 g, 28.20 mmol, 0.1 eq) at 20° C. under N₂. Then tert-butyl 2-bromoacetate (55 g, 281.97 mmol, 1 eq) was added at 50° C. under N₂. The reaction mixture was stirred at 50 ° C. for 2 hours. The mixture (theoretical amount: 0.5 M, 550 mL, in THF solution) was cooled and used in the next step without further purification.

### Step F: tert-Butyl 2-(1,2,3,5,6,7-hexahydro-s-indacen-4-yl)acetate

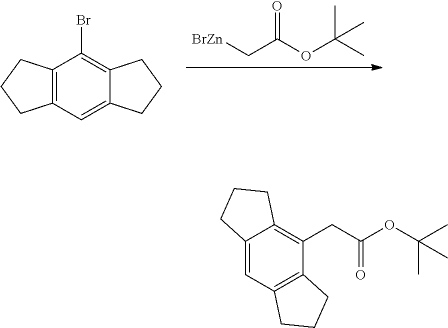

To a solution of 4-bromo-1,2,3,5,6,7-hexahydro-s-indacene (20 g, 84.34 mmol, 1 eq), Pd₂(dba)₃ (3.86 g, 4.22 mmol, 0.05 eq), and Xphos® (4.02 g, 8.43 mmol, 0.1 eq) in THF (1 mL) was added (2-(tert-butoxy)-2-oxoethyl) zinc (II) bromide (168.68 mmol, 500 mL, 0.5 M, in THF, 2 eq) at 0° C. Then the reaction mixture was stirred at 70° C. for 12 hours, quenched with saturated aqueous NH₄Cl solution (500 mL), and extracted with ethyl acetate (3×500 mL). The combined organic layers were dried over anhydrous Na₂SO₄, filtered and concentrated in vacuo. The residue was purified by column chromatography (SiO₂, only eluting with petroleum ether) to give the title compound (20 g, 87%) as a yellow oil.

¹H NMR (400 MHz, CDCl₃) δ 7.02 (s, 1H), 3.51 (s, 2H), 2.90-2.84 (m, 8H), 2.11-2.04 (m, 4H) and 1.44 (s, 9H).

### Step G: 2-(1,2,3,5,6,7-Hexahydro-s-indacen-4-yl)acetic acid

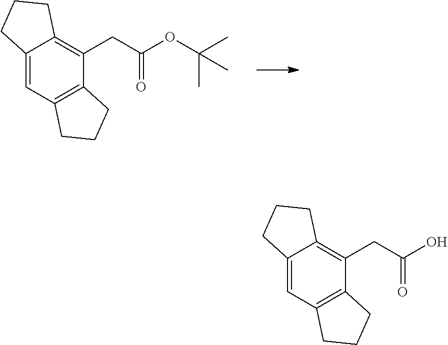 [SMILES: CC(C)(C)OC(=O)Cc1c2c(cc3c1CCC3)CCC2 ; O=C(O)Cc1c2c(cc3c1CCC3)CCC2]

To a solution of tert-butyl 2-(1,2,3,5,6,7-hexahydro-s-indacen-4-yl)acetate (20 g, 73.43 mmol, 1 eq) in DCM (200 mL) was added TFA (308 g, 2.70 mol, 36.79 eq). The reaction mixture was stirred at 25° C. for 1 hour, and then concentrated in vacuo. The residue was dissolved in DCM (300 mL) and the mixture was adjusted to pH=9 with an aqueous NaOH solution (2 N). The aqueous phase was separated and adjusted to pH=2-3 with an aqueous HCl solution (1 N). Large white solids were formed, and the mixture was filtered. The collected solid was dried to give the title compound (12 g, 76%) as a white solid.

¹H NMR (400 MHz, DMSO-d₆) δ 12.20 (s, 1H), 6.95 (s, 1H), 3.70 (s, 2H), 2.82-2.70 (m, 8H) and 2.03-1.94 (m, 4H).

### Step H: 2-(1,2,3,5,6,7-Hexahydro-s-indacen-4-yl)acetyl chloride

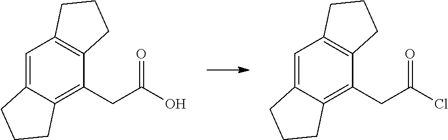 [SMILES: O=C(O)Cc1c2c(cc3c1CCC3)CCC2 ; O=C(Cl)Cc1c2c(cc3c1CCC3)CCC2]

A solution of 2-(1,2,3,5,6,7-hexahydro-s-indacen-4-yl)acetic acid (500 mg, 2.31 mmol, 1 eq) in SOCl₂ (10 mL) was heated to 80° C. for 1 hour. The reaction mixture was concentrated in vacuo to give the title compound (542.6 mg, 100%) as a yellow oil, which was used directly in the next step.

### Intermediate R10: 2-(5-(2-Methoxypyridin-4-yl)-2,3-dihydro-1H-inden-4-yl)acetic acid

### Step A: 4-(4-Bromo-2,3-dihydro-1H-inden-5-yl)-2-methoxypyridine

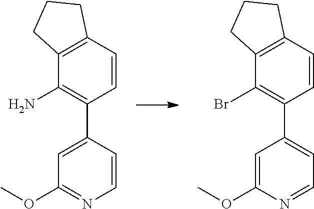 [SMILES: COc1cc(-c2ccc3c(c2N)CCC3)ccn1 ; COc1cc(-c2ccc3c(c2Br)CCC3)ccn1]

To a mixture of 5-(2-methoxypyridin-4-yl)-2,3-dihydro-1H-inden-4-amine (Intermediate R8, Step F) (28 g, 116.52 mmol, 1 eq) in MeCN (300 mL) was added isopentyl nitrite (16.38 g, 139.83 mmol, 1.2 eq) at 0° C. The reaction mixture was stirred at 0° C. for 30 minutes under N₂. CuBr (17.05 g, 118.85 mmol, 1.02 eq) was added at 0° C. and the resulting mixture was stirred at 60° C. for 1 hour. The reaction mixture was filtered and the filtrate was concentrated in vacuo. The residue was purified by column chromatography (SiO₂, petroleum ether:ethyl acetate, 1:0 to 20:1) to give the title compound (15 g, 37% yield, 87.7% purity on LCMS) as a yellow solid.

¹H NMR (400 MHz, CDCl₃) δ 8.20 (d, 1H), 7.21 (d, 1H), 7.06 (d, 1H), 6.94 (dd, 1H), 6.78 (s, 1H), 3.99 (s, 3H), 3.08 (t, 2H), 3.03 (t, 2H) and 2.20-2.10 (m, 2H).

LCMS: m/z 304.0 (M+H)⁺ (ES⁺).

### Step B: tert-Butyl 2-(5(2-methoxypyridin-4-yl)-2,3-dihydro-1H-inden-4-yl)acetate

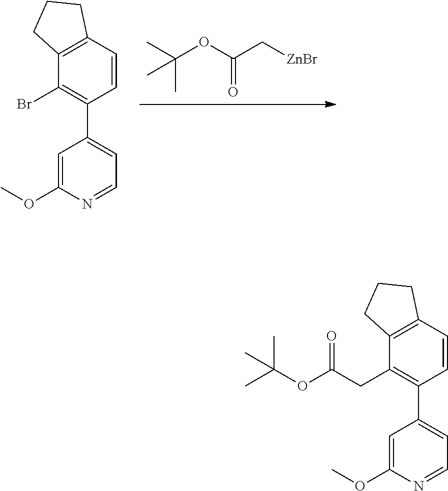

To a mixture of 4-(4-bromo-2,3-dihydro-1H-inden-5-yl)-2-methoxypyridine (15 g, 49.31 mmol, 1 eq), Xphos® (2.35 g, 4.93 mmol, 0.1 eq) and Pd₂(dba)₃ (2.26 g, 2.47 mmol, 0.05 eq) in THF (50 mL) was added a solution of (2-(tert-butoxy)-2-oxoethyl) zinc (II) bromide (Intermediate R9, Step E) in THF (0.5 M, 296 mL, 3 eq) at 20° C. under N₂. The reaction mixture was stirred at 70° C. for 12 hours under N₂. The mixture was poured into saturated aqueous NH₄Cl solution (200 mL). The aqueous phase was extracted with ethyl acetate (3×200 mL). The combined organic phases were washed with brine (20 mL), dried over anhydrous Na₂SO₄, filtered and concentrated in vacuo. The residue was purified by column chromatography (SiO₂, petroleum ether:ethyl acetate, 1:0 to 20:1) to give the title compound (15 g, 83% yield, 92.9% purity on LCMS) as a yellow oil.

¹H NMR (400 MHz, CDCl₃) δ 8.17 (dd, 1H), 7.20 (d, 1H), 7.04 (d, 1H), 6.86 (dd, 1H), 6.72 (s, 1H), 3.98 (s, 3H), 3.47 (s, 2H), 3.01 (t, 2H), 2.90 (t, 2H), 2.18-2.10 (m, 2H) and 1.43 (s, 9H).

LCMS: m/z 340.1 (M+H)⁺ (ES⁺).

### Step C: 2-(5-(2-Methoxypyridin-4-yl)-2,3-dihydro-1H-inden-4-yl)acetic acid

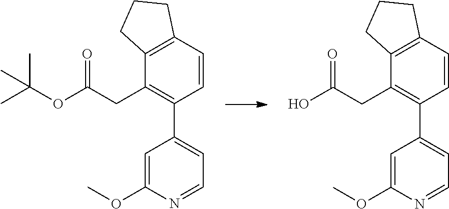 [SMILES: COc1cc(-c2ccc3c(c2CC(=O)OC(C)(C)C)CCC3)ccn1 ; COc1cc(-c2ccc3c(c2CC(=O)O)CCC3)ccn1]

To the solution of tert-butyl 2-(5-(2-methoxypyridin-4-yl)-2,3-dihydro-1H-inden-4-yl)acetate (16 g, 47.14 mmol, 1 eq) in DCM (100 mL) was added TFA (154 g, 1.35 mol, 28.65 eq) at 20° C. The reaction mixture was stirred at 20° C. for 12 hours, and then concentrated under reduced pressure. The residue was purified by column chromatography (SiO₂, petroleum ether:ethyl acetate, 20:1 to 5:1) to give the title compound (12 g, 87% yield, 97% purity on LCMS) as a yellow solid.

¹H NMR (400 MHz, CDCl₃) δ 8.89 (br s, 1H), 8.35 (d, 1H), 7.25 (s, 1H), 7.11 (d, 1H), 7.05 (d, 1H), 6.98 (s, 1H), 4.05 (s, 3 FT), 3.58 (s, 2H), 3.00 (t, 2H), 2.92 (t, 2H) and 2.19-2.10 (m, 2H).

LCMS: m/z 284.1 (M+H)⁺ (ES⁺).

### Intermediate R11: 2-(4-Fluoro-2,6-diisopropylphenyl)acetic acid

### Step A: 4-Fluoro-2,6-di(prop-1-en-2-yl)aniline

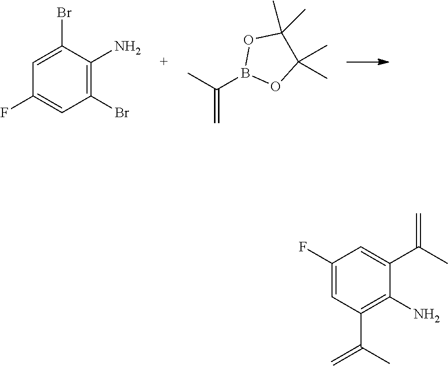

A solution of 2,6-dibromo-4-fluoroaniline (10 g, 1 eq), 4,4,5,5-tetramethyl-2-(prop-1-en-2-yl)-1,3,2-dioxaborolane (16.67 g, 2.67 eq), Cs₂CO₃ (36.35 g, 3 eq) and Pd(dppf)Cl₂ (2.72 g, 3.72 mmol, 0.1 eq) in dioxane (100 mL) and H₂O (10 mL) was degassed under reduced pressure. Then the reaction mixture was heated to 100° C. for 3 hours under nitrogen. The reaction mixture was quenched by addition of H₂O (200 mL), and diluted with EtOAc (150 mL). The mixture was extracted with EtOAc (2×150 mL). The combined organic layers were washed with brine (2×200 mL), dried over anhydrous Na₂SO₄, filtered and concentrated under reduced pressure. The residue was purified by column chromatography (SiO₂, petroleum ether:ethyl acetate, 1:0 to 100:1) to give the title compound (8 g, 89% yield, 78.9% purity on LCMS) as a yellow oil.

¹H NMR (400 MHz, CDCl₃) δ 6.68 (d, 2H), 5.32-5.31 (m, 2H), 5.08 (d, 2H), 3.84 (s, 2H) and 2.07 (d, 6H).

LCMS: m/z 192.2 (M+H)⁺(ES⁺).

### Step B: 4-Fluoro-2,6-diisopropylaniline

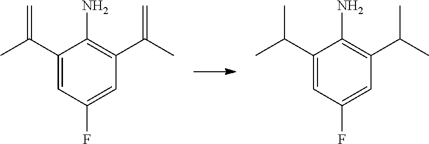

To a solution of 4-fluoro-2,6-di(prop-1-en-2-yl)aniline (8 g, 1 eq) in MeOH (150 mL) was added Pd/C (624 mg, 10 wt % loading on activated carbon). The mixture was degassed and purged with H₂ (20 psi). The reaction mixture was stirred at 25° C. for 12 hours under H₂ (20 psi), and then filtered. The filtrate was concentrated in vacuo. The residue was purified by column chromatography (SiO₂, only eluting with petroleum ether) to give the title compound (4 g, 63% yield, 100% purity on LCMS) as a colourless oil.

¹H NMR (400 MHz, CDCl₃) δ 6.76 (d, 2H), 3.56 (s, 2H), 2.99-2.89 (m, 2H) and 1.26 (d,12H).

LCMS: m/z 196.2 (M+H)⁺ (ES⁺).

### Step C: 2-Bromo-5-fluoro-1,3-diisopropylbenzene

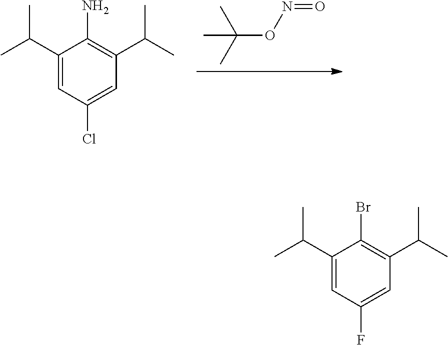

To a solution of 4-fluoro-2,6-diisopropylaniline (3.7 g, 18.95 mmol, 1 eq) in MeCN (180 mL) was added CuBr (4.08 g, 1.5 eq). Then tert-butyl nitrite (2.93 g, 1.5 eq) was added dropwise at 0° C. The resulting mixture was stirred at 60° C. for 1.5 hours, and then concentrated in vacuo. The residue was purified by column chromatography (SiO₂, only eluting with petroleum ether) to give the title compound (2.02 g, 41%) as a white solid.

¹H NMR (400 MHz, CDCl₃) δ 6.85 (d, 2H), 3.55-3.48 (m, 2H) and 1.24 (d, 12H).

### Step D: tert-Butyl 2-(4-fluoro-2,6-diisopropylphenyl)acetate

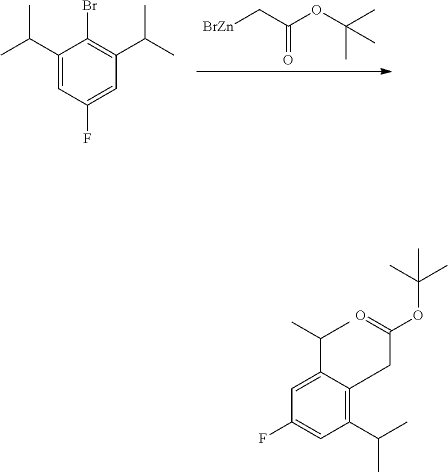

A solution of 2-bromo-5-fluoro-1,3-diisopropylbenzene (16 g, 61.74 mmol, 1 eq) in THF (100 mL) was cooled to 0° C. Then Pd₂(dba)₃ (2.83 g, 3.09 mmol, 0.05 eq), Xphos® (2.94 g, 6.17 mmol, 0.1 eq) and (2-(tert-butoxy)-2-oxoethyl) zinc (II) bromide (Intermediate R9, Step E) (0.5 M, 246.95 mL, in THF solution, 2 eq) were added. The reaction mixture was stirred at 70° C. for 12 hours, and then concentrated in vacuo. The residue was purified by column chromatography (SiO₂, petroleum ether:ethyl acetate, 100:0 to 10:1) to give the title compound (12 g, 59% yield, 90% purity on ¹H NMR) as a red oil.

¹H NMR (400 MHz, CDCl₃) δ 6.83 (d, 2H), 3.66 (s, 2H), 3.21-3.14 (m, 2H), 1.43 (s, 9H) and 1.21 (d, 12H).

### Step E: 2-(4-Fluoro-2,6-diisopropylphenyl)acetic acid

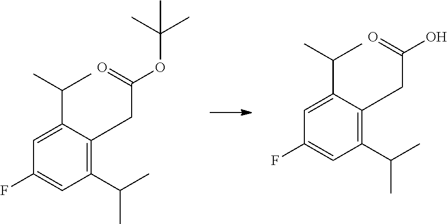 [SMILES: CC(C)c1cc(F)cc(C(C)C)c1CC(=O)OC(C)(C)C ; CC(C)c1cc(F)cc(C(C)C)c1CC(=O)O]

To a solution of tert-butyl 2-(4-fluoro-2,6-diisopropylphenyl)acetate (12 g, 40.76 mmol, 1 eq) in DCM (120 mL) was added TFA (184.80 g, 39.76 eq). The reaction mixture was stirred at 25° C. for 3 hours. Most of solvents were evaporated under reduced pressure. The residue was diluted with H₂O (300 mL) and the mixture was adjusted to pH=10 with 2 M aqueous NaOH solution. The mixture was washed with EtOAc (3×500 mL) and the organic phases were discarded. Then the aqueous layer was adjusted to pH=₃ with 1 M aqueous HCl solution and extracted with EtOAc (3×500 mL). The combined organic layers were washed with brine (2×200 mL), dried over Na₂SO₄, filtered and concentrated under reduced pressure to give the title compound (8 g, 82%) as a yellow solid.

¹H NMR (400 MHz, DMSO-d₆) δ 12.24 (br s, 1H), 6.91 (d, 2H), 3.78 (s, 2H), 3.16-3.06 (m, 2H) and 1.18 (d, 12H).

### Intermediate R12: 7-Chloro-5-isopropyl-2,3-dihydro-1H-inden-4-amine

### Step A: 5-Bromo-7-chloro-2,3-dihydro-1H-inden-4-amine

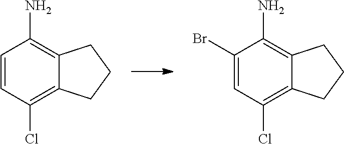 [SMILES: Nc1ccc(Cl)c2c1CCC2 ; Nc1c(Br)cc(Cl)c2c1CCC2]

7-Chloro-2,3-dihydro-1H-inden-4-amine, HCl (1.00 g, 4.90 mmol) (J Med Chem, 2015, vol. 58(2), pages 878-887) was dissolved in water (20 mL). Sat aq NaHCO₃ (10 mL) was added to neutralise the solution. The mixture was extracted with EtOAc (4×30 mL) and the combined organics were washed with brine (30 mL), dried (MgSO₄) and concentrated in vacuo. The residue was dissolved in DCM (10 mL) and cooled to 0° C. NBS (959 mg, 5.39 mmol) was added and the mixture stirred at 0° C. for 2 hours. The precipitate was filtered off and the filtrate washed with 1 N NaOH (2×20 mL), dried (MgSO₄) and concentrated in vacuo. The residue was purified by chromatography on silica gel (40 g column, 0-5% MeOH/DCM) to afford the title compound (895 mg, 67%) as a brown oil.

LCMS m/z 246/248 (M+H)⁻ (ES⁺).

### Step B: 7-Chloro-5-(prop-1-en-2-yl)-2,3-dihydro-1H-inden-4-amine

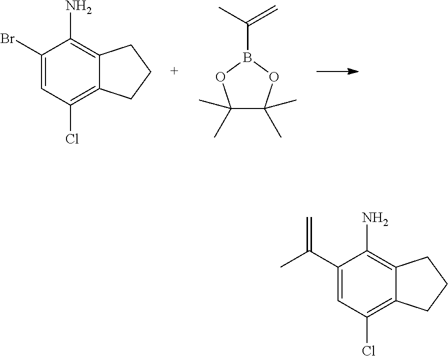

5-Bromo-7-chloro-2,3-dihydro-1H-inden-4-amine (440 mg, 1.78 mmol), 4,4,5,5-tetramethyl-2-(prop-1-en-2-yl)-1,3,2-dioxaborolane (900 mg, 5.35 mmol), potassium phosphate, dibasic (933 mg, 5.35 mmol), dicyclohexyl(2′,6′-dimethoxy-[1,1′-biphenyl]-2-yl)phosphine (88 mg, 0.214 mmol), Pd₂(dba)₃ (49.0 mg, 0.054 mmol) and water (0.5 mL) were added to degassed toluene (10 mL). The solution was refluxed for 16 hours under N₂ and then cooled to room temperature. The reaction was diluted with EtOAc (10 mL) and the organic phase was washed with sat aq NaHCO₃ (2×10 mL), dried (MgSO₄) and concentrated in vacuo. The product was purified by chromatography on silica gel (40 g column, 10-30% EtOAc/isohexane) to afford the title compound (80 mg, 20%) as a colourless oil.

LCMS m/z 208/210 (M+H)⁺ (ES⁺).

### Step C: 7-Chloro-5-isopropyl-2,3-dihydro-1H-inden-4-amine

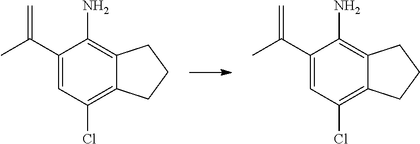

A mixture of 7-chloro-5-(prop-1-en-2-yl)-2,3-dihydro-1H-inden-4-amine (50 mg, 0.241 mmol) and 5% Pd/C (50% weight, Type 87L, 5.1 mg, 0.024 mmol) in ethanol (5 mL) was hydrogenated (5 bar) for 16 hours. The reaction mixture was filtered through Celite and the filtrate concentrated in vacuo. The residue was purified by chromatography on silica gel (40 g column, 5-20% ethyl acetate/isohexane) to afford the title compound (20 mg, 37%) as a colourless oil.

¹H NMR (CDCl₃) δ 6.88 (s, 1H), 2.91-2.80 (m, 3H), 2.76 (t, J=7.0 Hz, 2H), 2.05 (tt, J=8.2, 7.0 Hz, 2H), 1.17 (d, J=6.8 Hz, 6H). Two exchangeable protons not observed.

LCMS m/z 210/212 (M+H)⁺ (ES⁺).

### Intermediate R13: 4-Bromo-8-isocyanato-1,2,3,5,6,7-hexahydro-s-indacene

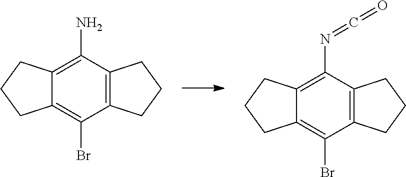 [SMILES: Nc1c2c(c(Br)c3c1CCC3)CCC2 ; O=C=Nc1c2c(c(Br)c3c1CCC3)CCC2]

Triphosgene (141 mg, 0.476 mmol) was added to a mixture of 8-bromo-1,2,3,4,6,7-hexahydro-s-indacen-4-amine (182 mg, 0.722 mmol) and Et₃N (302 μL, 2.16 mmol) in THF (8 mL). The reaction mixture was heated at reflux for 2 hours and then concentrated in vacuo and dried azeotropically with toluene (3×1 mL). The residue was taken up in toluene and filtered through a plug of silica, washing with toluene. The filtrate was concentrated to afford the title compound (197 mg, 97%) as a colourless solid.

LCMS m/z 365/367 (M+H)⁻ (ES⁻).

### Intermediate R14: 8-Isocyanato-1,2,3,5,6,7-hexahydro-s-indacene-4-carbonitrile

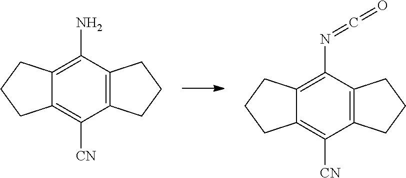 [SMILES: N#Cc1c2c(c(N)c3c1CCC3)CCC2 ; N#Cc1c2c(c(N=C=O)c3c1CCC3)CCC2]

Prepared according to the general procedure of 4-bromo-8-isocyanato-1,2,3,5,6,7-hexahydro-s-indacene (Intermediate R13) from 8-amino-1,2,3,5,6,7-hexahydro-s-indacene-4-carbonitrile (prepared as described in WO 2017/184623) to afford the title compound (101 mg, 87%) as a colourless solid.

LCMS m/z 312.1 (M+H)⁺(ES⁺).

### Intermediate R15: 5-Chloro-2-isocyanato-1,3-diisopropylbenzene

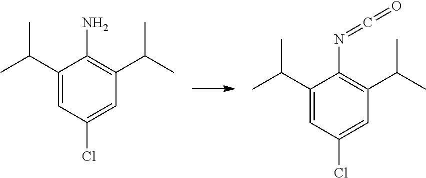 [SMILES: CC(C)c1cc(Cl)cc(C(C)C)c1N ; CC(C)c1cc(Cl)cc(C(C)C)c1N=C=O]

Prepared according to the general procedure of 4-bromo-8-isocyanato-1,2,3,5,6,7-hexahydro-s-indacene (Intermediate R13) from 4-chloro-2,6-diisopropylaniline to afford the title compound (1.05 g, 94%) as a pale orange oil.

LCMS m/z 325 (M+H)⁻ (ES⁻); 323 (M−H)⁻ (ES⁻).

### Intermediate R16: 5-Fluoro-2-isocyanato-1,3-diisopropylbenzene

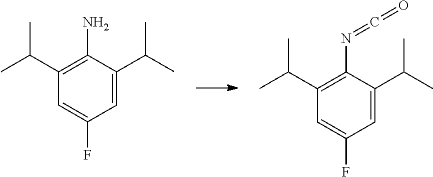 [SMILES: CC(C)c1cc(F)cc(C(C)C)c1N ; CC(C)c1cc(F)cc(C(C)C)c1N=C=O]

Prepared according to the general procedure of 4-bromo-8-isocyanato-1,2,3,5,6,7-hexahydro-s-indacene (Intermediate R13) from 4-fluoro-2,6-diisopropylaniline (Intermediate R11, Step B) to afford the title compound (1.04 g, 92%) as a dear colourless oil.

¹H NMR (DMSO-d₆) δ 7.01 (d, J=9.7 Hz, 2H), 3.21-3.08 (m, 2H), 1.20 (d, J=6.9 Hz, 12H).

LCMS m/z 309.4 (M+Na)+ (ES⁺).

### Intermediate R17: 8-Fluoro-1,2,3,5,6,7-hexahydro-s-indacen-4-amine

### Step A: N-(1,2,3,5,6,7-Hexahydro-s-indacen-4-yl)acetamide

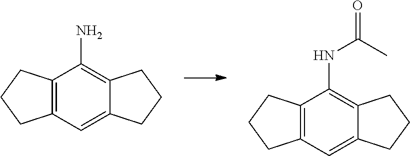 [SMILES: Nc1c2c(cc3c1CCC3)CCC2 ; CC(=O)Nc1c2c(cc3c1CCC3)CCC2]

Acetic anhydride (6.00 mL, 63.5 mmol) was added dropwise to a solution of 1,2,3,5,6,7-hexahydro-s-indacen-4-amine (10 g, 57.7 mmol) and Et₃N (9.65 mL, 69.3 mmol) in DCM (140 mL) at 0° C. The solution was stirred at room temperature overnight. Water (100 mL) was added and the solid collected by filtration, washed with water and dried in vacuo to afford the title compound (9.63 g, 77%) as an off-white solid.

¹H NMR (DMSO-d₆) δ 9.31 (s, 1H), 6.94 (s, 1H), 2.81 (t, J=7.4 Hz, 4H), 2.67 (t, J=7.4 Hz, 4H), 2.00 (s, 3H), 1.96 (p, J=7.4 Hz, 4H).

### Step B: N-(8-Fluoro-1,2,3,5,6,7-hexahydro-s-indacen-4-yl)acetamide

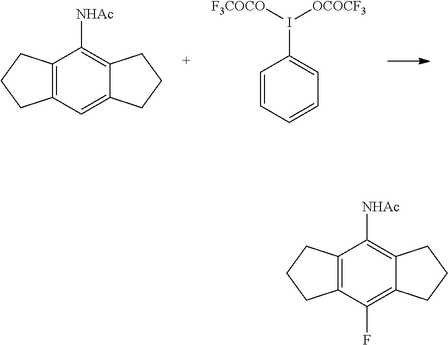

A solution of N-(1,2,3,5,6,7-hexahydro-s-indacen-4-yl)acetamide (4.0 g, 18.6 mmol) and HF-pyridine (20 mL, 222 mmol) in DCM (13 mL) was cooled in an ice bath. A solution of PhI(OCOCF₃)₂ (12 g, 27.9 mmol) in DCM (13 mL) was added dropwise and the reaction was stirred in an ice bath for 1 hour. The reaction mixture was quenched with sat aq calcium hydroxide and the phases were separated. The organics were passed through a hydrophobic frit and the solvent was removed in vacuo. The crude product was split into 2 batches and purified by chromatography on silica gel (220 g and 120 g column, 0-100% EtOAc/isohexane) to afford the title compound (747 mg, 16%) as a pale yellow solid.

¹H NMR (DMSO-d₆) δ 9.32 (br s, 1H), 2.84 (t, J=7.5 Hz, 4H), 2.71 (t, J=7.5 Hz, 4H), 2.03 (p, J=7.5 Hz, 4H), 1.99 (3H, s).

¹⁹F NMR (DMSO-d₆) δ −125.83.

### Step C: 8-Fluoro-1,2,3,5,6,7-hexahydro-s-indacen-4-amine

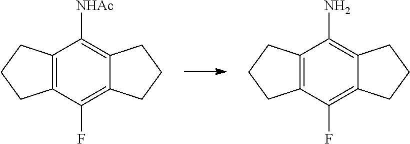 [SMILES: CC(=O)Nc1c2c(c(F)c3c1CCC3)CCC2 ; Nc1c2c(c(F)c3c1CCC3)CCC2]

A solution of N-(8-fluoro-1,2,3,5,6,7-hexahydro-s-indacen-4-yl)acetamide (0.350 g, 1.50 mmol) in EtOH (6.5 mL) and conc HCl (6.5 mL) was heated under reflux for 4 days. The solution was cooled to room temperature and 2 M NaOH was added (20 mL). The product was extracted into DCM (3×50 mL) and the organic extracts were passed through a hydrophobic frit and evaporated in vacuo. The crude product was purified by chromatography on silica gel (24 g column, 0-50% EtOAc/isohexane) to afford the title compound (240 mg, 79%) as a pale brown solid.

¹H NMR (CDCl₃) δ 3.83 (br s, 2H), 2.90 (t, J=7.4 Hz, 4H), 2.73 (t, J=7.4 Hz, 4H), 2.23-2.04 (m, 4H).

LCMS m/z 192.1 (M+H)⁺ (ES⁺).

### Preparation of Examples

### Example 1

### 5-Fluoro-N-((4-(2-hydroxypropan-2-yl)-2-methylphenyl) sulfonyl)-7-isopropyl-3,3-dimethyl-2,3-dihydro-1H-indene-1-carboxamide

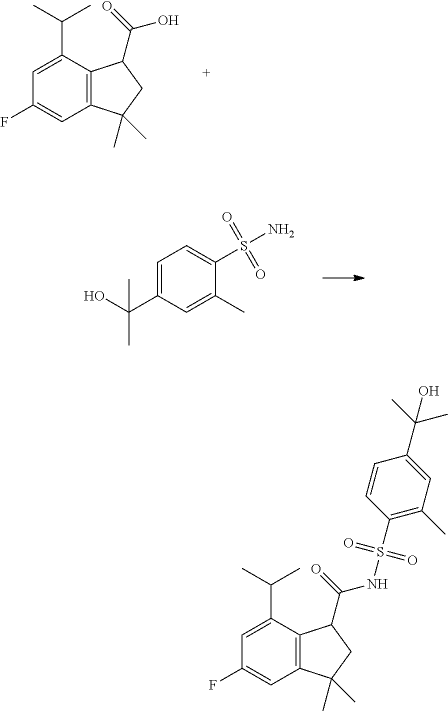 [SMILES: CC(C)c1cc(F)cc2c1C(C(=O)O)CC2(C)C ; Cc1cc(C(C)(C)O)ccc1S(N)(=O)=O ; Cc1cc(C(C)(C)O)ccc1S(=O)(=O)NC(=O)C1CC(C)(C)c2cc(F)cc(C(C)C)c21]

4-(2-Hydroxypropan-2-yl)-2-methylbenzenesulfonamide (Intermediate L1) (36 mg, 0.16 mmol) and 5-fluoro-7-isopropyl-3,3-dimethyl-2,3-dihydro-1H-indene-1-carboxylic acid (Intermediate R7, Step B) (39 mg, 0.16 mmol) were stirred in DCM (6 mL). EDC (60 mg, 0.31 mmol) and DMAP (38 mg, 0.31 mmol) were added to the reaction mixture. The mixture was stirred at room temperature overnight, diluted with DCM (5 mL) and washed with 1M aqueous HCl (3 mL). The organic phase was concentrated. The residue was dissolved in DMSO (0.5 mL) and KO^{t}Bu (52 mg, 0.47 mmol) was added. The mixture was purified by reversed phase prep-HPLC method 2 to afford the title compound (12 mg, 17%) as a white solid.

¹H NMR (300 MHz, Methanol-d₄) δ 7.95 (d, 1H), 7.36 (s, 1H), 7.31 (d, 1H), 6.69-6.60 (m, 1H), 6.60-6.51 (m, 1H), 3.96 (s, 1H), 2.71 (s, 4H), 2.27 (dd, 1H), 2.05 (dd, 1H), 1.49 (d, 6H), 1.24 (s, 3H), 1.14 (s, 3H), 1.04 (d, 3H), 0.80 (d, 3H).

LCMS: m/z 462 (M+H)⁺ (ES⁺); 460 (M−H)⁻ (ES⁻).

### Example 2

### 2-(4-Fluoro-2,6-diisopropylphenyl)-N-((4-(2-hydroxypropan-2-yl)-2-methylphenyl)sulfonyl)acetamide, potassium salt

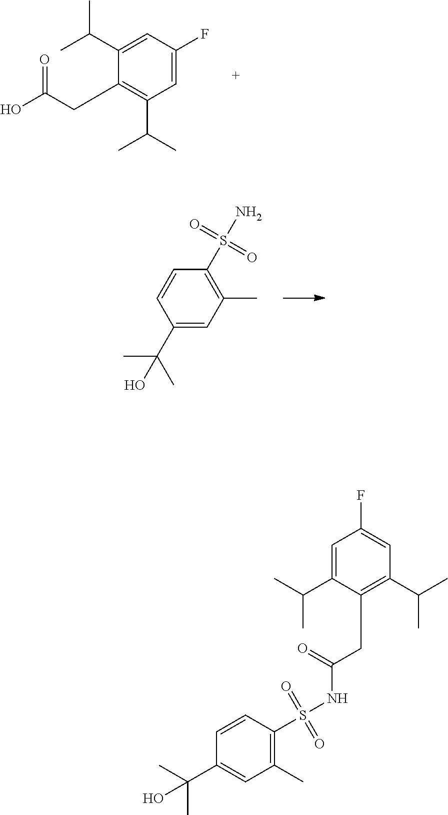 [SMILES: CC(C)c1cc(F)cc(C(C)C)c1CC(=O)O ; Cc1cc(C(C)(C)O)ccc1S(N)(=O)=O ; Cc1cc(C(C)(C)O)ccc1S(=O)(=O)NC(=O)Cc1c(C(C)C)cc(F)cc1C(C)C]

4-(2-Hydroxypropan-2-yl)-2-methylbenzenesulfonamide (Intermediate L1) (50 mg, 0.17 mmol) and 2-(4-fluoro-2,6-diisopropylphenyl)acetic acid (Intermediate R11) (42 mg, 0.17 mmol) were stirred in DCM (6 mL). EDC (67 mg, 0.35 mmol) and DMAP (43 mg, 0.35 mmol) were added to the reaction mixture. The mixture was stirred overnight, diluted with DCM (5 mL) and washed with 1M aqueous HCl (mL). The organic phase was concentrated. The residue was dissolved in DMSO (0.5 mL) and KO^{t}Bu (59 mg, 0.52 mmol) was added. The mixture was purified by reversed phase prep-HPLC method 2 to afford the title compound (41 mg, 52%) as a white solid.

¹H NMR (300 MHz, Methanol-d₄) δ 7.90 (d, 1H), 7.34 (d, 1H), 7.28 (dd, 1H), 6.69 (d, 2H), 3.64 (s, 2H), 3.19-2.98 (m, 2H), 2.68 (s, 3H), 1.48 (d, 6H), 1.07 (d, 12H).

LCMS: m/z 450 (M+H)⁺ (ES⁺); 448 (M−H)⁻ (ES⁻).

### Example 3

### 3-(4-Fluoro-2-isopropyl-6-(2-methoxypyridin-4-yl)phenyl)-N-((4-(2-hydroxypropan-2-yl)-2-methylphenyl)sulfonyl)propanamide

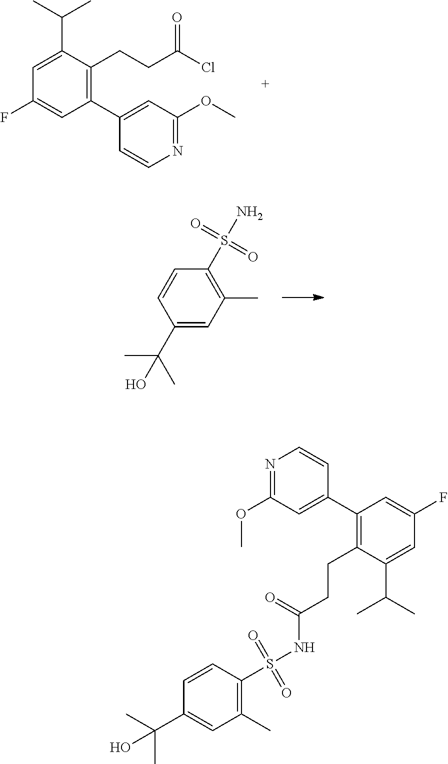 [SMILES: COc1cc(-c2cc(F)cc(C(C)C)c2CCC(=O)Cl)ccn1 ; Cc1cc(C(C)(C)O)ccc1S(N)(=O)=O ; COc1cc(-c2cc(F)cc(C(C)C)c2CCC(=O)NS(=O)(=O)c2ccc(C(C)(C)O)cc2C)ccn1]

4-(2-Hydroxypropan-2-yl)-2-methylbenzenesulfonamide (Intermediate L1) (85 mg, 0.37 mmol) and Et₃N (38 mg, 0.37 mmol) were stirred in DCM (6 mL). Then 3-(4-fluoro-2-isopropyl-6-(2-methoxypyridin-4-yl)phenyl)propanoyl chloride (Intermediate R₅) (63 mg, 0.19 mmol) in DCM (1 mL) was added dropwise and stirred overnight. EDC (36 mg, 0.19 mmol) and DMAP (23 mg, 0.35 mmol) were added to the reaction mixture. The mixture was stirred overnight, diluted with DCM (5 mL) and washed with 1M aqueous HCl (3 mL). The organic phase was concentrated. The residue was dissolved in DMSO (0.5 mL) and purified by reversed phase prep-HPLC method 2 to afford the title compound (22 mg, 22%) as a white solid.

¹H NMR (300 MHz, Methanol-d₄) δ 8.14-8.09 (m, 1H), 7.87 (d, 1H), 7.34 (d, 2H), 7.10-7.00 (m, 1H), 6.83 (dd, 1H), 6.72-6.61 (m, 2H), 3.92 (s, 3H), 3.23 (q, 1H), 2.83-2.66 (m, 2H), 2.54 (s, 3H), 2.22 (dd, 2H), 1.51 (s, 6H), 1.22 (d, 6H).

LCMS: m/z 529 (M+H)⁻ (ES⁻); 527 (M−H)⁻ (ES⁻).

### Example 4

### N-((4-((Dimethylamino)methyl)phenyl)sulfonyl)-3-(4-fluoro-2-isopropyl-6-(2-methoxypyridin-4-yl)phenyl)propanamide, potassium salt

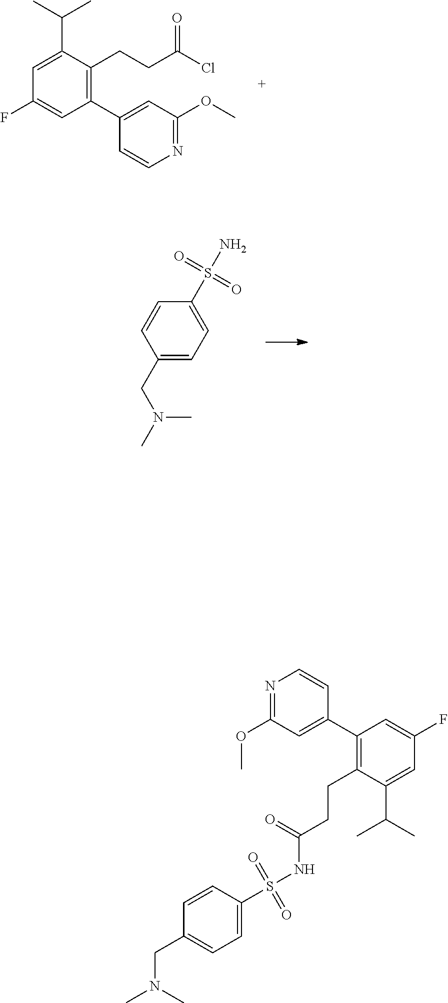 [SMILES: COc1cc(-c2cc(F)cc(C(C)C)c2CCC(=O)Cl)ccn1 ; CN(C)Cc1ccc(S(N)(=O)=O)cc1 ; COc1cc(-c2cc(F)cc(C(C)C)c2CCC(=O)NS(=O)(=O)c2ccc(CN(C)C)cc2)ccn1]

4-((Dimethylamino)methyl)benzenesulfonamide (Intermediate L13) (80 mg, 0.37 mmol) and KO^{t}Bu (63 mg, 0.56 mmol) were stirred in THF (6 mL). Then 3-(4-fluoro-2-isopropyl-6-(2-methoxypyridin-4-yl)phenyl)propanoyl chloride (Intermediate R5) (63 mg, 0.19 mmol) in THF (1 mL) was added dropwise and stirred overnight. EDC (36 mg, 0.19 mmol) and DMAP (23 mg, 0.35 mmol) were added to the reaction mixture. The mixture was stirred for 2 hours and concentrated. The residue was dissolved in DMSO (0.5 mL) and purified by reversed phase prep-HPLC method 2 to afford the title compound (19 mg, 20%) as a white solid.

¹H NMR (300 MHz, Methanol-d₄) δ 8.11 (d, 1H), 7.81 (d, 2H), 7.38 (d, 2H), 7.03 (dd, 1H), 6.86 (d, 1H), 6.65 (m, 2H), 3.93 (s, 3H), 3.54 (s, 2H), 3.30 (m, 1H), 2.78-2.63 (m, 2H), 2.25 (s, 6H), 2.23-2.13 (m, 2H), 1.22 (d, 6H).

LCMS: m/z 514 (M+H)⁺ (ES⁺); 512 (M−H)⁻ (ES⁻).

### Example 5

### 3-(4-Fluoro-2-isopropyl-6-(2-methoxypyridin-4-yl)phenyl)-N-((4-(2-hydroxypropan-2-yl)thiophen-2-yl)sulfonyl)propanamide

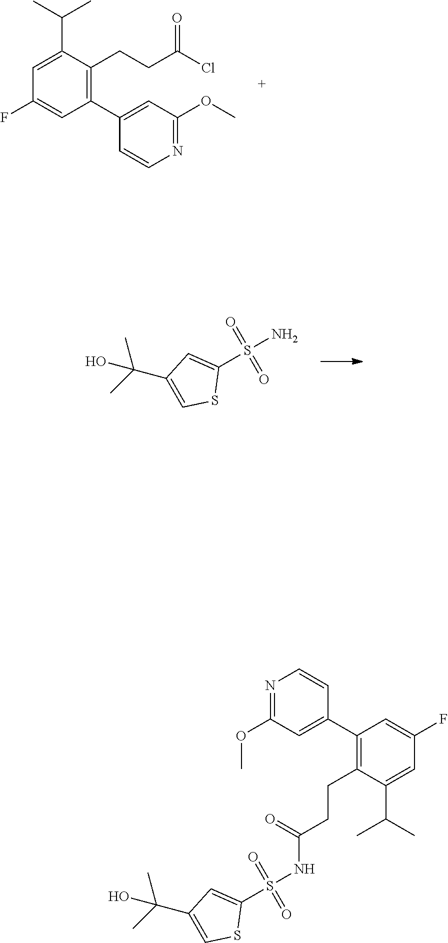 [SMILES: COc1cc(-c2cc(F)cc(C(C)C)c2CCC(=O)Cl)ccn1 ; CC(C)(O)c1csc(S(N)(=O)=O)c1 ; COc1cc(-c2cc(F)cc(C(C)C)c2CCC(=O)NS(=O)(=O)c2cc(C(C)(C)O)cs2)ccn1]

4-(2-Hydroxypropan-2-yl)thiophene-2-sulfonamide (120 mg, 0.55 mmol) and Et₃N (55 mg, 0.55 mmol) were stirred in DCM (6 mL). Then 3-(4-fluoro-2-isopropyl-6-(2-methoxypyridin-4-yl)phenyl)propanoyl chloride (Intermediate R₅) (90 mg, 0.27 mmol) in DCM (1 mL) was added dropwise. The mixture was stirred overnight and concentrated. The residue was purified by reversed phase prep-HPLC method 3 to afford the title compound (5 mg, 4%) as a white solid.

1H NMR (300 MHz, Methanol-d₄) δ 8.18-8.10 (m, 1H), 7.77 (d, 1H), 7.66 (d, 1H), 7.09 (dd, 1H), 6.88 (dd, 1H), 6.78-6.68 (m, 2H), 3.96 (s, 3H), 3.17 (m, 1H), 2.89-2.70 (m, 2H), 2.36-2.22 (m, 2H), 1.52 (s, 6H), 1.24 (d, 6H).

LCMS: m/z 521 (M+H)⁺(ES⁺); 519 (M−H)⁻ (ES⁻).

### Example 6

### N-((1-Cyclopropyl-1H-pyrazol-3-yl)sulfonyl)-6-fluoro-8-isopropyl-4,4-dimethyl-1,2,3,4-tetrahydronaphthalene-1-carboxamide

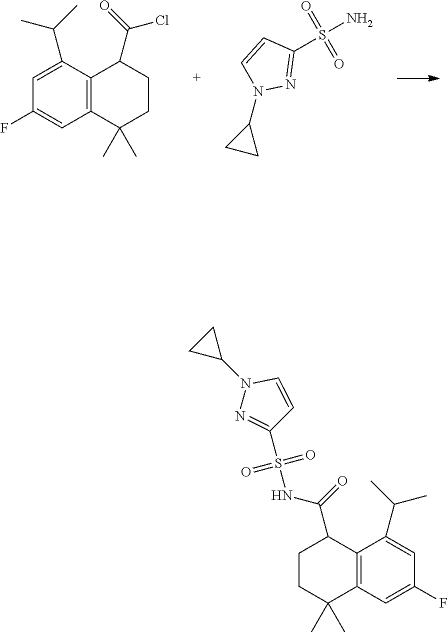 [SMILES: CC(C)c1cc(F)cc2c1C(C(=O)Cl)CCC2(C)C ; NS(=O)(=O)c1ccn(C2CC2)n1 ; CC(C)c1cc(F)cc2c1C(C(=O)NS(=O)(=O)c1ccn(C3CC3)n1)CCC2(C)C]

1-Cyclopropyl-1H-pyrazole-3-sulfonamide (70 mg, 0.37 mmol) and Et₃N (38 mg, 0.37 mmol) were stirred in DCM (6 mL). Then 6-fluoro-8-isopropyl-4,4-dimethyl-1,2,3,4-tetrahydronaphthalene-1-carbonyl chloride (Intermediate R6) (53 mg, 0.19 mmol) in DCM (1 mL) was added dropwise. The mixture was stirred overnight. DMAP (23 mg, 0.19 mmol) was added to the reaction mixture. The mixture was stirred for another day and then concentrated. The residue was purified by reversed phase prep-HPLC method 3 to afford the title compound (8 mg, 10%) as a white solid.

¹H NMR (300 MHz, Methanol-d₄) δ 7.76 (d, 1H), 6.90 (dd, 1H), 6.75 (m, 2H), 3.88 (t, 1H), 3.77 (tt, 1H), 2.82-2.65 (m, 1H), 2.07 (dt, 2H), 1.73-1.58 (m, 1H), 1.58-1.39 (m, 1H), 1.24 (d, 6H), 1.20-1.01 (m, 6H), 0.89 (d, 4H).

LCMS: m/z 434 (M+H)⁺ (ES⁺); 432(M−H)⁻ (ES⁻).

### Example 7

### N-((1-Cyclopropyl-1H-pyrazol-4-yl)sulfonyl)-5-fluoro-7-isopropyl-3,3-dimethyl-2,3-dihydro-1H-indene-1-carboxamide

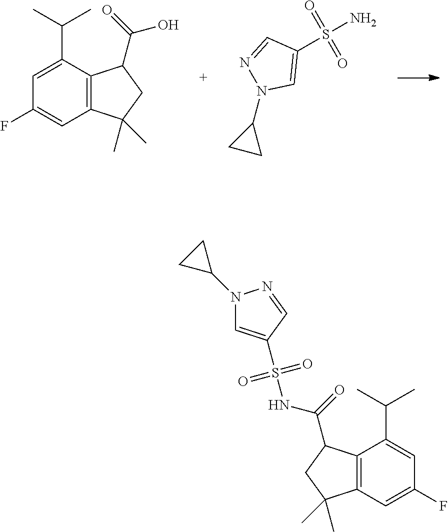 [SMILES: CC(C)c1cc(F)cc2c1C(C(=O)O)CC2(C)C ; NS(=O)(=O)c1cnn(C2CC2)c1 ; CC(C)c1cc(F)cc2c1C(C(=O)NS(=O)(=O)c1cnn(C3CC3)c1)CC2(C)C]

5-Fluoro-7-isopropyl-3,3-dimethyl-2,3-dihydro-1H-indene-1-carboxylic acid (Intermediate R7, Step B) (60 mg, 0.24 mmol), DIPEA (93 mg, 0.72 mmol) and HATU (110 mg, 0.29 mmol) were stirred in DCM (6 mL) for 50 minutes. Then 1-cyclopropyl-1H-pyrazole-4-sulfonamide (45 mg, 0.24 mmol) was added. The mixture was stirred overnight, diluted with DCM and washed with water. The organic phase was concentrated. The residue was purified by reversed phase prep-HPLC method 3 to afford the title compound (main rotamer) (5 mg, 5%) as a white solid.

¹H NMR (300 MHz, Methanol-d₄) δ 8.25 (s, 1H), 7.83 (s, 1H), 6.81-6.61 (m, 2H), 4.01-3.89 (m, 1H), 3.71 (dt, 1H), 2.47 (dt, 1H), 2.31 (dd, 1H), 2.06-1.92 (m, 1H), 1.21 (d, 6H), 1.11-1.00 (m, 6H), 0.89 (d, 4H).

LCMS: m/z 420 (M+H)⁺ (ES⁺); 418 (M−H)⁻ (ES⁻).

### Example 8

### 5-Fluoro-N-((4-(2-hydroxypropan-2-yl)thiophen-2-yl)sulfonyl)-7-isopropyl-3,3-dimethyl-2,3-dihydro-1H-indene-1-carboxamide, potassium salt (rotamer P1) and

### Example 9

### 5-Fluoro-N-((4-(2-hydroxypropan-2-yl)thiophen-2-yl)sulfonyl)-7-isopropyl-3,3-dimethyl-2,3-dihydro-1H-indene-1-carboxamide, potassium salt (rotamer P2)

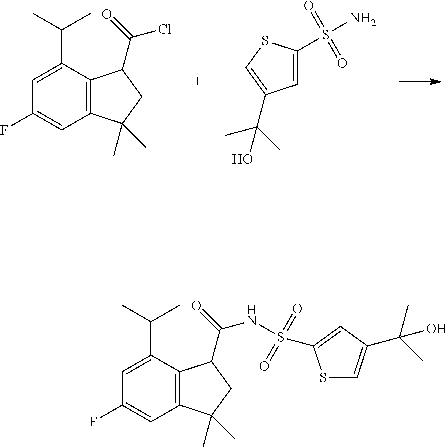 [SMILES: CC(C)c1cc(F)cc2c1C(C(=O)Cl)CC2(C)C ; CC(C)(O)c1csc(S(N)(=O)=O)c1 ; CC(C)c1cc(F)cc2c1C(C(=O)NS(=O)(=O)c1cc(C(C)(C)O)cs1)CC2(C)C]

4-(2-Hydroxypropan-2-yl)thiophene-2-sulfonamide (110 mg, 0.48 mmol) and KO^{t}Bu (54 mg, 0.48 mmol) were stirred in THF (6 mL). Then 5-fluoro-7-isopropyl-3,3-dimethyl-2,3-dihydro-1H-indene-1-carbonyl chloride (Intermediate R7) (65 mg, 0.24 mmol) in THF (1 mL) was added dropwise. The reaction mixture was stirred overnight and concentrated. The residue was purified by reversed phase prep-HPLC method 3 to afford the title compounds as separate rotamers. Rotamer P1 was the first eluting rotamer (10 mg, 9%) and rotamer P2 was the second eluting rotamer (2 mg, 2%), both as white solids.

Rotamer P1:

¹H NMR (300 MHz, Methanol-d₄) δ 7.80 (d, 1H), 7.64 (d, 1H), 6.75 (dd, 1H), 6.67 (dd, 1H), 3.97 (ddd, 1H), 2.62-2.44 (m, 1H), 2.32 (dd, 1H), 1.98 (dd, 1H), 1.50 (s, 6H), 1.21 (d, 6H), 1.08 (d, 3H), 0.94 (d, 3H).

LCMS: m/z 454 (M+H)⁻ (ES⁻); 452 (M−H)⁻ (ES⁻).

Rotamer P2:

LCMS: m/z 454 (M+H)⁺ (ES⁺); 452 (M−H)⁻ (ES⁻).

### Example 10

### 5-Fluoro-7-isopropyl-N-((1-isopropyl-1H-pyrazol-3-yl) sulfonyl)-3,3-dimethyl-2,3-dihydro-1H-indene-1-carboxamide, potassium salt (rotamer P2)

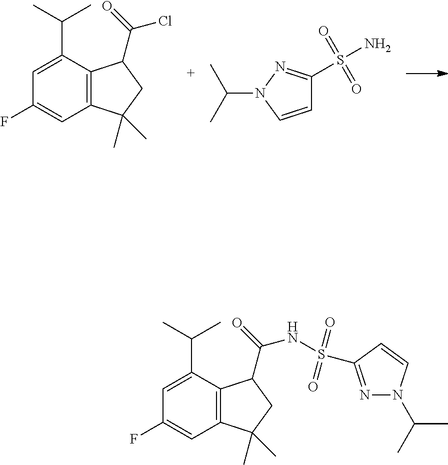 [SMILES: CC(C)c1cc(F)cc2c1C(C(=O)Cl)CC2(C)C ; CC(C)n1ccc(S(N)(=O)=O)n1 ; CC(C)c1cc(F)cc2c1C(C(=O)NS(=O)(=O)c1ccn(C(C)C)n1)CC2(C)C]

1-Isopropyl-1H-pyrazole-3-sulfonamide (Intermediate L8) (91 mg, 0.48 mmol) and KO^{t}Bu (81 mg, 0.72 mmol) were stirred in THF (6 mL). Then 5-fluoro-7-isopropyl-3,3-dimethyl-2,3-dihydro-1H-indene-1-carbonyl chloride (Intermediate R7) (65 mg, 0.24 mmol) in THF (1 mL) was added dropwise. The reaction mixture was stirred overnight and concentrated. The residue was purified by reversed phase prep-HPLC method 3. The title compound was isolated as the second eluting rotamer (3 mg, 3%) as a white solid.

¹H NMR (300 MHz, Methanol-d₄) δ 7.79 (d, 1H), 7.03 (d, 1H), 6.85-6.75 (m, 2H), 4.69-4.54 (m, 1H), 4.00 (t, 1H), 3.13 (p, 1H), 2.27-2.03 (m, 2H), 1.54-1.44 (m, 6H), 1.29 (s, 3H), 1.25-1.13 (m, 9H).

LCMS: m/z 422 (M+H)⁺ (ES⁺); 418 (M−H)⁻ (ES⁻).

### Example 11

### 3-(N-(5-Fluoro-7-isopropyl-3,3-dimethyl-2,3-dihydro-1H-indene-1-carbonyl)sulfamoyl)-N,N,1-trimethyl-1H-pyrazole-5-carboxamide

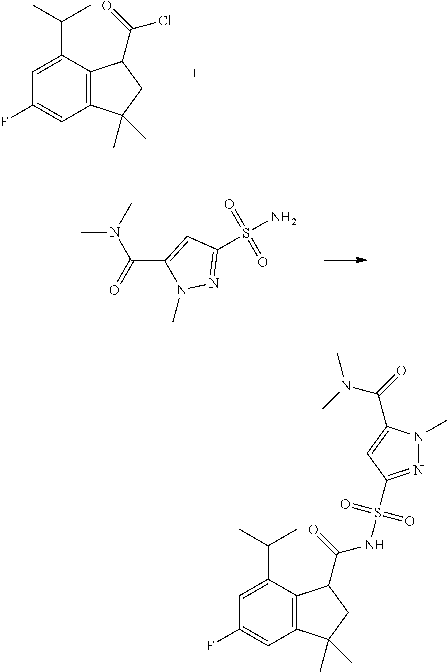 [SMILES: CC(C)c1cc(F)cc2c1C(C(=O)Cl)CC2(C)C ; CN(C)C(=O)c1cc(S(N)(=O)=O)nn1C ; CC(C)c1cc(F)cc2c1C(C(=O)NS(=O)(=O)c1cc(C(=O)N(C)C)n(C)n1)CC2(C)C]

N,N,1-Trimethyl-3-sulfamoyl-1H-pyrazole-5-carboxamide (120 mg, 0.52 mmol) and Et₃N (110 mg, 1 mmol) were stirred in DCM (6 mL). Then 5-fluoro-7-isopropyl-3,3-dimethyl-2,3-dihydro-1H-indene-1-carbonyl chloride (Intermediate R7) (70 mg, 0.26 mmol) in DCM (1 mL) was added dropwise. The mixture was stirred overnight and then concentrated. The residue was purified by reversed phase prep-HPLC method 3 to afford the title compound (4 mg, 3%) as a white solid.

¹H NMR (300 MHz, Methanol-d₄) δ 6.95 (s, 1H), 6.73 (dd, 1H), 6.64 (dd, 1H), 3.96 (s, 3H), 3.95 (m, 1H), 3.09 (d, 6H), 2.80-2.66 (m, 1H), 2.31 (dd, 1H), 2.05 (dd, 1H), 1.21 (d, 6H), 1.10 (d, 3H), 0.99 (d, 3H).

LCMS: m/z 465 (M+H)⁺ (ES⁺); 463 (M−H)⁻ (ES⁻).

### Example 12

### 1-(2-(Bis(methyl-d3)amino)ethyl)-N-((1,2,3,5,6,7-hexahydro-s-indacen-4-yl)carbamoyl)-1H-pyrazole-3-sulfonamide, potassium salt

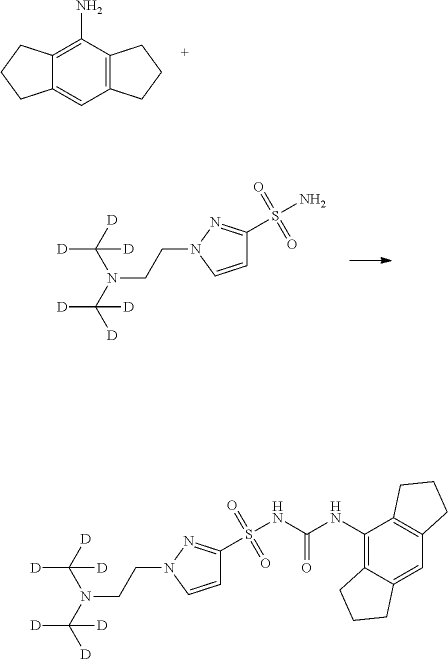 [SMILES: Nc1c2c(cc3c1CCC3)CCC2 ; [2H]C([2H])([2H])N(CCn1ccc(S(N)(=O)=O)n1)C([2H])([2H])[2H] ; [2H]C([2H])([2H])N(CCn1ccc(S(=O)(=O)NC(=O)Nc2c3c(cc4c2CCC4)CCC3)n1)C([2H])([2H])[2H]]

1-(2-(Bis(methyl-d3)amino)ethyl)-1H-pyrazole-3-sulfonamide, 2,2,2-trifluoroacetate salt (Intermediate L5) (367 mg, 1.08 mmol) and KO^{t}Bu (365 mg, 3.25 mmol) were stirred in THF (6 mL). Then 4-isocyanato-1,2,3,5,6,7-hexahydro-s-indacene (Intermediate R1) (230 mg, 1.08 mmol) in THF (1 mL) was added dropwise. The mixture was stirred overnight and concentrated. The residue was dissolved in DMSO (1 mL) and purified by reversed phase prep-HPLC method 2 to afford the title compound (114 mg, 25%) as a white solid.

¹H NMR (300 MHz, Methanol-d₄) δ 7.67 (d, 1H), 6.85 (s, 1H), 6.63 (d, 1H), 4.29 (t, 2H), 2.80 (td, 6H), 2.71 (t, 4H), 1.98 (p, 4H).

LCMS: m/z 424 (M+H)⁺ (ES⁺); 422 (M−H)⁻ (ES⁻).

### Example 13

### N-((1,2,3,5,6,7-Hexahydro-s-indacen-4-yl)carbamoyl)-1H-pyrazole-3-sulfonamide, potassium salt

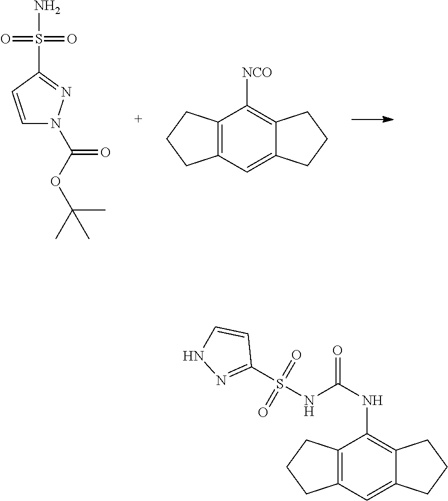

To a solution of tert-butyl 3-sulfamoyl-1H-pyrazole-1-carboxylate (Intermediate L3) (62 mg, 0.25 mmol) in THF (1 mL) was added KO^{t}Bu (28 mg, 0.25 mmol). The suspension that formed was stirred for 1 hour, before adding a solution of 4-isocyanato-1,2,3,5,6,7-hexahydro-s-indacene (Intermediate R1) (50 mg, 0.25 mmol) in THF (1 mL). The resulting reaction mixture was stirred overnight at room temperature. The mixture was concentrated under reduced pressure and the residue was analysed by HPLC to observe a mixture of the title compound and the BOC-protected derivative. The crude product was dissolved in DMSO (0.5 mL) and purified by reversed phase prep-HPLC method 2 to afford the title compound (17 mg, 20%) as a white solid.

¹H NMR (CD₃OD) δ 7.60 (s, 1H), 6.85 (d, 1H), 6.69 (t, 1H), 2.81 (t, 4H), 2.71 (t, 4H), 1.98 (m, 4H).

LCMS: m/z 347 (M+H)⁺ (ES⁺); 346 (M−H)⁻ (ES).

### Example 14

### N-((1,2,3,5-Tetrahydro-s-indacen-4-yl)carbamoyl)-1H-pyrazole-3-sulfonamide, potassium salt

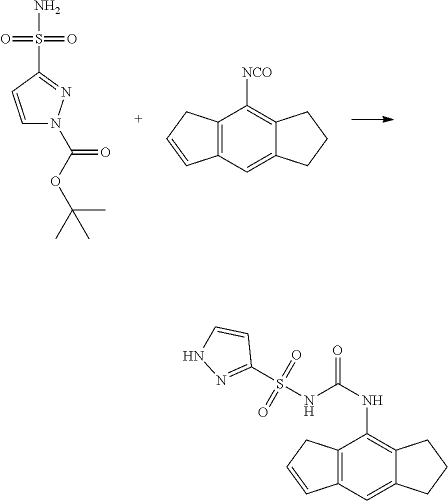

To a solution of tert-butyl 3-sulfamoyl-1H-pyrazole-1-carboxylate (Intermediate L3) (41 mg, 0.17 mmol) in THF (1 mL) was added KO^{t}Bu (19 mg, 0.17 mmol). The suspension that formed was stirred for 1 hour, before adding a solution of 8-isocyanato-1,2,3,5-tetrahydro-s-indacene (Intermediate R2) (33 mg, 0.17 mmol) in THF (0.8 mL). The resulting reaction mixture was stirred overnight at room temperature. The mixture was concentrated under reduced pressure and the residue was analysed by HPLC to observe a mixture of the title compound and the BOC-protected derivative. The crude product was dissolved in DMSO (0.5 mL) and submitted for purification by reversed phase column chromatography to afford the title compound (13 mg, 22%) as a white solid.

¹H NMR (CD₃OD) δ 7.61 (s, 1H), 7.04 (s, 1H), 6.73 (m, 2H), 6.41 (s, 1H), 2.85 (dt, 6H), 2.03 (m, 2H).

LCMS: m/z 345 (M+H)⁺ (ES⁺); 343 (M−H)⁻ (ES⁻).

### Example 15

### 1-Isopropyl-N-((3-oxo-1,2,3,5,6,7-hexahydro-s-indacen-4-yl)carbamoyl)-1H-pyrazole-3-sulfonamide, potassium salt

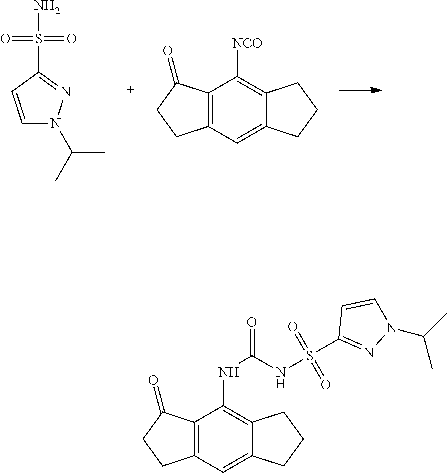

To a solution of 1-isopropyl-1H-pyrazole-3-sulfonamide (Intermediate L8) (57 mg, 0.30 mmol) in THF (1 mL) was added KO^{t}Bu (34 mg, 0.30 mmol). The suspension that formed was stirred for 1 hour, before adding a solution of 8-isocyanato-3,5,6,7-tetrahydro-s-indacen-1(2H)-one (Intermediate R4) (64 mg, 0.30 mmol) in THF (1 mL). The resulting reaction mixture was stirred overnight at room temperature. The mixture was concentrated under reduced pressure. The residue was dissolved in DMSO (1 mL) and submitted for purification by reversed phase column chromatography to afford the title compound (25 mg, 20%) as a white solid.

¹H NMR (CD₃OD) δ 7.61 (d, 1H), 7.00 (s, 1H), 6.66 (d, 1H), 4.53 (p, 1H), 3.00 (m, 2H), 2.88 (t, 2H), 2.77 (t, 2H), 2.64 (m, 2H), 1.96 (p, 2H), 1.47 (d, 6H).

LCMS: m/z 403 (M+H)⁺ (ES⁺); 401 (M−H)⁻ (ES⁻).

### Example 16

### 1-(2-(3-(But-3-yn-1-yl)-3H-diazirin-3-yl)ethyl)-N-((1,2,3,5,6,7-hexahydro-s-indacen-4-yl)carbamoyl)-1H-pyrazole-3-sulfonamide, potassium salt

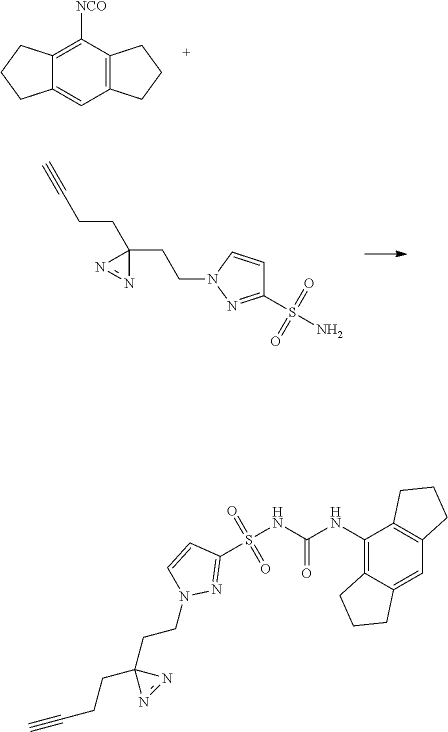

1-(2-(3-(But-3-yn-1-yl)-3H-diazirin-3-yl)ethyl)-1H-pyrazole-3-sulfonamide, 2,2,2-trifluoroacetate salt (Intermediate L4) (12 mg, 0.047 mmol) and KO^{t}Bu (8 mg, 0.071 mmol) were stirred in THF (3 mL). Then 4-isocyanato-1,2,3,5,6,7-hexahydro-s-indacene (Intermediate R1) (9 mg, 0.047 mmol) in THF (1 mL) was added dropwise. The resulting reaction mixture was stirred overnight and concentrated. The residue was dissolved in DMSO (1 mL) and purified by reversed phase prep-HPLC method 2 to afford the title compound (10 mg, 45%) as a white solid.

¹H NMR (300 MHz, Methanol-d₄) δ 6.85 (s, 1H), 6.67 (d, 1H), 4.07 (t, 2H), 3.72 (s, 1H), 2.76 (dt, 9H), 1.96 (ddt, 8H), 1.47 (t, 2H).

LCMS: m/z 467 (M+H)⁺(ES⁺); 465 (M−H)⁻ (ES⁻).

### Example 17

### 4-((2-(Aminomethyl)-3-fluoroallyl)oxy)-N-((1,2,3,5,6,7-hexahydro-s-indacen-4-yl)carbamoyl)benzenesulfonamide, 2,2,2-trifluoroacetate salt

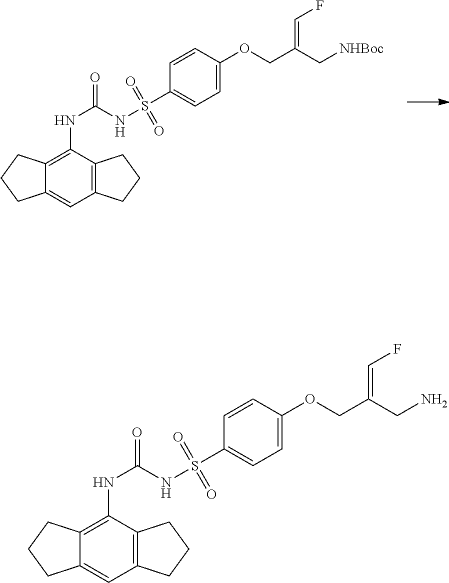 [SMILES: CC(C)(C)OC(=O)NC/C(=C\F)COc1ccc(S(=O)(=O)NC(=O)Nc2c3c(cc4c2CCC4)CCC3)cc1 ; NC/C(=C\F)COc1ccc(S(=O)(=O)NC(=O)Nc2c3c(cc4c2CCC4)CCC3)cc1]

tert-Butyl-(3-fluoro-2-((4-(N-((1,2,3,5,6,7-hexahydro-s-indacen-4-yl)carbamoyl) sulfamoyl)phenoxy)methyl)allyl)carbamate, potassium salt (Example 18) (7 mg, 0.013 mmol) was dissolved in DCM (0.3 mL) and TFA (0.3 mL). The mixture was stirred for 4 hours at room temperature and concentrated. The residue was taken up in water and freeze-dried to afford the title compound (3 mg, 43%) as a white solid.

¹H NMR (300 MHz, Methanol-d₄) δ 7.86 (m, 2H), 7.13 (m, 1H), 7.02 (m, 2H), 6.85 (m, 1H), 4.68-4.51 (m, ²H), 3.61-3.43 (m, 2H), 2.87-2.61 (m, 8H), 1.97 (p, 4H).

LCMS: m/z 460 (M+H)⁺ (ES⁺); 458 (M−H)⁻ (ES⁻).

### Example 18

### tert-Butyl-(3-fluoro-2-((4-(N-((1,2,3,5,6,7-hexahydro-s-indacen-4-yl)carbamoyl)sulfamoyl)phenoxy)methyl)allyl)carbamate, potassium salt

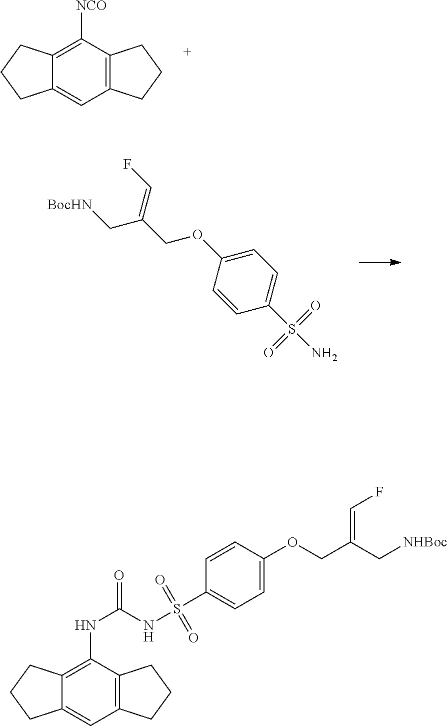

tert-Butyl-(3-fluoro-2-((4-sulfamoylphenoxy)methyl)allyl)carbamate (prepared as described in WO 2013/163675, 95 mg, 0.26 mmol) and KO^{t}Bu (30 mg, 0.26 mmol) were stirred in THF (3 mL). Then 4-isocyanato-1,2,3,5,6,7-hexahydro-s-indacene (Intermediate R1) (52 mg, 0.26 mmol) in THF (1 mL) was added dropwise. The mixture was stirred overnight and concentrated. The residue was dissolved in DMSO (1 mL) and purified by reversed phase prep-HPLC method 2 to afford the title compound (9 mg, 6%) as a white solid.

¹H NMR (300 MHz, Methanol-d₄) δ 7.87 (d, 2H), 7.07-6.93 (m, 3H), 6.79 (d, 1H), 4.55-4.46 (m, 1H), 3.92 (s, 1H), 2.80 (t, 4H), 2.75-2.60 (m, 6H), 1.98 (q, 4H), 1.39 (s, 9H).

LCMS: m/z 560 (M+H)⁺ (ES⁺); 558 (M−H)⁻ (ES⁻).

### Example 19

### 1-Ethyl-N-((1,2,3,5,6,7-hexahydro-s-indacen-4-yl)carbamoyl)-1H-1,2,3-triazole-4-sulfonamide, potassium salt

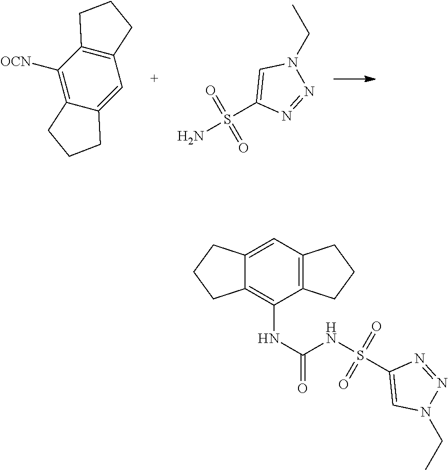

1-Ethyl-1H-1,2,3-triazole-4-sulfonamide (Chemspace, 49 mg, 0.28 mmol) and KO^{t}Bu (31 mg, 0.28 mmol) were stirred in THF (6 mL). Then 4-isocyanato-1,2,3,5,6,7-hexahydro-s-indacene (Intermediate R1) (55 mg, 0.28 mmol) in THF (1 mL) was added dropwise. The mixture was stirred overnight and concentrated. The residue was dissolved in DMSO (1 mL) and purified by reversed phase prep-HPLC method 2 to afford the title compound (65 mg, 63%) as a white solid.

¹H NMR (300 MHz, Methanol-d₄) δ 8.29 (s, 1H), 6.87 (s, 1H), 4.47 (q, 2H), 2.77 (m, 8H), 2.00 (m, 4H), 1.54 (t, 3H).

LCMS: m/z 376 (M+H)⁺ (ES⁺); 374 (M−H)⁻ (ES⁻).

### Example 20

### N-((1,2,3,5,6,7-Hexahydro-s-indacen-4-yl)carbamoyl)-1-(propan-2-yl-d7)-1H-pyrazole-3-sulfonamide, potassium salt

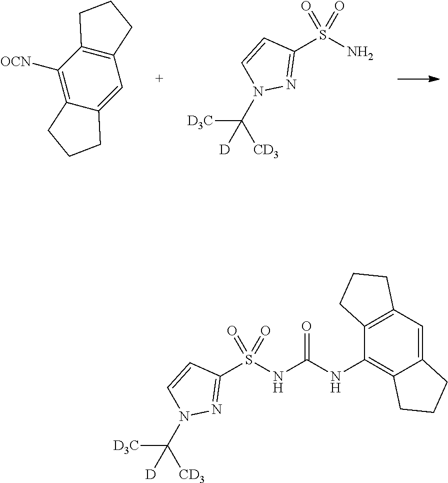

1-(Propan-2-yl-d7)-1H-pyrazole-3-sulfonamide (Intermediate L2) (146 mg, 0.47 mmol) and KO^{t}Bu (106 mg, 0.94 mmol) were stirred in THF (6 mL). Then 4-isocyanato-1,2,3,5,6,7-hexahydro-s-indacene (Intermediate R1) (100 mg, 0.47 mmol) in THF (1 mL) was added dropwise. The mixture was stirred overnight and concentrated. The residue was dissolved in DMSO (1 mL) and purified by reversed phase prep-HPLC method 2 to afford the title compound (112 mg, 60%) as a white solid.

¹H NMR (300 MHz, Methanol-d₄) δ 7.65 (t, 1H), 6.84 (s, 1H), 6.63 (d, 1H), 2.75 (m, 8H), 2.19-1.79 (m, 4H).

LCMS: m/z 396 (M+H)⁺ (ES⁺); 394 (M−H)⁻ (ES⁻).

### Example 21

### N-((1-Hydroxy-1,2,3,5,6,7-hexahydro-s-indacen-4-yl) carbamoyl)-1-isopropyl-1H-pyrazole-3-sulfonamide

### Step A: 1-Isopropyl-N-((1-oxo-1,2,3,5,6,7-hexahydro-s-indacen-4-yl)carbamoyl)-1H-pyrazole-3-sulfonamide

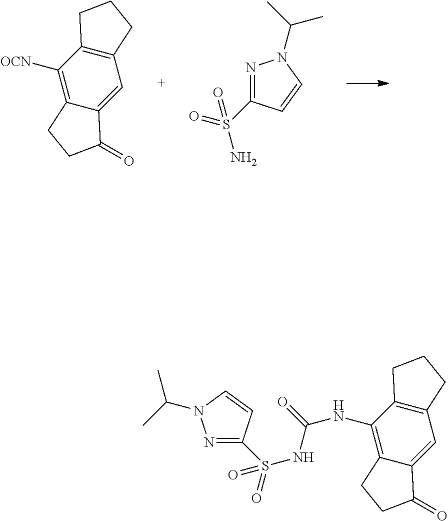

A solution of 1-isopropyl-1H-pyrazole-3-sulfonamide (Intermediate L8) (45 mg, 0.22 mmol) in THF (4 mL) was cooled to 0° C. Then KO^{t}Bu (27 mg, 0.24 mmol) was added and the mixture was stirred for 30 minutes at 21° C. Next a solution of 4-isocyanato-3,5,6,7-tetrahydro-s-indacen-1(2H)-one (Intermediate R3) (46 mg, 0.24 mmol) in THF (4 mL) was added and the mixture was stirred for 18 hours at 21° C. The mixture was filtered to afford the title compound (70 mg, 80%) as a white solid.

LCMS: m/z 403 (M+H)⁺ (ES⁺); 401 (M−H)⁻ (ES⁻).

### Step B: N-((1-Hydroxy-1,2,3,5,6,7-hexahydro-s-indacen-4-yl)carbamoyl)-1-isopropyl-1H-pyrazole-3-sulfonamide

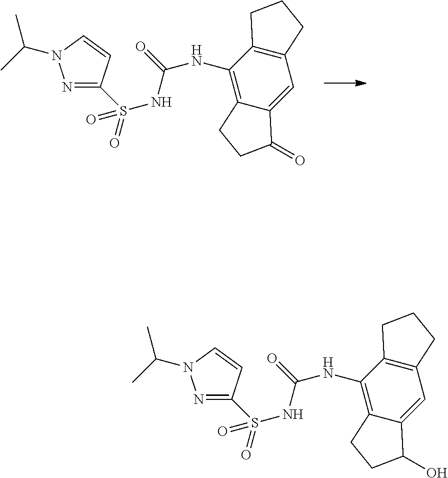 [SMILES: CC(C)n1ccc(S(=O)(=O)NC(=O)Nc2c3c(cc4c2CCC4=O)CCC3)n1 ; CC(C)n1ccc(S(=O)(=O)NC(=O)Nc2c3c(cc4c2CCC4O)CCC3)n1]

1-Isopropyl-N-((1-oxo-1,2,3,5,6,7-hexahydro-s-indacen-4-yl)carbamoyl)-1H-pyrazole-3-sulfonamide (70 mg, 0.17 mmol) in methanol (4 mL) was cooled to 0° C. Next sodium borohydride (66 mg, 1.74 mmol) was added and the mixture was stirred for 18 hours at room temperature. Water (2 mL) was added and the mixture was evaporated. The residue was purified by reversed phase prep-HPLC method 3 to afford the title compound (64 mg, 91%) as a white solid.

¹H NMR (300 MHz, D₂O) δ 7.65 (d, 1H), 7.08 (s, 1H), 6.59 (d, 1H), 5.10 (t, 1H), 4.49 (m, 1H), 2.80 (t, 2H), 2.71 (m, 1H), 2.60 (t, 2H), 2.52 (m, 1H), 2.33 (m, 1H), 1.93 (m, 2H), 1.81 (m, 1H), 1.39 (d, 6H).

LCMS: m/z 463 (M+59)⁺ (ES⁺ ); 403 (M−H)⁻ (ES⁻).

### Example 22

### 1-Isopropyl-N-((1,2,3,7-tetrahydro-s-indacen-4-yl)carbamoyl)-1H-pyrazole-3-sulfonamide, potassium salt and 1-isopropyl-N-((1,2,3,5-tetrahydro-s-indacen-4-yl)carbamoyl)-1H-pyrazole-3-sulfonamide, potassium salt

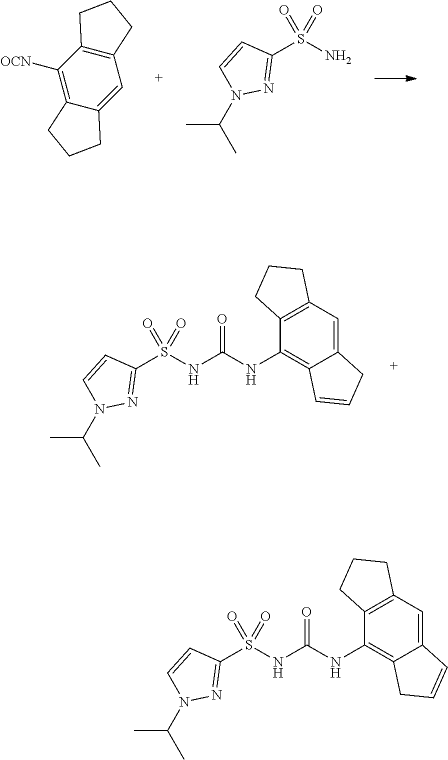

To a solution of 1-isopropyl-1H-pyrazole-3-sulfonamide (Intermediate L8) (31 mg, 0.16 mmol) in THF (4 mL) was added KO^{t}Bu (18 mg, 0.16 mmol) at 0° C. The mixture was stirred for 1 hour at 21° C. Next 8-isocyanato-1,2,3,5-tetrahydro-s-indacene (Intermediate R2) (35 mg, 0.18 mmol) in THF (3 mL) was added and the mixture was stirred for 18 hours at 21° C. The solvents were evaporated and the residue was purified by reversed phase column chromatography to afford the title compound (65 mg, 100%) as a white solid.

¹H NMR (300 MHz, D₂O), mixture of two isomers, ratio 3/2, δ 7.65 (d, 1H), 7.22 and 7.16 (s, 1H), 6.77 and 6.68 (d, J=32.6, 5.6 Hz, 1H), 6.59 (d, J=2.4 Hz, 1H), 6.55-6.37 (m, 1H), 4.49 (dt, J=13.4, 6.6 Hz, 1H), 3.31 and 3.16 (s, 2H), 2.74 (dt, J=54.3, 7.4 Hz, 4H), 1.95 (m, J=7.5 Hz, 2H), 1.39 (d, J=6.7 Hz, 7H).

LCMS: m/z 387 (M+H)⁺ (ES⁺); 385 (M−H)⁻ (ES⁻).

### Example 23

### 1-Isopropyl-N-((5-(2-methoxypyridin-4-yl)-2,3-dihydro-1H-inden-4-yl)carbamoyl)-1H-pyrazole-3-sulfinamide

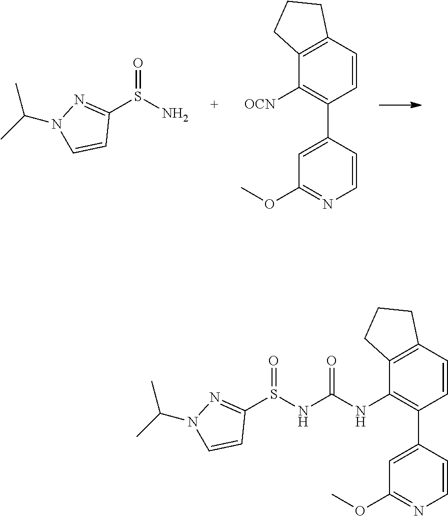

To a solution of 1-isopropyl-1H-pyrazole-3-sulfinamide (Intermediate L6) (0.1 g, 577.25 μmol, 1 eq) in THF (2 mL) was added t-BuONa (56 mg, 577.25 μmol, 1 eq). The mixture was stirred at 25° C. for 30 minutes. Then 4-(4-isocyanato-2,3-dihydro-1H-inden-5-yl)-2-methoxypyridine (Intermediate R8) (150 mg, 563.29 μmol, 0.98 eq) was added. The resulting mixture was stirred at 25° C. for 1 hour, and then concentrated in vacuo. The residue was purified by prep-HPLC (column: Waters Xbridge C18, 150 mm×25 mm×5 μm; mobile phase: [A: water (0.05% ammonium hydroxide v/v), B: MeCN]; B %: 20%-50%, 10 min) to give the title compound (81.42 mg, 31% yield, 98% purity on LCMS) as a white solid.

¹H NMR (400 MHz, DMSO-d₆) δ 8.15 (d, 1H), 7.92 (s, 1H), 7.22-7.19 (m, 1H), 7.14-7.11 (m, 1H), 6.95 (d, 1H), 6.77 (s, 1H), 6.59 (s, 1H), 4.60-4.53 (m, 1H), 3.87 (s, 3H), 2.94 (t, 2H), 2.82 (t, 2H), 2.04-2.00 (m, 2H) and 1.44 (d, 6H).

LCMS: m/z 440.2 (M+H)⁺ (ES⁺).

### Example 24

### 2-(1,2,3,5,6,7-Hexahydro-s-indacen-4-yl)-N-(1-isopropyl-1H-pyrazole-3-sulfonimidoyl)acetamide

### Step A: 2-(1,2,3,5,6,7-Hexahydro-s-indacen-4-yl)-N-((1-isopropyl-1H-pyrazol-3-yl)sulfinyl)acetamide

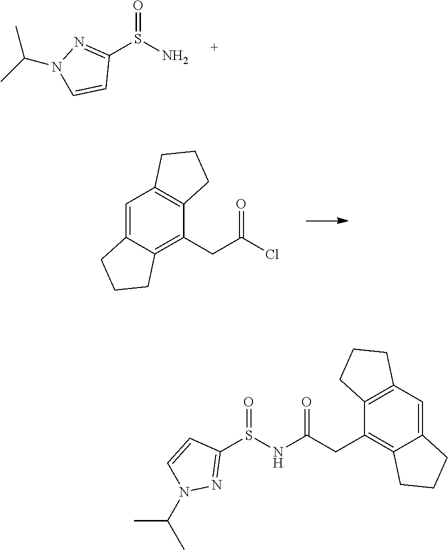 [SMILES: CC(C)n1ccc(S(N)=O)n1 ; O=C(Cl)Cc1c2c(cc3c1CCC3)CCC2 ; CC(C)n1ccc(S(=O)NC(=O)Cc2c3c(cc4c2CCC4)CCC3)n1]

To a solution of 1-isopropyl-1H-pyrazole-3-sulfinamide (Intermediate L6) (400 mg, 2.31 mmol, 1 eq) in THF (20 mL) was added n-BuLi (2 M, 1.27 mL, 1.1 eq) at −70° C. The reaction mixture was stirred for 30 minutes. To this reaction mixture was added a solution of 2-(1,2,3,5,6,7-hexahydro-s-indacen-4-yl)acetyl chloride (Intermediate R9) (542 mg, 2.31 mmol, 1 eq) in THF (1 mL) at 20° C. The reaction mixture was stirred at −70° C. for 2.5 hours, poured into water (50 mL), and extracted with EtOAc (2×50 mL). The combined organic layers were dried over Na₂SO₄, filtered and concentrated in vacuo. The residue was purified by reversed phase flash chromatography (0.01% TFA-MeCN) to give the title compound (170 mg, 20%) as a yellow solid.

¹H NMR (400 MHz, DMSO-d₆) δ 11.31 (br s, 1H), 8.05 (d, 1H), 6.97 (s, 1H), 6.75 (d, 1H), 4.65-4.58 (m, 1H), 3.62 (s, 2H), 2.83-2.74 (m, 8H), 2.02-1.94 (m, 4H) and 1.46 (m, 6H).

LCMS: m/z 372.1 (M+H)⁺ (ES⁺).

### Step B: 2-(1,2,3,5,6,7-Hexahydro-s-indacen-4-yl)-N-(1-isopropyl-1H-pyrazole-3-sulfonimidoyl)acetamide

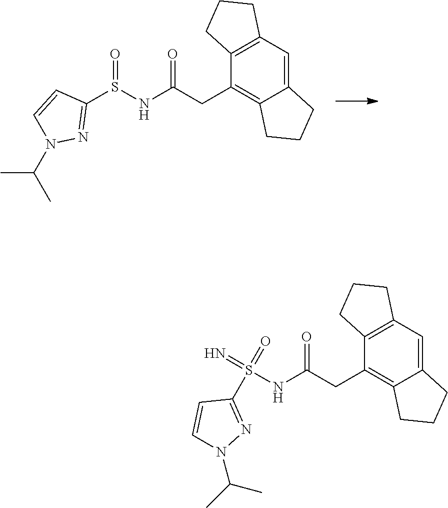 [SMILES: CC(C)n1ccc(S(=O)NC(=O)Cc2c3c(cc4c2CCC4)CCC3)n1 ; CC(C)n1ccc(S(=N)(=O)NC(=O)Cc2c3c(cc4c2CCC4)CCC3)n1]

To a solution of 2-(1,2,3,5,6,7-hexahydro-s-indacen-4-yl)-N-((1-isopropyl-1H-pyrazol-3-yl)sulfinyl)acetamide (150 mg, 403.77 μmol, 1 eq) in THF (5 mL) was added 1-chloro-1H-benzo[d][1,2,3]triazole (68 mg, 444.15 μmol, 1.1 eq). The reaction mixture was stirred at 20° C. for 0.5 hour. The resulting mixture was added dropwise at −70° C. into a solution of NH₃ in THF, which had been prepared by bubbling NH₃ (15 psi) into THF (20 mL) at −70° C. for 10 minutes. The reaction mixture was stirred at 20° C. for 2 hours, and then concentrated in vacuo. The residue was diluted with EtOAc (100 mL) and washed with water (2×50 mL). The organic layer was dried over Na₂SO₄, filtered and concentrated in vacuo. The residue was purified by prep-TLC (SiO₂, petroleum ether:ethyl acetate, 2:1) and then further purified by prep-HPLC (column: Phenomenex Gemini C18, 150 mm×25 mm×10 μm; mobile phase: [A: water (10 mM NH₄HCO₃); B: MeCN]; B %: 25%-55%, 10 min) to give the title compound (28.28 mg, 17% yield, 95.2% purity on LCMS) as a yellow oil.

¹H NMR (400 MHz, DMSO-d₆) δ7.45 (d, 1H), 7.01 (s, 1H), 6.54 (d, 1H), 6.19 (br s, 1H), 4.60-4.50 (m, 1H), 3.71 (s, 2H), 2.90-2.80 (m, 8H), 2.09-2.02 (m, 4H) and 1.53 (d, 6H).

LCMS: m/z 387.1 (M+H)⁺(ES⁺).

### Example 25

### N-(1-Isopropylazetidine-3-sulfonimidoyl)-2-(5-(2-methoxypyridin-4-yl)-2,3-dihydro-1H-inden-4-yl)acetamide

### Step A: tert-Butyl 3-(N-(2-(5-(2-methoxypyridin-4-yl)-2,3-dihydro-1H-inden-4-yl)acetyl)sulfinamoyl)azetidine-1-carboxylate

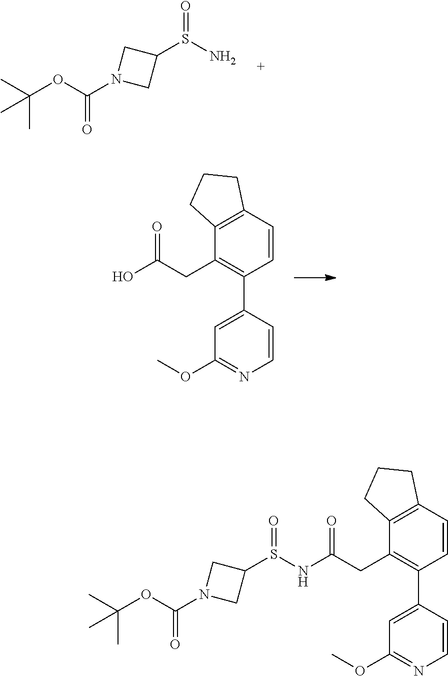 [SMILES: CC(C)(C)OC(=O)N1CC(S(N)=O)C1 ; COc1cc(-c2ccc3c(c2CC(=O)O)CCC3)ccn1 ; COc1cc(-c2ccc3c(c2CC(=O)NS(=O)C2CN(C(=O)OC(C)(C)C)C2)CCC3)ccn1]

To a solution (A) of 2-(5-(2-methoxypyridin-4-yl)-2,3-dihydro-1H-inden-4-yl)acetic acid (Intermediate R10) (600 mg, 2.12 mmol, 1 eq) in THF (10 mL) was added CDI (343 mg, 2.12 mmol, 1 eq), and the reaction mixture was stirred at 70° C. for 1 hour. To another solution (B) of tert-butyl 3-sulfinamoylazetidine-1-carboxylate (Intermediate L7) (560 mg, 2.54 mmol, 1.2 eq) in THF (10 mL) was added n-BuLi (2.5 M, 1.02 mL, 1.2 eq) at −70° C., and the reaction mixture was stirred at −70° C. for 0.5 hour. The solution (A) was added into the solution (B) at −70° C. The resulting mixture was warmed to 20° C. and stirred another 1 hour. The reaction mixture was quenched with water (80 mL) and extracted with ethyl acetate (100 mL). The organic layer was washed with brine (60 mL), dried over Na₂SO₄, filtered and concentrated in vacuo. The residue was purified by reversed phase flash chromatography (0.1% NH₃.H₂O-MeCN) to give the title compound (400 mg, 39%) as yellow oil.

¹H NMR (400 MHz, DMSO-d₆) δ 10.82 (br s, 1H), 8.18 (d, 1H), 7.23 (d, 1H), 7.03 (d, 1H), 6.87-6.85 (m, 1H), 6.66 (s, 1H), 4.13-4.11 (m, 3H), 3.88 (s, 3H), 3.87-3.84 (m, 1H), 3.75-3.70 (m, 1H), 3.54-3.47 (m, 2H), 2.94 (t, 2H), 2.79 (t, 2H), 2.07-2.02 (m, 2H) and 1.39 (s, 9H).

LCMS: m/z 486.2 (M+H)⁺ (ES⁺).

### Step B: tert-Butyl 3-(N-(2-(5-(2-methoxypyridin-4-yl)-2,3-dihydro-1H-inden-4-yl)acetyl)sulfamimidoyl)azetidine-1-carboxylate

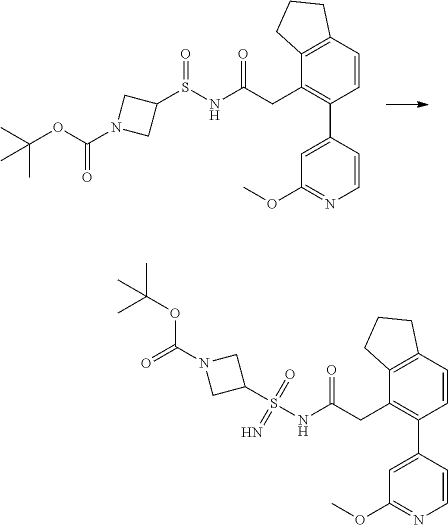 [SMILES: COc1cc(-c2ccc3c(c2CC(=O)NS(=O)C2CN(C(=O)OC(C)(C)C)C2)CCC3)ccn1 ; COc1cc(-c2ccc3c(c2CC(=O)NS(=N)(=O)C2CN(C(=O)OC(C)(C)C)C2)CCC3)ccn1]

To a solution of tert-butyl 3-(N-(2-(5-(2-methoxypyridin-4-yl)-2,3-dihydro-1H-inden-4-yl)acetyl)sulfinamoyl)azetidine-1-carboxylate (380 mg, 782.55 μmol, 1 eq) in THF (10 mL) was added 1-chloro-1H-benzo[d][1,2,3]triazole (132 mg, 860.80 μmol, 1.1 eq). The reaction mixture was stirred at 20° C. for 0.5 hours. The resulting mixture was added dropwise at −70° C. into a solution of NH₃ in THF, which had been prepared by bubbling NH₃ (15 psi) into THF (30 mL) at −70° C. for 10 minutes. The reaction mixture was stirred at 20° C. for 2 hours, and then concentrated in vacuo. The residue was purified by column chromatography (SiO₂, petroleum ether:ethyl acetate, 5:1 to 1:1) to give the title compound (102 mg, 21% yield, 80.1% purity on LCMS) as a yellow oil.

¹H NMR (400 MHz, CDCl₃) δ 8.10-8.08 (m, 1H), 7.17-7.08 (m, 1H), 6.96-6.93 (m, 1H), 6.79-6.72 (m, 1H), 6.63-6.55 (m, 1H), 4.12-4.01 (m, 5H), 3.89 (s, 3H), 3.52-3.48 (m, 2H), 2.92-2.79 (m, 4H), 2.07-2.01 (m, 2H) and 1.36 (s, 9H).

LCMS: m/z 501.1 (M+H)⁺ (ES⁺).

### Step C: N-(Azetidine-3-sulfonimidoyl)-2-(5-(2-methoxypyridin-4-yl)-2,3-dihydro-1H-inden-4-yl)acetamide

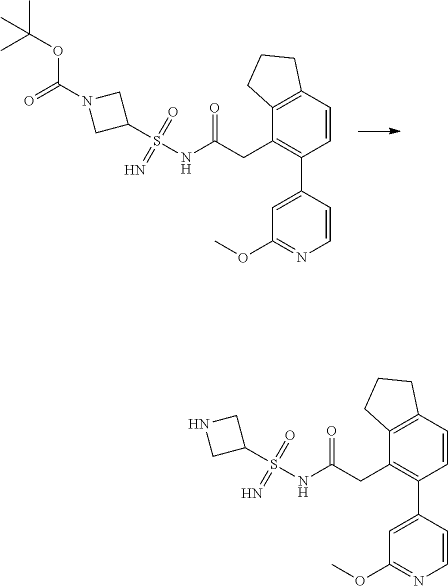 [SMILES: COc1cc(-c2ccc3c(c2CC(=O)NS(=N)(=O)C2CN(C(=O)OC(C)(C)C)C2)CCC3)ccn1 ; COc1cc(-c2ccc3c(c2CC(=O)NS(=N)(=O)C2CNC2)CCC3)ccn1]

To a solution of tert-butyl 3-(N-(2-(5-(2-methoxypyridin-4-yl)-2,3-dihydro-1H-inden-4-yl)acetyl)sulfamimidoyl)azetidine-1-carboxylate (90 mg, 179.78 μmol, 1 eq) in DCM (8 mL) was added TFA (0.8 mL). The reaction mixture was stirred at 25° C. for 1 hour. N₂ was bubbled through the reaction mixture to remove the solvent, and the residue was purified by reversed phase flash chromatography (0.1% NH₃.H₂O-MeCN) to give the title compound (30 mg, 42%) as a yellow oil.

¹H NMR (400 MHz, DMSO-d₆) δ 8.19-8.16 (m, 1H), 7.19 (dd, 1H), 7.04-7.00 (m, 1H), 6.90 (dd, 1H), 6.72 (d, 1H), 4.56-4.48 (m, 1H), 3.91 (s, 3H), 3.89-3.51 (m, 4H), 3.49 (d, 2H), 2.93-2.90 (m, 2H), 2.81-2.78 (m, 2H) and 2.08-2.00 (m, 2H).

LCMS: m/z 401.2 (M+H)⁺ (ES⁺).

### Step D: N-(1-Isopropylazetidine-3-sulfonimidoyl)-2-(5-(2-methoxypyridin-4-yl)-2,3-dihydro-1H-inden-4-yl)acetamide

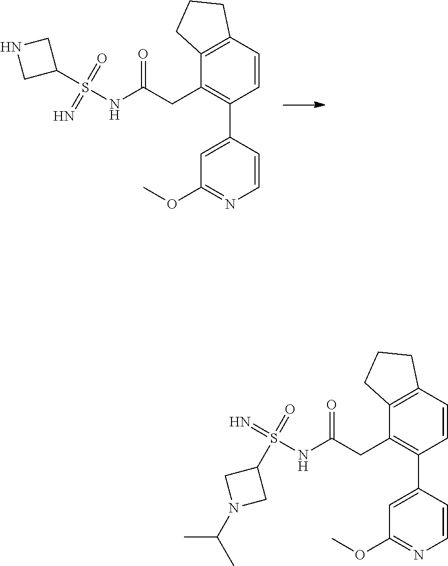 [SMILES: COc1cc(-c2ccc3c(c2CC(=O)NS(=N)(=O)C2CNC2)CCC3)ccn1 ; COc1cc(-c2ccc3c(c2CC(=O)NS(=N)(=O)C2CN(C(C)C)C2)CCC3)ccn1]

To a solution of N-(azetidine-3-sulfonimidoyl)-2-(5-(2-methoxypyridin-4-yl)-2,3-dihydro-1H-inden-4-yl)acetamide (26 mg, 64.92 μmol, 1 eq) in DMSO (0.5 mL) was added DIPEA (17 mg, 129.84 μmol, 2 eq) and 2-iodopropane (22 mg, 129.84 μmol, 2 eq). The reaction mixture was stirred at 30° C. for 21 hours. The reaction mixture was purified directly by reversed phase flash chromatography (0.1% TFA in water-MeCN). The product was further purified by prep-HPLC (column: Phenomenex Gemini C18, 150 mm×25 mm×10 μm; mobile phase: [A: water (0.04% NH₃.H₂O+10 mM NH₄HCO₃), B: MeCN]; B %: 20%-64%, 43 min) to give the title compound (3 mg, 10%) as a white solid.

¹H NMR (400 MHz, CD₃Cl) δ 8.17 (d, 1H), 7.20 (d, 1H), 7.04 (d, 1H), 6.88 (dd, 1H), 6.73 (s, 1H), 4.20-4.15 (m, 1H), 3.98 (s, 3H), 3.62 (s, 2H), 3.59-3.53 (m, 2H), 3.46-3.42 (m, 2H), 2.99 (t, 2H), 2.89 (t, 2H), 2.36-2.34 (m, 1H), 2.15-2.11 (m, 2H) and 0.92 (d, 6H).

LCMS: m/z 443.2 (M+H)⁺ (ES⁺).

### Example 26

### 2-(4-Fluoro-2,6-diisopropylphenyl)-N-((1-isopropyl-1H-pyrazol-3-yl)sulfonyl)acetamide

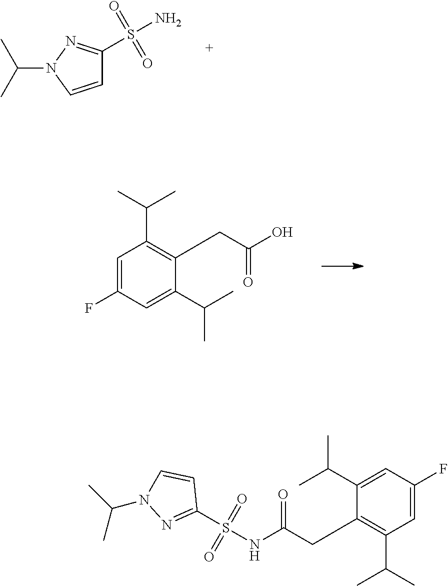 [SMILES: CC(C)n1ccc(S(N)(=O)=O)n1 ; CC(C)c1cc(F)cc(C(C)C)c1CC(=O)O ; CC(C)c1cc(F)cc(C(C)C)c1CC(=O)NS(=O)(=O)c1ccn(C(C)C)n1]

To a solution of 1-isopropyl-1H-pyrazole-3-sulfonamide (Intermediate L8) (50 mg, 264.22 μmol, 1 eq) in DMF (1.5 mL) and DCM (1.5 mL) was added EDCI (101 mg, 528.44 μmol, 2 eq), DMAP (64 mg, 528.44 μmol, 2 eq) and 2-(4-fluoro-2,6-diisopropylphenyl)acetic acid (Intermediate R11) (62 mg, 264.22 μmol, 1 eq). The reaction mixture was stirred at 25° C. for 2 hours, diluted with water (5 mL) and extracted with DCM (3×5 mL). The combined organic layers were washed with 1 N aqueous HCl solution until their pH was 7. Then the organic layers were dried over Na₂SO₄, filtered and concentrated under reduced pressure. The residue was purified by prep-HPLC (column: Phenomenex Luna C18, 150 mm×25 mm×10 μm, mobile phase: [A: water (0.1% TFA); B: MeCN], B %: 50%-78%, 10 min) to give the title compound (24.15 mg, 22% yield, 99% purity on LCMS) as a white solid.

¹H NMR (400 MHz, DMSO-d₆) δ 12.33 (s, 1H), 7.96 (d, 1H), 6.87 (d, 2H), 6.71 (d, 1H), 4.63-4.55 (m, 1H), 3.75 (s, 2H), 2.92-2.85 (m, 2H), 1.41 (d, 6H) and 1.03 (d, 12H).

LCMS: m/z 410.3 (M+H)⁺ (ES⁺).

### Example 27

### 2-(4-Fluoro-2,6-diisopropylphenyl)-N-((2-(2-hydroxypropan-2-yl)thiazol-5-yl)sulfonyl)acetamide

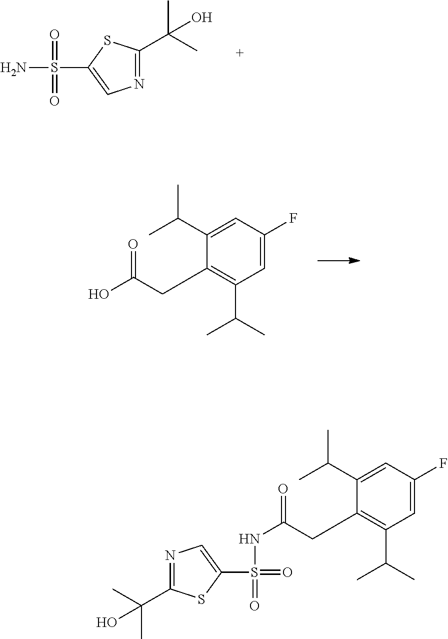 [SMILES: CC(C)(O)c1ncc(S(N)(=O)=O)s1 ; CC(C)c1cc(F)cc(C(C)C)c1CC(=O)O ; CC(C)c1cc(F)cc(C(C)C)c1CC(=O)NS(=O)(=O)c1cnc(C(C)(C)O)s1]

To a solution of 2-(2-hydroxypropan-2-yl)thiazole-5-sulfonamide (Intermediate L9) (80 mg, 359.90 μmol, 1 eq), 2-(4-fluoro-2,6-diisopropylphenyl)acetic acid (Intermediate R11) (94 mg, 395.89 μmol, 1.1 eq) and DMAP (65 mg, 539.85 μmol, 1.5 eq) in DMF (2 mL) was added EDCI (103 mg, 539.85 μmol, 1.5 eq) at 25° C. The reaction mixture was stirred at 25° C. for 1 hour, and then concentrated in vacuo. The residue was purified by prep-HPLC (column: Phenomenex Luna C18, 150 mm×25 mm×10 μm; mobile phase: [A: water (0.1% TFA), B: MeCN]; B %: 42%-72%, 10 min) to give the title compound (65.13 mg, 41% yield, 100% purity on LCMS) as a white solid.

¹H NMR (400 MHz, DMSO-d₆) δ 12.73 (br s, 1H), 8.25 (s, 1H), 6.89 (dd, 2H), 3.77 (s, 2H), 2.89-2.85 (m, 2H), 1.46 (s, 6H) and 1.00 (d, 12H).

LCMS: m/z 443.1 (M+H)⁺ (ES⁺).

### Example 28

### N-((5-Bromo-2-((1-methyl-1H-tetrazol-5-yl)thio)phenyl) carbamoyl)-1-isopropyl-1H-pyrazole-3-sulfonamide

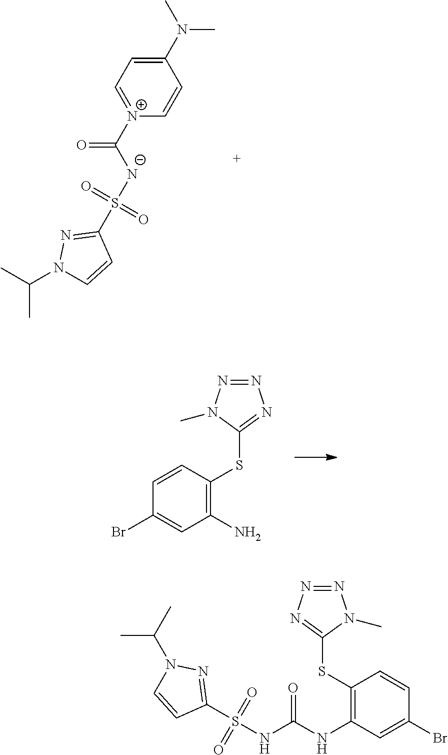 [SMILES: CC(C)n1ccc(S(=O)(=O)[N-]C(=O)[n+]2ccc(N(C)C)cc2)n1 ; Cn1nnnc1Sc1ccc(Br)cc1N ; CC(C)n1ccc(S(=O)(=O)NC(=O)Nc2cc(Br)ccc2Sc2nnnn2C)n1]

A solution of (4-(dimethylamino)pyridin-1-ium-1-carbonyl)((1-isopropyl-1H-pyrazol-3-yl)sulfonyl)amide (Intermediate L21) (30 mg, 88.92 μmol, 1 eq) and 5-bromo-2-((1-methyl-1H-tetrazol-5-yl)thio)aniline (28 mg, 97.81 μmol, 1.1 eq) in MeCN (1 mL) was stirred at 70° C. for 1 hour, and then concentrated in vacuo. The residue was purified by prep-HPLC (column: Phenomenex Gemini C18, 150 mm×25 mm×10 μm; mobile phase: [A: water (0.05% ammonium hydroxide v/v), B: MeCN]; B %: 10%-40%, 12 min) to give the title compound (13.34 mg, 29% yield, 96.1% purity on LCMS) as a white solid.

LCMS: m/z 502.9 (M+H)⁺ (ES⁺).

### Example 29

### 2-(4-Fluoro-2,6-diisopropylphenyl)-N-((5-(2-hydroxypropan-2-yl)-1-isopropyl-1H-pyrazol-3-yl)sulfonyl)acetamide

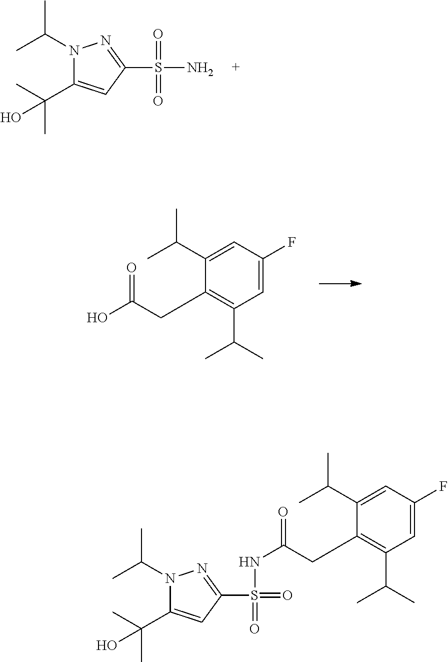 [SMILES: CC(C)n1nc(S(N)(=O)=O)cc1C(C)(C)O ; CC(C)c1cc(F)cc(C(C)C)c1CC(=O)O ; CC(C)c1cc(F)cc(C(C)C)c1CC(=O)NS(=O)(=O)c1cc(C(C)(C)O)n(C(C)C)n1]

To a solution of 5-(2-hydroxypropan-2-yl)-1-isopropyl-1H-pyrazole-3-sulfonamide (Intermediate L24) (100 mg, 404.34 μmol, 1 eq), 2-(4-fluoro-2,6-diisopropylphenyl)acetic acid (Intermediate R11) (106 mg, 444.78 μmol, 1.1 eq) and DMAP (74 mg, 606.52 μmol, 1.5 eq) in DMF (2 mL) was added EDCI (116 mg, 606.52 μmol, 1.5 eq). The reaction mixture was stirred at 25° C. for 1 hour, and then concentrated in vacuo. The residue was purified by prep-HPLC (column: Phenomenex Synergi C18, 150 mm×25 mm×10 μm; mobile phase: [A: water (0.1% TFA), B: MeCN]; B %: 49%-79%, 10 min) to give the title compound (38.27 mg, 20% yield, 100% purity on LCMS) as a yellow solid.

¹H NMR (400 MHz, DMSO-d₆) δ 12.30 (br s, 1H), 6.87 (d, 2H), 6.46 (s, 1H), 5.54 (s, 1H), 5.31-5.27 (m, 1H), 3.76 (s, 2H), 2.91-2.87 (m, 2H), 1.47 (s, 6H), 1.38 (d, 6H) and 1.04 (d, 12H).

LCMS: m/z 468.2 (M+H)⁺ (ES⁺).

### Example 30

### 2-(4-Fluoro-2,6-diisopropylphenyl)-N-((5-(2-hydroxypropan-2-yl)thiazol-2-yl)sulfonyl)acetamide

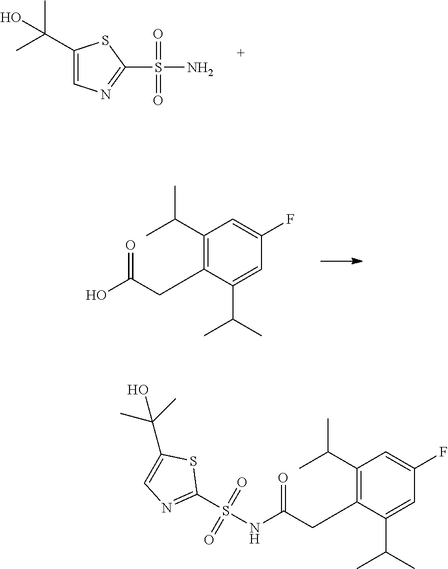 [SMILES: CC(C)(O)c1cnc(S(N)(=O)=O)s1 ; CC(C)c1cc(F)cc(C(C)C)c1CC(=O)O ; CC(C)c1cc(F)cc(C(C)C)c1CC(=O)NS(=O)(=O)c1ncc(C(C)(C)O)s1]

To a solution of 5-(2-hydroxypropan-2-yl)thiazole-2-sulfonamide (Intermediate L10) (80 mg, 359.90 μmol, 1 eq), 2-(4-fluoro-2,6-diisopropylphenyl)acetic acid (Intermediate R11) (94 mg, 395.89 μmol, 1.1 eq) and DMAP (66 mg, 539.85 μmol, 1.5 eq) in DMF (1.5 mL) was added EDCI (104 mg, 539.85 μmol, 1.5 eq) at 25° C. The reaction mixture was stirred at 25° C. for 1 hour. The reaction mixture was purified directly by prep-HPLC (column: Phenomenex Luna C18, 150 mm×25 mm×5 μm; mobile phase: [A: water (0.075% TFA v/v), B: MeCN]; B %: 50%-80%, 9 min) to give the title compound (95 mg, 59% yield, 99.4% purity on LCMS) as a white solid.

¹H NMR (400 MHz, DMSO-d₆) δ 12.97 (br s, 1H), 7.89 (s, 1H), 6.86 (d, 2H), 6.00 (s, 1H), 3.78 (s, 2H), 2.92-2.85 (m, 2H), 1.50 (s, 6H) and 1.02 (d, 12H).

LCMS: m/z 443.1 (M+H)⁺ (ES⁺).

### Example 31

### 1-isopropyl-N-((1-oxo-1,2,3,5,6,7-hexahydro-s-indacen-4-yl)carbamoyl)-1H-pyrazole-3-sulfonamide, sodium salt

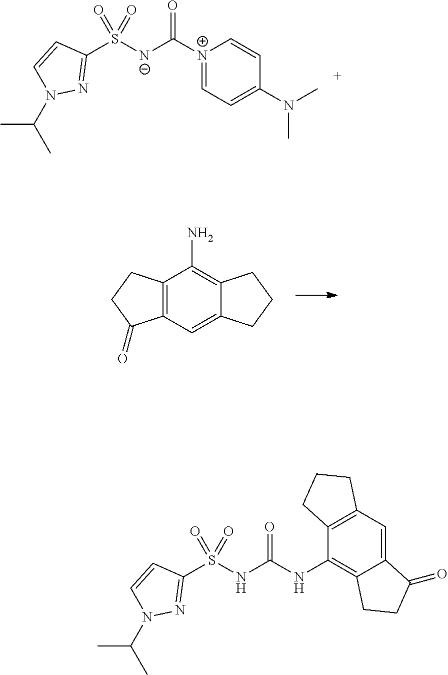

Prepared according to the general procedure of N-((8-fluoro-1,2,3,5,6,7-hexahydro-s-indacen-4-yl)carbamoyl)-1-isopropyl-1H-pyrazole-3-sulfonamide, sodium salt (Example 61) from (4-(dimethylamino)pyridin-1-ium-1-carbonyl)((1-isopropyl-1H-pyrazol-3-yl)sulfonyl)amide (Intermediate L21) and 4-amino-2,3,6,7-tetrahydro-s-indacen-1(5H)-one to afford the title compound (46 mg, 40%) as a white solid.

¹H NMR (DMSO-d₆) δ 7.90 (s, 1H), 7.71 (d, J=2.3 Hz, 1H), 7.17 (s, 1H), 6.40 (d, J=2.3 Hz, 1H), 4.50 (sept, J=6.7 Hz, 1H), 2.92-2.88 (m, 2H), 2.85 (t, J=7.4 Hz, 2H), 2.78 (t, J=7.4 Hz, 2H), 2.55-2.52 (m, 2H), 1.96 (p, J=7.5 Hz, 2H), 1.40 (d, J=6.7 Hz, 6H). One exchangeable proton not observed.

LCMS m/z 403.2 (M+H)⁺ (ES⁺); 401.1 (M−H)⁻ (ES⁻).

### Example 32

### 2-(4-Fluoro-2,6-diisopropylphenyl)-N-((5-(2-hydroxypropan-2-yl)-1-methyl-1H-pyrazol-3-yl)sulfonyl)acetamide, ammonium salt

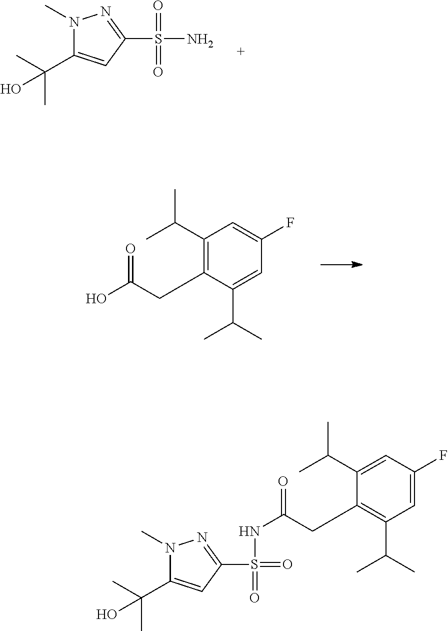 [SMILES: Cn1nc(S(N)(=O)=O)cc1C(C)(C)O ; CC(C)c1cc(F)cc(C(C)C)c1CC(=O)O ; CC(C)c1cc(F)cc(C(C)C)c1CC(=O)NS(=O)(=O)c1cc(C(C)(C)O)n(C)n1]

To a solution of 5-(2-hydroxypropan-2-yl)-1-methyl-1H-pyrazole-3-sulfonamide (Intermediate L12) (80 mg, 364.86 μmol, 1 eq), 2-(4-fluoro-2,6-diisopropylphenyl)acetic acid (Intermediate R11) (96 mg, 401.35 μmol, 1.1 eq) and DMAP (67 mg, 547.29 μmol, 1.5 eq) in DMF (2 mL) was added EDCI (105 mg, 547.29 μmol, 1.5 eq). The reaction mixture was stirred at 25° C. for 1 hour. The reaction mixture was filtered and the filtrate was purified by prep-HPLC (column: Waters Xbridge C18, 150 mm×25 mm×5 μm; mobile phase: [A: water (0.05% ammonium hydroxide v/v), B: MeCN]; B %: 5%-35%, 10 min) to give the title compound (38.5 mg, 23% yield, 100% purity on LCMS) as a white solid.

¹H NMR (400 MHz, DMSO-d₆) δ 12.4 (br s, 1H), 7.07 (br s. 1H), 6.84 (d, 2H), 6.39 (s, 1H), 5.42 (s, 1H), 3.98 (s, 3H), 3.66 (s, 2H), 3.02-2.99 (m, 2H), 1.45 (s, 6H) and 1.05 (d, 12H).

LCMS: m/z 440.2 (M+H)⁺ (ES⁺).

### Example 33

### N-((4-((Dimethylamino)methyl)phenyl)sulfonyl)-2-(4-fluoro-2,6-diisopropylphenyl)acetamide, 2,2,2-trifluoroacetate salt

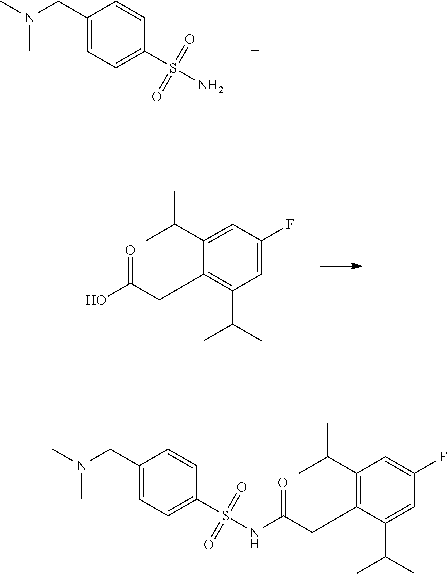 [SMILES: CN(C)Cc1ccc(S(N)(=O)=O)cc1 ; CC(C)c1cc(F)cc(C(C)C)c1CC(=O)O ; CC(C)c1cc(F)cc(C(C)C)c1CC(=O)NS(=O)(=O)c1ccc(CN(C)C)cc1]

To a solution of 2-(4-fluoro-2,6-diisopropylphenyl)acetic acid (Intermediate R11) (222 mg, 933.34 μmol, 1 eq) in DMF (2 mL) was added CDI (166 mg, 1.03 mmol, 1.1 eq) at 20° C. The reaction mixture was stirred for 30 minutes, and then added to a solution of NaH (56 mg, 1.40 mmol, 60 wt % in mineral oil, 1.5 eq) and 4-((dimethylamino)methyl)benzenesulfonamide (Intermediate L13) (200 mg, 933.34 μmol, 1 eq) in DMF (2 mL) at 0° C. The resulting mixture was stirred at 20° C. for 2 hours. Then the reaction mixture was poured into ice-water (30 mL), stirred for 10 minutes and extracted with ethyl acetate (3×15 mL). The combined organic layers were washed with brine (10 mL), dried over anhydrous sodium sulfate, filtered and concentrated in vacuo. The residue was purified by prep-HPLC (column: Phenomenex Synergi C18, 150 mm×25 mm×10 μm; mobile phase: [A: water (0.1% TFA), B: MeCN]; B %: 24%-54%, 10 min) to give the title compound (38.9 mg, 8% yield, 100% purity on LCMS) as a white solid.

¹H NMR (400 MHz, CDCl₃) δ 8.08 (d, 2H), 7.66 (d, 2H), 6.83 (d, 2H), 4.26 (s, 2H), 3.76 (s, 2H), 2.89-2.83 (m, 2H), 2.81 (s, 6H) and 1.09 (d, 12H).

LCMS: m/z 435.2 (M+H)⁺ (ES⁺).

### Example 34

### N-((7-Chloro-5-isopropyl-2,3-dihydro-1H-inden-4-yl) carbamoyl)-1-isopropyl-1H-pyrazole-3-sulfonamide

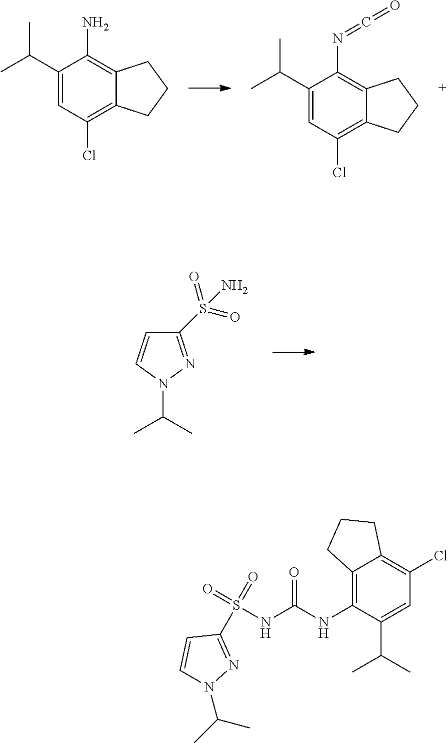 [SMILES: CC(C)c1cc(Cl)c2c(c1N)CCC2 ; CC(C)c1cc(Cl)c2c(c1N=C=O)CCC2 ; CC(C)n1ccc(S(N)(=O)=O)n1 ; CC(C)c1cc(Cl)c2c(c1NC(=O)NS(=O)(=O)c1ccn(C(C)C)n1)CCC2]

Triphosgene (22.6 mg, 0.076 mmol) was added to 7-chloro-5-isopropyl-2,3-dihydro-1H-inden-4-amine (Intermediate R12) (20 mg, 0.095 mmol) and Et₃N (0.016 mL, 0.114 mmol) in THF (2 mL). The mixture was stirred at room temperature for 15 hours. An additional portion of triphosgene (22.6 mg, 0.076 mmol) was added and the mixture was stirred for an additional 2 hours. The mixture was concentrated in vacuo and dried azeotropically with toluene (3×1 mL). THF (2 mL) was added to the residue, followed by 1-isopropyl-1H-pyrazole-3-sulfonamide (Intermediate L8) (18.1 mg, 0.095 mmol). After 30 minutes, NaH (60 wt % in mineral oil, 9.54 mg, 0.238 mmol) was added and the mixture was heated at 60° C. for 15 hours. After cooling to room temperature, sat aq NH₄Cl (10 mL) was added and the mixture was extracted with EtOAc (3×10 mL). The organic layer was washed with brine (5 mL), dried (MgSO₄) and evaporated in vacuo. The residue was purified by chromatography on silica gel (25 g column, 5-100% EtOAc/isohexane) to afford the title compound (7.5 mg, 18%) as a white solid.

¹H NMR (DMSO-d₆) δ 10.98 (br s, 1H), 7.98-7.95 (m, 2H), 7.07-7.04 (m, 1H), 6.70 (s, 1H), 4.62-4.59 (m, 1H), 2.97-2.95 (m, 1H), 2.85 (t, J=7.5 Hz, 2H), 2.66-2.64 (m, 2H), 1.97-1.95 (m, 2H), 1.44 (d, J=6.7 Hz, 6H), 1.30-0.82 (m, 6H).

LCMS m/z 425/427 (M+H)⁺ (ES⁺).

### Example 35

### N-((8-Chloro-1,2,3,5,6,7-hexahydro-s-indacen-4-yl)carbamoyl)-1-isopropyl-1H-pyrazole-3-sulfonamide

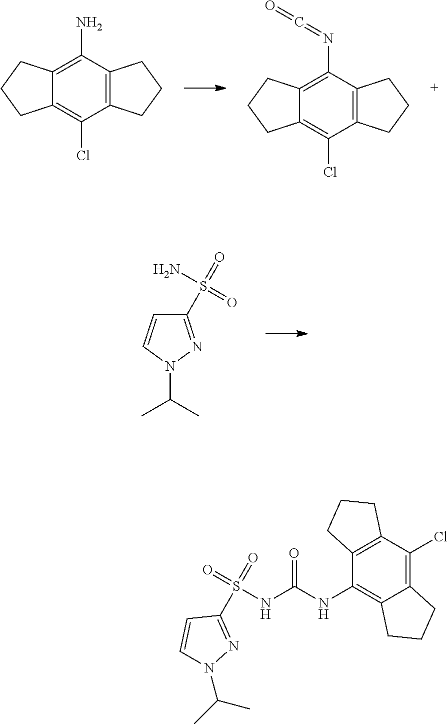 [SMILES: Nc1c2c(c(Cl)c3c1CCC3)CCC2 ; O=C=Nc1c2c(c(Cl)c3c1CCC3)CCC2 ; CC(C)n1ccc(S(N)(=O)=O)n1 ; CC(C)n1ccc(S(=O)(=O)NC(=O)Nc2c3c(c(Cl)c4c2CCC4)CCC3)n1]

Triphosgene (42 mg, 0.142 mmol) was added to a mixture of 8-chloro-1,2,3,5,6,7-hexahydro-s-indacen-4-amine (48 mg, 0.231 mmol) and Et₃N (0.088 mL, 0.634 mmol) in THF (2 mL). The mixture was heated at reflux for 4 hours, concentrated in vacuo and dried azeotropically with toluene (3×1 mL) to afford 4-chloro-8-isocyanato-1,2,3,5,6,7-hexahydro-s-indacene.

1-Isopropyl-1H-pyrazole-3-sulfonamide (Intermediate L8) (40 mg, 0.211 mmol) was dissolved in THF (2 mL) and NaH (60 wt % in mineral oil, 10 mg, 0.250 mmol) was added. The reaction was stirred at room temperature for 30 minutes and the crude 4-chloro-8-isocyanato-1,2,3,5,6,7-hexahydro-s-indacene in THF (2 mL) was added. The reaction was stirred at room temperature for 2 days. Sat aq NH₄Cl (10 mL) was added and the mixture extracted with EtOAc (3×10 mL). The organic phase was washed with brine (5 mL), dried (MgSO₄) and concentrated in vacuo. The product was purified by chromatography on silica gel (25 g column, 5-100% EtOAc/isohexane).The isolated product was triturated with MTBE (10 mL) and filtered to afford the title compound (17 mg, 18%) as a colourless solid.

¹H NMR (DMSO-d₆) δ 10.91 (s, 1H), 8.09 (s, 1H), 7.98 (d, J=2.4 Hz, 1H), 6.73 (d, J=2.4 Hz, 1H), 4.62 (sept, J=6.7 Hz, 1H), 2.84 (t, J=7.4 Hz, 4H), 2.68 (t, J=7.5 Hz, 4H), 1.99 (1), J=7.5 Hz, 4H), 1.44 (d, J=6.7 Hz, 6H).

LCMS m/z 423.0/425.0 (M+H)⁺ (ES⁺).

### Example 36

### N-((8-Bromo-1,2,3,5,6,7-hexahydro-s-indacen-4-yl) carbamoyl)-1-isopropyl-1H-pyrazole-4-sulfonamide

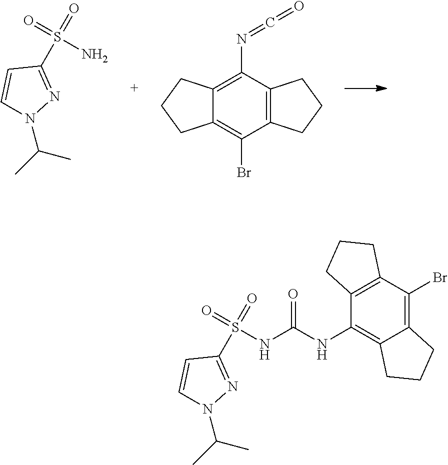 [SMILES: CC(C)n1ccc(S(N)(=O)=O)n1 ; O=C=Nc1c2c(c(Br)c3c1CCC3)CCC2 ; CC(C)n1ccc(S(=O)(=O)NC(=O)Nc2c3c(c(Br)c4c2CCC4)CCC3)n1]

1-Isopropyl-1H-pyrazole-4-sulfonamide (38 mg, 0.201 mmol) was dissolved in THF (2 mL) and NaH (60 wt % in mineral oil, 10 mg, 0.250 mmol) was added. The reaction mixture was stirred at room temperature for 30 minutes. 4-Bromo-8-isocyanato-1,2,3,5,6,7-hexahydro-s-indacene (Intermediate R13) (62 mg, 0.22 mmol) in THF (2 mL) was added and the reaction mixture was stirred at room temperature overnight, before being diluted with water (2 mL) and concentrated in vacuo. The crude product was purified by reversed phase chromatography on RP Flash C18 (12 g column, 5-40% MeCN/10 mM ammonium bicarbonate) to afford the title compound (18 mg, 18%) as a colourless solid.

¹H NMR (DMSO-d₆) δ 10.65 (s, 1H), 8.44 (s, 1H), 8.14 (s, 1H), 7.86 (s, 1H), 4.61 (sept, J=6.7 Hz, 1H), 2.82 (t, J=7.4 Hz, 4H), 2.70 (t, J=7.5 Hz, 4H), 1.98 (p, J=7.5 Hz, 4H), 1.42 (d, J=6.6 Hz, 6H).

LCMS m/z 467/469 (M+H)⁺ (ES⁺).

### Example 37

### N-((8-Cyano-1,2,3,5,6,7-hexahydro-s-indacen-4-yl) carbamoyl)-1-isopropyl-1H-pyrazole-3-sulfonamide

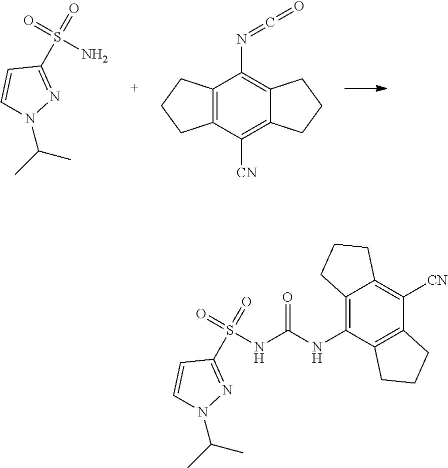 [SMILES: CC(C)n1ccc(S(N)(=O)=O)n1 ; N#Cc1c2c(c(N=C=O)c3c1CCC3)CCC2 ; CC(C)n1ccc(S(=O)(=O)NC(=O)Nc2c3c(c(C#N)c4c2CCC4)CCC3)n1]

1-Isopropyl-1H-pyrazole-3-sulfonamide (Intermediate L8) (28 mg, 0.148 mmol) was dissolved in THF (2 mL) and NaH (60 wt % in mineral oil, 8 mg, 0.200 mmol) was added. The reaction was stirred at room temperature for 30 minutes and 8-isocyanato-1,2,3,5,6,7-hexahydro-s-indacene-4-carbonitrile (Intermediate R14) (37 mg, 0.165 mmol) in THF (2 mL) was added. The reaction mixture was stirred at room temperature for 4 hours, diluted with water (2 mL) and concentrated in vacuo. Water (1.5 mL) was added to the residue and it was washed with MTBE (2×3 mL). The aqueous layer was filtered through a syringe filter and purified by reversed phase chromatography on RP Flash C18 (12 g column, 5-40% MeCN/10 mM ammonium bicarbonate) to afford the title compound (45 mg, 73%) as a colourless solid.

¹H NMR (DMSO-d₆) δ 11.06 (s, 1H), 8.43 (s, 1H), 7.98 (d, J=2.4 Hz, 1H), 6.74 (d, J=2.4 Hz, 1H), 4.61 (sept, J=6.8 Hz, 1H), 2.95 (t, J=7.4 Hz, 4H), 2.66 (t, J=7.4 Hz, 4H), 2.03 (p, J=7.5 Hz, 4H), 1.43 (d, J=6.7 Hz, 6H).

LCMS m/z 414.4 (M+H)⁺ (ES⁺).

### Example 38

### N-((8-Bromo-1,2,3,5,6,7-hexahydro-s-indacen-4-yl) carbamoyl)-1-isopropyl-1H-pyrazole-3-sulfonamide

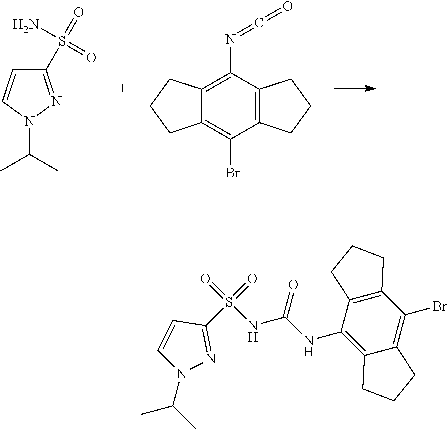 [SMILES: CC(C)n1ccc(S(N)(=O)=O)n1 ; O=C=Nc1c2c(c(Br)c3c1CCC3)CCC2 ; CC(C)n1ccc(S(=O)(=O)NC(=O)Nc2c3c(c(Br)c4c2CCC4)CCC3)n1]

Prepared according to the general procedure of N-((8-bromo-1,2,3,5,6,7-hexahydro-s-indacen-4-yl)carbamoyl)-1-isopropyl-1H-pyrazole-4-sulfonamide (Example 36) from 1-isopropyl-1H-pyrazole-3-sulfonamide (Intermediate L8) and 4-bromo-8-isocyanato-1,2,3,5,6,7-hexahydro-s-indacene (Intermediate R13) to afford the title compound (57 mg, 55%) as a colourless solid.

¹H NMR (DMSO-d₆) δ 10.91 (s, 1H), 8.08 (s, 1H), 7.97 (d, J=2.4 Hz, 1H), 6.72 (d, J=2.4 Hz, 1H), 4.69-4.49 (m, 1H), 2.81 (t, J=7.4 Hz, 4H), 2.71 (t, J=7.5 Hz, 4H), 2.04-1.91 (m, 4H), 1.43 (d, J=6.7 Hz, 6H).

LCMS m/z 467/469 (M+H)⁺ (ES⁺).

### Example 39

### N-((1,2,3,5,6,7-Hexahydro-s-indacen-4-yl)carbamoyl)-5-(2-methoxypropan-2-yl)-1-methyl-1H-pyrazole-3-sulfonamide

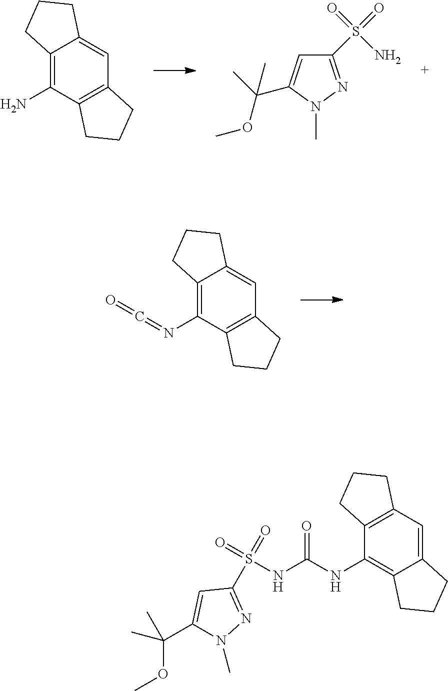 [SMILES: Nc1c2c(cc3c1CCC3)CCC2 ; COC(C)(C)c1cc(S(N)(=O)=O)nn1C ; O=C=Nc1c2c(cc3c1CCC3)CCC2 ; COC(C)(C)c1cc(S(=O)(=O)NC(=O)Nc2c3c(cc4c2CCC4)CCC3)nn1C]

Triphosgene (11 mg, 0.037 mmol) was added to a mixture of 1,2,3,5,6,7-hexahydro-s-indacen-4-amine (11 mg, 0.063 mmol) and Et₃N (24 μL, 0.172 mmol) in THF (2 mL). The reaction mixture was stirred for 15 hours, evaporated in vacuo and azeotroped with toluene (3×1 mL). THF (2 mL) was added and the mixture was filtered. The filtrate was added to a mixture of 5-(2-methoxypropan-2-yl)-1-methyl-1H-pyrazole-3-sulfonamide (Intermediate L14) (50 mg, 0.054 mmol) and NaH (60 wt % in mineral oil, 6 mg, 0.150 mmol) in THF (2 mL). The reaction was stirred for 20 hours at room temperature, before being quenched with water (2 mL). The THF was evaporated in vacuo and the aqueous residue was washed with TBME (2×3 mL), buffered with sodium dihydrogenphosphate (73.1 mg) and purified by reversed phase chromatography on RP Flash C18 (12 g column, 5-50% MeCN/10 mM ammonium bicarbonate) to afford the title compound (5 mg, 21%) as a white solid.

¹H NMR (DMSO-d₆) δ 10.81 (s, 1H), 8.02 (s, 1H), 6.94 (s, 1H), 6.68 (s, 1H), 3.99 (s, 3H), 3.00 (s, 3H), 2.79 (t, J=7.4, Hz, 4H), 2.58 (t, J=7.4 Hz, 4H), 1.94 (p, J=7.5 Hz, 4H), 1.53 (s, 6H).

LCMS m/z 433.5 (M+H)⁺ (ES⁺); 431.3 (M−H)⁻ (ES⁻).

### Example 40

### N-((4-Chloro-2,6-diisopropylphenyl)carbamoyl)-1-(2,2,2-trifluoroethyl)-1H-pyrazole-4-sulfonamide

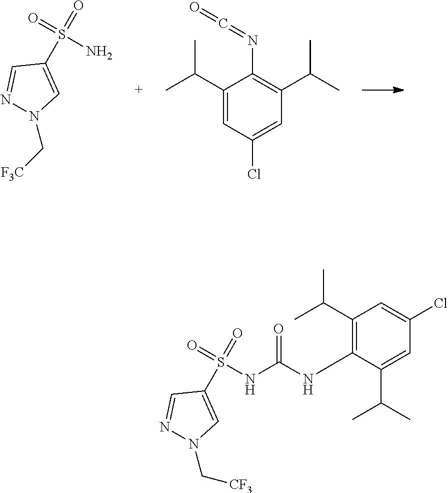 [SMILES: NS(=O)(=O)c1cnn(CC(F)(F)F)c1 ; CC(C)c1cc(Cl)cc(C(C)C)c1N=C=O ; CC(C)c1cc(Cl)cc(C(C)C)c1NC(=O)NS(=O)(=O)c1cnn(CC(F)(F)F)c1]

NaH (60 wt % in mineral oil, 20.14 mg, 0.504 mmol) was added to 1-(2,2,2-trifluoroethyl)-1H-pyrazole-4-sulfonamide (Intermediate L15) (55.4 mg, 0.242 mmol) in THF (2 mL) and the reaction mixture was stirred at room temperature for 30 minutes. 5-Chloro-2-isocyanato-1,3-diisopropylbenzene (Intermediate R15) was added. The reaction mixture was heated at 60° C. for 15 hours and quenched with water (2 mL). The THF was evaporated in vacuo. The aqueous residue was washed with TBME (2×3 mL) and purified by reversed phase chromatography RP Flash C18 (12 g column, 0-50% MeCN/10 mM ammonium bicarbonate) to afford the title compound (15.5 mg, 16%) as a white solid.

¹H NMR (DMSO-d₆) δ 8.14 (br s, 1H), 7.72 (s, 1H), 7.46 (s, 1H), 7.03 (s, 2H), 5.15-5.11 (m, 2H), 3.08-3.05 (m, 2H), 1.02 (d, J=6.9 Hz, 12H). One exchangeable proton not observed.

LCMS m/z 467/469 (M+H)⁺ (ES⁺).

### Example 41

### N-((1,2,3,5,6,7-Hexahydro-s-indacen-4-yl)carbamoyl)-1-(2,2,2-trifluoroethyl)-1H-pyrazole-4-sulfonamide, sodium salt

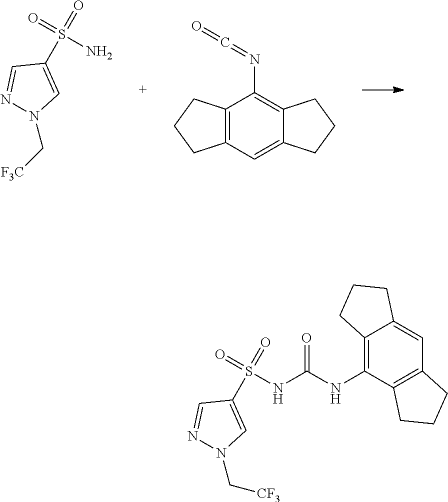 [SMILES: NS(=O)(=O)c1cnn(CC(F)(F)F)c1 ; O=C=Nc1c2c(cc3c1CCC3)CCC2 ; O=C(Nc1c2c(cc3c1CCC3)CCC2)NS(=O)(=O)c1cnn(CC(F)(F)F)c1]

NaH (60 wt % in mineral oil, 7.85 mg, 0.196 mmol) was added to 1-(2,2,2-trifluoroethyl)-1H-pyrazole-4-sulfonamide (Intermediate L15) (30 mg, 0.131 mmol) in THF (5 mL) and the reaction mixture was stirred for 1 hour at room temperature. 4-Isocyanato-1,2,3,5,6,7-hexahydro-s-indacene (Intermediate R1) (26.1 mg, 0.131 mmol) was added and the reaction mixture was stirred at room temperature for 15 hours. The suspension was filtered and washed with THF (1 mL). The collected solid was triturated with EtOAc (10 mL) and dried in vacuo to afford the title compound (44 mg, 73%) as white solid.

¹H NMR (DMSO-d₆) δ 8.05 (s, 1H), 7.67 (s, 1H), 7.33 (s, 1H), 6.76 (s, 1H), 5.14-5.11 (m, 2H), 2.75 (t, J=7.4 Hz, 4H), 2.66 (t, J=7.2 Hz, 4H), 1.92-1.89 (m, 4H).

LCMS m/z 429 (M+H)⁺ (ES⁺).

### Example 42

### N-((1,2,3,5,6,7-Hexahydro-s-indacen-4-yl)carbamoyl)-1-(2,2,2-trifluoroethyl)-1H-pyrazole-3-sulfonamide

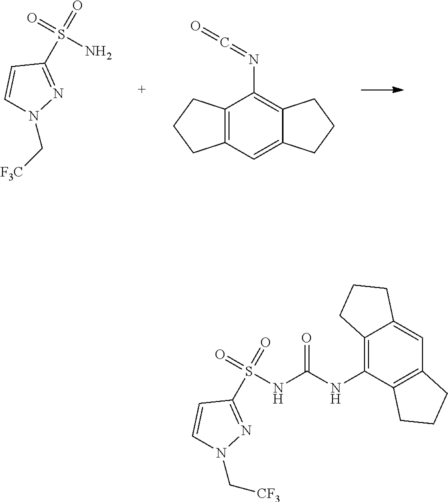 [SMILES: NS(=O)(=O)c1ccn(CC(F)(F)F)n1 ; O=C=Nc1c2c(cc3c1CCC3)CCC2 ; O=C(Nc1c2c(cc3c1CCC3)CCC2)NS(=O)(=O)c1ccn(CC(F)(F)F)n1]

NaH (60 wt % in mineral oil, 7.85 mg, 0.196 mmol) was added to 1-(2,2,2-trifluoroethyl)-1H-pyrazole-3-sulfonamide (Intermediate L16) (30 mg, 0.131 mmol) in THF (5 mL) and the reaction mixture was stirred for 1 hour at room temperature. 4-Isocyanato-1,2,3,5,6,7-hexahydro-s-indacene (Intermediate R1) (26.1 mg, 0.131 mmol) was added and the reaction mixture was stirred at room temperature for 15 hours. The reaction was quenched with water (2 mL) and THF was removed by evaporation in vacuo. The aqueous residue was washed with TBME (2×3 mL) and purified by reversed phase chromatography on RP Flash C18 (12 g column, 0-50% MeCN/10 mM ammonium bicarbonate) to afford the title compound (7.2 mg, 13%) as a white solid.

¹H NMR (DMSO-d₆) δ 7.75 (d, J=2.4 Hz, 1H), 7.50 (s, 1H), 6.76 (s, 1H), 6.50 (d, J=2.4 Hz, 1H), 5.14-5.11 (m, 2H), 2.74 (t, J=7.4 Hz, 4H), 2.64 (t, J=7.4 Hz, 4H), 1.90-1.88 (m, 4H). One exchangeable proton not observed.

LCMS m/z 429 (M+H)⁺ (ES⁺).

### Example 43

### N-((1,2,3,5,6,7-Hexahydro-s-indacen-4-yl)carbamoyl)-1-isopropyl-3-methyl-1H-pyrazole-4-sulfonamide, sodium salt

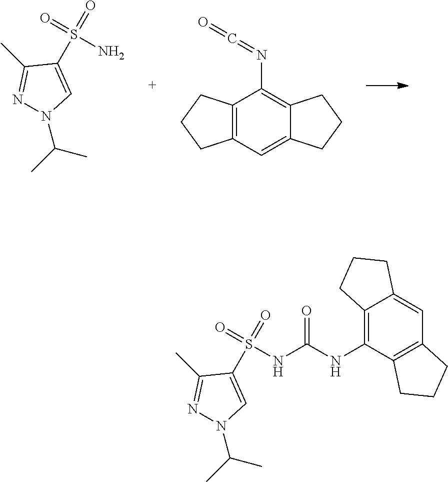 [SMILES: Cc1nn(C(C)C)cc1S(N)(=O)=O ; O=C=Nc1c2c(cc3c1CCC3)CCC2 ; Cc1nn(C(C)C)cc1S(=O)(=O)NC(=O)Nc1c2c(cc3c1CCC3)CCC2]

NaO^{t}Bu (2 M in THF, 0.101 mL, 0.201 mmol) was added to 1-isopropyl-3-methyl-1H-pyrazole-4-sulfonamide (Intermediate L17) (39 mg, 0.192 mmol) in THF (3 mL). The reaction was stirred for 1 hour at room temperature. 4-Isocyanato-1,2,3,5,6,7-hexahydro-s-indacene (Intermediate R1) (38.2 mg, 0.192 mmol) was added. The reaction mixture was stirred at room temperature for 15 hours and filtered, washing with THF (1 mL). The collected solid was triturated with EtOAc (5 mL) and dried in vacuo to afford the title compound (48 mg, 58%) as a white solid.

¹H NMR (DMSO-d₆) δ 7.75 (s, 1H), 7.46 (s, 1H), 6.76 (s, 1H), 4.38-4.31 (m, 1H), 2.74 (t, J=7.3 Hz, 4H), 2.63 (t, J=7.4 Hz, 4H), 2.24 (s, 3H), 1.93-1.85 (m, 4H), 1.35 (d, J=6.6 Hz, 6H).

LCMS m/z 403 (M+H)⁺ (ES⁺).

### Example 44

### 1-(Cyclopropylmethyl)-N-((1,2,3,5,6,7-hexahydro-s-indacen-4-yl)carbamoyl)-1H-pyrazole-4-sulfonamide, sodium salt

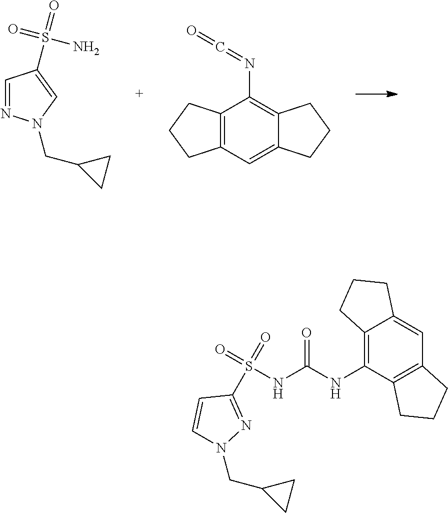 [SMILES: NS(=O)(=O)c1cnn(CC2CC2)c1 ; O=C=Nc1c2c(cc3c1CCC3)CCC2 ; O=C(Nc1c2c(cc3c1CCC3)CCC2)NS(=O)(=O)c1ccn(CC2CC2)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 1-(cyclopropylmethyl)-1H-pyrazole-4-sulfonamide (Intermediate L18) and 4-isocyanato-1,2,3,5,6,7-hexahydro-s-indacene (Intermediate R1) to afford the title compound (60 mg, 75%) as a white solid.

¹H NMR (DMSO-d₆) δ 7.94 (d, J=0.7 Hz, 1H), 7.53 (d, J=0.7 Hz, 1H), 7.34 (s, ¹H), 6.76 (s, 1H), 3.92 (d, J=7.1 Hz, 2H), 2.75 (t, J=7.4 Hz, 4H), 2.67 (t, J=7.3 Hz, 4H), 1.90 (p, J=7.4 Hz, 4H), 1.27-1.13 (m, 1H), 0.57-0.45 (m, 2H), 0.38-0.32 (m, 2H).

LCMS m/z 401 (M+H)⁺ (ES⁺).

### Example 45

### 1-Ethyl-N-((1,2,3,5,6,7-hexahydro-s-indacen-4-yl)carbamoyl)-1H-pyrazole-3-sulfonamide, sodium salt

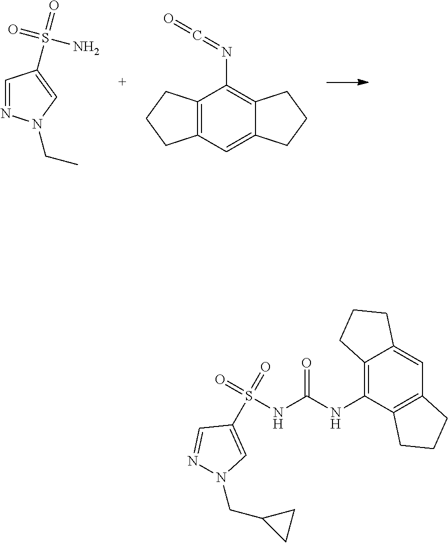 [SMILES: CCn1cc(S(N)(=O)=O)cn1 ; O=C=Nc1c2c(cc3c1CCC3)CCC2 ; O=C(Nc1c2c(cc3c1CCC3)CCC2)NS(=O)(=O)c1cnn(CC2CC2)c1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 1-ethyl-1H-pyrazole-3-sulfonamide (Intermediate L19) and 4-isocyanato-1,2,3,5,6,7-hexahydro-s-indacene (Intermediate R1) to afford the title compound (40.1 mg, 28%) as a white solid.

¹H NMR (DMSO-d₆) δ 7.66 (d, J=2.2 Hz, 1H), 7.52 (s, 1H), 6.77 (s, 1H), 6.36 (d, J=2.2 Hz, 1H), 4.11 (q, J=7.3 Hz, 2H), 2.75 (t, J=7.3 Hz, 4H), 2.65 (t, J=7.4 Hz, 4H), 1.90 (p, J=7.4 Hz, 4H), 1.36 (t, J=7.3 Hz, 3H).

LCMS m/z 375 (M+H)⁺ (ES⁺).

### Example 46

### N-((1,2,3,5,6,7-Hexahydro-s-indacen-4-yl)carbamoyl)-1-isopropyl-1H-1,2,4-triazole-3-sulfonamide, sodium salt

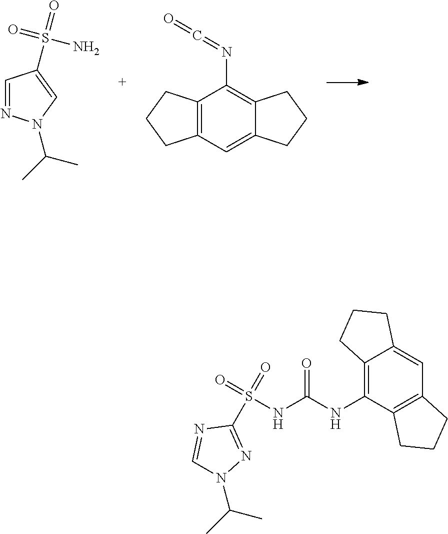 [SMILES: CC(C)n1cc(S(N)(=O)=O)cn1 ; O=C=Nc1c2c(cc3c1CCC3)CCC2 ; CC(C)n1cnc(S(=O)(=O)NC(=O)Nc2c3c(cc4c2CCC4)CCC3)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 1-isopropyl-1H-1,2,4-triazole-3-sulfonamide (Intermediate L20) and 4-isocyanato-1,2,3,5,6,7-hexahydro-s-indacene (Intermediate R1) to afford the title compound (83 mg, 76%) as a colourless solid.

¹H NMR (DMSO-d₆) δ 8.49 (s, 1H), 7.62 (s, 1H), 6.77 (s, 1H), 4.59 (sept, J=6.7 Hz, 1H), 2.75 (t, J=7.4 Hz, 4H), 2.64 (t, J=7.3 Hz, 4H), 1.97-1.81 (m, 4H), 1.44 (d, J=6.7 Hz, 6H).

LCMS m/z 390.4 (M+H)⁺ (ES⁺).

### Example 47

### N-((1,2,3,5,6,7-Hexahydro-s-indacen-4-yl)carbamoyl)-1-isopropyl-1H-1,2,3-triazole-5-sulfonamide

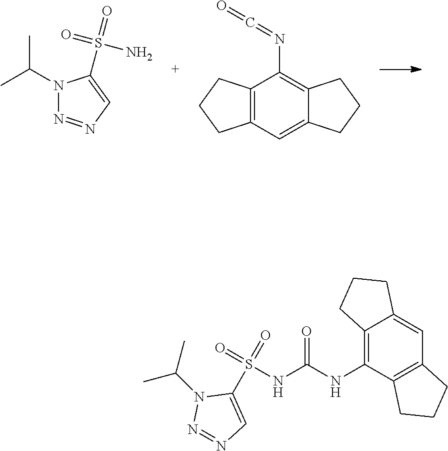 [SMILES: CC(C)n1nncc1S(N)(=O)=O ; O=C=Nc1c2c(cc3c1CCC3)CCC2 ; CC(C)n1nncc1S(=O)(=O)NC(=O)Nc1c2c(cc3c1CCC3)CCC2]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 1-isopropyl-1H-1,2,3-triazole-5-sulfonamide (Intermediate L22) and 4-isocyanato-1,2,3,5,6,7-hexahydro-s-indacene (Intermediate R1), and purified by reversed phase prep-HPLC method 1 to afford the title compound (15 mg, 14%) as a colourless solid.

¹H NMR (400 MHz, DMSO-d₆) δ 7.89 (s, 1H), 7.82 (s, 1H), 6.84 (s, 1H), 5.38 (sept, J=6.7 Hz, 1H), 2.76 (t, J=7.4 Hz, 4H), 2.61 (t, J=7.4 Hz, 4H), 1.91 (p, J=7.4 Hz, 4H), 1.51 (d, J=6.7 Hz, 6H). One exchangeable proton not observed.

LCMS m/z 390 (M+H)⁺ (ES⁺); 388 (M−H)⁻ (ES⁻).

### Example 48

### N-((1,2,3,5,6,7-Hexahydro-s-indacen-4-yl)carbamoyl)-2-isopropyl-2H-1,2,3-triazole-4-sulfonamide

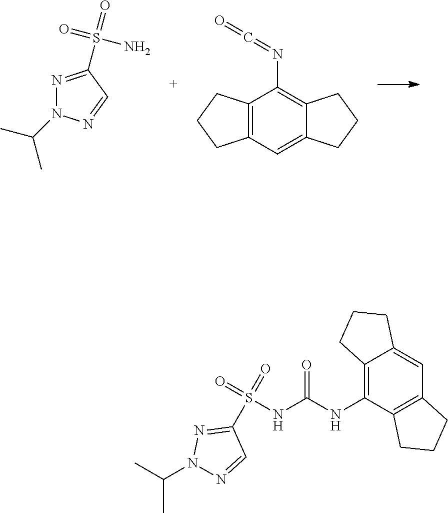 [SMILES: CC(C)n1ncc(S(N)(=O)=O)n1 ; O=C=Nc1c2c(cc3c1CCC3)CCC2 ; CC(C)n1ncc(S(=O)(=O)NC(=O)Nc2c3c(cc4c2CCC4)CCC3)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 2-isopropyl-2H-1,2,3-triazole-4-sulfonamide (Intermediate L23) and 4-isocyanato-1,2,3,5,6,7-hexahydro-s-indacene (Intermediate R1), and purified by reversed phase prep-HPLC method 1 to afford the title compound (15.5 mg, 15%) as a colourless solid.

¹H NMR (DMSO-d₆) δ 11.25 (s, 1H), 8.20 (s, 1H), 8.06 (s, 1H), 6.92 (s, 1H), 4.91 (sept, J=6.7 Hz, 1H), 2.78 (t, J=7.3 Hz, 4H), 2.57 (t, J=7.4 Hz, 4H), 1.93 (p, J=7.4 Hz, 4H), 1.51 (d, J=6.7 Hz, 6H).

LCMS m/z 390 (M+H)⁺ (ES⁺); 388 (M−H)⁻ (ES⁻).

### Example 49

### N-((1,2,3,5,6,7-Hexahydro-s-indacen-4-yl)carbamoyl)-5-(2-hydroxypropan-2-yl)-1-isopropyl-1H-pyrazole-3-sulfonamide

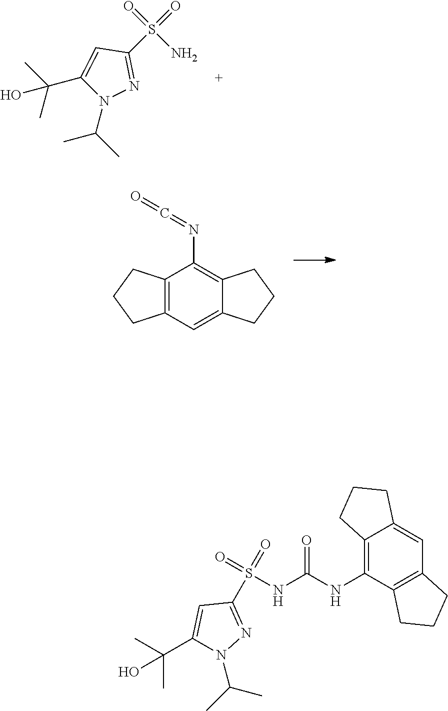

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 5-(2-hydroxypropan-2-yl)-1-isopropyl-1H-pyrazole-3-sulfonamide (Intermediate L24) and 4-isocyanato-1,2,3,5,6,7-hexahydro-s-indacene (Intermediate R1), and purified by reversed phase chromatography on RP Flash C18 (12 g column, 5-50% MeCN/10 mM ammonium bicarbonate) to afford the title compound (27.1 mg, 30%) as a clear colourless solid.

¹H NMR (DMSO-d₆) δ 10.82 (s, 1H), 8.01 (s, 1H), 6.94 (s, 1H), 6.50 (s, 1H), 5.57 (s, 1H), 5.31 (sept, J=6.6 Hz, 1H), 2.78 (t, J=7.4 Hz, 4H), 2.59 (t, J=7.4 Hz, 4H), 1.93 (p, J=7.4 Hz, 4H), 1.51 (s, 6H), 1.39 (d, J=6.6 Hz, 6H).

LCMS m/z 447.5 (M+H)⁺ (ES⁺).

### Example 50

### N-((4-Fluoro-2,6-diisopropylphenyl)carbamoyl)-5-(2-hydroxypropan-2-yl)-1-isopropyl-1H-pyrazole-3-sulfonamide

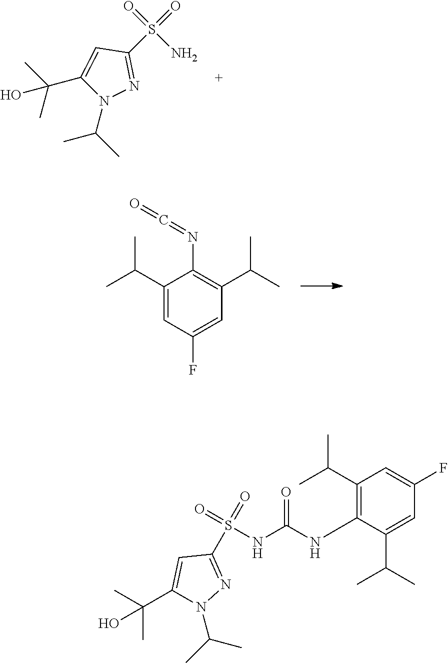 [SMILES: CC(C)n1nc(S(N)(=O)=O)cc1C(C)(C)O ; CC(C)c1cc(F)cc(C(C)C)c1N=C=O ; CC(C)c1cc(F)cc(C(C)C)c1NC(=O)NS(=O)(=O)c1cc(C(C)(C)O)n(C(C)C)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 5-(2-hydroxypropan-2-yl)-1-isopropyl-1H-pyrazole-3-sulfonamide (Intermediate L24) and 5-fluoro-2-isocyanato-1,3-diisopropylbenzene (Intermediate R16) and purified by reversed phase chromatography on RP Flash C18 (12 g column, 5-50% MeCN/10 mM ammonium bicarbonate) to afford the title compound (7.5 mg, 8%) as a clear colourless solid.

¹H NMR (DMSO-d₆) δ 11.03 (s, 1H), 7.79 (s, 1H), 6.91 (d, J=9.9 Hz, 2H), 6.45 (s, 1H), 5.52 (s, 1H), 5.29 (sept, J=6.6 Hz, 1H), 3.03-2.92 (m, 2H), 1.49 (s, 6H), 1.40 (d, J=6.5 Hz, 6H), 1.05 (app br s, 12H).

LCMS m/z 469.5 (M+H)⁺ (ES⁺).

### Example 51

### N-((1,2,3,5,6,7-Hexahydro-s-indacen-4-yl)carbamoyl)-5-(1-methoxycyclobutyl)-1-methyl-1H-pyrazole-3-sulfonamide

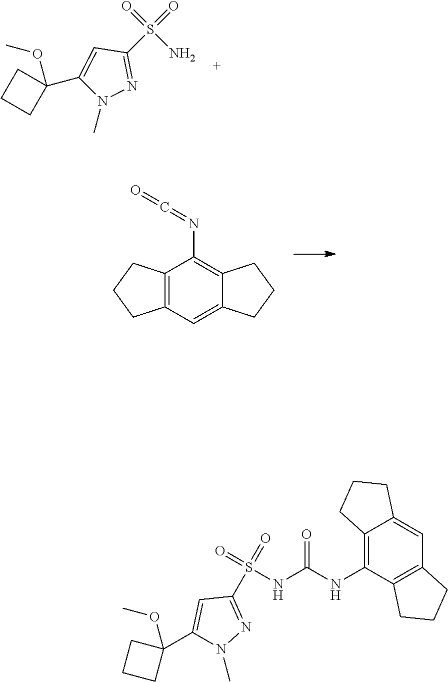 [SMILES: COC1(c2cc(S(N)(=O)=O)nn2C)CCC1 ; O=C=Nc1c2c(cc3c1CCC3)CCC2 ; COC1(c2cc(S(=O)(=O)NC(=O)Nc3c4c(cc5c3CCC5)CCC4)nn2C)CCC1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 5-(1-methoxycyclobutyl)-1-methyl-1H-pyrazole-3-sulfonamide (Intermediate L25) and 4-isocyanato-1,2,3,5,6,7-hexahydro-s-indacene (Intermediate R1), and purified by reversed phase chromatography on RP Flash C18 (12 g column, 5-100% MeCN/10 mM ammonium bicarbonate) to afford the title compound (31 mg, 28%) as a white solid.

¹H NMR (DMSO-d₆) δ 7.97 (s, 1H), 6.90 (s, 1H), 6.85 (s, 1H), 3.80 (s, 3H), 2.87 (s, 3H), 2.77 (t, J=7.4, 4H), 2.58 (t, J=7.4, 4H), 2.45-2.27 (m, 4H), 1.91 (p, J=7.5, 41⁻), 1.87-1.77 (m, 1H), 1.66-1.51 (m, 1H). One exchangeable proton not observed.

LCMS m/z 445.4 (M+H)⁺ (ES⁺).

### Example 52

### N-((1,2,3,5,6,7-hexahydro-s-indacen-4-yl)carbamoyl)-5-(1-methoxycyclopentyl)-1-methyl-1H-pyrazole-3-sulfonamide

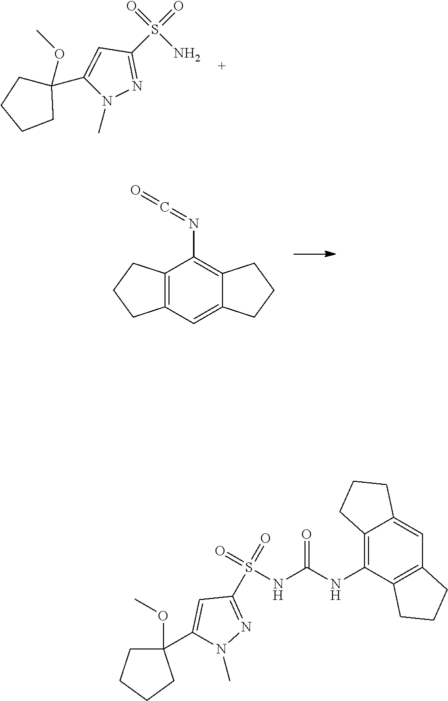 [SMILES: COC1(c2cc(S(N)(=O)=O)nn2C)CCCC1 ; O=C=Nc1c2c(cc3c1CCC3)CCC2 ; COC1(c2cc(S(=O)(=O)NC(=O)Nc3c4c(cc5c3CCC5)CCC4)nn2C)CCCC1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 5-(1-methoxycyclopentyl)-1-methyl-1H-pyrazole-3-sulfonamide (Intermediate L26) and 4-isocyanato-1,2,3,5,6,7-hexahydro-s-indacene (Intermediate R1), and purified by reversed phase chromatography on RP Flash C18 (12 g column, 5-100% MeCN/10 mM ammonium bicarbonate) to afford the title compound (9 mg, 12%) as a cream-coloured solid.

¹H NMR (DMSO-d₆) δ 7.99 (s, 1H), 6.91 (s, 1H), 6.70 (s, 1H), 3.94 (s, 3H), 2.89 (s, 3H), 2.78 (t, J=7.4 Hz, 4H), 2.57 (t, J=7.4 Hz, 4H), 2.23-2.14 (m, 2H), 1.98-1.83 (m, 6H), 1.76-1.62 (m, 4H). One exchangeable proton not observed.

LCMS m/z 459.5 (M+H)⁺ (ES⁺).

### Example 53

### N-((1,2,3,5,6,7-Hexahydro-s-indacen-4-yl)carbamoyl)-5-(1-methoxyethyl)-1-methyl-1H-pyrazole-3-sulfonamide

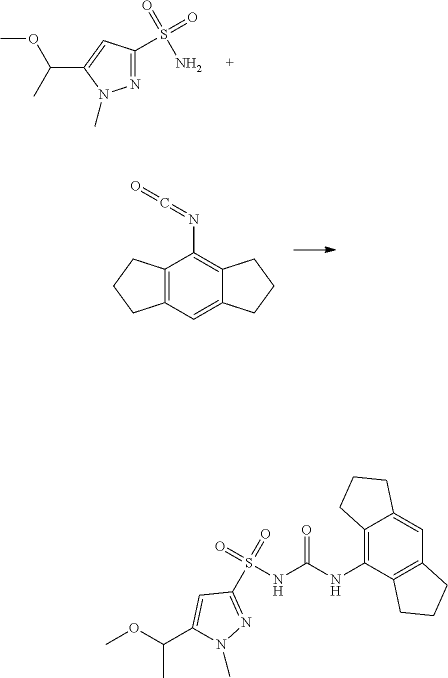 [SMILES: COC(C)c1cc(S(N)(=O)=O)nn1C ; O=C=Nc1c2c(cc3c1CCC3)CCC2 ; COC(C)c1cc(S(=O)(=O)NC(=O)Nc2c3c(cc4c2CCC4)CCC3)nn1C]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 5-(1-methoxyethyl)-1-methyl-1H-pyrazole-3-sulfonamide (Intermediate L27) and 4-isocyanato-1,2,3,5,6,7-hexahydro-s-indacene (Intermediate R1), and purified by reversed phase prep-HPLC method 1 to afford the title compound (33 mg, 24%) as a white solid.

¹H NMR (DMSO-d₆) δ 10.81 (s, 1H), 8.01 (s, 1H), 6.93 (s, 1H), 6.69 (s, 1H), 4.65 (q, J=6.5 Hz, 1H), 3.89 (s, 3H), 3.20 (s, 3H), 2.78 (t, J=7.4 Hz, 4H), 2.59 (t, J=7.4 Hz, 4H), 1.94 (p, J=7.4 Hz, 4H), 1.42 (d, J=6.5 Hz, 3H).

LCMS m/z 419.4 (M+H)⁺ (ES⁺).

### Example 54

### N-((1,2,3,5,6,7-Hexahydro-s-indacen-4-yl)carbamoyl)-5-(1-hydroxyethyl)-1-isopropyl-1H-pyrazole-3-sulfonamide

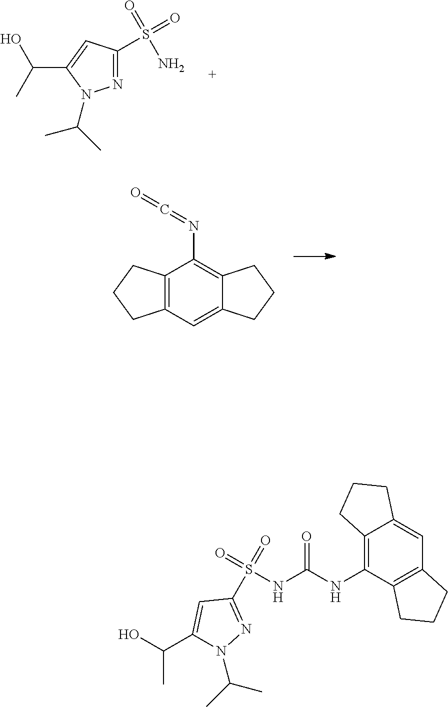 [SMILES: CC(O)c1cc(S(N)(=O)=O)nn1C(C)C ; O=C=Nc1c2c(cc3c1CCC3)CCC2 ; CC(O)c1cc(S(=O)(=O)NC(=O)Nc2c3c(cc4c2CCC4)CCC3)nn1C(C)C]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 5-(1-hydroxyethyl)-1-isopropyl-1H-pyrazole-3-sulfonamide (Intermediate L28) and 4-isocyanato-1,2,3,5,6,7-hexahydro-s-indacene (Intermediate R1), and purified by reversed phase chromatography on RP Flash C18 (12 g column, 5-100% MeCN/10 mM ammonium bicarbonate) to afford the title compound (18 mg, 10%) as a white solid.

¹H NMR (DMSO-d₆) δ 10.84 (br s, 1H), 7.97 (s, 1H), 6.92 (s, 1H), 6.57 (s, 1H), 5.49 (d, J=6.1 Hz, 1H), 4.92-4.79 (m, 2H), 2.79 (t, J=7.4 Hz, 4H), 2.61 (t, J=7.4 Hz, 4H), 1.94 (p, J=7.4 Hz, 4H), 1.49-1.34 (m, 9H).

LMCS m/z 433.4 (M+H)⁺ (ES⁺).

### Example 55

### 4-Fluoro-N-((1,2,3,5,6,7-hexahydro-s-indacen-4-yl) carbamoyl)-1-isopropyl-1H-pyrazole-3-sulfonamide

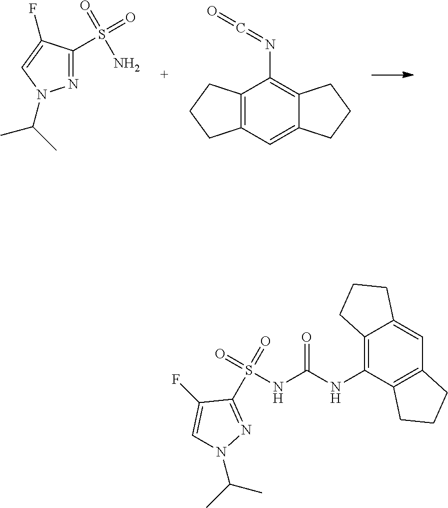 [SMILES: CC(C)n1cc(F)c(S(N)(=O)=O)n1 ; O=C=Nc1c2c(cc3c1CCC3)CCC2 ; CC(C)n1cc(F)c(S(=O)(=O)NC(=O)Nc2c3c(cc4c2CCC4)CCC3)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 4-fluoro-1-isopropyl-1H-pyrazole-3-sulfonamide (Intermediate L29) and 4-isocyanato-1,2,3,5,6,7-hexahydro-s-indacene (Intermediate R1), and purified by reversed phase prep-HPLC method 1 to afford the title compound (23 mg, 34%) as a white solid.

¹H NMR (DMSO-d₆) δ 11.09 (s, 1H), 8.12 (s, 1H), 7.93 (s, 1H), 6.91 (s, 1H), 4.48 (app. p, J=6.7 Hz, 1H), 2.78 (t, J=7.4 Hz, 4H), 2.59 (t, J=7.4 Hz, 4H), 1.93 (p, J=7.4 Hz, 4H), 1.40 (d, J=6.7 Hz, 6H).

LCMS m/z 407.7, 429.1 (M+H, M+Na)⁺ (ES⁺).

### Example 56

### 1-(1-(Azetidin-1-yl)-2-methylpropan-2-yl)-N-((1,2,3,5,6,7-hexahydro-s-indacen-4-yl)carbamoyl)-1H-pyrazole-3-sulfonamide

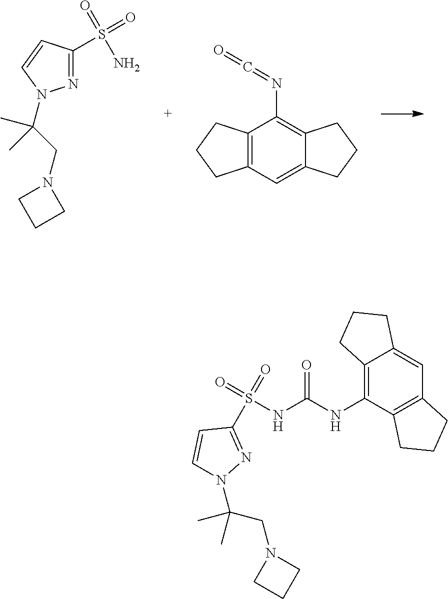 [SMILES: CC(C)(CN1CCC1)n1ccc(S(N)(=O)=O)n1 ; O=C=Nc1c2c(cc3c1CCC3)CCC2 ; CC(C)(CN1CCC1)n1ccc(S(=O)(=O)NC(=O)Nc2c3c(cc4c2CCC4)CCC3)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 1-(1-(azetidin-1-yl)-2-methylpropan-2-yl)-1H-pyrazole-3-sulfonamide (Intermediate L30) and 4-isocyanato-1,2,3,5,6,7-hexahydro-s-indacene (Intermediate R1), and purified by reversed phase prep-HPLC method 1 to afford the title compound (23 mg, 34%) as a white solid.

¹H NMR (DMSO-d₆) δ 10.71 (s, 1H), 7.95-7.94 (m, 2H), 6.92 (s, 1H), 6.69 (d, J=2.4 Hz, 1H), 2.96 (app br s, 4H), 2.80-2.70 (m, 6H), 2.61 (t, J=7.4 Hz, 4H), 1.94 (p, J=7.5 Hz, 4H), 1.78 (p, J=7.1 Hz, 2H), 1.50 (s, 6H).

LCMS m/z 458.4 (M+H)⁺ (ES⁺); 456.3 (M−H)⁻ (ES⁻).

### Example 57

### 3-((Dimethylamino)methyl)-N-((1,2,3,5,6,7-hexahydro-s-indacen-4-yl)carbamoyl)-5-methylbenzenesulfonamide, sodium salt

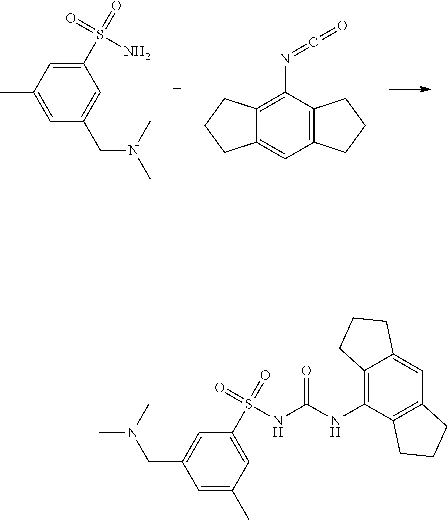

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 3-((dimethylamino)methyl)-5-methylbenzenesulfonamide (Intermediate L31) and 4-isocyanato-1,2,3,5,6,7-hexahydro-s-indacene (Intermediate R1) to afford the title compound (40 mg, 44%) as a white solid.

¹H NMR (DMSO-d₆) δ 7.51-7.44 (m, 3H), 7.10 (s, 1H), 6.76 (s, 1H), 3.34 (s, 2H), 2.74 (t, J=_{7.4} Hz, 4H), 2.64 (t, J=7.4 Hz, 4H), 2.31 (s, 3H), 2.14 (s, 6H), 1.94-1.84 (m, 4H).

LCMS m/z 428.3 (M+H)⁺(ES⁺).

### Example 58

### 1-(2,2-Difluoroethyl)-N-((1,2,3,5,6,7-hexahydro-s-indacen-4-yl)carbamoyl)-1H-pyrazole-4-sulfonamide, sodium salt

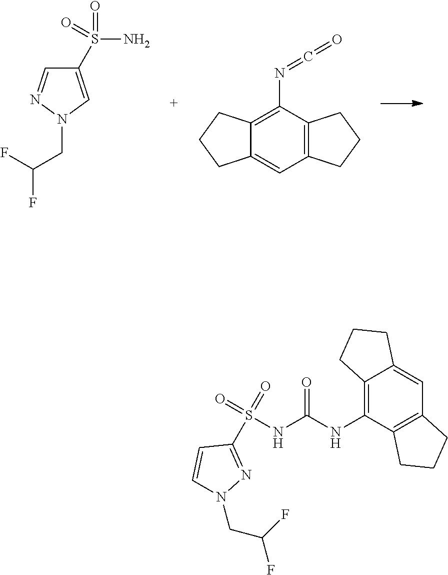 [SMILES: NS(=O)(=O)c1cnn(CC(F)F)c1 ; O=C=Nc1c2c(cc3c1CCC3)CCC2 ; O=C(Nc1c2c(cc3c1CCC3)CCC2)NS(=O)(=O)c1ccn(CC(F)F)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example ₄₃) from 1-(2,2-difluoroethyl)-1H-pyrazole-4-sulfonamide (Intermediate L32) and 4-isocyanato-1,2,3,5,6,7-hexahydro-s-indacene (Intermediate R1) to afford the title compound (42 mg, 40%) as a white solid.

¹H NMR (DMSO-d₆) δ 7.98 (s, 1H), 7.64 (s, 1H), 7.34 (s, 1H), 6.76 (s, 1H), 6.49-6.17 (m, 1H), 4.68-4.51 (m, 2H), 2.75 (t, J=7.4 Hz, 4H), 2.66 (t, J=7.5 Hz, 4H), 1.97-1.82 (m, 4H).

LCMS m/z 411.6 (M+H)⁺ (ES⁺).

### Example 59

### 1-Cyclobutyl-N-((1,2,3,5,6,7-hexahydro-s-indacen-4-yl) carbamoyl)-1H-pyrazole-3-sulfonamide

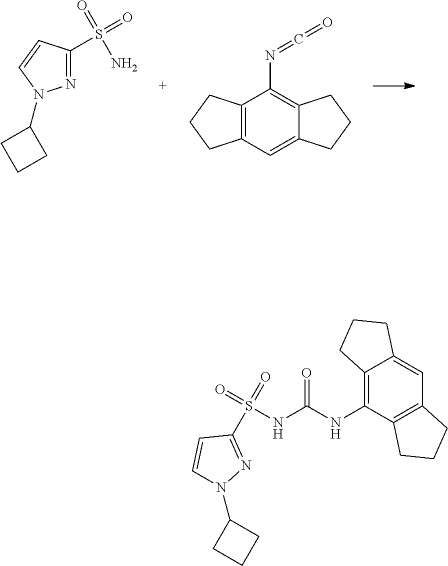 [SMILES: NS(=O)(=O)c1ccn(C2CCC2)n1 ; O=C=Nc1c2c(cc3c1CCC3)CCC2 ; O=C(Nc1c2c(cc3c1CCC3)CCC2)NS(=O)(=O)c1ccn(C2CCC2)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 1-cyclobutyl-1H-pyrazole-3-sulfonamide (Intermediate L33) and 4-isocyanato-1,2,3,5,6,7-hexahydro-s-indacene (Intermediate R1), and purified by reversed phase prep-HPLC method 1 to afford the title compound (33 mg, 34%) as a white solid.

¹H NMR (DMSO-d₆) δ 10.86 (s, 1H), 8.02 (d, J=2.4 Hz, 1H), 6.94 (s, 1H), 6.74 (d, J=2.4 Hz, 1H), 4.95 (p, J=8.4 Hz, 1H), 2.79 (t, J=7.4 Hz, 4H), 2.58 (t, J=7.4 Hz, 4H), 2.48-2.35 (m, 4H), 1.94 (p, J=7.4 Hz, 4H), 1.86-1.76 (m, 2H). One exchangeable proton not observed.

LCMS m/z 401.3 (M+H)⁺ (ES⁺).

### Example 60

### 1-(1-((Dimethylamino)methyl)cyclobutyl)-N-((1,2,3,5,6,7-hexahydro-s-indacen-4-yl)carbamoyl)-1H-pyrazole-3-sulfonamide

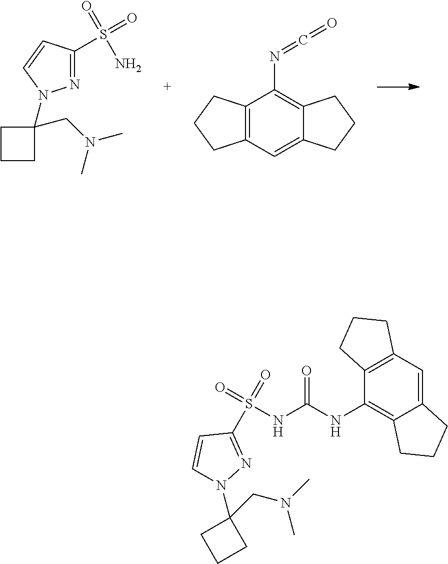 [SMILES: CN(C)CC1(n2ccc(S(N)(=O)=O)n2)CCC1 ; O=C=Nc1c2c(cc3c1CCC3)CCC2 ; CN(C)CC1(n2ccc(S(=O)(=O)NC(=O)Nc3c4c(cc5c3CCC5)CCC4)n2)CCC1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 1-(1-((dimethylamino)methyl)cyclobutyl)-1H-pyrazole-3-sulfonamide (Intermediate L₃₄) and 4-isocyanato-1,2,3,5,6,7-hexahydro-s-indacene (Intermediate R1), and purified by reversed phase prep-HPLC method 1 to afford the title compound (3 mg, 4%) as a white solid.

¹H NMR (DMSO-d₆) δ 7.92 (app s, 2H), 6.91 (s, 1H), 6.70 (app s, 1H), 2.81-2.75 (m, 6H), 2.60 (t, J=7.4 Hz, 4H), 2.49-2.44 (m, 2H), 2.35-2.28 (m, 2H), 1.97-1.92 (m, 4H), 1.91 (s, 6H), 1.89-1.82 (m, 2H). One exchangeable proton not observed.

LCMS m/z 458.2 (M+H)⁺(ES⁺); 456.3 (M−H)⁻ (ES⁻).

### Example 61

### N-((8-Fluoro-1,2,3,5,6,7-hexahydro-s-indacen-4-yl) carbamoyl)-1-isopropyl-1H-pyrazole-3-sulfonamide, sodium salt

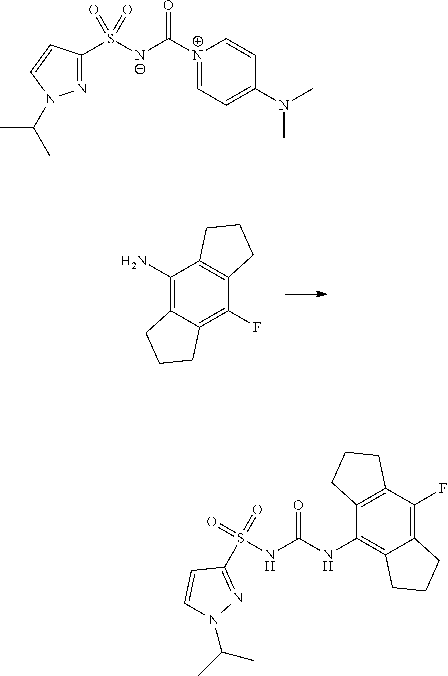 [SMILES: CC(C)n1ccc(S(=O)(=O)[N-]C(=O)[n+]2ccc(N(C)C)cc2)n1 ; Nc1c2c(c(F)c3c1CCC3)CCC2 ; CC(C)n1ccc(S(=O)(=O)NC(=O)Nc2c3c(c(F)c4c2CCC4)CCC3)n1]

Step A: A solution of 8-fluoro-1,2,3,5,6,7-hexahydro-s-indacen-4-amine (Intermediate R17) (80 mg, 0.418 mmol) and (4-(dimethylamino)pyridin-1-ium-1-carbonyl)((1-isopropyl-1H-pyrazol-3-yl)sulfonyl)amide (Intermediate L21) (141 mg, 0.418 mmol) in MeCN (5 mL) was stirred at 50° C. for 1.5 hours. The solvent was removed in vacuo and the crude product was purified by reversed phase prep-HPLC method 1 to afford N-((8-fluoro-1,2,3,5,6,7-hexahydro-s-indacen-4-yl)carbamoyl)-1-isopropyl-1H-pyrazole-3-sulfonamide (88 mg) as a pale yellow solid.

LCMS m/z 407.0 (M+H)⁺(ES⁺); 405.2 (M−H)⁻ (ES⁻).

Step B: N-((8-Fluoro-1,2,3,5,6,7-hexahydro-s-indacen-4-yl)carbamoyl)-1-isopropyl-1H-pyrazole-3-sulfonamide (85 mg, 0.21 mmol) was dissolved in aq NaOH (0.1 M, 2.2 mL, 0.22 mmol) containing 2 drops of THF and freeze-dried to afford the title compound (94 mg, 52%) as a white solid.

¹H NMR (DMSO-d₆) δ 7.71 (d, J=2.3 Hz, 1H), 7.54 (br s, 1H), 6.38 (d, J=2.3 Hz, 1H), 4.50 (sept, J=6.7 Hz, 1H), 2.78 (t, J=7.5 Hz, 4H), 2.68 (t, J=7.5 Hz, 4H), 1.95 (p, J=7.5 Hz, 4H), 1.40 (d, J=6.7 Hz, 6H).

LCMS m/z 407.5 (M+H)⁺ (ES⁺); 405.2 (M−H)⁻ (ES⁻).

### Example 62

### 5-Fluoro-N-((1,2,3,5,6,7-hexahydro-s-indacen-4-yl) carbamoyl)-1-isopropyl-1H-pyrazole-3-sulfonamide, sodium salt

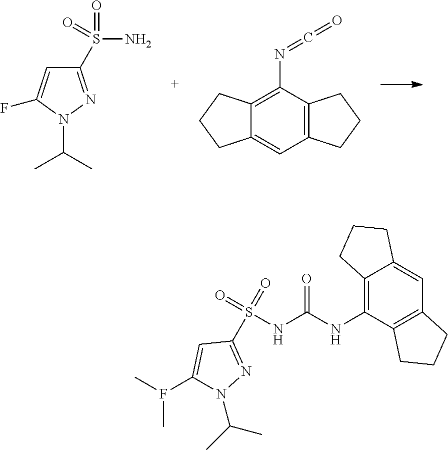

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from of 5-fluoro-1-isopropyl-1H-pyrazole-3-sulfonamide (Intermediate L35) and 4-isocyanato-1,2,3,5,6,7-hexahydro-s-indacene (Intermediate R1), and purified by reversed phase prep-HPLC method 1 to afford 5-fluoro-N-((1,2,3,5,6,7-hexahydro-s-indacen-4-yl)carbamoyl)-1-isopropyl-1H-pyrazole-3-sulfonamide (9.85 mg) as a solid. Aq NaOH (0.1 M, 0.24 mL) was added and the solution was freeze-dried to afford the title compound (10.3 mg, 12%) as a solid.

¹H NMR (DMSO-d₆) δ 7.89 (s, 1H), 6.89 (s, 1H), 6.40 (s, 1H), 4.67-4.57 (m, 1H), 2.78 (t, J=7.4 Hz, 4H), 2.62 (t, J=7.4 Hz, 4H), 1.93 (p, J=7.4 Hz, 4H), 1.40 (d, J=6.7 Hz, 6H).

LCMS m/z 407.2 (M+H)⁺ (ES⁺).

### Example 63

### 1-(2,2-Difluoroethyl)-N-((1,2,3,5,6,7-hexahydro-s-indacen-4-yl)carbamoyl)-1H-pyrazole-3-sulfonamide, sodium salt

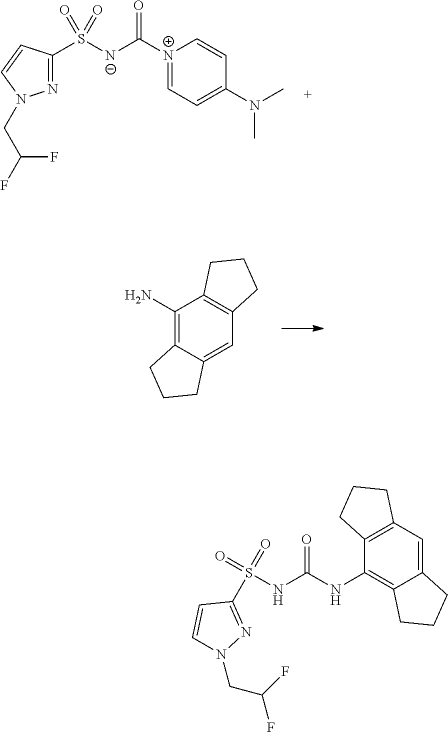 [SMILES: CN(C)c1cc[n+](C(=O)[N-]S(=O)(=O)c2ccn(CC(F)F)n2)cc1 ; Nc1c2c(cc3c1CCC3)CCC2 ; O=C(Nc1c2c(cc3c1CCC3)CCC2)NS(=O)(=O)c1ccn(CC(F)F)n1]

Prepared according to the general procedure of N-((8-fluoro-1,2,3,5,6,7-hexahydro-s-indacen-4-yl)carbamoyl)-1-isopropyl-1H-pyrazole-3-sulfonamide, sodium salt (Example 61) from ((1-(2,2-difluoroethyl)-1H-pyrazol-3-yl)sulfonyl)(4-(dimethylamino)pyridin-1-ium-1-carbonyl)amide (Intermediate L36) and 1,2,3,5,6,7-hexahydro-s-indacen-4-amine to afford the title compound (13.8 mg, 14%) as a solid.

¹H NMR (DMSO-d₆) δ 7.92-7.86 (m, 2H), 6.89 (s, 1H), 6.73-6.69 (m, 1H), 6.52-6.23 (m, 1H), 4.77-4.66 (m, 2H), 2.78 (t, J=7.4 Hz, 4H), 2.61 (t, J=7.4 Hz, 4H), 1.93 (1), J=7.4 Hz, 4H).

LCMS m/z 411.2 (M+H)⁺ (ES⁺).

### Example 64

### 1-Isopropyl-N-((3,5,6,7-tetrahydro-2H-indeno[5,6-b]furan-8-yl)carbamoyl)-1H-pyrazole-3-sulfonamide, sodium salt

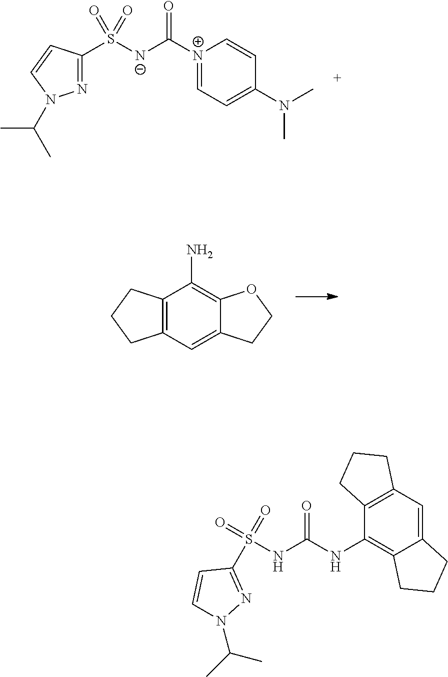 [SMILES: CC(C)n1ccc(S(=O)(=O)[N-]C(=O)[n+]2ccc(N(C)C)cc2)n1 ; Nc1c2c(cc3c1OCC3)CCC2 ; CC(C)n1ccc(S(=O)(=O)NC(=O)Nc2c3c(cc4c2CCC4)CCC3)n1]

Prepared according to the general procedure of N-((8-fluoro-1,2,3,5,6,7-hexahydro-s-indacen-4-yl)carbamoyl)-1-isopropyl-1H-pyrazole-3-sulfonamide, sodium salt (Example 61) from (4-(dimethylamino)pyridin-1-ium-1-carbonyl)((1-isopropyl-1H-pyrazol-3-yl)sulfonyl)amide (Intermediate L21) and 3,5,6,7-tetrahydro-2H-indeno[5,6-b]furan-8-amine to afford the title compound (18 mg, 79%) as a white solid.

¹H NMR (DMSO-d₆) δ 7.70 (d, J=2.3 Hz, 1H), 7.19 (s, 1H), 6.76 (s, 1H), 6.38 (d, J=2.3 Hz, 1H), 4.56-4.38 (m, 3H), 3.08 (t, J=8.6 Hz, 2H), 2.71 (t, J=7.3 Hz, 2H), 2.61 (t, J=7.4 Hz, 2H), 1.87 (p, J=7.4 Hz, 2H), 1.40 (d, J=6.7 Hz, 6H).

LCMS m/z 391.3 (M+H)⁺ (ES⁺).

### Example 65

### N-((1,2,3,5,6,7-Hexahydro-s-indacen-4-yl)carbamoyl)-1-(2-methyl-1-(pyrrolidin-1-yl)propan-2-yl)-1H-pyrazole-3-sulfonamide, sodium salt

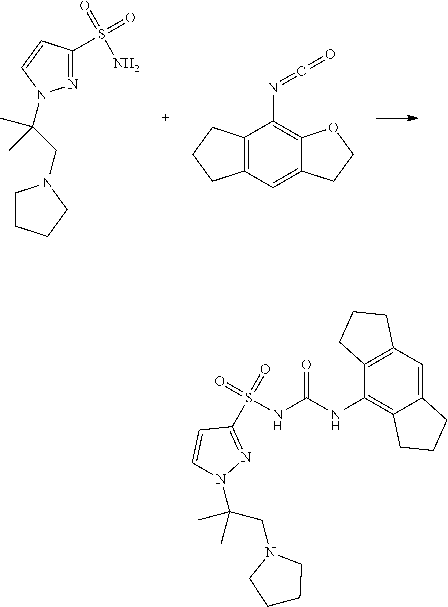 [SMILES: CC(C)(CN1CCCC1)n1ccc(S(N)(=O)=O)n1 ; O=C=Nc1c2c(cc3c1OCC3)CCC2 ; CC(C)(CN1CCCC1)n1ccc(S(=O)(=O)NC(=O)Nc2c3c(cc4c2CCC4)CCC3)n1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 1-(2-methyl-1-(pyrrolidin-1-yl)propan-2-yl)-1H-pyrazole-3-sulfonamide (Intermediate L37) and 4-isocyanato-1,2,3,5,6,7-hexahydro-s-indacene (Intermediate R1), and purified by reversed phase chromatography on RP Flash C18 (12 g column, 5-50% MeCN/10 mM ammonium bicarbonate) to afford N-((1,2,3,5,6,7-hexahydro-s-indacen-4-yl)carbamoyl)-1-(2-methyl-1-(pyrrolidin-1-yl)propan-2-yl)-1H-pyrazole-3-sulfonamide (46.4 mg) as a white solid. The crude product obtained was dissolved in aq NaOH (0.1 M, 0.980 mL, 0.098 mmol) and freeze-dried to afford the title compound (44 mg, 38%) as a white powder.

¹H NMR (DMSO-d₆) δ 7.68 (d, J=2.4 Hz, 1H), 7.52 (br s, 1H), 6.75 (s, 1H), 6.37 (d, J=2.3 Hz, 1H), 2.82-2.69 (m, 6H), 2.64 (t, J=7.4 Hz, 4H), 2.29-2.17 (m, 4H), 1.88 (p, J=7.4 Hz, 4H), 1.54-1.41 (m, 10H).

LCMS m/z 472.3 (M+H)⁺ (ES⁺); 470.1 (M−H)⁻ (ES⁻).

### Example 66

### 4-((Dimethylamino)methyl)-N-((1,2,3,5,6,7-hexahydro-s-indacen-4-yl)carbamoyl)benzenesulfonamide, sodium salt

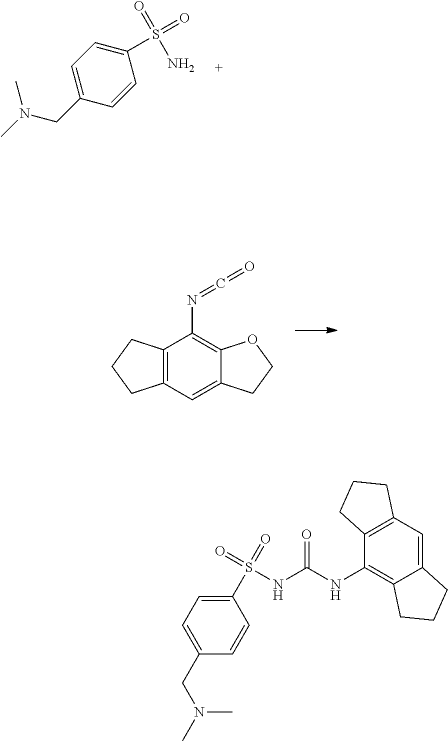 [SMILES: CN(C)Cc1ccc(S(N)(=O)=O)cc1 ; O=C=Nc1c2c(cc3c1OCC3)CCC2 ; CN(C)Cc1ccc(S(=O)(=O)NC(=O)Nc2c3c(cc4c2CCC4)CCC3)cc1]

Prepared according to the general procedure of N-((1,2,3,5,6,7-hexahydro-s-indacen-4-yl)carbamoyl)-1-isopropyl-3-methyl-1H-pyrazole-4-sulfonamide, sodium salt (Example 43) from 4-((dimethylamino)methyl)benzenesulfonamide (Intermediate L13) and 4-isocyanato-1,2,3,5,6,7-hexahydro-s-indacene (Intermediate R1), and purified by reversed phase chromatography on RP Flash C18 (12 g column, 5-50% MeCN/10 mM ammonium bicarbonate) to afford 4-((dimethylamino)methyl)-N-((1,2,3,5,6,7-hexahydro-s-indacen-4-yl)carbamoyl)benzenesulfonamide (51.6 mg) as a white powder. The crude product obtained was dissolved in aq NaOH (0.1 M, 1.25 mL, 0.125 mmol) and freeze-dried to afford the title compound (51 mg, 35%) as a white solid.

¹H NMR (DMSO-d₆) δ 7.76-7.60 (m, 2H), 7.41 (s, 1H), 7.31-7.21 (m, 2H), 6.75 (s, 1H), 3.38 (s, 2H), 2.73 (t, J=7.4 Hz, 4H), 2.61 (t, J=7.4 Hz, 4H), 2.13 (s, 6H), 1.87 (p, J=7.4 Hz, 4H).

LCMS m/z 414.3 (M+H)⁺ (ES⁺); 412.2 (M−H)⁻ (ES⁻).

### Example 67

### 1-Cyclopropyl-N-((3,5,6,7-tetrahydro-2H-indeno[5,6-b]furan-4-yl)carbamoyl)-1H-pyrazole-3-sulfonamide

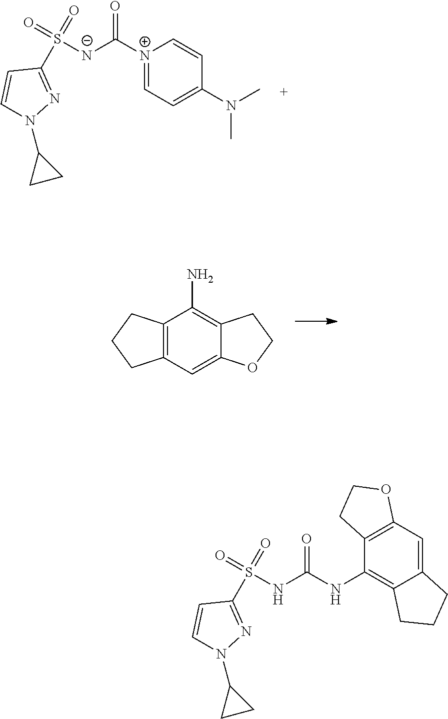 [SMILES: CN(C)c1cc[n+](C(=O)[N-]S(=O)(=O)c2ccn(C3CC3)n2)cc1 ; Nc1c2c(cc3c1CCO3)CCC2 ; O=C(Nc1c2c(cc3c1CCO3)CCC2)NS(=O)(=O)c1ccn(C2CC2)n1]

Prepared according to the general procedure of N-((8-fluoro-1,2,3,5,6,7-hexahydro-s-indacen-4-yl)carbamoyl)-1-isopropyl-1H-pyrazole-3-sulfonamide (Example 61, Step A) from ((1-cyclopropyl-1H-pyrazol-3-yl)sulfonyl)(4-(dimethylamino)pyridin-1-ium-1-carbonyl)amide (Intermediate L38) and 3,5,6,7-tetrahydro-2H-indeno[5,6-b]furan-4-amine to afford the title compound (19 mg, 26%) as a white solid.

¹H NMR (DMSO-d₆) δ 8.00 (s, 1H), 7.90 (app. t, J=2.0 Hz, 1H), 6.62 (app. t, J=2.0 Hz, 1H), 6.44 (s, 1H), 4.44 (t, J=8.6 Hz, 2H), 3.85-3.78 (m, 1H), 2.94 (t, J=8.6 Hz, 2H), 2.75 (t, J=7.5 Hz, 2H), 2.59 (t, J=7.3 Hz, 2H), 1.94 (p, J=7.4 Hz, 2H), 1.07-0.98 (m, 4H). One exchangeable proton not observable.

LCMS m/z 389.2 (M+H)⁺ (ES⁺).

The compounds of examples 68-97 were synthesised by methods analogous to those outlined above and below.

TABLE 1
¹H NMR and MS data

| Ex | Structure | ¹H NMR | MS | MW |
|---|---|---|---|---|
| 68 | 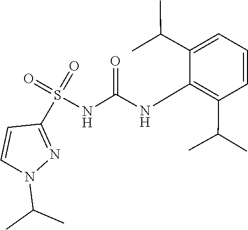 [SMILES: CC(C)c1cccc(C(C)C)c1NC(=O)NS(=O)(=O)c1ccn(C(C)C)n1] | ¹H NMR (DMSO-d₆) δ 7.59 (d, J = 2.3 Hz, 1H), 7.32 (br s, 1H), 7.01 (dd, J = 8.5, 6.6 Hz, 1H), 6.96-6.89 (m, 2H), 6.27 (br s, 1H), 4.39 (m, 1H), 3.07 (m, 2H), 1.32 (d, J = 6.7 Hz, 6H), 0.96 (d, J = 6.8 Hz, 12H). | m/z 393.1 (M + H)⁺ (ES⁺) | 392.52 |
| | N-((2,6-Diisopropylphenyl)carbamoyl)- | | | |
| | 1-isopropyl-1H-pyrazole-3- | | | |
| | sulfonamide, sodium salt | | | |
| 69 | 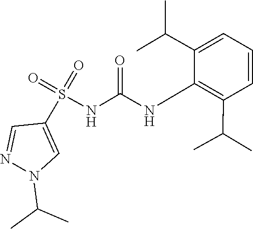 [SMILES: CC(C)c1cccc(C(C)C)c1NC(=O)NS(=O)(=O)c1cnn(C(C)C)c1] | ¹H NMR (DMSO-d₆) δ 10.67 (s, 1H), 8.39 (s, 1H), 7.84 (s, 1H), 7.82 (s, 1H), 7.28-7.19 (m, 1H), 7.11 (d, J = 7.7 Hz, 2H), 4.58 (sept, J = 6.6 Hz, 1H), 2.90 (sept, J = 6.9 Hz, 2H), 1.40 (d, J = 6.7 Hz, 6H), 1.16-0.86 (m, 12H). | m/z 393.1 (M + H)⁺ (ES⁺) | 392.52 |
| | N-((2,6-Diisopropylphenyl)carbamoyl)- | | | |
| | 1-isopropyl-1H-pyrazole-4-sulfonamide | | | |
| 70 | 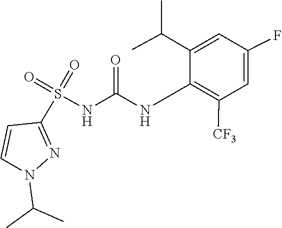 [SMILES: CC(C)c1cc(F)cc(C(F)(F)F)c1NC(=O)NS(=O)(=O)c1ccn(C(C)C)n1] | ¹H NMR (DMSO-d₆) δ 11.19 (s, 1H), 8.02 (s, 1H), 7.98 (d, J = 2.4 Hz, 1H), 7.55 (dd, J = 9.9, 3.0 Hz, 1H), 7.45 (dd, J = 8.5, 3.0 Hz, 1H), 6.71 (d, J = 2.4 Hz, 1H), 4.62 (sept, J = 6.6 Hz, 1H), 2.98-2.84 (m, 1H), 1.45 (d, J = 6.7 Hz, 6H), 1.21-0.91 (m, 6H). | m/z 437.4 (M + H)⁺ (ES⁺); 435.5 (M − H)⁻ (ES⁻) | 436.42 |
| | N-((4-Fluoro-2-isopropyl-6- | | | |
| | (trifluoromethyl)phenyl)carbamoyl)-1- | | | |
| | isopropyl-1H-pyrazole-3-sulfonamide | | | |
| 71 | 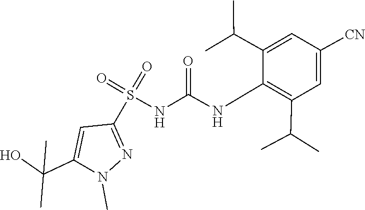 [SMILES: CC(C)c1cc(C#N)cc(C(C)C)c1NC(=O)NS(=O)(=O)c1cc(C(C)(C)O)n(C)n1] | ¹H NMR (DMSO-d₆) δ 11.14 (s, 1H), 8.15 (s, 1H), 7.59 (s, 2H), 6.52 (s, 1H), 5.48 (s, 1H), 4.03 (s, 3H), 3.12-2.93 (m, 2H), 1.48 (s, 6H), 1.08 (d, J = 6.8 Hz, 12H). | m/z 448.1 (ES⁺) | 447.55 |
| | N-((4-Cyano-2,6- | | | |
| | diisopropylphenyl)carbamoyl)-5-(2- | | | |
| | hydroxypropan-2-yl)-1-methyl-1H- | | | |
| | pyrazole-3-sulfonamide | | | |
| 72 | 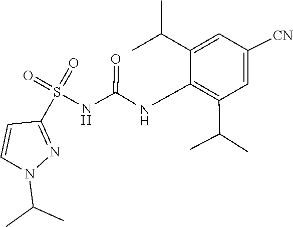 [SMILES: CC(C)c1cc(C#N)cc(C(C)C)c1NC(=O)NS(=O)(=O)c1ccn(C(C)C)n1] | ¹H NMR (DMSO-d₆) δ 11.19 (s, 1H), 8.14 (s, 1H), 7.98 (d, J = 2.4 Hz, 1H), 7.59 (s, 2H), 6.72 (d, J = 2.4 Hz, 1H), 4.69- 4.51 (m, 1H), 3.05-2.87 (m, 2H), 1.44 (d, J = 6.7 Hz, 6H), 1.06 (d, J = 6.8 Hz, 12H). | m/z 418.1 (M + H)⁺ (ES⁺) | 417.53 |
| | N-((4-Cyano-2,6-diisopropylphenyl) | | | |
| | carbamoyl)-1-isopropyl-1H-pyrazole-3- | | | |
| | sulfonamide | | | |
| 73 | 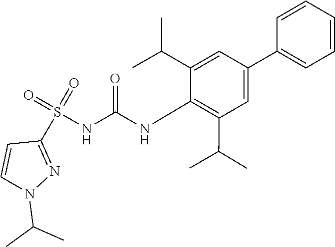 [SMILES: CC(C)c1cc(-c2ccccc2)cc(C(C)C)c1NC(=O)NS(=O)(=O)c1ccn(C(C)C)n1] | ¹H NMR (DMSO-d₆) δ 11.03 (br s, 1H), 7.97 (br s, 1H), 7.86 (br s, 1H), 7.63 (d, J = 7.5 Hz, 2H), 7.46 (t, J = 7.5 Hz, 2H), 7.26 (t, J = 7.5 H, 1H), 7.33 (s, 2H), 6.70 (br s, 1H), 4.61 (sept, J = 6.7 Hz, 1H), 3.00 (sept, J = 6.9 Hz, 2H), 1.45 (d, J = 6.7 Hz, 6H), 1.26-1.00 (m, 12H). | m/z 469 (M + H)⁺ (ES⁺); 467 (M − H)⁻ (ES⁻), | 468.62 |
| | N-((3,5-Diisopropyl-[1,1′-biphenyl]-4- | | | |
| | yl)carbamoyl)-1-isopropyl-1H-pyrazole- | | | |
| | 3-sulfonamide | | | |
| 74 | 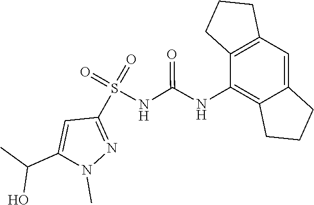 [SMILES: CC(O)c1cc(S(=O)(=O)NC(=O)Nc2c3c(cc4c2CCC4)CCC3)nn1C] | ¹H NMR (DMSO) δ 10.77 (br s, 1H), 7.96 (s, 1H), 6.91 (s, 1H), 6.59 (s, 1H), 5.47 (d, J = 5.6 Hz, 1H), 4.84 (m, 1H), 3.88 (s, 3H), 2.78 (t, J = 7.4 Hz, 4H), 2.62 (t, J = 7.4 Hz, 4H), 1.94 (p, J = 7.5 Hz, 4H), 1.41 (d, J = 6.5 Hz, 3H). | m/z 405.4 (M + H)⁺ (ES⁺); 403.3 (M − H)⁻ (ES⁻). | 404.49 |
| | N-((1,2,3,5,6,7-Hexahydro-s-indacen-4- | | | |
| | yl)carbamoyl)-5-(1-hydroxyethyl)-1- | | | |
| | methyl-1H-pyrazole-3-sulfonamide | | | |
| 75 | 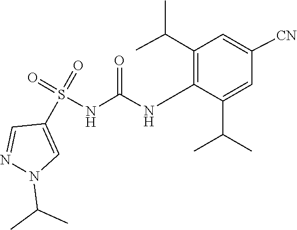 [SMILES: CC(C)c1cc(C#N)cc(C(C)C)c1NC(=O)NS(=O)(=O)c1cnn(C(C)C)c1] | ¹H NMR (Methanol-d₄) δ 8.27 (s, 1H), 7.91 (s, 1H), 7.51 (s, 2H), 4.71-4.46 (m, 1H), 3.20-2.88 (m, 2H), 1.50 (d, J = 6.7 Hz, 6H), 1.13 (d, J = 6.9 Hz, 12H). Two exchangeable protons not observed. | m/z 418.5 (M + H)⁺ (ES); 416.3 (M − H)⁻ (ES⁻). | 417.53 |
| | N-((4-Cyano-2,6-diisopropylphenyl) | | | |
| | carbamoyl)-1-isopropyl-1H-pyrazole-4- | | | |
| | sulfonamide | | | |
| 76 | 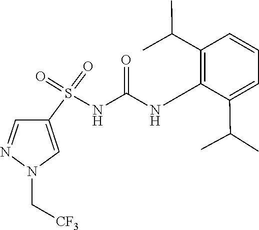 [SMILES: CC(C)c1cccc(C(C)C)c1NC(=O)NS(=O)(=O)c1cnn(CC(F)(F)F)c1] | ¹H NMR (DMSO-d₆) δ 7.75 (br s, 1H), 7.33 (s, 1H), 7.08 (s, 1H), 6.43-6.33 (m, 1H), 6.28 (d, J = 7.7 Hz, 2H), 4.18- 4.15 (m, 2H), 2.43-2.24 (m, 2H), 0.31 (d, J = 6.9 Hz, 12H). One exchangeable proton not observed. | m/z 433 (M + H)⁺ (ES⁺). | 432.46 |
| | N-((2,6-Diisopropylphenyl)carbamoyl)- | | | |
| | 1-(2,2,2-trifluoroethyl)-1H-pyrazole-4- | | | |
| | sulfonamide | | | |
| 77 | 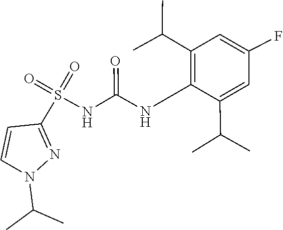 [SMILES: CC(C)c1cc(F)cc(C(C)C)c1NC(=O)NS(=O)(=O)c1ccn(C(C)C)n1] | ¹H NMR (DMSO-d₆) δ 7.69 (d, J = 2.3 Hz, 1H), 7.38 (br s, 1H), 6.78 (d, J = 10.1 Hz, 2H), 6.36 (br s, 1H), 4.49- 4.46 (m, 1H), 3.15-3.13 (m, 2H), 1.39 (d, J = 6.7 Hz, 6H), 1.02 (d, J = 6.8 Hz, 12H). | m/z 411 (M + H)⁻, (ES⁺). | 410.51 |
| | N-((4-Fluoro-2,6-diisopropylphenyl) | | | |
| | carbamoyl)-1-isopropyl-1H-pyrazole-3- | | | |
| | sulfonamide, sodium salt | | | |
| 78 | 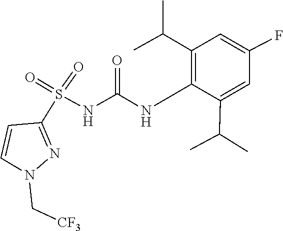 [SMILES: CC(C)c1cc(F)cc(C(C)C)c1NC(=O)NS(=O)(=O)c1ccn(CC(F)(F)F)n1] | ¹H NMR (DMSO-d₆) δ 7.77 (d, J = 2.4 Hz, 1H), 7.37 (s, 1H), 6.79 (d, J = 10.1 Hz, 2H), 6.50 (d, J = 2.4 Hz, 1H), 5.13-5.10 (m, 2H), 3.14- 3.06 (m, 2H), 1.02 (d, J = 6.8 Hz, 12H). | m/z 451 (M + H)⁺ (ES⁺). | 450.45 |
| | N-((4-Fluoro-2,6-diisopropylphenyl) | | | |
| | carbamoyl)-1-(2,2,2-trifluoroethyl)-1H- | | | |
| | pyrazole-3-sulfonamide, sodium salt | | | |
| 79 | 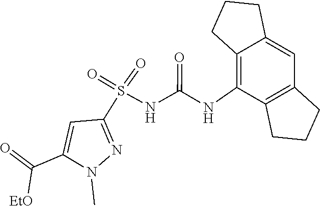 [SMILES: CCOC(=O)c1cc(S(=O)(=O)NC(=O)Nc2c3c(cc4c2CCC4)CCC3)nn1C] | ¹H NMR (DMSO-d₆) δ 11.02 (s, 1H), 8.13 (s, 1H), 7.24 (s, 1H), 6.94 (s, 1H), 4.32 (q, J = 7.1 Hz, 2H), 4.16 (s, 3H), 2.79 (t, J = 7.4 Hz, 4H), 2.60 (t, J = 7.4 Hz, 4H), 2.01-1.89 (m, 4H), 1.31 (t, J = 7.1 Hz, 3H). | m/z 433.4 (M + H)⁺ (ES⁺). | 432.50 |
| | Ethyl 3-(N-((1,2,3,5,6,7-hexahydro-s- | | | |
| | indacen-4-yl)carbamoyl)sulfamoyl)-1- | | | |
| | methyl-1H-pyrazole-5-carboxylate | | | |
| 80 | 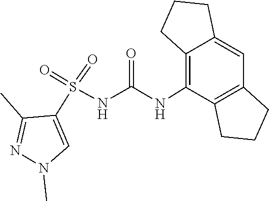 [SMILES: Cc1nn(C)cc1S(=O)(=O)NC(=O)Nc1c2c(cc3c1CCC3)CCC2] | ¹H NMR (DMSO-d₆) δ 7.69 (s, 1H), 7.39 (s, 1H), 6.75 (s, 1H), 3.69 (s, 3H), 2.74 (t, J = 7.4 Hz, 4H), 2.65 (t, J = 7.4 Hz, 4H), 2.22 (s, 3H), 1.93- 1.86 (m, 4H). | m/z 375 (M + H)⁺ (ES⁺). | 374.46 |
| | N-((1,2,3,5,6,7-Hexahydro-s-indacen-4- | | | |
| | yl)carbamoyl)-1,3-dimethyl-1H- | | | |
| | pyrazole-4-sulfonamide, sodium salt | | | |
| 81 | 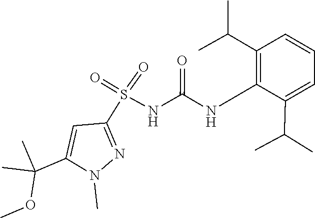 [SMILES: COC(C)(C)c1cc(S(=O)(=O)NC(=O)Nc2c(C(C)C)cccc2C(C)C)nn1C] | ¹H NMR (DMSO-d₆) δ 10.89 (br s, 1H), 7.82 (s, 1H), 7.26- 7.20 (m, 1H), 7.11 (d, J = 7.6 Hz, 2H), 6.63 (s, 1H), 3.98 (s, 3H), 3.02 (sept, J = 6.9 Hz, 2H), 2.97 (s, 3H), 1.50 (s, 6H), 1.06 (br s, 12H). | m/z 437 (M + H)⁺ (ES⁺); 435 (M − H)⁻ (ES⁻). | 436.57 |
| | N-((2,6-Diisopropylphenyl)carbamoyl)- | | | |
| | 5-(2-methoxypropan-2-yl)-1-methyl- | | | |
| | 1H-pyrazole-3-sulfonamide | | | |
| 82 | 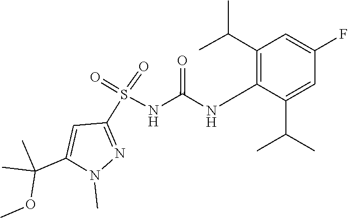 [SMILES: COC(C)(C)c1cc(S(=O)(=O)NC(=O)Nc2c(C(C)C)cc(F)cc2C(C)C)nn1C] | ¹H NMR (DMSO-d₆) δ 11.05 (s, 1H), 7.83 (s, 1H), 6.92 (d, J = 9.9 Hz, 2H), 6.63 (s, 1H), 3.98 (s, 3H), 3.01 (sept, J = 6.9 Hz, 2H), 2.97 (s, 3H), 1.50 (s, 6H), 1.08−1.02 (br m, 12H). | m/z 455 (M + H)⁺ (ES⁺); 453 (M − H)⁻ (ES⁻). | 454.56 |
| | N-((4-Fluoro-2,6-diisopropylphenyl) | | | |
| | carbamoyl)-5-(2-methoxypropan-2-yl)- | | | |
| | 1-methyl-1H-pyrazole-3-sulfonamide | | | |
| 83 | 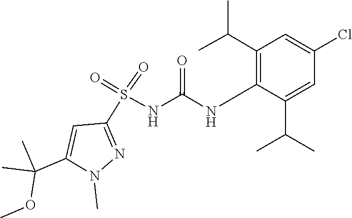 [SMILES: COC(C)(C)c1cc(S(=O)(=O)NC(=O)Nc2c(C(C)C)cc(Cl)cc2C(C)C)nn1C] | ¹H NMR (DMSO-d₆) δ 11.08 (s, 1H), 7.88 (s, 1H), 7.13 (s, 2H), 6.61 (s, 1H), 3.97 (s, 3H), 3.01 (sept, J = 6.9 Hz, 2H), 2.97 (s, 3H), 1.50 (s, 6H), 1.06 (br s, 12H). | m/z 472 (M + H)⁺ (ES⁺); 470 (M − H)⁻ (ES⁻). | 471.01 |
| | N-((4-Chloro-2,6-diisopropylphenyl) | | | |
| | carbamoyl)-5-(2-methoxypropan-2-yl)- | | | |
| | 1-methyl-1H-pyrazole-3-sulfonamide | | | |
| 84 | 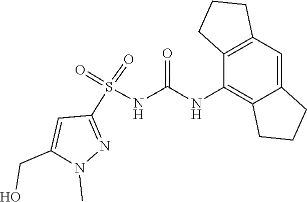 [SMILES: Cn1nc(S(=O)(=O)NC(=O)Nc2c3c(cc4c2CCC4)CCC3)cc1CO] | ¹H NMR (DMSO-d₆) δ 7.93 (s, 1H), 6.91 (s, 1H), 6.60 (s, 1H), 5.42 (t, J = 5.6 Hz, 1H), 4.51 (d, J = 5.5 Hz, 2H), 3.85 (s, 3H), 2.79 (t, J = 7.4 Hz, 4H), 2.63 (t, J = 7.4 Hz, 4H), 1.95 (p, J = 7.4 Hz, 4H). One exchangeable proton not observed. | m/z 391.3 (M + H)⁺ (ES⁺); 389.3 (M − H)⁻ (ES⁻). | 390.46 |
| | N-((1,2,3,5,6,7-Hexahydro-s-indacen-4- | | | |
| | yl)carbamoyl)-5-(hydroxymethyl)-1- | | | |
| | methyl-1H-pyrazole-3-sulfonamide | | | |
| 85 | 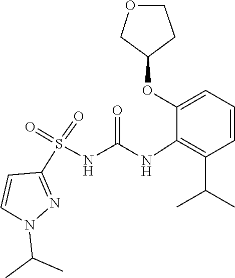 [SMILES: CC(C)c1cccc(O[C@@H]2CCOC2)c1NC(=O)NS(=O)(=O)c1ccn(C(C)C)n1] | ¹H NMR (DMSO-d₆) δ 10.92 (br s, 1H), 7.96 (d, J = 1.9 Hz, 1H), 7.49 (s, 1H), 7.18 (t, J = 8.0 Hz, 1H), 6.87 (dd, J = 8.0, 1.2 Hz, 1H), 6.81 (dd, J = 8.3, 1.2 Hz, 1H), 6.70 (d, J = 1.7 Hz, 1H), 4.95-4.87 (m, 1H), 4.60 (sept, J = 6.4 Hz, 1H), 3.87 (dd, J = 9.9, 4.8 Hz, 1H), 3.78-3.67 (m, 2H), 3.65 (d, J = 10.4 Hz, 1H), 2.94 (sept, J = 7.0 Hz, 1H), 2.18- 2.03 (m, 1H), 1.91-1.82 (m, 1H), 1.45 (d, J = 6.7 Hz, 6H), 1.05 (d, J = 6.9 Hz, 6H). | m/z 437 (M + H)⁺ (ES⁺); 435 (M − H)⁻ (ES⁻). | 436.53 |
| | (R)-1-Isopropyl-N-((2-isopropyl-6- | | | |
| | ((tetrahydrofuran-3-yl)oxy)phenyl) | | | |
| | carbamoyl)-1H-pyrazole-3-sulfonamide | | | |
| 86 | 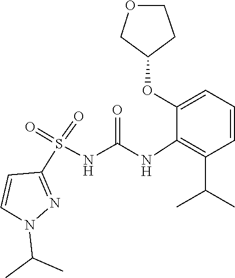 [SMILES: CC(C)c1cccc(O[C@H]2CCOC2)c1NC(=O)NS(=O)(=O)c1ccn(C(C)C)n1] | ¹H NMR (DMSO-d₆) δ 10.92 (br s, 1H), 7.96 (d, J = 1.9 Hz, 1H), 7.49 (s, 1H), 7.18 (t, J = 8.0 Hz, 1H), 6.87 (dd, J = 8.0, 1.2 Hz, 1H), 6.81 (dd, J = 8.3, 1.2 Hz, 1H), 6.70 (d, J = 1.7 Hz, 1H), 4.95-4.87 (m, 1H), 4.60 (sept, J = 6.4 Hz, 1H), 3.87 (dd, J = 9.9, 4.8 Hz, 1H), 3.78-3.67 (m, 2H), 3.65 (d, J = 10.4 Hz, 1H), 2.94 (sept, J = 7.0 Hz, 1H), 2.18- 2.03 (m, 1H), 1.91-1.82 (m, 1H), 1.45 (d, J = 6.7 Hz, 6H), 1.05 (d, J = 6.9 Hz, 6H). | m/z 437 (M + H)⁺ (ES⁺); 435 (M − H)⁻ (ES⁻). | 436.53 |
| | (S)-1-Isopropyl-N-((2-isopropyl-6- | | | |
| | ((tetrahydrofuran-3-yl)oxy)phenyl) | | | |
| | carbamoyl)-1H-pyrazole-3-sulfonamide | | | |
| 87 | 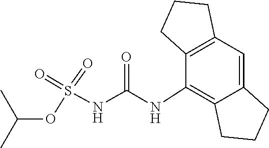 [SMILES: CC(C)OS(=O)(=O)NC(=O)Nc1c2c(cc3c1CCC3)CCC2] | ¹H NMR (DMSO-d₆) δ 7.55 (s, 1H), 6.79 (s, 1H), 4.51 (sept, J = 6.3 Hz, 1H), 2.76 (t, J = 7.4 Hz, 4H), 2.71 (t, J = 7.4 Hz, 4H), 1.92 (p, J = 7.4 Hz, 4H), 1.19 (d, J = 6.3 Hz, 6H). | m/z 337.0 (M − H)⁻ (ES⁻). | 338.42 |
| | Isopropyl (1,2,3,5,6,7-hexahydro-s- | | | |
| | indacen-4-yl)carbamoylsulfamate, | | | |
| | sodium salt | | | |
| 88 | 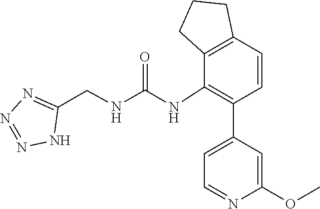 [SMILES: COc1cc(-c2ccc3c(c2NC(=O)NCc2nnn[nH]2)CCC3)ccn1] | ¹H NMR (DMSO-d₆) δ 8.13 (d, J = 5.3 Hz, 1H), 7.84 (s, 1H), 7.17 (d, J = 7.7 Hz, 1H), 7.11 (d, J = 7.6 Hz, 1H), 6.92 (dd, J = 5.3, 1.4 Hz, 1H), 6.74 (d, J = 1.4 Hz, 1H), 6.55 (t, J =5.4 Hz, 1H), 4.41 (d, J = 5.3 Hz, 2H), 3.86 (s, 3H), 2.92 (t, J = 7.4 Hz, 2H), 2.78 (t, J = 7.4 Hz, 2H), 2.00 (p, J = 7.5 Hz, 2H). One exchangeable proton not observed. | m/z 366.2 (M + H)⁺ (ES⁺); 364.4 (M − H)⁻ (ES⁻). | 365.40 |
| | 1((1H-Tetrazol-5-yl)methyl)-3-(5-(2- | | | |
| | methoxypyridin-4-yl)-2,3-dihydro-1H- | | | |
| | inden-4-yl)urea | | | |
| 89 | 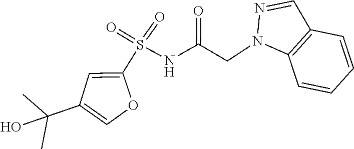 [SMILES: CC(C)(O)c1coc(S(=O)(=O)NC(=O)Cn2ncc3ccccc32)c1] | ¹H NMR (DMSO-d₆) δ 12.92 (br s, 1H), 8.07 (s, 1H), 7.84 (s, 1H), 7.75 (d, J = 8.1 Hz, 1H), 7.55 (d, J = 8.5 Hz, 1H), 7.41-7.33 (m, 1H), 7.25 (s, 1H), 7.14 (dd, J = 8.0, 6.9 Hz, 1H), 5.32 (S, 2H), 1.36 (s, 6H). One exchangeable proton not observed. | m/z 364.0 (M + H)⁺ (ES⁺). | 363.39 |
| | N-((4-(2-Hydroxypropan-2-yl)furan-2- | | | |
| | yl)sulfonyl)-2-(1H-indazol-1- | | | |
| | yl)acetamide | | | |
| 90 | 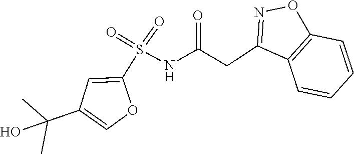 [SMILES: CC(C)(O)c1coc(S(=O)(=O)NC(=O)Cc2noc3ccccc23)c1] | ¹H NMR (DMSO-d₆) δ 7.81 (s, 1H), 7.73 (d, J = 8.5 Hz, 1H), 7.69 (d, J = 8.0 Hz, 1H), 7.67-7.62 (m, 1H), 7.38 (t, J = 7.5 Hz, 1H), 7.26 (s, 1H), 5.10 (s, 1H), 4.15 (s, 2H), 1.36 (s, 6H). One exchangeable proton not observed. | m/z 387.1 (M + Na)⁺ (ES⁺). | 364.37 |
| | 2-(Benzo[d]isoxazol-3-yl)-N-((4-(2- | | | |
| | hydroxypropan-2-yl)furan-2- | | | |
| | yl)sulfonyl)acetamide | | | |
| 91 | 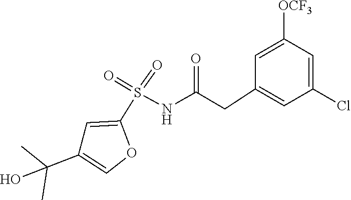 [SMILES: CC(C)(O)c1coc(S(=O)(=O)NC(=O)Cc2cc(Cl)cc(OC(F)(F)F)c2)c1] | | m/z 464.2 (M + Na)⁺ (ES⁺); 440.2 (M − H)⁻ (ES⁻); [R_{T} = 1.44 min]. | 441.03 |
| | 2-(3-Chloro-5-(trifluoromethoxy) | | | |
| | phenyl)-N-((4-(2-hydroxypropan-2- | | | |
| | yl)furan-2-yl)sulfonyl)acetamide | | | |
| 92 | 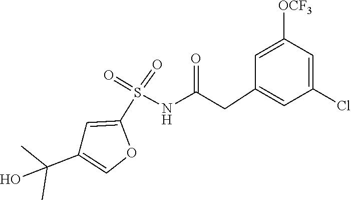 [SMILES: CC(C)(O)c1coc(S(=O)(=O)NC(=O)Cc2cc(Cl)cc(OC(F)(F)F)c2)c1] | | m/z 448.2 (M + Na)⁺ (ES⁺); 424.2 (M − H)⁻ (ES⁻); [R_{T} = 1.41 min]. | 425.81 |
| | 2-(3-Chloro-5-(trifluoromethyl) | | | |
| | phenyl)-N-((4-(2-hydroxypropan-2- | | | |
| | yl)furan-2-yl)sulfonyl)acetamide | | | |
| 93 | 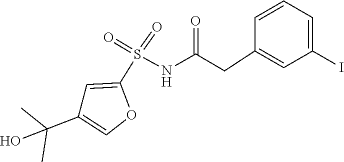 [SMILES: CC(C)(O)c1coc(S(=O)(=O)NC(=O)Cc2cccc(I)c2)c1] | | m/z 472.1 (M + Na)⁺ (ES⁺); [R_{T} = 1.33 min]. | 416.29 |
| | N-((4-(2-Hydroxypropan-2-yl)furan-2- | | | |
| | yl)sulfonyl)-2-(3-iodophenyl)acetamide | | | |
| 94 | 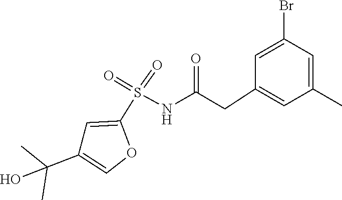 [SMILES: Cc1cc(Br)cc(CC(=O)NS(=O)(=O)c2cc(C(C)(C)O)co2)c1] | | m/z 438.1/44 0.1 (M + Na)⁺ (ES⁺); [R_{T} = 1.33 min]. | 416.29 |
| | 2-(3-Bromo-5-methylphenyl)-N-((4-(2- | | | |
| | hydroxypropan-2-yl)furan-2- | | | |
| | yl)sulfonyl)acetamide | | | |
| 95 | 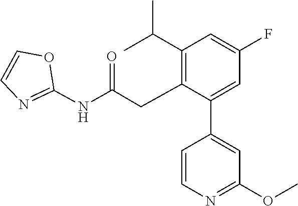 [SMILES: COc1cc(-c2cc(F)cc(C(C)C)c2CC(=O)Nc2ncco2)ccn1] | ¹H NMR (DMSO-d₆) δ 11.29 (s, 1H), 8.21 (d, 5.2 Hz, 1H), 7.85 (d, J = 1.0 Hz, 1H), 7.25 (dd, J = 10.5, 2.8 Hz, 1H), 7.08 (d, J = 1.0 Hz, 1H), 6.93 (dd, J = 8.9, 2.8 Hz, 1H), 6.89 (dd, J = 5.2, 1.5 Hz, 1H), 6.72 (s, 1H), 3.87 (s, 3H), 3.33 (s, 2H), 3.06-2.98 (m, 1H), 1.19 (d, J = 6.7 Hz, 6H). | 370.6 (M + H)⁺ (ES⁺). | 369.39 |
| | 2-(4-Fluoro-2-isopropyl-6-(2- | | | |
| | methoxypyridin-4-yl)phenyl)-N- | | | |
| | (oxazol-2-yl)acetamide | | | |
| 96 | 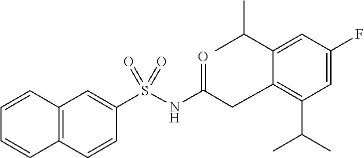 [SMILES: CC(C)c1cc(F)cc(C(C)C)c1CC(=O)NS(=O)(=O)c1ccc2ccccc2c1] | ¹H NMR (MeOD-d₄) δ 8.42 (br, s, 1H), 7.85-7.98 (m, 4H), 7.51-7.60 (m, 2H), 6.69 (d, 2H), 3.62 (s, 2H), 2.98- 3.15 (m, 2H), 1.12-0.88 (m, 12H). | m/z 428.2 (M + H)⁺ (ES⁺). | 427.53 |
| | 2-(4-Fluoro-2,6-diisopropylphenyl)-1- | | | |
| | (2-naphthylsulfonylamino)-1-ethanone | | | |
| 97 | 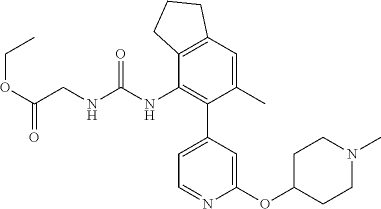 [SMILES: CCOC(=O)CNC(=O)Nc1c2c(cc(C)c1-c1ccnc(OC3CCN(C)CC3)c1)CCC2] | ¹H NMR (DMSO-d₆) δ 8.15 (d, J = 5.5 Hz, 1H), 7.44 (s, 1H), 7.04 (s, 1H), 6.70 (dd, J = 5.2, 1.4 Hz, 1H), 6.52 (s, 1H), 6.24 (t, J = 6.0 Hz, 1H), 5.06-4.98 (m, 1H), 4.08 (q, J = 7.1 Hz, 2H), 3.72 (d, J = 5.9 Hz, 2H), 2.88 (t, J = 7.4 Hz, 2H), 2.82-2.68 (m, 4H), 2.36-2.29 (m, 2H), 2.28 (s, 3H), 2.08-1.94 (m, 7H), 1.78- 1.68 (m, 2H), 1.19 (t, J = 7.1 Hz, 3H). | m/z 467.3 (M + H)⁺ (ES⁺). | 466.57 |
| | Ethyl 2-(3-(6-methyl-5-(2-((1- | | | |
| | methylpiperidin-4-yl)oxy)pyridin-4-yl)- | | | |
| | 2,3-dihydro-1H-inden-4- | | | |
| | yl)ureido)acetate | | | |

### Example 98

### 2-(3,5-bis(Trifluoromethyl)phenyl)-N-((4-(2-hydroxy-propan-2-yl)furan-2-yl)sulfonyl)acetamide

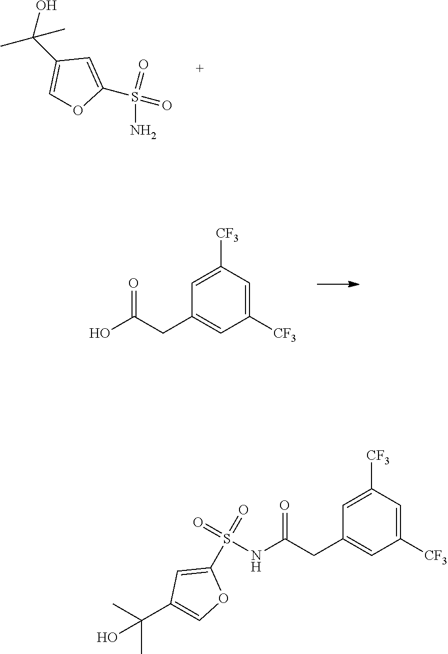 [SMILES: CC(C)(O)c1coc(S(N)(=O)=O)c1 ; O=C(O)Cc1cc(C(F)(F)F)cc(C(F)(F)F)c1 ; CC(C)(O)c1coc(S(=O)(=O)NC(=O)Cc2cc(C(F)(F)F)cc(C(F)(F)F)c2)c1]

To a solution of 2-(3,5-bis(trifluoromethyl)phenyl)acetic acid (0.1 mmol) in DMF (0.33 mL) was added a solution of 4-(2-hydroxypropan-2-yl)furan-2-sulfonamide (0.021 g, 0.100 mmol), DMAP (0.024 g, 0.200 mmol) and EDC (0.038 g, 0.200 mmol) in DMF (1 mL). The resultant mixture was shaken in a 96-well plate at room temperature for 16 hours. The crude product was purified by reversed phase prep-HPLC method 4 to afford the title compound (0.9 mg, 2%) as a white solid.

LCMS m/z 482.2 (M+Na)⁺ (ES⁺).

### EXAMPLES

### Biological Studies

NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

THP-1 Cells Pyroptosis Assay

The following method step-by-step assay was followed for compound screening.
1. Seed THP-1 cells (25,000 cells/well) containing 1.0 μg/ml LPS in 400 of RPMI medium (without FBS) in 96-well, black walled, clear bottom cell culture plates coated with poly-D-lysine (VWR #734-0317)
2. Add 5 μl compound (8 points half-log dilution, with 10 μM top dose) or vehicle (DMSO 0.1% FAC) to the appropriate wells
3. Incubate for 3 hrs at 37° C., 5% CO₂
4. Add 5 μl nigericin (Sigma #N7143) (FAC 5 μM) to all wells
5. Incubate for 1 hr at 37° C., 5% CO₂
6. At the end of the incubation period, spin plates at 300×g for 3 mins and remove supernatant
7. Then add 50 μl of resazurin (Sigma #R7017) (FAC 100 μM resazurin in RPMI medium without FBS) and incubate plates for a further 1-2 hrs at 37° C. and 5% CO₂
8. Plates were read in an Envision reader at Ex 560 nm and Em 590 nm
9. IC₅₀ data is fitted to a non-linear regression equation (log inhibitor vs response-variable slope 4-parameters)

96-Well Plate Map

| | 1 | 2 | 3 | 4 | 5 | 6 | 7 | 8 | 9 | 10 | 11 | 12 |
|---|---|---|---|---|---|---|---|---|---|---|---|---|
| A | High | Comp 1 | Comp 2 | Comp 3 | Comp 4 | Comp 5 | Comp 6 | Comp 7 | Comp 8 | Comp 9 | Comp 10 | Low |
| B | High | Comp 1 | Comp 2 | Comp 3 | Comp 4 | Comp 5 | Comp 6 | Comp 7 | Comp 8 | Comp 9 | Comp 10 | Low |
| C | High | Comp 1 | Comp 2 | Comp 3 | Comp 4 | Comp 5 | Comp 6 | Comp 7 | Comp 8 | Comp 9 | Comp 10 | Low |
| D | High | Comp 1 | Comp 2 | Comp 3 | Comp 4 | Comp 5 | Comp 6 | Comp 7 | Comp 8 | Comp 9 | Comp 10 | Low |
| E | High | Comp 1 | Comp 2 | Comp 3 | Comp 4 | Comp 5 | Comp 6 | Comp 7 | Comp 8 | Comp 9 | Comp 10 | Low |
| F | High | Comp 1 | Comp 2 | Comp 3 | Comp 4 | Comp 5 | Comp 6 | Comp 7 | Comp 8 | Comp 9 | Comp 10 | Low |
| G | High | Comp 1 | Comp 2 | Comp 3 | Comp 4 | Comp 5 | Comp 6 | Comp 7 | Comp 8 | Comp 9 | Comp 10 | Low |
| H | High | Comp 1 | Comp 2 | Comp 3 | Comp 4 | Comp 5 | Comp 6 | Comp 7 | Comp 8 | Comp 9 | Comp 10 | Low |

High MCC950 (10 uM)
Low Drug free control
Compound 8-point half-log dilution

The results of the pyroptosis assay performed are summarised in Table 2 below as THP IC₅₀.

Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.

Human whole blood in Li-heparin tubes was obtained from healthy donors from a volunteer donor panel.
1. Nate out 80 μl of whole blood containing 1 μg/ml of LPS in 96-well, clear bottom cell culture plate (Corning #3585)
2. Add 10 μl compound (8 points half-log dilution with 10 μM top dose) or vehicle (DMSO 0.1% FAC) to the appropriate wells
3. Incubate for 3 hrs at 37° C., 5% CO₂
4. Add 10 μl nigericin (Sigma #N7143) (10 μM FAC) to all wells
5. Incubate for 1 hr at 37° C., 5% CO₂
6. At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
7. IL-1β was measured according to the manufacturer protocol (Perkin Elmer-AlphaLisa IL-1 Kit AL220F-5000)
8. IC₅₀ data is fitted to a non-linear regression equation (log inhibitor vs response-variable slope 4-parameters)

The results of the human whole blood assay are summarised in Table 2 below as HWB IC₅₀.

TABLE 2
NLRP3 inhibitory activity (≤0.25 μM = ‘++++++’, ≤0.5
μM = ‘+++++’, ≤1 μM = ‘++++’, ≤5 μM = ‘+++’, ≤10
μM = ‘++’, >10 μM = ‘+’, not determined = ‘ND’).

| Example No | THP IC₅₀ | HWB IC₅₀ |
|---|---|---|
| 1 | ++ | ND |
| 2 | +++++ | + |
| 3 | +++ | + |
| 4 | ++ | ND |
| 5 | ++ | ND |
| 6 | +++ | ++ |
| 7 | ++ | ND |
| 8 | ++++ | +++ |
| 9 | ++ | ND |
| 10 | ++ | ND |
| 11 | ++ | ND |
| 12 | ++++++ | ++++ |
| 13 | +++ | + |
| 14 | ++++ | ++ |
| 15 | +++ | + |
| 16 | ++++++ | ++ |
| 17 | +++++ | +++ |
| 18 | +++ | ND |
| 19 | +++++ | ++++ |
| 20 | ++++++ | +++ |
| 21 | ++ | ++ |
| 22 | ++++++ | +++ |
| 23 | +++ | ND |
| 24 | +++ | + |
| 25 | ++ | ND |
| 26 | +++ | ND |
| 27 | +++ | ND |
| 28 | +++ | ++ |
| 29 | ++ | ND |
| 30 | +++ | ND |
| 31 | +++ | ++ |
| 32 | +++ | ND |
| 33 | +++ | +++ |
| 34 | +++++ | ++ |
| 35 | ++++++ | ++ |
| 36 | ++++++ | ++ |
| 37 | +++++ | ND |
| 38 | ++++++ | ND |
| 39 | ++++++ | ++++ |
| 40 | +++++ | +++ |
| 41 | +++++ | ++ |
| 42 | ++++++ | +++ |
| 43 | +++++ | ND |
| 44 | ++++++ | ND |
| 45 | ++++++ | +++ |
| 46 | +++++ | ++++ |
| 47 | ++++++ | +++ |
| 48 | ++++++ | +++ |
| 49 | ++++++ | ++++ |
| 50 | +++++ | ND |
| 51 | ++++++ | +++++ |
| 52 | ++++++ | ++++ |
| 53 | ++++++ | ++++ |
| 54 | ++++++ | ++++ |
| 55 | ++++++ | ++++ |
| 56 | ++++++ | +++++ |
| 57 | ++++++ | +++ |
| 58 | ++++++ | +++ |
| 59 | ++++++ | +++ |
| 60 | ++++++ | ++++++ |
| 61 | ++++++ | ++++ |
| 62 | ++++++ | + |
| 63 | ++++++ | +++ |
| 64 | +++ | ND |
| 65 | ++++++ | +++++ |
| 66 | ++++++ | +++++ |
| 67 | ++++++ | +++ |
| 68 | +++ | ND |
| 69 | ++++ | +++ |
| 70 | ++ | ND |
| 71 | ++++ | +++ |
| 72 | +++ | ++ |
| 73 | ++ | ND |
| 74 | ++++ | ND |
| 75 | +++ | ND |
| 76 | ++ | ND |
| 77 | +++ | ++ |
| 78 | +++ | ND |
| 79 | ++++ | ND |
| 80 | ++++ | ND |
| 81 | +++++ | +++++ |
| 82 | +++++ | ++++ |
| 83 | ++++++ | +++ |
| 84 | +++++ | ++++ |
| 85 | ++ | ND |
| 86 | +++ | ND |
| 87 | +++ | ND |
| 88 | ++ | ND |
| 89 | +++ | ND |
| 90 | +++ | ND |
| 91 | +++ | ND |
| 92 | ++ | ND |
| 93 | ++ | ND |
| 94 | +++ | ND |
| 95 | ++ | ND |
| 96 | +++ | ND |
| 97 | +++ | ND |
| 98 | +++ | ND |

PK Protocol

Pharmacokinetic parameters were determined in male Sprague Dawley rats (Charles River, UK, 250-300 g; or Vital River Laboratory Animal Technology Co Ltd, Beijing, China, 7-9 weeks old). Animals were individually housed during the study and maintained under a 12 h light/dark cycle. Animals had free access to food and water, except that orally dosed animals were food deprived overnight prior to the study.

For intravenous administration, compounds were formulated as a solution in DMSO:PBS [10:90] in 2 mL/kg dosing volume and administered via tail vein. For oral administration, compounds were formulated as a solution in DMSO:water [10:90] in 5 mL/kg dosing volume and administered orally.

Serial blood samples (about 200-230 μL) were taken from each animal at each of 8 time-points post dose (0.083, 0.25, 0.5, 1, 2, 4, 8 and 24 h). Samples were held on ice for no longer than 30 minutes before centrifugation (10,000 rpm (8,385 g) for 3 minutes; or 5,696 rpm (3,000 g) for 15 minutes) for plasma generation. Plasma was frozen on dry ice prior to bioanalysis. PK parameters were generated from LC-MS/MS data using Dotmatics or Phoenix WinNonlin 6.3 software.

TABLE 3
PK data (intravenous administration)

| Example | Dose | AUC | T_{1/2} | V_{dss} | Cl |
|---|---|---|---|---|---|
| No | (mg/kg) | (ng · hr/mL) | (hr) | (L/kg) | (mL/min/kg) |
| 51 | 1 | 4871.9 | 1.0 | 0.29 | 3.4 |
| 20 | 1 | 21569.6 | 4.7 | 0.25 | 0.8 |
| 46 | 1 | 10016.4 | 4.0 | 0.45 | 1.7 |
| 83 | 1 | 1127.4 | 3.9 | 2.23 | 14.8 |
| 39 | 3 | 3840.2 | 4.0 | 0.9 | 13.1 |

TABLE 4
PK data (oral administration) (ND = not determined)

| Ex- | | | AUC | | | Cl/F | Bioavail- |
|---|---|---|---|---|---|---|---|
| ample | Dose | Cₘₐₓ | (ng · | Tₘₐₓ | T_{1/2} | (mL/ | ability |
| No | (mg/kg) | (ng/mL) | hr/mL) | (hr) | (hr) | min/kg) | (%) |
| 45 | 3 | 7901.1 | 61633.7 | 2 | 2.5 | 0.8 | ND |
| 83 | 1.3 | 467.9 | 2751.2 | 3.17 | 3.7 | 8.5 | >100 |
| 39 | 3 | 1368.3 | 3921.2 | 0.83 | 2.2 | 13.0 | >100 |

As is evident from the results presented in Table 2, surprisingly in spite of the structural differences versus the prior art compounds, the compounds of the invention show high levels of NLRP3 inhibitory activity in the pyroptosis assay and in the human whole blood assay.

As is evident from the results presented in Tables 3 and 4, the compounds of the invention show advantageous pharmacokinetic properties, for example half-life T_{1/2}, area under the curve AUC, clearance Cl and/or bioavailability, compared to the prior art compounds.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (I): 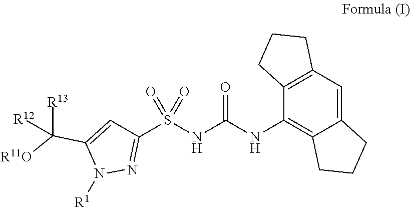
or a pharmaceutically acceptable salt, solvate or prodrug thereof, wherein:
R¹ is a C₁-C₃ alkyl group;
R¹¹ is a C₁-C₃ alkyl group;
R¹² is hydrogen or a C₁-C₃ alkyl group; and
R¹³ is hydrogen or a C₁-C₃ alkyl group; or
R¹² and R¹³ together with the carbon atom to which they are attached form a C₃-C₆ cycloalkyl group; or
a compound of formula (II): 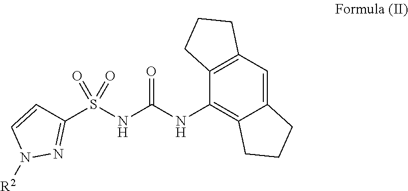
or a pharmaceutically acceptable salt, solvate or prodrug thereof, wherein R² is a C₁-C₃ haloalkyl group; or
a compound of formula (III): 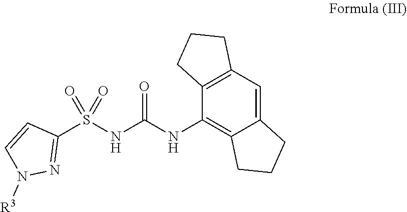
or a pharmaceutically acceptable salt, solvate or prodrug thereof, wherein:
R³ is a C₁-C₆ alkyl, C₃-C₆ cycloalkyl, (C₃-C₆ cycloalkylene)(C₁-C₂ alkyl) or (C₁-C₂ alkylene)(C₃-C₆ cycloalkyl) group, each of which is substituted with —NR^{a}R^{b};
R^{a} is a C₁-C₃ alkyl group; and
R^{b} is a C₁-C₃ alkyl group; or
R^{a} and R^{b} together with the nitrogen atom to which they are attached form a 4- to 6-membered saturated heterocycle;
provided that R³ comprises 8 or more atoms other than hydrogen or halogen; or
a compound of formula (IV): 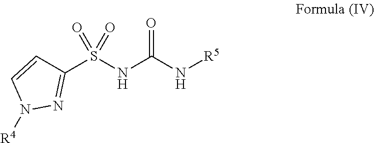
or a pharmaceutically acceptable salt, solvate or prodrug thereof, wherein:
R⁴ is a C₁-C₃ haloalkyl group;
R⁵ is selected from: 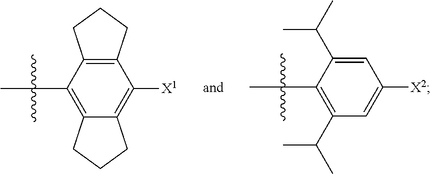
X¹ is H, F, Cl, Br or CN; and
X² is F, Cl, Br or CN; or
a compound of formula (V): 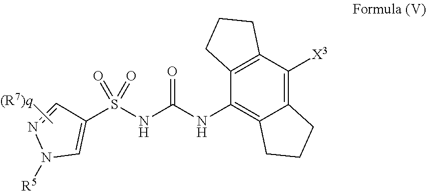
or a pharmaceutically acceptable salt, solvate or prodrug thereof, wherein:
R⁶ is a C₂-C₄ alkyl group;
R⁷ is a C₁-C₃ alkyl group;
q is 0 or 1; and
X³ is H, F, Cl, Br or CN; or
a compound of formula (VI): 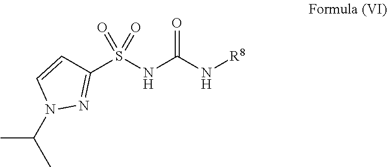
or a pharmaceutically acceptable salt, solvate or prodrug thereof, wherein:
R⁸ is selected from: 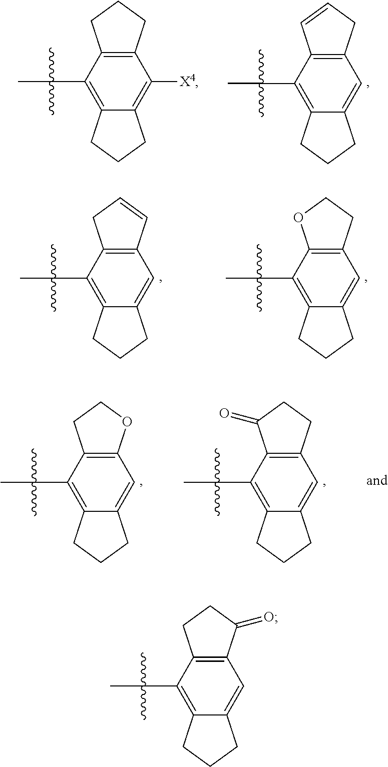
and
X⁴ is F, Cl, Br or CN; or
a compound of formula (VII): 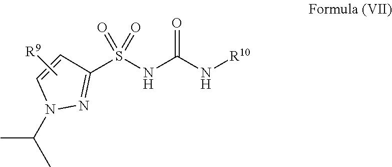
or a pharmaceutically acceptable salt, solvate or prodrug thereof, wherein:
R⁹ is halo or a group —CR⁹¹R⁹²(OH);
R⁹¹ is hydrogen or a C₁-C₃ alkyl group;
R⁹² is hydrogen or a C₁-C₃ alkyl group;
R¹⁰ is selected from: 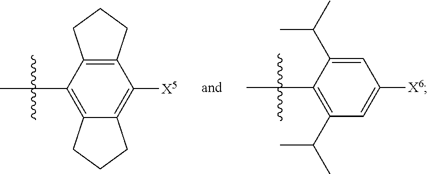
X⁵ is H, F, Cl, Br or CN; and
X⁶ is F, Cl, Br or CN; or
a compound of formula (VIII): 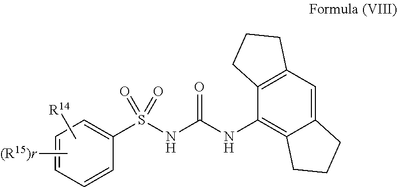
or a pharmaceutically acceptable salt, solvate or prodrug thereof, wherein:
R¹⁴ is a C₁-C₃ alkyl group substituted with —NR^{c}R^{d};
R^{c} is a C₁-C₃ alkyl group;
R^{d} is a C₁-C₃ alkyl group; or
R^{c} and R^{d} together with the nitrogen atom to which they are attached form a 4- to 6-membered saturated heterocycle;
R¹⁵ is a C₁-C₃ alkyl group; and
r is 0, or 2; or
a compound of formula (IX): 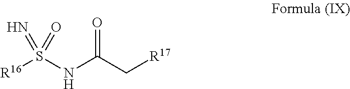
or a pharmaceutically acceptable salt, solvate or prodrug thereof, wherein:
R¹⁶ is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O or S in its carbon skeleton; and
R¹⁷ is a cyclic group substituted at the α-position, wherein R¹⁷ may optionally be further substituted; or
a compound or a pharmaceutically acceptable salt, solvate or prodrug thereof, selected from the group consisting of: 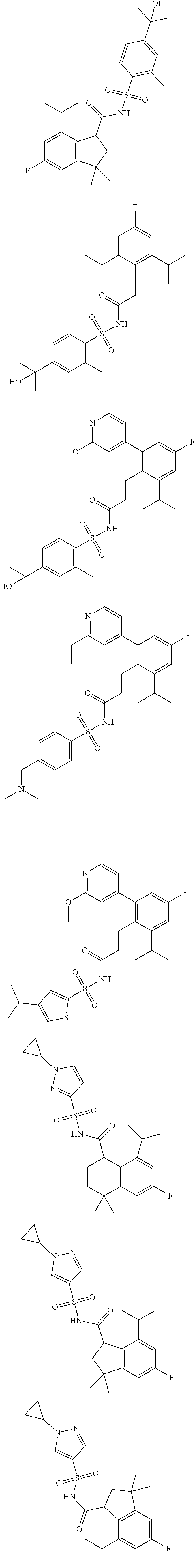 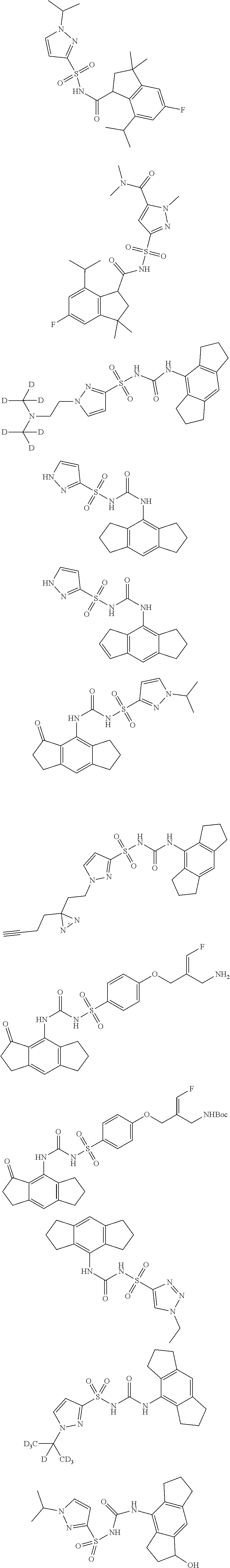 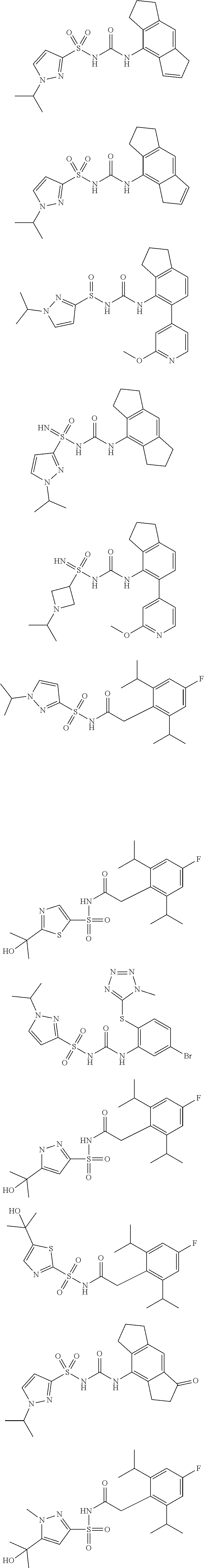 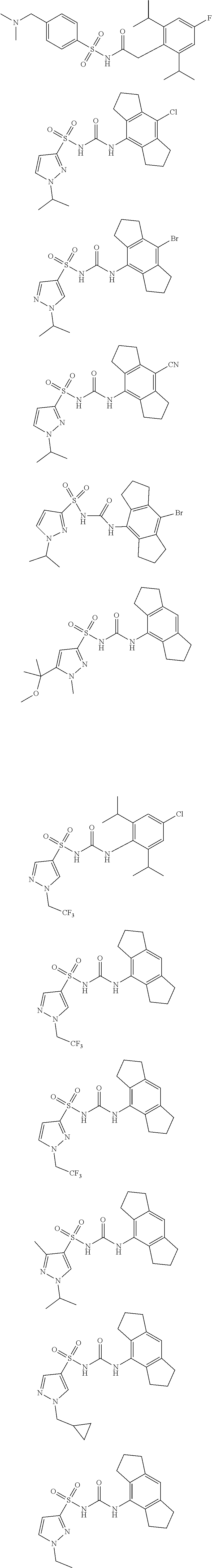 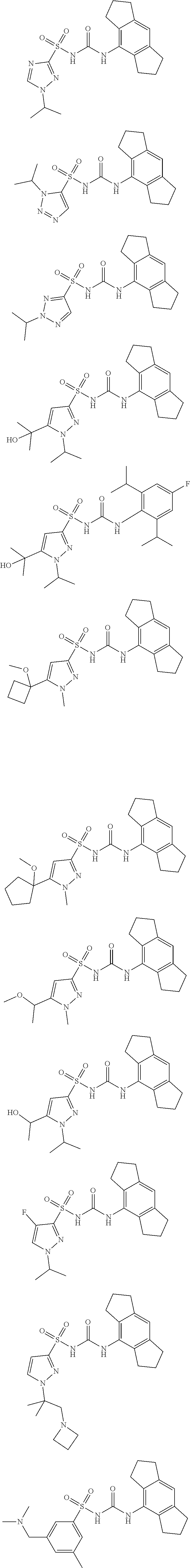 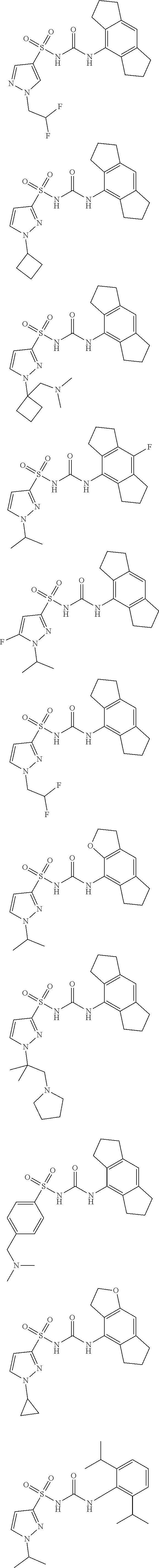 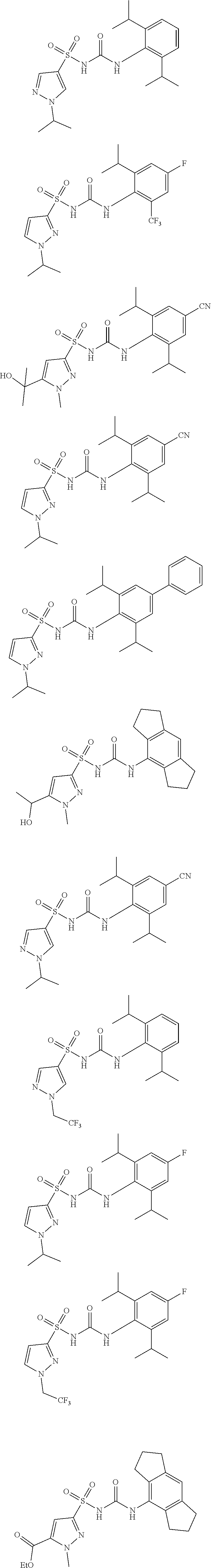 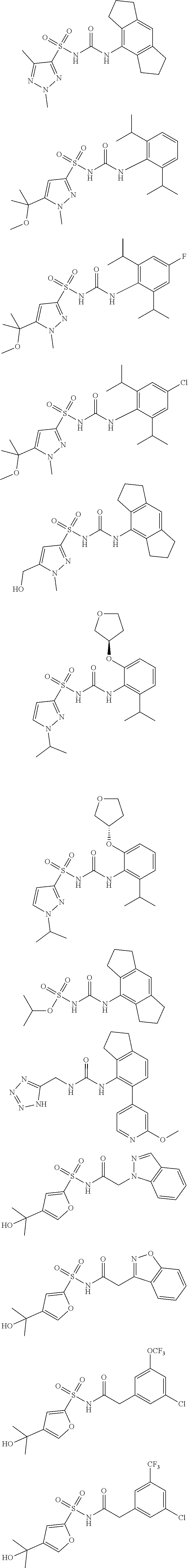 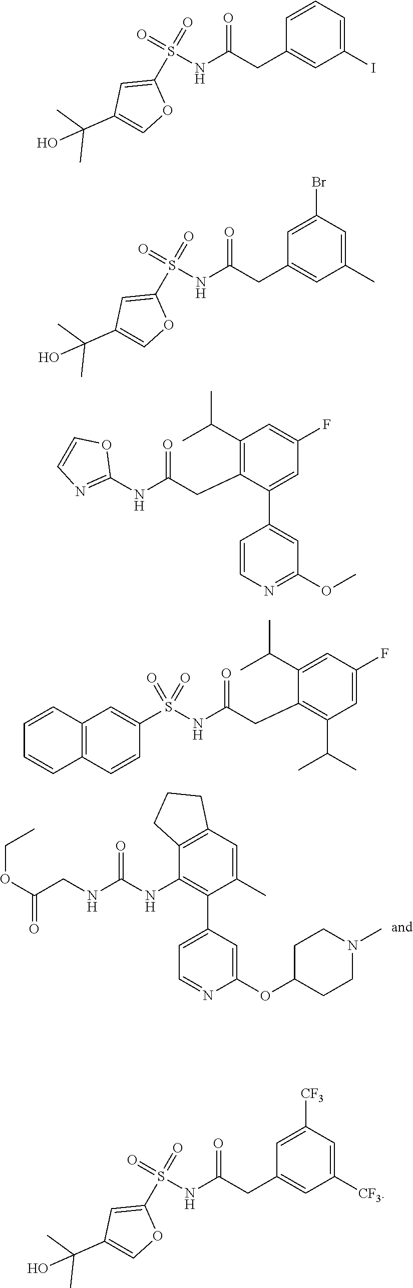
or a pharmaceutically acceptable salt, solvate or prodrug thereof.

2.-11. (canceled)

12. A pharmaceutical composition comprising a compound or a pharmaceutically acceptable salt, solvate or prodrug as claimed in claim 1, and a pharmaceutically acceptable excipient.

13. (canceled)

14. A method of treating or preventing a disease, disorder or condition in a subject, the method comprising the step of administering an effective amount of the compound, pharmaceutically acceptable salt, solvate or prodrug as claimed in claim 1 to the subject, thereby treating or preventing the disease, disorder or condition, optionally wherein the disease, disorder or condition is responsive to NLRP3 inhibition.

15. The method as claimed in claim 14, wherein the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3.

16. The method as claimed in claim 14 wherein the disease, disorder or condition is selected from:
(i) cryopyrin-associated periodic syndromes (CAPS);
(ii) Muckle-Wells syndrome (MWS);
(iii) familial cold autoinflammatory syndrome (FCAS);
(iv) neonatal onset multisystem inflammatory disease (NOMID);
(v) familial Mediterranean fever (FMF);
(vi) pyogenic arthritis, pyoderma gangrenosum and acne syndrome (PAPA);
(vii) hyperimmunoglobulinemia D and periodic fever syndrome (HIDS);
(viii) Tumour Necrosis Factor (TNF) Receptor-Associated Periodic Syndrome (TRAPS);
(ix) systemic juvenile idiopathic arthritis;
(x) adult-onset Still's disease (AOSD);
(xi) relapsing polychondritis;
(xii) Schnitzler's syndrome;
(xiii) Sweet's syndrome;
(xiv) Behcet's disease;
(xv) anti-synthetase syndrome;
(xvi) deficiency of interleukin 1 receptor antagonist (DIRA); and
(xvii) haploinsufficiency of A20 (HA20).

17. (canceled)

18. The method as claimed in claim 14, wherein the compound, pharmaceutically acceptable salt, solvate or prodrug is administered as a pharmaceutical composition further comprising a pharmaceutically acceptable excipient.

19. A method of inhibiting NLRP3 in a subject, comprising administering the compound or a pharmaceutically acceptable salt, solvate or prodrug thereof as claimed in claim 1 to the subject thereby inhibiting NLRP3.

20. A method of analysing inhibition of NLRP3 or an effect of inhibition of NLRP3 by a compound, comprising contacting a cell or non-human animal with the compound or a pharmaceutically acceptable salt, solvate or prodrug thereof as claimed in claim 1, and analysing inhibition of NLRP3 or an effect of inhibition of NLRP3 in the cell or non-human animal by the compound.
